(19) United States
(12) Patent Application Publication
(10) Pub. No.: US 2017/0368157 A1
(43) Pub. Date: Dec. 28, 2017

(54) Combination Of Listeria-Based Vaccine With Anti-OX40 Or Anti-GITR Antibodies

(71) Applicants: Advaxis, Inc., Princeton, NJ (US); Augusta University Research Institute, Inc., Augusta, GA (US)

(72) Inventors: Samir Khleif, Silver Springs, MD (US); Mikayel Mkrtichyan, Irvine, CA (US); Robert Petit, Newton, PA (US); Anu Wallecha, Yardley, PA (US)

(21) Appl. No.: 15/533,645

(22) PCT Filed: Dec. 18, 2015
(86) PCT No.: PCT/US2015/066896

§ 371 Date: Jun. 6, 2017

Related U.S. Application Data
(60) Provisional application No. 62/094,472, filed on Dec. 19, 2014.
(60) Provisional application No. 62/094,349, filed on Dec. 19, 2014.
(60) Provisional application No. 62/147,463, filed on Apr. 14, 2015.
(60) Provisional application No. 62/262,896, filed on Dec. 3, 2015.

(51) Int. Cl. A61K 39/00; C07K 16/28; C07K 14/025

(52) U.S. Cl. CPC A61K 39/0011; C07K 14/025; C07K 16/2818; A61K 2039/505; A61K 2039/572; A61K 2039/522; C07K 2319/40

(57) ABSTRACT

Disclosed herein are compositions comprising use of compositions comprising a live attenuated recombinant Listeria strain comprising a fusion protein of a truncated listeriolysin O (LLO) protein, a truncated ActA protein, or a PEST amino acid sequence fused to a heterologous antigen, including a tumor-associated antigen, wherein the compositions further comprise or are co-administered with an antibody or fragment thereof. Also disclosed are combination therapies comprising use of these compositions comprising live attenuated recombinant Listeria strains, in conjunction with an antibody or fragment thereof for use in treating, protecting against, and/or inducing an immune response against a tumor, especially wherein the treating, protection against and/or inducing an immune response increases percent survival in a subject.

### FIELD OF INTEREST

Disclosed herein are compositions comprising use of compositions comprising a live attenuated recombinant Listeria strain comprising a fusion protein of a truncated listeriolysin O (LLO) protein, a truncated ActA protein, or a PEST amino acid sequence fused to a heterologous antigen, including a tumor-associated antigen, wherein the compositions further comprise or are co-administered with an antibody or fragment thereof. Also disclosed are combination therapies comprising use of these compositions comprising live attenuated recombinant Listeria strains, in conjunction with an antibody or fragment thereof for use in treating, protecting against, and/or inducing an immune response against a tumor, especially wherein the treating, protection against and/or inducing an immune response increases percent survival in a subject.

### BACKGROUND

Listeria monocytogenes (Lm) is a Gram-positive facultative intracellular pathogen that causes listeriolysis. Once invading a host cell, Lm can escape from the phagolysosome through production of a pore-forming protein listeriolysin O (LLO) to lyse the vascular membrane, allowing it to enter the cytoplasm, where it replicates and spreads to adjacent cells based on the mobility of actin-polymerizing protein (ActA). In the cytoplasm, Lm-secreting proteins are degraded by the proteasome and processed into peptides that associate with MHC class I molecules in the endoplasmic reticulum. This unique characteristic makes it a very attractive cancer vaccine vector in that tumor antigen can be presented with MHC class I molecules to activate tumor-specific cytotoxic T lymphocytes (CTLs).

In addition, once phagocytized, Lm may then be processed in the phagolysosomal compartment and peptides presented on MHC Class II for activation of Lm-specific CD4-T cell responses. Alternatively, Lm can escape the phagosome and enter the cytosol where recognition of peptidoglycan by nuclear oligomerization domain-like receptors and Lm DNA by DNA sensor, AIM2, activate inflammatory cascades. This combination of inflammatory responses and efficient delivery of antigens to the MHC I and MHC II pathways makes Lm a powerful vaccine vector in treating, protecting against, and inducing an immune response against a tumor.

However, tumor cells often induce an immunosuppressive microenvironment, which favors the development of immunosuppressive populations of immune cells, such as myeloid-derived suppressor cells (MDSC) and regulatory T cells (Treg). Understanding the complexity of immunomodulation by tumors is important for the development of immunotherapy. Various strategies are being developed to enhance anti-tumor immune responses and to overcome ‘immune checkpoints’. In addition, administration of combination immunotherapies may provide a more efficacious and enduring response.

For example, one of several mechanisms of tumor-mediated immune suppression is the expression of T-cell co-inhibitory molecules by tumor. Upon engagement to their ligands these molecules can suppress effector lymphocytes in the periphery and in the tumor microenvironment.

Presently, there remains a need to provide effective combination therapies for tumor targeting methods that can eliminate tumor growth and cancer. The present invention addresses this need by providing a combination of a Listeria based vaccine with various therapies including addition of antibodies or fragments thereof, which may enhance or facilitate proliferation of memory and effector T cells, and activate costimulatory receptors on T cells or antigen presenting cells. It is thought that costimulation may be crucial to the development of an effective anti-tumor immune response against a particular tumor or cancer in addition the antigen presentation that results from administration of a listeria-based vaccine.

Targeted immunomodulatory therapy is focused primarily on the activation of costimulatory receptors, for example by using agonist antibodies that target members of the tumor necrosis factor receptor superfamily, including 4-1BB, OX40 and GITR (glucocorticoid-induced TNF receptor-related). The modulation of GITR has demonstrated potential in both antitumor and vaccine settings. Another target for agonist antibodies are co-stimulatory signal molecules for T cell activation. Targeting costimulatory signal molecules may lead to enhanced activation of T cells and facilitation of a more potent immune response. Co-stimulation may also help prevent inhibitory influences from check-point inhibition and increase antigen-specific T cell proliferation. Unfortunately, use of such agonist antibodies may lead to toxicity issues. Therefore, it is essential in the development of anti-tumor immunotherapy to establish a safe and efficacious dose of any agonist antibody combination with the listeria based immunotherapeutic composition being considered.

Thus, there remains a need to optimize the dosage and schedule for administrating a combination Listeria based immunotherapeutic composition with any immunotherapy agonist antibody. The present invention also addresses this need by providing a combination of a Listeria based vaccine with agonist antibodies in response to tumor development.

Given the complex nature of certain diseases, including cancer, a need exists for a combined approach in treating the same. As seen in the Detailed Description below, these combination therapies may improve the overall anti-tumor efficacy of immunotherapy.

### SUMMARY

In one aspect, the disclosure relates to an immunogenic composition comprising a recombinant Listeria strain comprising a nucleic acid molecule, said nucleic acid molecule comprising a first open reading frame encoding a fusion polypeptide, wherein said fusion polypeptide comprises a truncated Listeriolysin O (LLO) protein, a truncated ActA protein or a PEST amino acid sequence fused to a heterologous antigen or fragment thereof, said composition further comprising an antibody or fragment thereof. In another aspect, the antibody or fragment thereof is an agonist antibody or fragment thereof. In another aspect, the antibody or fragment thereof binds to an antigen or portion thereof comprising a T-cell receptor co-stimulatory molecule, an antigen presenting cell receptor binding co-stimulatory molecule or a member of the TNF receptor superfamily.

In another aspect, the disclosure relates to an immunogenic composition comprising a recombinant Listeria strain comprising a nucleic acid molecule, said nucleic acid molecule comprising a first open reading frame encoding a fusion polypeptide comprising a truncated Listeriolysin O protein, a truncated ActA protein or a PEST amino acid sequence fused to a heterologous antigen or a fragment thereof, said composition further comprising an antibody or fragment thereof. In another aspect, the antibody or fragment thereof is an agonist antibody or fragment thereof. In another aspect, the antibody or fragment thereof binds to an antigen or portion thereof comprising a T-cell receptor co-stimulatory molecule, an antigen presenting cell receptor binding co-stimulatory molecule or a member of the TNF receptor superfamily. In another related aspect, a nucleic acid molecule comprised in a Listeria strain encodes a truncated LLO protein. In another related aspect, a nucleic acid molecule comprised in a Listeria strain encodes a truncated LLO protein, a truncated ActA protein, or a PEST amino acid sequence.

In a related aspect, the present invention relates to a method of eliciting an enhanced anti-tumor T cell response in a subject, said method comprising the step of administering to said subject an effective amount of an immunogenic composition comprising a recombinant Listeria strain, said Listeria strain comprising a nucleic acid molecule, said nucleic acid molecule comprising a first open reading frame encoding a fusion polypeptide, wherein said fusion polypeptide comprises a truncated Listeriolysin O protein, a truncated ActA protein or a PEST amino acid sequence fused to a heterologous antigen or fragment thereof, wherein the method further comprises a step of administering an effective amount of a composition comprising an anti-TNF receptor antibody or fragment thereof to said subject.

In another related aspect, the disclosure relates to methods for eliciting an enhanced anti-tumor T cell response in a subject comprising the use of a recombinant Listeria strain comprising a nucleic acid molecule, said nucleic acid molecule comprising a first open reading frame encoding a truncated LLO protein, a truncated ActA protein, or a PEST amino acid sequence, wherein the method further comprises a step of administering an effective amount of a composition comprising an anti-TNF receptor antibody or fragment thereof to said subject.

In another related aspect, the disclosure relates to a method of increasing antigen-specific T cells in a subject, said method comprising the step of administering to said subject an effective amount of an immunogenic composition comprising a recombinant Listeria strain comprising a nucleic acid molecule, said nucleic acid molecule comprising a first open reading frame encoding a fusion polypeptide, wherein said fusion polypeptide comprises a truncated Listeriolysin O protein, a truncated ActA protein or a PEST amino acid sequence fused to a heterologous antigen or fragment thereof, wherein the method further comprises a step of administering an effective amount of a composition comprising an anti-TNF receptor antibody or fragment thereof to said subject.

In another related aspect, the disclosure relates to a method for increasing a T cell response in a subject comprise use of a recombinant Listeria strain comprising a nucleic acid molecule, said nucleic acid molecule comprising a first open reading frame encoding a truncated LLO protein, a truncated ActA protein, or a PEST amino acid sequence, wherein the method further comprises a step of administering an effective amount of a composition comprising an anti-TNF receptor antibody or fragment thereof to said subject.

In another related aspect, the disclosure relates to a method of treating a tumor or cancer in a subject, said method comprising the step of administering to said subject an effective amount of an immunogenic composition comprising a recombinant Listeria strain comprising a nucleic acid molecule, said nucleic acid molecule comprising a first open reading frame encoding a fusion polypeptide, wherein said fusion polypeptide comprises a truncated Listeriolysin O protein, a truncated ActA protein or a PEST amino acid sequence fused to a heterologous antigen or fragment thereof, wherein the method further comprises a step of administering an effective amount of a composition comprising an anti-TNF receptor antibody or fragment thereof to said subject.

In another related aspect, methods of this invention for treating a tumor or a cancer in a subject comprise use of a recombinant Listeria strain comprising a nucleic acid molecule, said nucleic acid molecule comprising a first open reading frame encoding a truncated listeriolysin O (LLO) protein, a truncated ActA protein, or a PEST amino acid sequence, wherein the method further comprises a step of administering an effective amount of a composition comprising an anti-TNF receptor antibody or fragment thereof to said subject.

In another related aspect, the present invention relates to a method of increasing survival in a subject, said method comprising the step of administering to said subject an effective amount of an immunogenic composition comprising a recombinant Listeria strain comprising a nucleic acid molecule, said nucleic acid molecule comprising a first open reading frame encoding a fusion polypeptide, wherein said fusion polypeptide comprises a truncated Listeriolysin O protein, a truncated ActA protein or a PEST amino acid sequence fused to a heterologous antigen or fragment thereof, wherein the method further comprises a step of administering an effective amount of a composition comprising an anti-TNF receptor antibody or fragment thereof to said subject. In another related aspect, methods of this invention for increasing survival in a subject comprise use of a recombinant Listeria strain comprising a nucleic acid molecule, said nucleic acid molecule comprising a first open reading frame encoding a truncated LLO protein, a truncated ActA protein, or a PEST amino acid sequence, wherein the method further comprises a step of administering an effective amount of a composition comprising an anti-TNF receptor antibody or fragment thereof to said subject.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A and 1B. Lm-E7 and Lm-LLO-E7 (ADXS11-001) use different expression systems to express and secrete E7. Lm-E7 was generated by introducing a gene cassette into the orfZ domain of the L. monocytogenes genome (FIG. 1A). The hly promoter drives expression of the hly signal sequence and the first five amino acids (AA) of LLO followed by HPV-16 E7. (FIG. 1B), Lm-LLO-E7 was generated by transforming the prfA-strain XFL-7 with the plasmid pGG-55. pGG-55 has the hly promoter driving expression of a nonhemolytic fusion of LLO-E7. pGG-55 also contains the prfA gene to select for retention of the plasmid by XFL-7 in vivo.

FIG. 2. Lm-E7 and Lm-LLO-E7 secrete E7. Lm-Gag (lane 1), Lm-E7 (lane 2), Lm-LLO-NP (lane 3), Lm-LLO-E7 (lane 4), XFL-7 (lane 5), and 10403S (lane 6) were grown overnight at 37° C. in Luria-Bertoni broth. Equivalent numbers of bacteria, as determined by OD at 600 nm absorbance, were pelleted and 18 ml of each supernatant was TCA precipitated. E7 expression was analyzed by Western blot. The blot was probed with an anti-E7 mAb, followed by HRP-conjugated anti-mouse (Amersham), then developed using ECL detection reagents.

FIG. 3. Tumor immunotherapeutic efficacy of LLO-E7 fusions. Tumor size in millimeters in mice is shown at 7, 14, 21, 28 and 56 days post tumor-inoculation. Naive mice: open-circles; Lm-LLO-E7: filled circles; Lm-E7: squares; Lm-Gag: open diamonds; and Lm-LLO-NP: filled triangles.

FIG. 4. Splenocytes from Lm-LLO-E7-immunized mice proliferate when exposed to TC-1 cells. C57BL/6 mice were immunized and boosted with Lm-LLO-E7, Lm-E7, or control rLm strains. Splenocytes were harvested 6 days after the boost and plated with irradiated TC-1 cells at the ratios shown. The cells were pulsed with ³H thymidine and harvested. Cpm is defined as (experimental cpm)−(no-TC-1 control).

FIGS. 5A and 5B. (FIG. 5A) Western blot demonstrating that Lm-ActA-E7 secretes E7. Lane 1: Lm-LLO-E7; lane 2: Lm-ActA-E7.001; lane 3; Lm-ActA-E7-2.5.3; lane 4: Lm-ActA-E7-2.5.4. (FIG. 5B) Tumor size in mice administered Lm-ActA-E7 (rectangles), Lm-E7 (ovals), Lm-LLO-E7 (X), and naive mice (non-vaccinated; solid triangles).

FIGS. 6A-6C. (FIG. 6A) schematic representation of the plasmid inserts used to create 4 LM vaccines. Lm-LLO-E7 insert contains all of the Listeria genes used. It contains the hly promoter, the first 1.3 kb of the hly gene (which encodes the protein LLO), and the HPV-16 E7 gene. The first 1.3 kb of hly includes the signal sequence (ss) and the PEST region. Lm-PEST-E7 includes the hly promoter, the signal sequence, and PEST and E7 sequences but excludes the remainder of the truncated LLO gene. Lm-ΔPEST-E7 excludes the PEST region, but contains the hly promoter, the signal sequence, E7, and the remainder of the truncated LLO. Lm-E7epi has only the hly promoter, the signal sequence, and E7. (FIG. 6B) Top panel: Listeria constructs containing PEST regions induce tumor regression. Bottom panel: Average tumor sizes at day 28 post-tumor challenge in 2 separate experiments. (FIG. 6C) Listeria constructs containing PEST regions induce a higher percentage of E7-specific lymphocytes in the spleen. Average and SE of data from 3 experiments are depicted.

FIGS. 7A and 7B. (FIG. 7A) Induction of E7-specific IFN-gamma-secreting CD8⁺ T cells in the spleens and the numbers penetrating the tumors, in mice administered TC-1 tumor cells and subsequently administered Lm-E7, Lm-LLO-E7, Lm-ActA-E7, or no vaccine (naive). (FIG. 7B) Induction and penetration of E7 specific CD8⁺ cells in the spleens and tumors of the mice described for (FIG. 7A).

FIGS. 8A and 8B. Listeria constructs containing PEST regions induce a higher percentage of E7-specific lymphocytes within the tumor. (FIG. 8A) representative data from 1 experiment. (FIG. 8B) average and SE of data from all 3 experiments.

FIG. 9. Data from Cohorts 1 and 2 indicting the efficacy observed in the patients in the clinical trial presented in Example 6.

FIGS. 10A and 10B. (FIG. 10A) Schematic representation of the chromosomal region of the Lmdd-143 and LmddA-143 after klk3 integration and actA deletion; (FIG. 10B) The klk3 gene is integrated into the Lmdd and LmddA chromosome. PCR from chromosomal DNA preparation from each construct using klk3 specific primers amplifies a band of 714 bp corresponding to the klk3 gene, lacking the secretion signal sequence of the wild type protein.

FIGS. 11A-11D. (FIG. 11A) Map of the pADV134 plasmid. (FIG. 11B) Proteins from LmddA-134 culture supernatant were precipitated, separated in a SDS-PAGE, and the LLO-E7 protein detected by Western-blot using an anti-E7 monoclonal antibody. The antigen expression cassette consists of hly promoter, ORF for truncated LLO and human PSA gene (klk3). (FIG. 11C) Map of the pADV142 plasmid. (FIG. 11D) Western blot showed the expression of LLO-PSA fusion protein using anti-PSA and anti-LLO antibody.

FIGS. 12A and 12B. (FIG. 12A) Plasmid stability in vitro of LmddA-LLO-PSA if cultured with and without selection pressure (D-alanine). Strain and culture conditions are listed first and plates used for CFU determination are listed after. (FIG. 12B) Clearance of LmddA-LLO-PSA in vivo and assessment of potential plasmid loss during this time. Bacteria were injected i.v. and isolated from spleen at the time point indicated. CFUs were determined on BHI and BHI+D-alanine plates.

FIGS. 13A and 13B. (FIG. 13A) In vivo clearance of the strain LmddA-LLO-PSA after administration of 10⁸ CFU in C57BL/6 mice. The number of CFU were determined by plating on BHI/str plates. The limit of detection of this method was 100 CFU. (FIG. 13B) Cell infection assay of J774 cells with 10403S, LmddA-LLO-PSA and XFL7 strains.

FIGS. 14A-14E. (FIG. 14A) PSA tetramer-specific cells in the splenocytes of naïve and LmddA-LLO-PSA immunized mice on day 6 after the booster dose. (FIG. 14B) Intracellular cytokine staining for IFN-γ in the splenocytes of naïve and LmddA-LLO-PSA immunized mice were stimulated with PSA peptide for 5 h. Specific lysis of EL4 cells pulsed with PSA peptide with in vitro stimulated effector T cells from LmddA-LLO-PSA immunized mice and naïve mice at different effector/target ratio using a caspase based assay (FIG. 14C) and a europium based assay (FIG. 14D). Number of IFNγ spots in naïve and immunized splenocytes obtained after stimulation for 24 h in the presence of PSA peptide or no peptide (FIG. 14E).

FIGS. 15A-15C. Immunization with LmddA-142 induces regression of Tramp-Cl-PSA (TPSA) tumors. Mice were left untreated (n=8) (FIG. 15A) or immunized i.p. with LmddA-142 (1×10⁸ CFU/mouse) (n=8) (FIG. 15B) or Lm-LLO-PSA (n=8), (FIG. 15C) on days 7, 14 and 21. Tumor sizes were measured for each individual tumor and the values expressed as the mean diameter in millimeters. Each line represents an individual mouse.

FIGS. 16A and 16B. (FIG. 16A) Analysis of PSA-tetramer⁺CD8⁺ T cells in the spleens and infiltrating T-PSA-23 tumors of untreated mice and mice immunized with either an Lm control strain or LmddA-LLO-PSA (LmddA-142). (FIG. 16B) Analysis of CD4⁺ regulatory T cells, which were defined as CD25⁺FoxP3⁺, in the spleens and infiltrating T-PSA-23 tumors of untreated mice and mice immunized with either an Lm control strain or LmddA-LLO-PSA.

FIGS. 17A and 17B. (FIG. 17A) Schematic representation of the chromosomal region of the Lmdd-143 and LmddA-143 after klk3 integration and actA deletion; (FIG. 17B) The klk3 gene is integrated into the Lmdd and LmddA chromosome. PCR from chromosomal DNA preparation from each construct using klk3 specific primers amplifies a band of 760 bp corresponding to the klk3 gene.

FIGS. 18A-C. (FIG. 18A) Lmdd-143 and LmddA-143 secretes the LLO-PSA protein. Proteins from bacterial culture supernatants were precipitated, separated in a SDS-PAGE and LLO and LLO-PSA proteins detected by Western-blot using an anti-LLO and anti-PSA antibodies; (FIG. 18B) LLO produced by Lmdd-143 and LmddA-143 retains hemolytic activity. Sheep red blood cells were incubated with serial dilutions of bacterial culture supernatants and hemolytic activity measured by absorbance at 590 nm; (FIG. 18C) Lmdd-143 and LmddA-143 grow inside the macrophage-like J774 cells. J774 cells were incubated with bacteria for 1 hour followed by gentamicin treatment to kill extracellular bacteria. Intracellular growth was measured by plating serial dilutions of J774 lysates obtained at the indicated timepoints. Lm 10403S was used as a control in these experiments.

FIG. 19. Immunization of mice with Lmdd-143 and LmddA-143 induces a PSA-specific immune response. C57BL/6 mice were immunized twice at 1-week interval with 1×10⁸ CFU of Lmdd-143, LmddA-143 or LmddA-142 and 7 days later spleens were harvested. Splenocytes were stimulated for 5 hours in the presence of monensin with 1 μM of the PSA₆₅₋₇₄ peptide. Cells were stained for CD8, CD3, CD62L and intracellular IFN-γ and analyzed in a FACS Calibur cytometer.

FIGS. 20A and 20B. Figures show a decrease in MDSCs and Tregs in tumors. The number of MDSCs (FIG. 20B) and Tregs (FIG. 20A) following Lm vaccination (LmddA-PSA and LmddA-E7).

FIGS. 21A-21D. Figures show suppressor assay data demonstrating that monocytic MDSCs from TPSA23 tumors (PSA expressing tumor) are less suppressive after Listeria vaccination. This change in the suppressive ability of the MDSCs is not antigen specific as the same decrease in suppression is seen with PSA-antigen specific T cells and also with non-specifically stimulated T cells. In FIGS. 21A and 21B Phorbol-Myristate-Acetate and Ionomycin (PMA/I) represents non-specific stimulation. In FIGS. 21C and 21D the term “peptide” represents specific antigen stimulation. Percent (%) CD3+CD8+ represents % effector (responder) T cells. The No MDSC group shows the lack of division of the responder T cells when they are left unstimulated and the last group (PMA/I or peptide added) shows the division of stimulated cells in the absence of MDSCs. FIGS. 21A and 21C show individual cell division cycles for each group. FIGS. 21B and 21D show pooled division cycles.

FIGS. 22A-22D show suppressor assay data demonstrating that Listeria has no effect on splenic monocytic MDSCs and they are only suppressive in an antigen-specific manner. In FIGS. 22A and 22B PMA/I represents non-specific stimulation. In FIGS. 22C and 22D the term “peptide” represents specific antigen stimulation. Percent (%) CD3+CD8+ represents % effector (responder) T cells. The No MDSC group shows the lack of division of the responder T cells when they are left unstimulated and the last group (PMA/I or peptide added) shows the division of stimulated cells in the absence of MDSCs. FIGS. 22A and 22C show individual cell division cycles for each group. FIGS. 22B and 22D show pooled division cycles.

FIGS. 23A-23D show suppressor assay data demonstrating that granulocytic MDSCs from tumors have a reduced ability to suppress T cells after Listeria vaccination. This change in the suppressive ability of the MDSCs is not antigen specific as the same decrease in suppression is seen with PSA-antigen specific T cells and also with non-specifically stimulated T cells. In FIGS. 23A and 23B PMA/I represents non-specific stimulation. In FIGS. 23C and 23D the term “peptide” represents specific antigen stimulation. Percent (%) CD3+CD8+ represents % effector (responder) T cells. The No MDSC group shows the lack of division of the responder T cells when they are left unstimulated and the last group (PMA/I or peptide added) shows the division of stimulated cells in the absence of MDSCs. FIGS. 23A and 23C show individual cell division cycles for each group. FIGS. 23B and 23D show pooled percentage division.

FIGS. 24A-24D show suppressor assay data demonstrating that Listeria has no effect on splenic granulocytic MDSCs and they are only suppressive in an antigen-specific manner. In FIGS. 24A and 24B PMA/I represents non-specific stimulation. In FIGS. 24C and 24D the term “peptide” represents specific antigen stimulation. Percent (%) CD3+CD8+ represents % effector (responder) T cells. The No MDSC group shows the lack of division of the responder T cells when they are left unstimulated and the last group (PMA/I or peptide added) shows the division of stimulated cells in the absence of MDSCs. FIGS. 24A and 24C show individual cell division cycles for each group. FIGS. 24B and 24D show pooled percentage division.

FIGS. 25A-25D show suppressor assay data demonstrating that Tregs from tumors are still suppressive. There is a slight decrease in the suppressive ability of Tregs in a non-antigen specific manner, in this tumor model. In FIGS. 25A and 25B PMA/I represents non-specific stimulation. In FIGS. 25C and 25D the term “peptide” represents specific antigen stimulation. Percent (%) CD3+CD8+ represents % effector (responder) T cells. The No Treg group shows the lack of division of the responder T cells when they are left unstimulated and the last group (PMA/I or peptide added) shows the division of stimulated cells in the absence of Tregs. FIGS. 25A and 25C show individual cell division cycles for each group. FIGS. 25B and 25D show pooled percentage division.

FIGS. 26A-26D shows suppressor assay data demonstrating that splenic Tregs are still suppressive. In FIGS. 26A and 26B PMA/I represents non-specific stimulation. In FIGS. 26C and 26D the term “peptide” represents specific antigen stimulation. Percent (%) CD3+CD8+ represents % effector (responder) T cells. The No Treg group shows the lack of division of the responder T cells when they are left unstimulated and the last group (PMA/I or peptide added) shows the division of stimulated cells in the absence of Tregs. FIGS. 26A and 26C show individual cell division cycles for each group. FIGS. 26B and 26D show pooled percentage division.

FIGS. 27A-27D show suppressor assay data demonstrating that conventional CD4+ T cells have no effect on cell division regardless whether they are found in the tumors or spleens of mice. In FIGS. 27A and 27B PMA/I represents non-specific stimulation. In FIGS. 27C and 27D the term “peptide” represents specific antigen stimulation. Percent (%) CD3+CD8+ represents % effector (responder) T cells. The No Treg group shows the lack of division of the responder T cells when they are left unstimulated and the last group (PMA/I or peptide added) shows the division of stimulated cells in the absence of Tregs. FIGS. 27C-27D show data from pooled percentage division.

FIGS. 28A-28D show suppressor assay data demonstrating that monocytic MDSCs from 4T1 tumors (Her2 expressing tumors) have decreased suppressive ability after Listeria vaccination. This change in the suppressive ability of the MDSCs is not antigen specific as the same decrease in suppression is seen with Her2/neu-antigen specific T cells and also with non-specifically stimulated T cells. In FIGS. 28A and 28B PMA/I represents non-specific stimulation. In FIGS. 28C and 28D the term “peptide” represents specific antigen stimulation. Percent (%) CD8+ represents % effector (responder) T cells. The No MDSC group shows the lack of division of the responder T cells when they are left unstimulated and the last group (PMA/I or peptide added) shows the division of stimulated cells in the absence of MDSCs. FIGS. 28A and 28C show individual cell division cycles for each group. FIGS. 28B and 28D show pooled percentage division.

FIGS. 29A-29D show suppressor assay data demonstrating that there is no Listeria-specific effect on splenic monocytic MDSCs. In FIGS. 29A and 29B PMA/I represents non-specific stimulation. In FIGS. 29C and 29D the term “peptide” represents specific antigen stimulation. Percent (%) CD8+ represents % effector (responder) T cells. The No MDSC group shows the lack of division of the responder T cells when they are left unstimulated and the last group (PMA/I or peptide added) shows the division of stimulated cells in the absence of MDSC. FIGS. 29A and 29C show individual cell division cycles for each group. FIGS. 29B and 29D show pooled percentage division.

FIGS. 30A-30D show suppressor assay data demonstrating that granulocytic MDSCs from 4T1 tumors (Her2 expressing tumors) have decreased suppressive ability after Listeria vaccination. This change in the suppressive ability of the MDSCs is not antigen specific as the same decrease in suppression is seen with Her2/neu-antigen specific T cells and also with non-specifically stimulated T cells. In FIGS. 30A and 30B PMA/I represents non-specific stimulation. In FIGS. 30C and 30D the term “peptide” represents specific antigen stimulation. Percent (%) CD8+ represents % effector (responder) T cells. The No MDSC group shows the lack of division of the responder T cells when they are left unstimulated and the last group (PMA/I or peptide added) shows the division of stimulated cells in the absence of MDSCs. FIGS. 30A and 30C show individual cell division cycles for each group. FIGS. 30B and 30D shows pooled percentage division.

FIGS. 31A-31D present suppressor assay data demonstrating that there is no Listeria-specific effect on splenic granulocytic MDSCs. In FIGS. 31A and 31B PMA/I represents non-specific stimulation. In FIGS. 31C and 31D the term “peptide” represents specific antigen stimulation. Percent (%) CD8+ represents % effector (responder) T cells. The No MDSC group shows the lack of division of the responder T cells when they are left unstimulated and the last group (PMA/I or peptide added) shows the division of stimulated cells in the absence of MDSCs. FIGS. 31A and 31C show individual cell division cycles for each group. FIGS. 31B and 31D show pooled percentage division.

FIGS. 32A-32D present suppressor assay data demonstrating that decrease in the suppressive ability of Tregs from 4T1 tumors (Her2 expressing tumors) after Listeria vaccination. In FIGS. 32A and 32B PMA/I represents non-specific stimulation. In FIGS. 32C and 32D the term “peptide” represents specific antigen stimulation. Percent (%) CD8+ represents % effector (responder) T cells. This decrease is not antigen specific, as the change in Treg suppressive ability is seen with both Her2/neu-specific and non-specific responder T cells. FIGS. 32A and 32C show individual cell division cycles for each group. FIGS. 32B and 32D show pooled percentage division.

FIGS. 33A-33D show suppressor assay data demonstrating that there is no Listeria-specific effect on splenic Tregs. The responder T cells are all capable of dividing regardless of whether or not they are antigen specific. In FIGS. 33A and 33B PMA/I represents non-specific stimulation. In FIGS. 33C and 33D the term “peptide” represents specific antigen stimulation. Percent (%) CD8+ represents % effector (responder) T cells. FIGS. 33A and 33C show individual cell division cycles for each group. FIGS. 33B and 33D show pooled percentage division.

FIGS. 34A-34D show suppressor assay data demonstrating that suppressive ability of the granulocytic MDSC is due to the overexpression of tLLO and is independent of the partnering fusion antigen. Left-hand panels (FIGS. 34A and 34C) show individual cell division cycles for each group. Right-hand panels (FIGS. 34B and 34D) show pooled percentage division.

FIGS. 35A-35D show suppressor assay data also demonstrating that suppressive ability of the monocytic MDSC is due to the overexpression of tLLO and is independent of the partnering fusion antigen. Left-hand panels (FIGS. 35A and 35C) show individual cell division cycles for each group. Right-hand panels (FIGS. 35B and 35D) show pooled percentage division.

FIGS. 36A-36D show suppressor assay data demonstrating that granulocytic MDSC purified from the spleen retain their ability to suppress the division of the antigen-specific responder T cells after Lm vaccination (FIGS. 36A and 36B). However, after non-specific stimulation, activated T cells (with PMA/ionomycin) are still capable of dividing (FIGS. 36C and 36D). Left-hand panels show individual cell division cycles for each group. Right-hand panels show pooled percentage division.

FIGS. 37A-37D show suppressor assay data demonstrating that monocytic MDSC purified from the spleen retain their ability to suppress the division of the antigen-specific responder T cells after Lm vaccination (FIGS. 37A and 37B). However, after non-specific activation (stimulated by PMA/ionomycin), T cells are still capable of dividing (FIGS. 37C and 37D). Left-hand panels show individual cell division cycles for each group. Right-hand panels show pooled percentage division.

FIGS. 38A-38D show suppressor assay data demonstrating that Tregs purified from the tumors of any of the Lm-treated groups have a slightly diminished ability to suppress the division of the responder T cells, regardless of whether the responder cells are antigen specific (FIGS. 38A and 38B) or non-specifically (FIGS. 38C and 38D) activated. Left-hand panels show individual cell division cycles for each group. Right-hand panels show pooled percentage division.

FIGS. 39A-39D show suppressor assay data demonstrating that Tregs purified from the spleen are still capable of suppressing the division of both antigen specific (FIGS. 39A-39B) and non-specifically (FIGS. 39C and 39D) activated responder T cells.

FIGS. 40A-40D show suppressor assay data demonstrating that tumor Tcon cells are not capable of suppressing the division of T cells regardless of whether the responder cells are antigens specific (FIGS. 40A and 40B) or non-specifically activated (FIGS. 40C and 40D).

FIGS. 41A-41D show suppressor assay data demonstrating that spleen Tcon cells are not capable of suppressing the division of T cells regardless of whether the responder cells are antigens specific (FIGS. 41A and 41B) or non-specifically activated (FIGS. 41C and 41D).

FIGS. 42A-42C. (FIG. 42A) Schematic of the treatment schedule for mice undergoing combination Listeria-based vaccine (ADXS11-001, which is Lm-LLO-E7) with anti-OX40 antibodies, wherein tumor growth and mouse survival were monitored throughout the experiment. (FIG. 42B) Schematic of the treatment schedule for mice undergoing combination Listeria-based vaccine (ADXS11-001, which is Lm-LLO-E7) with anti-GITR antibodies, wherein tumor growth and mouse survival were monitored throughout the experiment. For both (FIG. 42A) and (FIG. 42B) at day 0 mice were injected with 7×10⁵ TC-1 tumor cells to initiate tumor formation. Vaccinations began at day 10. Controls included LmddA-LLO and Listeria strain XFL7. (FIG. 42A) shows that anti-OX40 antibodies were administered twice a week throughout the time period of the experiment. (FIG. 42B) shows that anti-GITR antibodies were administered twice a week for a total of three doses. (FIG. 42C) identifies the twelve administrative regimens, including no treatment (NT).

FIG. 43A-B. TC-1 tumors were implanted subcutaneously (s.c.) on the ventral side of C57BL/6 mice. When tumor volume reached about 0.06 cm³, two doses of Lm based E7 specific tumor vaccines (1×10⁸ colony-forming units/mouse) were given intraperitoneally (i.p.) at an interval of 7 days. GITR (5 mg/Kg b.wt.; total four doses) and OX40 (1 mg/Kg b.wt.; through out the experiment) antibodies were injected i.p. twice a week starting with the vaccine. Tumor size was measured twice weekly. Tumor growth (FIG. 43A) and percent survival (FIG. 43B) are shown. N=5/group. Results are shown as mean±SE from one representative experiment. Experiment was repeated two times. *p≧0.05, **p≧0.01, ****p≧0.0001.

FIG. 44 A-B. TC-1 tumors were implanted subcutaneously (s.c.) on the ventral side of C57BL/6 mice. When tumor volume reached about 0.06 cm³, two doses of Lm based E7 specific tumor vaccines (1×10⁸ colony-forming units/mouse) were given intraperitoneally (i.p.) at an interval of 7 days. OX40 (1 mg/Kg b.wt.; through out the experiment) antibodies were injected i.p. twice a week starting with the vaccine. Tumor size was measured twice weekly. Tumor growth (FIG. 44A) and percent survival (FIG. 44B) are shown. N=5/group. Results are shown as mean±SE from one representative experiment. Experiment was repeated two times. *p≧0.05, **p≧0.01, ****p≧0.0001.

FIG. 45. Schematic of vaccine administration investigation for combination anti-GITR Ab with Listeria-based vaccine therapy.

FIGS. 46A and 46B. FIG. 46A presents a bar graph showing the number of tumor-infiltrating CD4+ T cells dependent on the different therapy groups. FIG. 46B presents a bar graph showing the number of tumor-infiltrating Treg (CD4+FoxP3+) cells dependent on the different therapy groups.

FIGS. 47A and 47B. FIG. 47A presents a bar graph showing the number of tumor-infiltrating total non Treg (CD4+FoxP3−) cells dependent on the different therapy groups. FIG. 47B presents a bar graph showing the percent of tumor-infiltrating Treg FoxP3+ of CD4+ cells dependent on the different therapy groups.

FIGS. 48A and 48B. FIG. 48A presents a bar graph showing the number of tumor-infiltrating CD8+ T cells dependent on the different therapy groups. FIG. 48B presents a bar graph showing the number of tumor-infiltrating E7-specific CD8+ T cells (antigen specific) dependent on the different therapy groups.

FIGS. 49A and 49B. FIG. 49A presents a bar graph showing the ratio of CD8+/Treg cells, dependent on the different therapy groups. FIG. 49B presents a bar graph showing the ratio of E7+CD8+/Treg cells, dependent on the different therapy groups.

FIGS. 50A, 50B and 50CB. FIG. 50A presents a bar graph showing the number of tumor-infiltrating myeloid-derived suppressor cells (MDSCs) dependent on the different therapy groups. FIG. 50B presents a bar graph showing the ratio of tumor-infiltrating CD8/MDSCs, dependent on the different therapy groups. FIG. 50C presents a bar graph showing the ratio of antigen specific tumor-infiltrating E7-CD8/MDSCs, dependent on the different therapy groups.

FIG. 51. Schematic of vaccine administration investigation for combination anti-OX40 Ab with Listeria-based vaccine therapy.

FIGS. 52A and 52B. FIG. 52A presents a bar graph showing the number of tumor-infiltrating CD4+ T cells dependent on the different therapy groups. FIG. 52B presents a bar graph showing the number of tumor-infiltrating Treg (CD4+FoxP3+) cells dependent on the different therapy groups.

FIGS. 53A and 53B. FIG. 53A presents a bar graph showing the number of tumor-infiltrating total non Treg (CD4+FoxP3−) cells dependent on the different therapy groups. FIG. 53B presents a bar graph showing the percent of tumor-infiltrating Treg FoxP3+ of CD4+ cells dependent on the different therapy groups.

FIGS. 54A and 54B. FIG. 54A presents a bar graph showing the number of tumor-infiltrating CD8+ T cells dependent on the different therapy groups. FIG. 54B presents a bar graph showing the number of tumor-infiltrating E7-specific CD8+ T cells (antigen specific) dependent on the different therapy groups.

FIGS. 55A and 55B. FIG. 55A presents a bar graph showing the ratio of CD8+/Treg cells, dependent on the different therapy groups. FIG. 55B presents a bar graph showing the ratio of E7+CD8+/Treg cells, dependent on the different therapy groups.

FIGS. 56A, 56B and 56C. FIG. 56A presents a bar graph showing the number of tumor-infiltrating myeloid-derived suppressor cells (MDSCs) dependent on the different therapy groups. FIG. 56B presents a bar graph showing the ratio of tumor-infiltrating CD8/MDSCs, dependent on the different therapy groups. FIG. 56C presents a bar graph showing the ratio of antigen specific tumor-infiltrating E7-CD8/MDSCs, dependent on the different therapy groups.

FIGS. 57A and 57B. Construction of ADXS31-164. (FIG. 57A) Plasmid map of pAdv164, which harbors bacillus subtilis dal gene under the control of constitutive Listeria p60 promoter for complementation of the chromosomal dal-dat deletion in LmddA strain. It also contains the fusion of truncated LLO₍₁₋₄₄₁₎ to the chimeric human Her2/neu gene, which was constructed by the direct fusion of 3 fragments the Her2/neu: EC1 (aa 40-170), EC2 (aa 359-518) and ICI (aa 679-808). (FIG. 57B) Expression and secretion of tLLO-ChHer2 was detected in Lm-LLO-ChHer2 (Lm-LLO-138) and LmddA-LLO-ChHer2 (ADXS31-164) by western blot analysis of the TCA precipitated cell culture supernatants blotted with anti-LLO antibody. A differential band of ˜104 KD corresponds to tLLO-ChHer2. The endogenous LLO is detected as a 58 KD band. Listeria control lacked ChHer2 expression.

FIGS. 58A-58C. Immunogenic properties of ADXS31-164 (FIG. 58A) Cytotoxic T cell responses elicited by Her2/neu Listeria-based vaccines in splenocytes from immunized mice were tested using NT-2 cells as stimulators and 3T3/neu cells as targets. Lm-control was based on the LmddA background that was identical in all ways but expressed an irrelevant antigen (HPV16-E7). (FIG. 58B) IFN-γ secreted by the splenocytes from immunized FVB/N mice into the cell culture medium, measured by ELISA, after 24 hours of in vitro stimulation with mitomycin C treated NT-2 cells. (FIG. 58C) IFN-γ secretion by splenocytes from HLA-A2 transgenic mice immunized with the chimeric vaccine, in response to in vitro incubation with peptides from different regions of the protein. A recombinant ChHer2 protein was used as positive control and an irrelevant peptide or no peptide groups constituted the negative controls as listed in the figure legend. IFN-γ secretion was detected by an ELISA assay using cell culture supernatants harvested after 72 hours of co-incubation. Each data point was an average of triplicate data +/−standard error. * P value <0.001.

FIG. 59. Tumor Prevention Studies for Listeria-ChHer2/neu Vaccines Her2/neu transgenic mice were injected six times with each recombinant Listeria-ChHer2 or a control Listeria vaccine. Immunizations started at 6 weeks of age and continued every three weeks until week 21. Appearance of tumors was monitored on a weekly basis and expressed as percentage of tumor free mice. *p<0.05, N=9 per group.

FIG. 60. Effect of immunization with ADXS31-164 on the % of Tregs in Spleens. FVB/N mice were inoculated s.c. with 1×10⁶ NT-2 cells and immunized three times with each vaccine at one week intervals. Spleens were harvested 7 days after the second immunization. After isolation of the immune cells, they were stained for detection of Tregs by anti CD3, CD4, CD25 and FoxP3 antibodies. Dot-plots of the Tregs from a representative experiment showing the frequency of CD25⁺/FoxP3⁺ T cells, expressed as percentages of the total CD3⁺ or CD3⁺CD4⁺ T cells across the different treatment groups.

FIGS. 61A and 61B. Effect of immunization with ADXS31-164 on the % of tumor infiltrating Tregs in NT-2 tumors. FVB/N mice were inoculated s.c. with 1×10⁶ NT-2 cells and immunized three times with each vaccine at one week intervals. Tumors were harvested 7 days after the second immunization. After isolation of the immune cells, they were stained for detection of Tregs by anti CD3, CD4, CD25 and FoxP3 antibodies. (FIG. 61A). dot-plots of the Tregs from a representative experiment. (FIG. 61B). Frequency of CD25⁺/FoxP3⁺ T cells, expressed as percentages of the total CD3⁺ or CD3⁺CD4⁺ T cells (left panel) and intratumoral CD8/Tregs ratio (right panel) across the different treatment groups. Data is shown as mean±SEM obtained from 2 independent experiments.

FIGS. 62A-62C. Vaccination with ADXS31-164 can delay the growth of a breast cancer cell line in the brain. Balb/c mice were immunized thrice with ADXS31-164 or a control Listeria vaccine. EMT6-Luc cells (5,000) were injected intracranially in anesthetized mice. (FIG. 62A) Ex vivo imaging of the mice was performed on the indicated days using a Xenogen X-100 CCD camera. (FIG. 62B) Pixel intensity was graphed as number of photons per second per cm2 of surface area; this is shown as average radiance. (FIG. 62C) Expression of Her2/neu by EMT6-Luc cells, 4T1-Luc and NT-2 cell lines was detected by Western blots, using an anti-Her2/neu antibody. J774.A2 cells, a murine macrophage like cell line was used as a negative control.

FIG. 63. Shows the treatment schedule for pre-established FVB/N Her2/neu, NT-2 tumor mouse model.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

In the following detailed description, numerous specific details are set forth in order to provide a thorough understanding of the invention. However, it will be understood by those skilled in the art that the present disclosure may be practiced without these specific details. In other instances, well-known methods, procedures, and components have not been described in detail so as not to obscure the disclosure.

Disclosed, in one embodiment, is an immunogenic composition comprising a recombinant Listeria strain comprising a nucleic acid molecule, the nucleic acid molecule comprising a first open reading frame encoding a fusion polypeptide, wherein said fusion polypeptide comprises a truncated listeriolysin O (LLO) protein, a truncated ActA protein, or a PEST amino acid sequence fused to a heterologous antigen or fragment thereof and wherein the composition further comprises an antibody or fragment thereof.

In one embodiment, an antibody or fragment thereof disclosed herein is an agonist antibody. In another embodiment, the antibody or fragment thereof is an anti-TNF receptor antibody. In another embodiment, the antibody or fragment thereof is an agonist anti-TNF receptor antibody.

In another embodiment, disclosed herein is an immunogenic composition comprising a recombinant Listeria strain comprising a nucleic acid molecule, the nucleic acid molecule comprising a first open reading frame encoding a truncated listeriolysin O (LLO) protein, a truncated ActA protein, or a PEST amino acid sequence, wherein the composition further comprises an agonist anti-TNF receptor antibody or fragment thereof. In a further embodiment, a nucleic acid molecule comprised in a Listeria strain does not encode a fusion polypeptide.

In another embodiment, disclosed herein is an immunogenic composition comprising an agonist antibody or fragment thereof, and a recombinant Listeria strain comprising a nucleic acid molecule, the nucleic acid molecule comprising a first open reading frame encoding fusion polypeptide, wherein the fusion polypeptide comprises a truncated listeriolysin O (LLO) protein, a truncated ActA protein, or a PEST amino acid sequence fused to a heterologous antigen or fragment thereof.

In another embodiment, disclosed herein is an immunogenic composition comprising an agonist anti-TNF receptor antibody or fragment thereof and a recombinant Listeria strain comprising a nucleic acid molecule, the nucleic acid molecule comprising a first open reading frame encoding a truncated listeriolysin O (LLO) protein, a truncated ActA protein, or a PEST amino acid sequence. In a further embodiment, the nucleic acid molecule comprised in the Listeria strain does not encode a fusion polypeptide.

In one embodiment, the agonist antibody or fragment thereof binds to a heterologous antigen or portion thereof comprising a T-cell receptor co-stimulatory molecule. Hence, in another embodiment, disclosed herein is an immunogenic composition comprising an agonist antibody or fragment thereof that binds a T-cell receptor co-stimulatory molecule, and a recombinant Listeria strain comprising a nucleic acid molecule, the nucleic acid molecule comprising a first open reading frame encoding a fusion polypeptide, wherein the fusion polypeptide comprises a truncated listeriolysin O (LLO) protein, a truncated ActA protein, or a PEST amino acid sequence.

In yet another embodiment, disclosed herein is an immunogenic composition comprising an agonist antibody or fragment thereof that binds a T-cell receptor co-stimulatory molecule, and a recombinant Listeria strain comprising a nucleic acid molecule, the nucleic acid molecule comprising a first open reading frame encoding a truncated listeriolysin O (LLO) protein, a truncated ActA protein, or a PEST amino acid sequence. In a further embodiment, the nucleic acid molecule comprised in the Listeria strain does not encode a fusion polypeptide.

In another embodiment, the disclosed agonist antibody or fragment thereof binds to an antigen or portion thereof comprising an antigen presenting cell receptor binding a co-stimulatory molecule. Hence, in another embodiment, disclosed herein is an immunogenic composition comprising an agonist antibody or fragment thereof that binds an antigen presenting cell receptor binding a co-stimulatory molecule, and a recombinant Listeria strain comprising a nucleic acid molecule, the nucleic acid molecule comprising a first open reading frame encoding a fusion polypeptide, wherein the fusion polypeptide comprises a truncated listeriolysin O (LLO) protein, a truncated ActA protein, or a PEST amino acid sequence fused to a heterologous antigen or fragment thereof. In another embodiment, the immunogenic composition comprises an agonist antibody or fragment thereof that binds an antigen presenting cell receptor binding a co-stimulatory molecule, and a recombinant Listeria strain comprising a nucleic acid molecule, the nucleic acid molecule comprising a first open reading frame encoding a truncated listeriolysin O (LLO) protein, a truncated ActA protein, or a PEST amino acid sequence. In a further embodiment, the nucleic acid molecule comprised in the Listeria strain does not encode a fusion polypeptide.

In another embodiment, the agonist antibody or fragment thereof binds to an antigen or portion thereof comprising a member of the Tumor Necrosis Factor (TNF) receptor superfamily. Hence, in another embodiment, disclosed herein is an immunogenic composition comprising an agonist antibody or fragment thereof that binds a TNF receptor superfamily, and a recombinant Listeria strain comprising a nucleic acid molecule, the nucleic acid molecule comprising a first open reading frame encoding a fusion polypeptide, wherein the fusion polypeptide comprises a truncated listeriolysin O (LLO) protein, a truncated ActA protein, or a PEST amino acid sequence fused to a heterologous antigen or fragment thereof. In another embodiment, an immunogenic composition comprises an agonist antibody or fragment thereof that binds a TNF receptor superfamily, and a recombinant Listeria strain comprising a nucleic acid molecule, the nucleic acid molecule comprising a first open reading frame encoding a truncated listeriolysin O (LLO) protein, a truncated ActA protein, or a PEST amino acid sequence. In a further embodiment, the nucleic acid molecule comprised in the Listeria strain does not encode a fusion polypeptide.

In one embodiment, disclosed is method of eliciting an enhanced anti-tumor T cell response in a subject, the method comprising the step of administering to the subject an effective amount of an immunogenic composition comprising a recombinant Listeria strain comprising a nucleic acid molecule, the nucleic acid molecule comprising a first open reading frame encoding a fusion polypeptide, wherein the fusion polypeptide comprises a truncated listeriolysin O (LLO) protein, a truncated ActA protein, or a PEST amino acid sequence fused to a heterologous antigen or fragment thereof, wherein the method further comprises a step of administering an effective amount of a composition comprising an antibody or fragment thereof to the subject. In another embodiment, a recombinant Listeria strain administered as part of a method for eliciting an enhanced anti-tumor T cell response comprises, a nucleic acid molecule comprising a first open reading frame encoding a truncated listeriolysin O (LLO) protein, a truncated ActA protein, or a PEST amino acid sequence. In a further embodiment, the first open reading frame does not encode a fusion polypeptide.

In another embodiment, disclosed is a method for inhibiting tumor-mediated immunosuppression in a subject, the method comprising the step of administering to the subject an effective amount of an immunogenic composition comprising a recombinant Listeria strain comprising a nucleic acid molecule, the nucleic acid molecule comprising a first open reading frame encoding a fusion polypeptide, wherein the fusion polypeptide comprises a truncated listeriolysin O (LLO) protein, a truncated ActA protein, or a PEST amino acid sequence fused to a heterologous antigen or fragment thereof, wherein the method further comprises a step of administering an effective amount of a composition comprising an antibody or fragment thereof to the subject. In another embodiment, a recombinant Listeria strain administered as part of a method for inhibiting tumor-mediated immunosuppression in a subject comprises, a nucleic acid molecule comprising a first open reading frame encoding a truncated listeriolysin O (LLO) protein, a truncated ActA protein, or a PEST amino acid sequence. In a further embodiment, the first open reading frame does not encode a fusion polypeptide.

In another embodiment, disclosed is a method increasing the ratio of T effector cells to regulatory T cells (Tregs) in the spleen and tumor of a subject, the method comprising the step of administering to the subject an immunogenic composition comprising a recombinant Listeria strain comprising a nucleic acid molecule, the nucleic acid molecule comprising a first open reading frame encoding a fusion polypeptide, wherein the fusion polypeptide comprises a truncated listeriolysin O (LLO) protein, a truncated ActA protein, or a PEST amino acid sequence fused to a heterologous antigen or fragment thereof, wherein the method further comprises a step of administering an effective amount of a composition comprising an antibody or fragment thereof to the subject. In another embodiment, a recombinant Listeria strain administered as part of a method of increasing the ratio of T effector cells to regulatory T cells (Tregs) in the spleen and tumor of the subject comprises a nucleic acid molecule comprising a first open reading frame encoding a truncated listeriolysin O (LLO) protein, a truncated ActA protein, or a PEST amino acid sequence. In a further embodiment, the first open reading frame does not encode a fusion polypeptide.

In another embodiment, disclosed is a method for increasing antigen-specific T-cells in a subject, the method comprising the step of administering to the subject an immunogenic composition comprising a recombinant Listeria strain comprising a nucleic acid molecule, the nucleic acid molecule comprising a first open reading frame encoding a fusion polypeptide, wherein the fusion polypeptide comprises a truncated listeriolysin O (LLO) protein, a truncated ActA protein, or a PEST amino acid sequence fused to a heterologous antigen or fragment thereof, wherein the method further comprises a step of administering an effective amount of a composition comprising an antibody or fragment thereof to the subject. In another embodiment, a recombinant Listeria strain administered as part of a method for increasing T cells in a subject comprises, a nucleic acid molecule comprising a first open reading frame encoding a truncated listeriolysin O (LLO) protein, a truncated ActA protein, or a PEST amino acid sequence. In a further embodiment, the first open reading frame does not encode a fusion polypeptide.

In another embodiment, disclosed is a method for increasing survival time of a subject having a tumor or suffering from cancer, the method comprising the step of administering to the subject an immunogenic composition comprising a recombinant Listeria strain comprising a nucleic acid molecule, the nucleic acid molecule comprising a first open reading frame encoding a fusion polypeptide, wherein the fusion polypeptide comprises a truncated listeriolysin O (LLO) protein, a truncated ActA protein, or a PEST amino acid sequence fused to a heterologous antigen or fragment thereof, wherein the method further comprises a step of administering an effective amount of a composition comprising an antibody or fragment thereof to the subject. In another embodiment, a recombinant Listeria strain administered as part of a method for increasing survival time of a subject having a tumor or suffering from a cancer comprises, a nucleic acid molecule comprising a first open reading frame encoding a truncated listeriolysin O (LLO) protein, a truncated ActA protein, or a PEST amino acid sequence. In a further embodiment, the first open reading frame does not encode a fusion polypeptide.

In another embodiment, disclosed is a method of treating a tumor or a cancer in a subject, the method comprising the step of administering to the subject an immunogenic composition comprising a recombinant Listeria strain comprising a nucleic acid molecule, the nucleic acid molecule comprising a first open reading frame encoding a fusion polypeptide, wherein the fusion polypeptide comprises a truncated listeriolysin O (LLO) protein, a truncated ActA protein, or a PEST amino acid sequence fused to a heterologous antigen or fragment thereof, wherein the method further comprises a step of administering an effective amount of a composition comprising an antibody or fragment thereof to the subject. In another embodiment, a recombinant Listeria strain administered as part of a method for treating a tumor or a cancer in a subject comprises, a nucleic acid molecule comprising a first open reading frame encoding a truncated listeriolysin O (LLO) protein, a truncated ActA protein, or a PEST amino acid sequence. In a further embodiment, the first open reading frame does not encode a fusion polypeptide.

### Recombinant Listeria Strains

In one embodiment, a recombinant Listeria strain of the present invention comprises a nucleic acid molecule, the nucleic acid molecule comprising a first open reading frame encoding a fusion polypeptide, wherein the fusion polypeptide comprises a truncated listeriolysin O (LLO) protein, a truncated ActA protein, or a PEST amino acid sequence fused to a heterologous antigen or fragment thereof. In another embodiment, a recombinant Listeria strain of the present invention comprises a nucleic acid molecule, the nucleic acid molecule comprising a first open reading frame encoding a truncated listeriolysin O (LLO) protein, a truncated ActA protein, or a PEST amino acid sequence. In one embodiment, the recombinant Listeria strain is attenuated.

In another embodiment, a truncated listeriolysin O (LLO) protein, a truncated ActA protein, or a PEST amino acid sequence is not fused to a heterologous antigen or a fragment thereof.

In one embodiment, a truncated listeriolysin O (LLO) protein comprises a PEST sequence. In another embodiment, a truncated listeriolysin O (LLO) protein comprises a putative PEST sequence. In one embodiment, a truncated actA protein comprises a PEST-containing amino acid sequence. In another embodiment, a truncated actA protein comprises a putative PEST-containing amino acid sequence.

In one embodiment, a PEST amino acid (AA) sequence comprises a truncated LLO sequence. In another embodiment, the PEST amino acid sequence is KENSISSMAPPASPPASPKTPIEKKHADEIDK (SEQ ID NO: 1). In another embodiment, fusion of an antigen to other LM PEST AA sequences from Listeria also enhances immunogenicity of the antigen.

The N-terminal LLO protein fragment of methods and compositions of the present invention comprises, in another embodiment, SEQ ID No: 3. In another embodiment, the fragment comprises an LLO signal peptide. In another embodiment, the fragment comprises SEQ ID No: 4. In another embodiment, the fragment consists approximately of SEQ ID No: 4. In another embodiment, the fragment consists essentially of SEQ ID No: 4. In another embodiment, the fragment corresponds to SEQ ID No: 4. In another embodiment, the fragment is homologous to SEQ ID No: 4. In another embodiment, the fragment is homologous to a fragment of SEQ ID No: 4. The ΔLLO used in some of the Examples was 416 AA long (exclusive of the signal sequence), as 88 residues from the amino terminus which is inclusive of the activation domain containing cysteine 484 were truncated. It will be clear to those skilled in the art that any ΔLLO without the activation domain, and in particular without cysteine 484, are suitable for methods and compositions of the present invention. In another embodiment, fusion of a heterologous antigen to any ΔLLO, including the PEST AA sequence, SEQ ID NO: 1, enhances cell mediated and anti-tumor immunity of the antigen. Each possibility represents a separate embodiment of the present invention.

It will be appreciated by the skilled artisan that the term “PEST-sequence containing peptide” may encompass a PEST sequence peptide or peptide fragments of an LLO protein or an ActA protein thereof. PEST sequence peptides are known in the art and are described in U.S. Pat. No. 7,635,479, and in US patent Publication Serial No. 2014/0186387, both of which are hereby incorporated in their entirety herein.

In another embodiment, a PEST sequence of prokaryotic organisms can be identified routinely in accordance with methods such as described by Rechsteiner and Roberts (TBS 21:267-271, 1996) for L. monocytogenes. Alternatively, PEST amino acid sequences from other prokaryotic organisms can also be identified based by this method. Other prokaryotic organisms wherein PEST amino acid sequences would be expected to include, but are not limited to, other Listeria species. For example, the L. monocytogenes protein ActA contains four such sequences. These are KTEEQPSEVNTGPR (SEQ ID NO: 5), KASVTDTSEGDLDSSMQSADESTPQPLK (SEQ ID NO: 6), KNEEVNASDFPPPPTDEELR (SEQ ID NO: 7), and RGGIPTSEEFSSLNSGDFTDDENSETTEEEIDR (SEQ ID NO: 8). Also Streptolysin O from Streptococcus sp. contain a PEST sequence. For example, Streptococcus pyogenes Streptolysin O comprises the PEST sequence KQNTASTETTTTNEQPK (SEQ ID NO: 9) at amino acids 35-51 and Streptococcus equisimilis Streptolysin O comprises the PEST-like sequence KQNTANTETTTTNEQPK (SEQ ID NO: 10) at amino acids 38-54. Further, it is believed that the PEST sequence can be embedded within the antigenic protein. Thus, for purposes of the present invention, by “fusion” when in relation to PEST sequence fusions, it is meant that the antigenic protein comprises both the antigen and the PEST amino acid sequence either linked at one end of the antigen or embedded within the antigen. In other embodiments, a PEST sequence or PEST containing polypeptide is not part of a fusion protein, nor does the polypeptide include a heterologous antigen.

In another embodiment, the construct or nucleic acid molecule is expressed from an episomal or plasmid vector, with a nucleic acid sequence encoding a PEST sequence-containing polypeptide or a PEST-sequence peptide. In another embodiment, the plasmid is stably maintained in the recombinant Listeria strain in the absence of antibiotic selection. In another embodiment, the plasmid does not confer antibiotic resistance upon the recombinant Listeria. In another embodiment, the fragment is a functional fragment. In another embodiment, the fragment is an immunogenic fragment.

The LLO protein utilized to construct vaccines of the present invention has, in another embodiment, the sequence: MKKINILVFTTLILVSLPIAQQTEAKDASAFNKENSISSMAPPASPPASPKTPIEK KHADEIDKYIQGLDYNKNNVLVYHGDAVTNVPPRKGYKDGNEYIVVEKKKKSINQNN ADIQVVNAISSLTYPGALVKANSELVENQPDVLPVKRDSLTLSIDLPGMTNQDNKIVVK NATKSNVNNAVNTLVERWNEKYAQAYPNVSAKIDYDDEMAYSESQLIAKFGTAFKA VNNSLNVNFGAISEGKMQEEVISFKQIYYNVNVNEPTRPSRFFGKAVTKEQLQALGVN AENPPAYISSVAYGRQVYLKLSTNSHSTKVKAAFDAAVSGKSVSGDVELTNIIKNSSFK AVIYGGSAKDEVQIIDGNLGDLRDILKKGATFNRETPGVPIAYTTNFLKDNELAVIKNN SEYIETTSKAYTDGKINIDHSGGYVAQFNISWDEVNYDPEGNEIVQHKNWSENNKSKL AHFTSSIYLPGNARNINVYAKECTGLAWEWWRTVIDDRNLPLVKNRNISIWGTTLYPK YSNKVDNPIE (GenBank Accession No. P13128; SEQ ID NO: 2; nucleic acid sequence is set forth in GenBank Accession No. X15127). The first 25 AA of the proprotein corresponding to this sequence are the signal sequence and are cleaved from LLO when it is secreted by the bacterium. Thus, in this embodiment, the full length active LLO protein is 504 residues long. In another embodiment, the above LLO fragment is used as the source of the LLO fragment incorporated in a vaccine of the present invention. Each possibility represents a separate embodiment of the present invention.

In another embodiment, the N-terminal fragment of an LLO protein utilized in compositions and methods of the present invention has the sequence:

(SEQ ID NO: 3)
MKKIMLVFITLILVSLPIAQQTEAKDASAFNKENSISSVAPPASPPAS
PKTPIEKKHADEIDKYIQGLDYNKNNVLVYHGDAVTNVPPRKGYKDGN
EYIVVEKKKKSINQNNADIQVVNAISSLTYPGALVKANSELVENQPDV
LPVKRDSLTLSIDLPGMTNQDNKIVVKNATKSNVNNAVNTLVERWNEK
YAQAYSNVSAKIDYDDEMAYSESQLIAKFGTAFKAVNNSLNVNFGAIS
EGKMQEEVISFKQIYYNVNVNEPTRPSRFFGKAVTKEQLQALGVNAEN
PPAYISSVAYGRQVYLKLSTNSHSTKVKAAFDAAVSGKSVSGDVELTN
IIKNSSFKAVIYGGSAKDEVQIIDGNLGDLRDILKKGATFNRETPGVP
IAYTTNFLKDNELAVIKNNSEYIETTSKAYTDGKINIDHSGGYVAQFN
ISWDEVNYD.

In another embodiment, the LLO fragment corresponds to about AA 20-442 of an LLO protein utilized herein.

In another embodiment, the LLO fragment has the sequence:

(SEQ ID NO: 4)
MKKEVILVFITLILVSLPIAQQTEAKDASAFNKENSISSVAPPASPPA
SPKTPIEKKHADEIDKYIQGLDYNKNNYLVYHGDAVTNYPPRKGYKDG
NEYIVVEKKKKSINQNNADIQVVNAISSLTYPGALVKANSELVENQPD
YLPVKRDSLTLSIDLPGMTNQDNKIVVKNATKSNVNNAVNTLVERWNE
KYAQAYSNVSAKIDYDDEMAYSESQLIAKFGTAFKAVNNSLNVNFGAI
SEGKMQEEVISFKQIYYNVNVNEPTRPSRFFGKAVTKEQLQALGVNAE
NPPAYISSVAYGRQVYLKLSTNSHSTKVKAAFDAAVSGKSVSGDVELT
NIIKNSSFKAVIYGGSAKDEVQIIDGNLGDLRDILKKGATFNRETPGV
PIAYTTNFLKDNELAVIKNNSEYIETTSKAYTD.

In another embodiment, the terms “N-terminal LLO fragment” “truncated LLO”, “ΔLLO” or their grammatical equivalents are used interchangeably herein and refers to a fragment of LLO that is non-hemolytic. In another embodiment, the terms refer to an LLO fragment that comprises a putative PEST sequence.

In another embodiment, the LLO fragment is rendered non-hemolytic by deletion or mutation of the activation domain. In another embodiment, the LLO fragment is rendered non-hemolytic by deletion or mutation of region comprising cysteine 484. In another embodiment, the LLO is rendered non-hemolytic by a deletion or mutation of the cholesterol binding domain (CBD) as detailed in U.S. Pat. No. 8,771,702, which is incorporated by reference herein.

In another embodiment, the LLO fragment comprises the first 441 AA of the wild-type LLO protein. In another embodiment, the LLO fragment comprises the first 420 AA of the wild-type LLO. In another embodiment, the LLO fragment is a non-hemolytic form of the wild-type LLO protein.

In another embodiment, the LLO fragment consists of about residues 1-25. In another embodiment, the LLO fragment consists of about residues 1-50. In another embodiment, the LLO fragment consists of about residues 1-75. In another embodiment, the LLO fragment consists of about residues 1-100. In another embodiment, the LLO fragment consists of about residues 1-125. In another embodiment, the LLO fragment consists of about residues 1-150. In another embodiment, the LLO fragment consists of about residues 1175. In another embodiment, the LLO fragment consists of about residues 1-200. In another embodiment, the LLO fragment consists of about residues 1-225. In another embodiment, the LLO fragment consists of about residues 1-250. In another embodiment, the LLO fragment consists of about residues 1-275. In another embodiment, the LLO fragment consists of about residues 1-300. In another embodiment, the LLO fragment consists of about residues 1-325. In another embodiment, the LLO fragment consists of about residues 1-350. In another embodiment, the LLO fragment consists of about residues 1-375. In another embodiment, the LLO fragment consists of about residues 1-400. In another embodiment, the LLO fragment consists of about residues 1-425.

In another embodiment, the LLO fragment contains residues of a homologous LLO protein that correspond to one of the above AA ranges. The residue numbers need not, in another embodiment, correspond exactly with the residue numbers enumerated above; e.g. if the homologous LLO protein has an insertion or deletion, relative to an LLO protein utilized herein, then the residue numbers can be adjusted accordingly. In another embodiment, the LLO fragment is any other LLO fragment known in the art.

In another embodiment, a homologous LLO refers to identity to an LLO sequence disclosed herein of greater than 70%. In another embodiment, a homologous LLO refers to identity an LLO sequence disclosed herein of greater than 72%. In another embodiment, a homologous refers to identity to an LLO sequence disclosed herein of greater than 75%. In another embodiment, a homologous refers to identity to an LLO sequence disclosed herein of greater than 78%. In another embodiment, a homologous refers to identity to an LLO sequence disclosed herein of greater than 80%. In another embodiment, a homologous refers to identity to an LLO sequence disclosed herein of greater than 82%. In another embodiment, a homologous refers to identity to an LLO sequence disclosed herein of greater than 83%. In another embodiment, a homologous refers to identity to an LLO sequence disclosed herein of greater than 85%. In another embodiment, a homologous refers to identity to an LLO sequence disclosed herein of greater than 87%. In another embodiment, a homologous refers to identity to an LLO sequence disclosed herein of greater than 88%. In another embodiment, a homologous refers to identity to an LLO sequence disclosed herein of greater than 90%. In another embodiment, a homologous refers to identity to an LLO sequence disclosed herein of greater than 92%. In another embodiment, a homologous refers to identity to an LLO sequence disclosed herein of greater than 93%. In another embodiment, a homologous refers to identity to an LLO sequence disclosed herein of greater than 95%. In another embodiment, a homologous refers to identity to an LLO sequence disclosed herein of greater than 96%. In another embodiment, a homologous refers to identity to an LLO sequence disclosed herein of greater than 97%. In another embodiment, a homologous refers to identity to an LLO sequence disclosed herein of greater than 98%. In another embodiment, a homologous refers to identity to an LLO sequence disclosed herein of greater than 99%. In another embodiment, a homologous refers to identity to an LLO sequence disclosed herein of 100%.

In one embodiment, an ActA protein comprises the sequence set forth in SEQ ID NO: 11:

MGLNRFMRAMMVVFITANCITINPDIIFAATDSEDSSLNTDEWEEEKTEEQPS EVNTGPRYETAREVSSRDIKELEKSNKVRNTNKADLIAMLKEKAEKGPNINNNNSEQT ENAAINEEASGADRPAIQVERRHPGLPSDSAAEIKKRRKAIASSDSELESLTYPDKPTKV NKKKVAKESVADASESDLDSSMQSADESSPQPLKANQQPFFPKVFKKIKDAGKWVRD KIDENPEVKKAIVDKSAGLIDQLLTKKKSEEVNASDFPPPPTDEELRLALPETPMLLGFN APATSEPSSFEFPPPPTDEELRLALPETPMLLGFNAPATSEPSSFEFPPPPTEDELEIIRETA SSLDSSFTRGDLASLRNAINRHSQNFSDFPPIPTEEELNGRGGRPTSEEFSSLNSGDFTDD ENSETTEEEIDRLADLRDRGTGKHSRNAGFLPLNPFASSPVPSLSPKVSKISDRALISDIT KKTPFKNPSQPLNVFNKKTTTKTVTKKPTPVKTAPKLAELPATKPQETVLRENKTPFIE KQAETNKQSINMPSLPVIQKEATESDKEEMKPQTEEKMVEESESANNANGKNRSAGIE EGKLIAKSAEDEKAKEEPGNHTTLILAMLAIGVFSLGAFIKIIQLRKNN. The first 29 AA of the proprotein corresponding to this sequence are the signal sequence and are cleaved from ActA protein when it is secreted by the bacterium. In one embodiment, an ActA polypeptide or peptide comprises the signal sequence, AA 1-29 of SEQ ID NO: 11 above. In another embodiment, an ActA polypeptide or peptide does not include the signal sequence, AA 1-29 of SEQ ID NO: 11 above.

In one embodiment, a truncated ActA protein comprises an N-terminal fragment of an ActA protein. In another embodiment, a truncated ActA protein is an N-terminal fragment of an ActA protein. In one embodiment, a truncated ActA protein comprises the sequence set forth in SEQ ID NO: 12:

MRAMMVVFITANCITINPDITAATDSEDSSLNTDEWEEEKTEEQPSEVNTGP RYETAREVSSRDIKELEKSNKVRNTNKADLIAMLKEKAEKGPNINNNNSEQTENAAINE EASGADRPAIQVERRHPGLPSDSAAEIKKRRKAIASSDSELESLTYPDKPTKVNKKKVA KESVADASESDLDSSMQSADESSPQPLKANQQPFFPKVFKKIKDAGKWVRDKIDENPE VKKAIVDKSAGLIDQLLTKKKSEEVNASDFPPPPTDEELRLALPETPMLLGFNAPATSEP SSFEFPPPPTDEELRLALPETPMLLGFNAPATSEPSSFEFPPPPTEDELEIIRETASSLDSSFT RGDLASLRNAINRHSQNFSDFPPIPTEEELNGRGGRP. In another embodiment, the ActA fragment comprises the sequence set forth in SEQ ID NO: 12.

In another embodiment, a truncated ActA protein comprises the sequence set forth in SEQ ID NO: 13:

MGLNRFMRAMMVVFITANCITINPDIIFAATDSEDSSLNTDEWEEEKT
EEQPSEVNTGPRYETAREVSSRDIKELEKSNKVRNTNKADLIAMLKEK
AEKG.

In another embodiment, the ActA fragment is any other ActA fragment known in the art. Each possibility represents a separate embodiment of the present invention.

In another embodiment, the recombinant nucleotide encoding a truncated ActA protein comprises the sequence set forth in SEQ ID NO: 14:

atgcgtgcgatgatggtggttttcattactgccaattgcattacgattaaccccgacataatatttgcagcgacagatagcgaa gattctagtctaaacacagatgaatgggaagaagaaaaaacagaagagcaaccaagcgaggtaaatacgggaccaagatacgaaactg cacgtgaagtaagttcacgtgatattaaagaactagaaaaatcgaataaagtgagaaatacgaacaaagcagacctaatagcaatgttgaaa gaaaaagcagaaaaaggtccaaatatcaataataacaacagtgaacaaactgagaatgcggctataaatgaagaggcttcaggagccgac cgaccagctatacaagtggagcgtcgtcatccaggattgccatcggatagcgcagcggaaattaaaaaaagaaggaaagccatagcatca tcggatagtgagcttgaaagccttacttatccggataaaccaacaaaagtaaataagaaaaaagtggcgaaagagtcagttgcggatgcttc tgaaagtgacttagattctagcatgcagtcagcagatgagtcttcaccacaacctttaaaagcaaaccaacaaccatttttccctaaagtattta aaaaaataaaagatgcggggaaatgggtacgtgataaaatcgacgaaaatcctgaagtaaagaaagcgattgttgataaaagtgcagggtt aattgaccaattattaaccaaaaagaaaagtgaagaggtaaatgcttcggacttcccgccaccacctacggatgaagagttaagacttgcttt gccagagacaccaatgcttcttggttttaatgctcctgctacatcagaaccgagctcattcgaatttccaccaccacctacggatgaagagtta agacttgctttgccagagacgccaatgcttcttggttttaatgctcctgctacatcggaaccgagctcgttcgaatttccaccgcctccaacaga agatgaactagaaatcatccgggaaacagcatcctcgctagattctagattacaagaggggatttagctagtttgagaaatgctattaatcgc catagtcaaaatttctctgatttcccaccaatcccaacagaagaagagttgaacgggagaggcggtagacca. In another embodiment, the recombinant nucleotide has the sequence set forth in SEQ ID NO: 14. In another embodiment, the recombinant nucleotide comprises any other sequence that encodes a fragment of an ActA protein.

In another embodiment, “truncated ActA” or “ΔActA” refers to a fragment of ActA that comprises the PEST domain. In another embodiment, the terms refer to an ActA fragment that comprises a PEST sequence.

In another embodiment, the PEST sequence is another PEST AA sequence derived from a prokaryotic organism. In another embodiment, the PEST sequence is any other PEST sequence known in the art.

In another embodiment, the ActA fragment consists of about the first 100 AA of the ActA protein.

In another embodiment, the ActA fragment consists of about residues 1-25. In another embodiment, the ActA fragment consists of about residues 1-50. In another embodiment, the ActA fragment consists of about residues 1-75. In another embodiment, the ActA fragment consists of about residues 1-100. In another embodiment, the ActA fragment consists of about residues 1-125. In another embodiment, the ActA fragment consists of about residues 1-150. In another embodiment, the ActA fragment consists of about residues 1-175. In another embodiment, the ActA fragment consists of about residues 1-200. In another embodiment, the ActA fragment consists of about residues 1-225. In another embodiment, the ActA fragment consists of about residues 1-250. In another embodiment, the ActA fragment consists of about residues 1-275. In another embodiment, the ActA fragment consists of about residues 1-300. In another embodiment, the ActA fragment consists of about residues 1-325. In another embodiment, the ActA fragment consists of about residues 1-338. In another embodiment, the ActA fragment consists of about residues 1-350. In another embodiment, the ActA fragment consists of about residues 1-375. In another embodiment, the ActA fragment consists of about residues 1-400. In another embodiment, the ActA fragment consists of about residues 1-450. In another embodiment, the ActA fragment consists of about residues 1-500. In another embodiment, the ActA fragment consists of about residues 1-550. In another embodiment, the ActA fragment consists of about residues 1-600. In another embodiment, the ActA fragment consists of about residues 1-639. In another embodiment, the ActA fragment consists of about residues 30-100. In another embodiment, the ActA fragment consists of about residues 30-125. In another embodiment, the ActA fragment consists of about residues 30-150. In another embodiment, the ActA fragment consists of about residues 30-175. In another embodiment, the ActA fragment consists of about residues 30-200. In another embodiment, the ActA fragment consists of about residues 30-225. In another embodiment, the ActA fragment consists of about residues 30-250. In another embodiment, the ActA fragment consists of about residues 30-275. In another embodiment, the ActA fragment consists of about residues 30-300. In another embodiment, the ActA fragment consists of about residues 30-325. In another embodiment, the ActA fragment consists of about residues 30-338. In another embodiment, the ActA fragment consists of about residues 30-350. In another embodiment, the ActA fragment consists of about residues 30-375. In another embodiment, the ActA fragment consists of about residues 30-400. In another embodiment, the ActA fragment consists of about residues 30-450. In another embodiment, the ActA fragment consists of about residues 30-500. In another embodiment, the ActA fragment consists of about residues 30-550. In another embodiment, the ActA fragment consists of about residues 1-600. In another embodiment, the ActA fragment consists of about residues 30-604. Each possibility represents a separate embodiment of the present invention.

In another embodiment, the ActA fragment contains residues of a homologous ActA protein that correspond to one of the above AA ranges. The residue numbers need not, in another embodiment, correspond exactly with the residue numbers enumerated above; e.g. if the homologous ActA protein has an insertion or deletion, relative to an ActA protein utilized herein, then the residue numbers can be adjusted accordingly. In another embodiment, the ActA fragment is any other ActA fragment known in the art.

In another embodiment, a homologous ActA refers to identity to an ActA sequence disclosed herein of greater than 70%. In another embodiment, a homologous ActA refers to identity to an ActA sequence disclosed herein of greater than 72%. In another embodiment, a homologous ActA refers to identity to an ActA sequence disclosed herein of greater than 75%. In another embodiment, a homologous ActA refers to identity to an ActA sequence disclosed herein of greater than 78%. In another embodiment, a homologous refers to identity to an ActA sequence disclosed herein of greater than 80%. In another embodiment, a homologous refers to identity to an ActA sequence disclosed herein of greater than 82%. In another embodiment, a homologous refers to identity to an ActA sequence disclosed herein of greater than 83%. In another embodiment, a homologous refers to identity to an ActA sequence disclosed herein of greater than 85%. In another embodiment, a homologous refers to identity to an ActA sequence disclosed herein of greater than 87%. In another embodiment, a homologous refers to identity to an ActA sequence disclosed herein of greater than 88%. In another embodiment, a homologous refers to identity to an ActA sequence disclosed herein of greater than 90%. In another embodiment, a homologous refers to identity to one of SEQ ID No: 11 of greater than 92%. In another embodiment, a homologous refers to identity to an ActA sequence disclosed herein of greater than 93%. In another embodiment, a homologous refers to identity to an ActA sequence disclosed herein of greater than 95%. In another embodiment, a homologous refers to identity to an ActA sequence disclosed herein of greater than 96%. In another embodiment, a homologous refers to identity to an ActA sequence disclosed herein of greater than 97%. In another embodiment, a homologous refers to identity to an ActA sequence disclosed herein of greater than 98%. In another embodiment, a homologous refers to identity to an ActA sequence disclosed herein of greater than 99%. In another embodiment, a homologous refers to identity to an ActA sequence disclosed herein of 100%.

It will be appreciated to the skilled artisan that the term “homology,” when in reference to any nucleic acid sequence disclosed herein may refer to a percentage of nucleotides in a candidate sequence that is identical with the nucleotides of a corresponding native nucleic acid sequence.

Homology is, in one embodiment, determined by a computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

In another embodiment, “homology” refers to identity to a sequence selected from the sequences disclosed herein of greater than 68%. In another embodiment, “homology” refers to identity to a sequence selected from the sequences disclosed herein of greater than 70%. In another embodiment, “homology” refers to identity to a sequence selected from the sequences disclosed herein of greater than 72%. In another embodiment, the identity is greater than 75%. In another embodiment, the identity is greater than 78%. In another embodiment, the identity is greater than 80%. In another embodiment, the identity is greater than 82%. In another embodiment, the identity is greater than 83%. In another embodiment, the identity is greater than 85%. In another embodiment, the identity is greater than 87%. In another embodiment, the identity is greater than 88%. In another embodiment, the identity is greater than 90%. In another embodiment, the identity is greater than 92%. In another embodiment, the identity is greater than 93%. In another embodiment, the identity is greater than 95%. In another embodiment, the identity is greater than 96%. In another embodiment, the identity is greater than 97%. In another embodiment, the identity is greater than 98%. In another embodiment, the identity is greater than 99%. In another embodiment, the identity is 100%.

In another embodiment, homology is determined via determination of candidate sequence hybridization, methods of which are well described in the art (See, for example, “Nucleic Acid Hybridization” Hames, B. D., and Higgins S. J., Eds. (1985); Sambrook et at, 2001, Molecular Cloning, A Laboratory Manual, Cold Spring Harbor Press, N.Y.; and Ausubel et al., 1989, Current Protocols in Molecular Biology, Green Publishing Associates and Wiley Interscience, N.Y). For example methods of hybridization may be carried out under moderate to stringent conditions, to the complement of a DNA encoding a native caspase peptide. Hybridization conditions being, for example, overnight incubation at 42° C. in a solution comprising: 10-20% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared salmon sperm DNA.

In one embodiment, the recombinant Listeria strain disclosed herein lacks antibiotic resistance genes. In another embodiment, the recombinant Listeria strain disclosed herein comprises a plasmid comprising a nucleic acid encoding an antibiotic resistance gene.

In one embodiment, the recombinant Listeria disclosed herein is capable of escaping the phagolysosome.

In one embodiment, the heterologous antigen or antigenic polypeptide is integrated in frame with LLO in the Listeria chromosome. In another embodiment, the integrated nucleic acid molecule is integrated in frame with ActA into the actA locus. In another embodiment, the chromosomal nucleic acid encoding ActA is replaced by a nucleic acid molecule encoding an antigen.

In one embodiment, a heterologous antigen is a tumor-associated antigen. In another embodiment, the tumor-associated antigen is a naturally occurring tumor-associated antigen. In another embodiment, the tumor-associated antigen is a synthetic tumor-associated antigen. In yet another embodiment, the tumor-associated antigen is a chimeric tumor-associated antigen.

In one embodiment, a recombinant Listeria disclosed herein comprises a nucleic acid molecule. In another embodiment, the nucleic acid molecule disclosed herein comprises a first open reading frame encoding recombinant polypeptide comprising a heterologous antigen or fragment thereof. In another embodiment, the recombinant polypeptide further comprises a truncated LLO protein, a truncated ActA protein or PEST sequence peptide fused to the heterologous antigen. In another embodiment, the truncated LLO protein is a N-terminal LLO or fragment thereof. In another embodiment, the truncated ActA protein is a N-terminal ActA protein or fragment thereof.

In another embodiment, the nucleic acid molecule disclosed herein comprises a first open reading frame encoding a recombinant polypeptide comprising a truncated LLO protein, a truncated ActA protein or a PEST sequence peptide, wherein the truncated LLO protein, a truncated ActA protein or a PEST sequence peptide is not fused to a heterologous antigen. In another embodiment, the first open reading frame encodes a truncated LLO protein comprising an N-terminal LLO or fragment thereof. In another embodiment, the first open reading frame encodes a truncated ActA protein comprising a N-terminal ActA protein or fragment thereof. In another embodiment, the first open reading frame encodes a truncated LLO protein consisting essentially of an N-terminal LLO or fragment thereof. In another embodiment, the first open reading frame encodes a truncated ActA protein consisting essentially of an N-terminal ActA protein or fragment thereof. In another embodiment, the first open reading frame encodes a truncated LLO protein consisting of an N-terminal LLO or fragment thereof. In another embodiment, the first open reading frame encodes a truncated ActA protein consisting of an N-terminal ActA protein or fragment thereof.

In one embodiment, the terms “antigen,” “antigen fragment,” “antigen portion,” “heterologous protein,” “heterologous antigen,” “heterologous protein antigen,” “protein antigen,” “antigen,” “antigenic polypeptide,” or their grammatical equivalents, are used interchangeably herein and are meant to refer to a polypeptide, peptide or recombinant peptide as described herein that is processed and presented on MHC class I and/or class II molecules present in a subject's cells leading to the mounting of an immune response when present in, or in another embodiment, detected by, the host. In one embodiment, the antigen may be foreign to the host. In another embodiment, the antigen might be present in the host but the host does not elicit an immune response against it because of immunologic tolerance.

In one embodiment, a nucleic acid molecule disclosed herein further comprises a second open reading frame encoding a metabolic enzyme. In another embodiment, the metabolic enzyme complements an endogenous gene that is lacking in the chromosome of the recombinant Listeria strain. In another embodiment, the metabolic enzyme encoded by the second open reading frame is an alanine racemase enzyme (Dal). In another embodiment, the metabolic enzyme encoded by the second open reading frame is a D-amino acid transferase enzyme (Dat). In another embodiment, the Listeria strains disclosed herein comprise a mutation in the endogenous dal/dat genes. In another embodiment, the Listeria lacks the dal/dat genes. In another embodiment, the dal/dat genes are deleted in the Listeria chromosome. In another embodiment, the dal/dat genes are truncated in the Listeria chromosome.

In another embodiment, a nucleic acid molecule of the disclosed methods and compositions is operably linked to a promoter/regulatory sequence. In another embodiment, the first open reading frame of the disclosed methods and compositions is operably linked to a promoter/regulatory sequence. In another embodiment, the second open reading frame of the disclosed methods and compositions is operably linked to a promoter/regulatory sequence. In another embodiment, each of the open reading frames are operably linked to a promoter/regulatory sequence.

“Metabolic enzyme” refers, in another embodiment, to an enzyme involved in synthesis of a nutrient required by the host bacteria. In another embodiment, the term refers to an enzyme required for synthesis of a nutrient required by the host bacteria. In another embodiment, the term refers to an enzyme involved in synthesis of a nutrient utilized by the host bacteria. In another embodiment, the term refers to an enzyme involved in synthesis of a nutrient required for sustained growth of the host bacteria. In another embodiment, the enzyme is required for synthesis of the nutrient.

In another embodiment, the recombinant Listeria is an attenuated auxotrophic strain. In another embodiment, the recombinant Listeria is an Lm-LLO-E7 strain described in U.S. Pat. No. 8,114,414, which is incorporated by reference herein in its entirety.

In one embodiment the attenuated strain is Lm dal(−)dat(−) (Lmdd). In another embodiment, the attenuated strains is Lm dal(−)dat(−)ΔactA (LmddA). LmddA is based on a Listeria strain which is attenuated due to the deletion of virulence gene actA and retains the plasmid for a desired heterologous antigen or truncated LLO expression in vivo and in vitro by complementation of the dal gene.

In another embodiment, the attenuated strain is LmΔactA. In another embodiment, the attenuated strain is LmΔPrfA. In another embodiment, the attenuated strain is LmΔPlcB. In another embodiment, the attenuated strain is LmΔPlcA. In another embodiment, the strain is the double mutant or triple mutant of any of the above-mentioned strains. In another embodiment, this strain exerts a strong adjuvant effect which is an inherent property of Listeria-based vaccines. In another embodiment, this strain is constructed from the EGD Listeria backbone. In another embodiment, the strain used in the invention is a Listeria strain that expresses a non-hemolytic LLO.

In another embodiment, the Listeria strain is an auxotrophic mutant. In another embodiment, the Listeria strain is deficient in a gene encoding a vitamin synthesis gene. In another embodiment, the Listeria strain is deficient in a gene encoding pantothenic acid synthase.

In one embodiment, the generation of AA strains of Listeria deficient in D-alanine, for example, may be accomplished in a number of ways that are well known to those of skill in the art, including deletion mutagenesis, insertion mutagenesis, and mutagenesis which results in the generation of frameshift mutations, mutations which cause premature termination of a protein, or mutation of regulatory sequences which affect gene expression. In another embodiment, mutagenesis can be accomplished using recombinant DNA techniques or using traditional mutagenesis technology using mutagenic chemicals or radiation and subsequent selection of mutants. In another embodiment, deletion mutants are preferred because of the accompanying low probability of reversion of the auxotrophic phenotype. In another embodiment, mutants of D-alanine which are generated according to the protocols presented herein may be tested for the ability to grow in the absence of D-alanine in a simple laboratory culture assay. In another embodiment, those mutants which are unable to grow in the absence of this compound are selected for further study.

In another embodiment, in addition to the aforementioned D-alanine associated genes, other genes involved in synthesis of a metabolic enzyme, as disclosed herein, may be used as targets for mutagenesis of Listeria.

In another embodiment, the metabolic enzyme complements an endogenous metabolic gene that is lacking in the remainder of the chromosome of the recombinant bacterial strain. In one embodiment, the endogenous metabolic gene is mutated in the chromosome. In another embodiment, the endogenous metabolic gene is deleted from the chromosome. In another embodiment, the metabolic enzyme is an amino acid metabolism enzyme. In another embodiment, the metabolic enzyme catalyzes a formation of an amino acid used for a cell wall synthesis in the recombinant Listeria strain. In another embodiment, the metabolic enzyme is an alanine racemase enzyme. In another embodiment, the metabolic enzyme is a D-amino acid transferase enzyme.

In one embodiment, the auxotrophic Listeria strain comprises an episomal expression vector comprising a metabolic enzyme that complements the auxotrophy of the auxotrophic Listeria strain. In another embodiment, the construct is contained in the Listeria strain in an episomal fashion. In another embodiment, the foreign antigen is expressed from a vector harbored by the recombinant Listeria strain. In another embodiment, the episomal expression vector lacks an antibiotic resistance marker. In one embodiment, an antigen of the methods and compositions as disclosed herein is fused to a polypeptide comprising a PEST sequence.

In another embodiment, the Listeria strain is deficient in an amino acid (AA) metabolism enzyme. In another embodiment, the Listeria strain is deficient in a D-glutamic acid synthase gene. In another embodiment, the Listeria strain is deficient in the dal gene. In another embodiment, the Listeria strain is deficient in the dga gene. In another embodiment, the Listeria strain is deficient in a gene involved in the synthesis of diaminopimelic acid CysK. In another embodiment, the gene is vitamin-B12 independent methionine synthase. In another embodiment, the gene is trpA. In another embodiment, the gene is trpB. In another embodiment, the gene is trpE. In another embodiment, the gene is asnB. In another embodiment, the gene is gltD. In another embodiment, the gene is gltB. In another embodiment, the gene is leuA. In another embodiment, the gene is argG. In another embodiment, the gene is thrC. In another embodiment, the Listeria strain is deficient in one or more of the genes described hereinabove.

In another embodiment, the Listeria strain is deficient in a synthase gene. In another embodiment, the gene is an AA synthesis gene. In another embodiment, the gene is folP. In another embodiment, the gene is dihydrouridine synthase family protein. In another embodiment, the gene is ispD. In another embodiment, the gene is ispF. In another embodiment, the gene is phosphoenolpyruvate synthase. In another embodiment, the gene is hisF. In another embodiment, the gene is hisH. In another embodiment, the gene is fliI. In another embodiment, the gene is ribosomal large subunit pseudouridine synthase. In another embodiment, the gene is ispD. In another embodiment, the gene is bifunctional GMP synthase/glutamine amidotransferase protein. In another embodiment, the gene is cobS. In another embodiment, the gene is cobB. In another embodiment, the gene is cbiD. In another embodiment, the gene is uroporphyrin-III C-methyltransferase/uroporphyrinogen-III synthase. In another embodiment, the gene is cobQ. In another embodiment, the gene is uppS. In another embodiment, the gene is truB. In another embodiment, the gene is dxs. In another embodiment, the gene is mvaS. In another embodiment, the gene is dapA. In another embodiment, the gene is ispG. In another embodiment, the gene is folC. In another embodiment, the gene is citrate synthase. In another embodiment, the gene is argJ. In another embodiment, the gene is 3-deoxy-7-phosphoheptulonate synthase. In another embodiment, the gene is indole-3-glycerol-phosphate synthase. In another embodiment, the gene is anthranilate synthase/glutamine amidotransferase component. In another embodiment, the gene is menB. In another embodiment, the gene is menaquinone-specific isochorismate synthase. In another embodiment, the gene is phosphoribosylformylglycinamidine synthase I or II. In another embodiment, the gene is phosphoribosylaminoimidazole-succinocarboxamide synthase. In another embodiment, the gene is carB. In another embodiment, the gene is carA. In another embodiment, the gene is thyA. In another embodiment, the gene is mgsA. In another embodiment, the gene is aroB. In another embodiment, the gene is hepB. In another embodiment, the gene is rluB. In another embodiment, the gene is ilvB. In another embodiment, the gene is ilvN. In another embodiment, the gene is alsS. In another embodiment, the gene is fabF. In another embodiment, the gene is fabH. In another embodiment, the gene is pseudouridine synthase. In another embodiment, the gene is pyrG. In another embodiment, the gene is truA. In another embodiment, the gene is pabB. In another embodiment, the gene is an atp synthase gene (e.g. atpC, atpD-2, aptG, atpA-2, etc).

In another embodiment, the gene is phoP. In another embodiment, the gene is aroA. In another embodiment, the gene is aroC. In another embodiment, the gene is aroD. In another embodiment, the gene is plcB.

In another embodiment, the Listeria strain is deficient in a peptide transporter. In another embodiment, the gene is ABC transporter/ATP-binding/permease protein. In another embodiment, the gene is oligopeptide ABC transporter/oligopeptide-binding protein. In another embodiment, the gene is oligopeptide ABC transporter/permease protein. In another embodiment, the gene is zinc ABC transporter/zinc-binding protein. In another embodiment, the gene is sugar ABC transporter. In another embodiment, the gene is phosphate transporter. In another embodiment, the gene is ZIP zinc transporter. In another embodiment, the gene is drug resistance transporter of the EmrB/QacA family. In another embodiment, the gene is sulfate transporter. In another embodiment, the gene is proton-dependent oligopeptide transporter. In another embodiment, the gene is magnesium transporter. In another embodiment, the gene is formate/nitrite transporter. In another embodiment, the gene is spermidine/putrescine ABC transporter. In another embodiment, the gene is Na/Pi-cotransporter. In another embodiment, the gene is sugar phosphate transporter. In another embodiment, the gene is glutamine ABC transporter. In another embodiment, the gene is major facilitator family transporter. In another embodiment, the gene is glycine betaine/L-proline ABC transporter. In another embodiment, the gene is molybdenum ABC transporter. In another embodiment, the gene is techoic acid ABC transporter. In another embodiment, the gene is cobalt ABC transporter. In another embodiment, the gene is ammonium transporter. In another embodiment, the gene is amino acid ABC transporter. In another embodiment, the gene is cell division ABC transporter. In another embodiment, the gene is manganese ABC transporter. In another embodiment, the gene is iron compound ABC transporter. In another embodiment, the gene is maltose/maltodextrin ABC transporter. In another embodiment, the gene is drug resistance transporter of the Bcr/CflA family. In another embodiment, the gene is a subunit of one of the above proteins.

In one embodiment, disclosed herein is a nucleic acid molecule that is used to transform the Listeria in order to arrive at a recombinant Listeria. In another embodiment, the nucleic acid disclosed herein used to transform Listeria lacks a virulence gene. In another embodiment, the nucleic acid molecule is integrated into the Listeria genome and carries a non-functional virulence gene. In another embodiment, the virulence gene is mutated in the recombinant Listeria. In yet another embodiment, the nucleic acid molecule is used to inactivate the endogenous gene present in the Listeria genome. In yet another embodiment, the virulence gene is an actA gene, an inlA gene, and inlB gene, an inlC gene, inlJ gene, a plbC gene, a bsh gene, or a prfA gene. It is to be understood by a skilled artisan, that the virulence gene can be any gene known in the art to be associated with virulence of the recombinant Listeria.

In one embodiment, the Listeria strain comprises a mutation in one or more endogenous genes. In another embodiment the Listeria strain is a dal mutant, a dat mutant, an inlA mutant, an inlB mutant, an inlC mutant, an inlJ mutant, prfA mutant, actA mutant, a dal/dat mutant, a prfA mutant, a plcB deletion mutant, or a double mutant in both plcA and plcB or actA and inlB or dal and dat, or a triple mutant in dal/dat and actA. In another embodiment, the Listeria disclosed herein comprises a mutation in any one of these genes or in a combination of these genes. In another embodiment, a Listeria disclosed herein lack each one of these genes. In another embodiment, the Listeria disclosed herein lacks at least one and up to ten of any gene disclosed herein, including the actA, prfA, and dal/dat genes.

In another embodiment, a Listeria strain comprising a dal and dat mutation is complemented by a metabolic enzyme encoded by a second open reading frame in a nucleic acid sequence present in a plasmid within the Listeria strain. In another embodiment, a Listeria strain comprising a prfA mutation is complemented by a mutant PrfA protein comprising a D133V amino acid mutation. In another embodiment, the mutant D133V PrfA protein is encoded by a second open reading frame in a nucleic acid sequence present in a plasmid within the Listeria strain.

In one embodiment, the live attenuated Listeria is a recombinant Listeria. In another embodiment, the recombinant Listeria comprises a mutation in a genomic internalin C (inlC) gene. In another embodiment, the recombinant Listeria comprises a mutation in a genomic actA gene and a genomic internalin C gene. In one embodiment, translocation of Listeria to adjacent cells is inhibited by the deletion of the actA gene and/or the inlC gene, which are involved in the process, thereby resulting in unexpectedly high levels of attenuation with increased immunogenicity and utility as a vaccine backbone.

In one embodiment, the metabolic gene, the virulence gene, etc. is lacking in a chromosome of the Listeria strain. In another embodiment, the metabolic gene, virulence gene, etc. is lacking in the chromosome and in any episomal genetic element of the Listeria strain. In another embodiment, the metabolic gene, virulence gene, etc. is lacking in the genome of the virulence strain. In one embodiment, the virulence gene is mutated in the chromosome. In another embodiment, the virulence gene is deleted from the chromosome.

In one embodiment, the recombinant Listeria strain disclosed herein is attenuated. In another embodiment, the recombinant Listeria lacks the actA virulence gene. In another embodiment, the recombinant Listeria lacks the prfA virulence gene. In another embodiment, the recombinant Listeria lacks the inlB gene. In another embodiment, the recombinant Listeria lacks both, the actA and inlB genes. In another embodiment, the recombinant Listeria strain disclosed herein comprises an inactivating mutation of the endogenous actA gene. In another embodiment, the recombinant Listeria strain disclosed herein comprises an inactivating mutation of the endogenous inlB gene. In another embodiment, the recombinant Listeria strain disclosed herein comprise an inactivating mutation of the endogenous inlC gene. In another embodiment, the recombinant Listeria strain disclosed herein comprises an inactivating mutation of the endogenous actA and inlB genes. In another embodiment, the recombinant Listeria strain disclosed herein comprise an inactivating mutation of the endogenous actA and inlC genes. In another embodiment, the recombinant Listeria strain disclosed herein comprises an inactivating mutation of the endogenous actA, inlB, and inlC genes. In another embodiment, the recombinant Listeria strain disclosed herein comprises an inactivating mutation of the endogenous actA, inlB, and inlC genes. In another embodiment, the recombinant Listeria strain disclosed herein comprise an inactivating mutation of the endogenous actA, inlB, and inlC genes. In another embodiment, the recombinant Listeria strain disclosed herein comprises an inactivating mutation in any single gene or combination of the following genes: actA, dal, dat, inlB, inlC, prfA, plcA, plcB.

It will be appreciated by the skilled artisan that the term “mutation” and grammatical equivalents thereof, include any type of mutation or modification to the sequence (nucleic acid or amino acid sequence), and includes a deletion, a truncation, an inactivation, a disruption, a replacement or a translocation. These types of mutations are readily known in the art.

In one embodiment, in order to select for an auxotrophic bacteria, such as an auxotrophic Listeria, comprising a plasmid encoding a metabolic enzyme or a complementing gene disclosed herein, transformed auxotrophic bacteria are grown on a media that will select for expression of the amino acid metabolism gene or the complementing gene. In another embodiment, a bacteria auxotrophic for D-glutamic acid synthesis is transformed with a plasmid comprising a gene for D-glutamic acid synthesis, and the auxotrophic bacteria will grow in the absence of D-glutamic acid, whereas auxotrophic bacteria that have not been transformed with the plasmid, or are not expressing the plasmid encoding a protein for D-glutamic acid synthesis, will not grow. In another embodiment, a bacterium auxotrophic for D-alanine synthesis will grow in the absence of D-alanine when transformed and expressing the plasmid of the present invention if the plasmid comprises an isolated nucleic acid encoding an amino acid metabolism enzyme for D-alanine synthesis. Such methods for making appropriate media comprising or lacking necessary growth factors, supplements, amino acids, vitamins, antibiotics, and the like are well known in the art, and are available commercially (Becton-Dickinson, Franklin Lakes, N.J.).

In another embodiment, once the auxotrophic bacteria comprising the complementing plasmid have been selected on appropriate media, the bacteria are propagated in the presence of a selective pressure. Such propagation comprises growing the bacteria in media without the auxotrophic factor. The presence of the plasmid expressing an amino acid metabolism enzyme in the auxotrophic bacteria ensures that the plasmid will replicate along with the bacteria, thus continually selecting for bacteria harboring the plasmid. The skilled artisan, when equipped with the present disclosure and methods herein will be readily able to scale-up the production of the recombinant Listeria strain by adjusting the volume of the media in which the auxotrophic bacteria comprising the plasmid are growing.

The skilled artisan will appreciate that, in another embodiment, other auxotroph strains and complementation systems are adopted for the use with the disclosure.

In one embodiment, the N-terminal LLO protein fragment and heterologous antigen are fused directly to one another. In another embodiment, the genes encoding the N-terminal LLO protein fragment and heterologous antigen are fused directly to one another. In another embodiment, the N-terminal LLO protein fragment and heterologous antigen are operably attached via a linker peptide. In another embodiment, the N-terminal LLO protein fragment and heterologous antigen are attached via a heterologous peptide. In another embodiment, the N-terminal LLO protein fragment is N-terminal to the heterologous antigen. In another embodiment, the N-terminal LLO protein fragment is expressed and used alone, i.e., in unfused form. In another embodiment, an N-terminal LLO protein fragment is the N-terminal-most portion of the fusion protein. In another embodiment, a truncated LLO is truncated at the C-terminal to arrive at an N-terminal LLO. In another embodiment, a truncated LLO is a non-hemolytic LLO.

As disclosed herein, there was an unexpected change in the suppressive ability of the granulocytic MDSC and this is due to the overexpression of tLLO that is independent of the partnering fusion antigen (see Example 19).

In one embodiment, the N-terminal ActA protein fragment and heterologous antigen are fused directly to one another. In another embodiment, the genes encoding the N-terminal ActA protein fragment and heterologous antigen are fused directly to one another. In another embodiment, the N-terminal ActA protein fragment and heterologous antigen are operably attached via a linker peptide. In another embodiment, the N-terminal ActA protein fragment and heterologous antigen are attached via a heterologous peptide. In another embodiment, the N-terminal ActA protein fragment is N-terminal to the heterologous antigen. In another embodiment, the N-terminal ActA protein fragment is expressed and used alone, i.e., in unfused form. In another embodiment, the N-terminal ActA protein fragment is the N-terminal-most portion of the fusion protein. In another embodiment, a truncated ActA is truncated at the C-terminal to arrive at an N-terminal ActA.

In one embodiment, the recombinant Listeria strain disclosed herein expresses the recombinant polypeptide. In another embodiment, the recombinant Listeria strain comprises a plasmid that encodes the recombinant polypeptide. In another embodiment, a recombinant nucleic acid disclosed herein is in a plasmid in the recombinant Listeria strain disclosed herein. In another embodiment, the plasmid is an episomal plasmid that does not integrate into the recombinant Listeria strain's chromosome. In another embodiment, the plasmid is an integrative plasmid that integrates into the Listeria strain's chromosome. In another embodiment, the plasmid is a multicopy plasmid.

one embodiment, the recombinant Listeria strain of the compositions and methods as disclosed herein express a heterologous antigenic polypeptide that is expressed by a tumor cell. In one embodiment, a tumor-associated antigen is a prostate specific antigen (PSA). In another embodiment, a tumor-associated antigen is a human papilloma virus (HPV) antigen. In yet another embodiment, a tumor-associated antigen is a Her2/neu chimeric antigen as described in US Patent Pub. No. US2011/014279, which is incorporated by reference herein in its entirety. In still another embodiment, a tumor-associated antigen is an angiogenic antigen.

In one embodiment, the recombinant Listeria strain of the compositions and methods as disclosed herein comprise a first or second nucleic acid molecule that encodes a Prostate Specific Antigen (PSA), which in one embodiment, is a marker for prostate cancer that is highly expressed by prostate tumors. In one embodiment, PSA is a kallikrein serine protease (KLK3) secreted by prostatic epithelial cells, which in one embodiment, is widely used as a marker for prostate cancer. As used herein, the terms PSA and KLK3 are interchangeable having all the same meanings and qualities.

In one embodiment, the recombinant Listeria strain as disclosed herein comprises a nucleic acid molecule encoding a tumor associated antigen. In one embodiment, a tumor associated antigen comprises an KLK3 polypeptide or a fragment thereof. In one embodiment, the recombinant Listeria strain as disclosed herein comprises a nucleic acid molecule encoding KLK3 protein.

In another embodiment, the KLK3 protein has the sequence:

MWVPVVFLTLSVTWIGAAPLILSRIVGGWECEKHSQPWQVLVASRGRAVCG GVLVHPQWVLTAAHCIRNKSVILLGRHSLFHPEDTGQVFQVSHSFPHPLYDMSLLKNR FLRPGDDSSHDLMLLRLSEPAELTDAVKVMDLPTQEPALGTTCYASGWGSIEPEEFLTP KKLQCVDLHVISNDVCAQVHPQKVTKFMLCAGRWTGGKSTCSGDSGGPLVCNGVLQ GITSWGSEPCALPERPSLYTKVVHYRKWIKDTIVANP (SEQ ID No: 15; GenBank Accession No. CAA32915). In another embodiment, the KLK3 protein is a homologue of SEQ ID No: 15. In another embodiment, the KLK3 protein is a variant of SEQ ID No: 15. In another embodiment, the KLK3 protein is an isomer of SEQ ID No: 15. In another embodiment, the KLK3 protein is a fragment of SEQ ID No: 15. Each possibility represents a separate embodiment of the methods and compositions as disclosed herein.

In another embodiment, the KLK3 protein has the sequence:

IVGGWECEKHSQPWQVLVASRGRAVCGGVLVHPQWVLTAAHCIRNKSVILL GRHSLFHPEDTGQVFQVSHSFPHPLYDMSLLKNRFLRPGDDSSHDLMLLRLSEPAELTD AVKVMDLPTQEPALGTTCYASGWGSIEPEEFLTPKKLQCVDLHVISNDVCAQVHPQKV TKFMLCAGRWTGGKSTCSGDSGGPLVCYGVLQGITSWGSEPCALPERPSLYTKVVHY RKWIKDTIVANP (SEQ ID No: 16). In another embodiment, the KLK3 protein is a homologue of SEQ ID No: 16. In another embodiment, the KLK3 protein is a variant of SEQ ID No: 16. In another embodiment, the KLK3 protein is an isomer of SEQ ID No: 16. In another embodiment, the KLK3 protein is a fragment of SEQ ID No: 16. Each possibility represents a separate embodiment of the methods and compositions as disclosed herein.

In another embodiment, the KLK3 protein has the sequence: IVGGWECEKHSQPWQVLVASRGRAVCGGVLVHPQWVLTAAHCIRNKSVILLGRHSLF HPEDTGQVFQVSHSFPHPLYDMSLLKNRFLRPGDDSSHDLMLLRLSEPAELTDAVKVM DLPTQEPALGTTCYASGWGSIEPEEFLTPKKLQCVDLHVISNDVCAQVHPQKVTKFML CAGRWTGGKSTCSGDSGGPLVCNGVLQGITSWGSEPCALPERPSLYTKVVHYRKWIK DTIVANP (SEQ ID No: 17; GenBank Accession No. AAA59995.1). In another embodiment, the KLK3 protein is a homologue of SEQ ID No: 17. In another embodiment, the KLK3 protein is a variant of SEQ ID No: 17. In another embodiment, the KLK3 protein is an isomer of SEQ ID No: 17. In another embodiment, the KLK3 protein is a fragment of SEQ ID No: 17. Each possibility represents a separate embodiment of the methods and compositions as disclosed herein.

In another embodiment, the KLK3 protein is encoded by a nucleotide molecule having the sequence: ggtgtcttaggcacactggtcttggagtgcaaaggatctaggcacgtgaggctttgtatgaagaatcggggatcgtacccaccccctgtttct gtttcatcctgggcatgtctectctgcctttgteccctagatgaagtctccatgagctacaagggcctggtgcatccagggtgatctagtaattg cagaacagcaagtgctagctctccctccccttccacagctctgggtgtgggagggggttgtccagcctccagcagcatggggagggcctt ggtcagcctctgggtgccagcagggcaggggcggagtcctggggaatgaaggttaatagggacctgggggaggctccccagcccca agcttaccacctgcacccggagagctgtgtcaccatgtgggtcccggttgtcttcctcaccctgtccgtgacgtggattggtgagaggggcc atggttggggggatgcaggagagggagccagccctgactgtcaagctgaggctctttcccccccaacccagcaccccagcccagacag ggagctgggctcttttctgtctctcccagccccacttcaagcccatacccccagtcccctccatattgcaacagtcctcactcccacaccaggt ccccgctccctcccacttaccccagaactttcttcccatttgcccagccagctccctgctcccagctgctttactaaaggggaagttcctgggc atctccgtgtttctctttgtggggctcaaaacctccaaggacctctctcaatgccattggttccttggaccgtatcactggtccatctcctgagcc cctcaatcctatcacagtctactgacttttcccattcagctgtgagtgtccaaccctatcccagagaccttgatgcttggcctcccaatcttgccc taggatacccagatgccaaccagacacctccttctttcctagccaggctatctggcctgagacaacaaatgggtccctcagtctggcaatgg gactctgagaactcctcattccctgactcttagccccagactcttcattcagtggcccacattttccttaggaaaaacatgagcatccccagcca caactgccagctctctgagtccccaaatctgcatcctatcaaaacctaaaaacaaaaagaaaaacaaataaaacaaaaccaactcagacca gaactgtatctcaacctgggacttcctaaacatccaaaaccttcctcttccagcaactgaacctcgccataaggcacttatccctggttcctag caccccttatcccctcagaatccacaacttgtaccaagtttcccttctcccagtccaagaccccaaatcaccacaaaggacccaatccccaga ctcaagatatggtctgggcgctgtcttgtgtctcctaccctgatccctgggttcaactctgctcccagagcatgaagcctctccaccagcacca gccaccaacctgcaaacctagggaagattgacagaattcccagcctacccagctccccctgcccatgtcccaggactcccagccaggttc tctgcccccgtgtcttttcaaacccacatcctaaatccatctcctatccgagtcccccagttccccctgtcaaccctgattcccctgatctagcac cccctctgcaggcgctgcgcccctcatcctgtctcggattgtgggaggctgggagtgcgagaagcattcccaaccctggcaggtgcttgtg gcctctcgtggcagggcagtctgcggcggtgttctggtgcacccccagtgggtcctcacagctgcccactgcatcaggaagtgagtaggg gcctggggtctggggagcaggtgtctgtgtcccagaggaataacagctgggcattttccccaggataacctctaaggccagccttgggact gggggagagagggaaagttctggttcaggtcacatggggaggcagggttggggctggaccaccctccccatggctgcctgggtctccat ctgtgtccctctatgtctctttgtgtcgctttcattatgtctcttggtaactggcttcggttgtgtctctccgtgtgactattttgttctctctctccctctct tctctgtcttcagtctccatatctccccctctctctgtccttctctggtccctctctagccagtgtgtctcaccctgtatctctctgccaggctctgtct ctcggtctctgtctcacctgtgccttctccctactgaacacacgcacgggatgggcctgggggaccctgagaaaaggaagggctttggctg ggcgcggtggctcacacctgtaatcccagcactttgggaggccaaggcaggtagatcacctgaggtcaggagttcgagaccagcctggc caactggtgaaaccccatctctactaaaaatacaaaaaattagccaggcgtggtggcgcatgcctgtagtcccagctactcaggagctgag ggaggagaattgcattgaacctggaggttgaggttgcagtgagccgagaccgtgccactgcactccagcctgggtgacagagtgagact ccgcctcaaaaaaaaaaaaaaaaaaaaaaaaaaaaaagaaaagaaaagaaaagaaaaggaagtgttttatccctgatgtgtgtgggtatg agggtatgagagggcccctctcactccattccttctccaggacatccctccactcttgggagacacagagaagggctggttccagctggag ctgggaggggcaattgagggaggaggaaggagaagggggaaggaaaacagggtatgggggaaaggaccctggggagcgaagtgg aggatacaaccttgggcctgcaggcaggctacctacccacttggaaacccacgccaaagccgcatctacagctgagccactctgaggcct cccctccccggcggtccccactcagctccaaagtctctctcccttttctctcccacactttatcatcccccggattcctctctacttggttctcattc ttcctttgacttcctgcttccctttctcattcatctgtttctcactttctgcctggttttgttcttctctctctctttctctggcccatgtctgtttctctatgttt ctgtcttttctttctcatcctgtgtattttcggctcaccttgtttgtcactgttctcccctctgccctttcattctctctgcccttttaccctcttccttttccc ttggttctctcagttctgtatctgcccttcaccctctcacactgctgtttcccaactcgttgtctgtattttggcctgaactgtgtcttcccaaccctgt gttttctcactgtttctttttctcttttggagcctcctccttgctcctctgtcccttctctctttccttatcatcctcgctcctcattcctgcgtctgcttcctc cccagcaaaagcgtgatcttgctgggtcggcacagcctgtttcatcctgaagacacaggccaggtatttcaggtcagccacagcttcccaca cccgctctacgatatgagcctcctgaagaatcgattcctcaggccaggtgatgactccagccacgacctcatgctgctccgcctgtcagagc ctgccgagctcacggatgctgtgaaggtcatggacctgcccacccaggagccagcactggggaccacctgctacgcctcaggctgggg cagcattgaaccagaggagtgtacgcctgggccagatggtgcagccgggagcccagatgcctgggtctgagggaggaggggacagga ctcctgggtctgagggaggagggccaaggaaccaggtggggtccagcccacaacagtgtttttgcctggcccgtagtcttgaccccaaag aaacttcagtgtgtggacctccatgttatttccaatgacgtgtgtgcgcaagttcaccctcagaaggtgaccaagttcatgctgtgtgctggac gctggacagggggcaaaagcacctgctcggtgagtcatccctactcccaagatcttgagggaaaggtgagtgggaccttaattctgggctg gggtctagaagccaacaaggcgtctgcctcccctgctccccagctgtagccatgccacctccccgtgtctcatctcattccctccttccctctt ctttgactccctcaaggcaataggttattcttacagcacaactcatctgttcctgcgttcagcacacggttactaggcacctgctatgcacccag cactgccctagagcctgggacatagcagtgaacagacagagagcagcccctcccttctgtagcccccaagccagtgaggggcacaggc aggaacagggaccacaacacagaaaagctggagggtgtcaggaggtgatcaggctctcggggagggagaaggggtggggagtgtga ctgggaggagacatcctgcagaaggtgggagtgagcaaacacctgcgcaggggaggggagggcctgcggcacctgggggagcaga gggaacagcatctggccaggcctgggaggaggggcctagagggcgtcaggagcagagaggaggttgcctggctggagtgaaggatc ggggcagggtgcgagagggaacaaaggacccctcctgcagggcctcacctgggccacaggaggacactgcttttcctctgaggagtca ggaactgtggatggtgctggacagaagcaggacagggcctggctcaggtgtccagaggctgcgctggcctcctatgggatcagactgca gggagggagggcagcagggatgtggagggagtgatgatggggctgacctgggggtggctccaggcattgtccccacctgggcccttac ccagcctccctcacaggctcctggccctcagtctctcccctccactccattctccacctacccacagtgggtcattctgatcaccgaactgacc atgccagccctgccgatggtcctccatggctccctagtgccctggagaggaggtgtctagtcagagagtagtcctggaaggtggcctctgt gaggagccacggggacagcatcctgcagatggtcctggcccttgtcccaccgacctgtctacaaggactgtcctcgtggaccctcccctct gcacaggagctggaccctgaagtcccttcctaccggccaggactggagcccctacccctctgttggaatccctgcccaccttcttctggaag tcggctctggagacatttctctcttcttccaaagctgggaactgctatctgttatctgcctgtccaggtctgaaagataggattgcccaggcaga aactgggactgacctatctcactctctccctgcttttacccttagggtgattctgggggcccacttgtctgtaatggtgtgcttcaaggtatcacg tcatggggcagtgaaccatgtgccctgcccgaaaggccttccctgtacaccaaggtggtgcattaccggaagtggatcaaggacaccatc gtggccaacccctgagcacccctatcaagtccctattgtagtaaacttggaaccttggaaatgaccaggccaagactcaagcctccccagtt ctactgacctttgtccttaggtgtgaggtccagggttgctaggaaaagaaatcagcagacacaggtgtagaccagagtgatcttaaatggtgt aattttgtcctctctgtgtcctggggaatactggccatgcctggagacatatcactcaatttctctgaggacacagttaggatggggtgtctgtgt tatttgtgggatacagagatgaaagaggggtgggatcc (SEQ ID No: 18; GenBank Accession No. X14810). In another embodiment, the KLK3 protein is encoded by residues 401 . . . 446, 1688 . . . 1847, 3477 . . . 3763, 3907 . . . 4043, and 5413 . . . 5568 of SEQ ID No: 18. In another embodiment, the KLK3 protein is encoded by a homologue of SEQ ID No: 18. In another embodiment, the KLK3 protein is encoded by a variant of SEQ ID No: 18. In another embodiment, the KLK3 protein is encoded by an isomer of SEQ ID No: 18. In another embodiment, the KLK3 protein is encoded by a fragment of SEQ ID No: 18. Each possibility represents a separate embodiment of the methods and compositions as disclosed herein.

In another embodiment, the KLK3 protein has the sequence: MWVPVVFLTLSVTWIGAAPLILSRIVGGWECEKHSQPWQVLVASRGRAVCGGVLVHP QWVLTAAHCIRNKSVILLGRHSLFHPEDTGQVFQVSHSFPHPLYDMSLLKNRFLRPGD DSSHDLMLLRLSEPAELTDAVKVMDLPTQEPALGTTCYASGWGSIEPEEFLTPKKLQC VDLHVISNDVCAQVHPQKVTKFMLCAGRWTGGKSTCSWVILITELTMPALPMVLHGS LVPWRGGV (SEQ ID No: 19; GenBank Accession No. NP_001019218) In another embodiment, the KLK3 protein is a homologue of SEQ ID No: 19. In another embodiment, the KLK3 protein is a variant of SEQ ID No: 19. In another embodiment, the KLK3 protein is an isomer of SEQ ID No: 19. In another embodiment, the KLK3 protein is a fragment of SEQ ID No: 19. Each possibility represents a separate embodiment as disclosed herein.

In another embodiment, the KLK3 protein is encoded by a nucleotide molecule having the sequence: agccccaagcttaccacctgcacccggagagctgtgtcaccatgtgggtcccggttgtcacctcaccctgtccgtgacgtggattggtgctg cacccctcatcctgtctcggattgtgggaggctgggagtgcgagaagcattcccaaccctggcaggtgcttgtggcctctcgtggcagggc agtctgcggcggtgttctggtgcacccccagtgggtcctcacagctgcccactgcatcaggaacaaaagcgtgatcttgctgggtcggcac agcctgtacatcctgaagacacaggccaggtatttcaggtcagccacagcttcccacacccgctctacgatatgagcctcctgaagaatcg attcctcaggccaggtgatgactccagccacgacctcatgctgctccgcctgtcagagcctgccgagctcacggatgctgtgaaggtcatg gacctgcccacccaggagccagcactggggaccacctgctacgcctcaggctggggcagcattgaaccagaggagttcttgaccccaa agaaacttcagtgtgtggacctccatgttatttccaatgacgtgtgtgcgcaagttcaccctcagaaggtgaccaagttcatgctgtgtgctgg acgctggacagggggcaaaagcacctgctcgtgggtcattctgatcaccgaactgaccatgccagccctgccgatggtcctccatggctc cctagtgccctggagaggaggtgtctagtcagagagtagtcctggaaggtggcctctgtgaggagccacggggacagcatcctgcagat ggtcctggcccttgtcccaccgacctgtctacaaggactgtcctcgtggaccctcccctctgcacaggagctggaccctgaagtcccttccc caccggccaggactggagcccctacccctctgttggaatccctgcccaccttcttctggaagtcggctctggagacatttctctcttcttccaa agctgggaactgctatctgttatctgcctgtccaggtctgaaagataggattgcccaggcagaaactgggactgacctatctcactctctccct gcttttacccttagggtgattctgggggcccacttgtctgtaatggtgtgcttcaaggtatcacgtcatggggcagtgaaccatgtgccctgcc cgaaaggccttccctgtacaccaaggtggtgcattaccggaagtggatcaaggacaccatcgtggccaacccctgagcacccctatcaac cccctattgtagtaaacttggaaccttggaaatgaccaggccaagactcaagcctccccagttctactgacctttgtccttaggtgtgaggtcc agggttgctaggaaaagaaatcagcagacacaggtgtagaccagagtgtttcttaaatggtgtaattttgtcctctctgtgtcctggggaatact ggccatgcctggagacatatcactcaatttctctgaggacacagataggatggggtgtctgtgttatttgtggggtacagagatgaaagagg ggtgggatccacactgagagagtggagagtgacatgtgctggacactgtccatgaagcactgagcagaagctggaggcacaacgcacc agacactcacagcaaggatggagctgaaaacataacccactctgtcctggaggcactgggaagcctagagaaggctgtgagccaagga gggagggtcttcctttggcatgggatggggatgaagtaaggagagggactggaccccctggaagctgattcactatggggggaggtgtat tgaagtcctccagacaaccctcagatttgatgatttcctagtagaactcacagaaataaagagctgttatactgtg (SEQ ID No: 20; GenBank Accession No. NM_001030047). In another embodiment, the KLK3 protein is encoded by residues 42-758 of SEQ ID No: 20. In another embodiment, the KLK3 protein is encoded by a homologue of SEQ ID No: 20. In another embodiment, the KLK3 protein is encoded by a variant of SEQ ID No: 20. In another embodiment, the KLK3 protein is encoded by an isomer of SEQ ID No: 20. In another embodiment, the KLK3 protein is encoded by a fragment of SEQ ID No: 20.

In another embodiment, the KLK3 protein has the sequence: MWVPVVFLTLSVTWIGAAPLILSRIVGGWECEKHSQPWQVLVASRGRAVCGGVLVHP QWVLTAAHCIRK (SEQ ID No: 21; GenBank Accession No. NP_001025221). In another embodiment, the KLK3 protein is a homologue of SEQ ID No: 21. In another embodiment, the KLK3 protein is a variant of SEQ ID No: 21. In another embodiment, the sequence of the KLK3 protein comprises SEQ ID No: 21. In another embodiment, the KLK3 protein is an isomer of SEQ ID No: 21. In another embodiment, the KLK3 protein is a fragment of SEQ ID No: 21. Each possibility represents a separate embodiment of the methods and compositions as disclosed herein.

In another embodiment, the KLK3 protein is encoded by a nucleotide molecule having the sequence: agccccaagcttaccacctgcacccggagagctgtgtcaccatgtgggtcccggttgtcttcctcacccttccgtgacgtggattggtgctgc acccctcatcctgtctcggattgtgggaggctgggagtgcgagaagcattcccaaccctggcaggtgcttgtggcctctcgtggcagggca gtctgcggcggtgttctggtgcacccccagtgggtcctcacagctgcccactgcatcaggaagtgagtaggggcctggggtctggggag caggtgtctgtgtcccagaggaataacagctgggcattttccccaggataacctctaaggccagccttgggactgggggagagagggaaa gttctggttcaggtcacatggggaggcagggttggggctggaccaccctccccatggctgcctgggtctccatctgtgttcctctatgtctcttt gtgtcgctttcattatgtctcttggtaactggcttcggttgtgtctctccgtgtgactattttgttctctctctccctctcttctctgtcttcagt (SEQ ID No: 22). In another embodiment, the KLK3 protein is encoded by residues 42-758 of SEQ ID No: 22. In another embodiment, the KLK3 protein is encoded by a homologue of SEQ ID No: 22. In another embodiment, the KLK3 protein is encoded by a variant of SEQ ID No: 22. In another embodiment, the KLK3 protein is encoded by an isomer of SEQ ID No: 22. In another embodiment, the KLK3 protein is encoded by a fragment of SEQ ID No: 22. Each possibility represents a separate embodiment of the methods and compositions as disclosed herein.

In another embodiment, the KLK3 protein that is the source of the KLK3 peptide has the sequence:

(SEQ ID No: 23)
MWVPVVFLTLSVTWIGAAPLILSRIVGGWECEKHSQPWQVLVASRGRA
VCGGVLVHPQWVLTAAHCIRNKSVILLGRHSLFHPEDTGQVFQVSHSE
PHPLYDMSLLKNRFLRPGDDSSIEPEEFLTPKKLQCVDLHVISNDVCA
QVHPQKVTKFMLCAGRWTGGKSTCSGDSGGPLVCNGVLQGITSWGSEP
CALPERPSLYTKVVHYRKWIKDTIVANP.

In another embodiment, the KLK3 protein is a homologue of SEQ ID No: 23. In another embodiment, the KLK3 protein is a variant of SEQ ID No: 23. In another embodiment, the KLK3 protein is an isomer of SEQ ID No: 23. In another embodiment, the KLK3 protein is a fragment of SEQ ID No: 23.

In another embodiment, the KLK3 protein is encoded by a nucleotide molecule having the sequence: agccccaagcttaccacctgcacccggagagctgtgtcaccatgtgggtcccggttgtcacctcaccctgtccgtgacgtggattggtgctg cacccctcatcctgtctcggattgtgggaggctgggagtgcgagaagcattcccaaccctggcaggtgcttgtggcctctcgtggcagggc agtctgcggcggtgttctggtgcacccccagtgggtcctcacagctgcccactgcatcaggaacaaaagcgtgatcttgctgggtcggcac agcctgtttcatcctgaagacacaggccaggtatttcaggtcagccacagcttcccacacccgctctacgatatgagcctcctgaagaatcg attcctcaggccaggtgatgactccagcattgaaccagaggagttcttgaccccaaagaaacttcagtgtgtggacctccatgttatttccaat gacgtgtgtgcgcaagttcaccctcagaaggtgaccaagttcatgctgtgtgctggacgctggacagggggcaaaagcacctgctcgggt gattctgggggcccacttgtctgtaatggtgtgcttcaaggtatcacgtcatggggcagtgaaccatgtgccctgcccgaaaggccttccctg tacaccaaggtggtgcattaccggaagtggatcaaggacaccatcgtggccaacccctgagcacccctatcaaccccctattgtagtaaact tggaaccttggaaatgaccaggccaagactcaagcctccccagttctactgacctttgtccttaggtgtgaggtccagggttgctaggaaaa gaaatcagcagacacaggtgtagaccagagtgtttcttaaatggtgtaattttgtcctctctgtgtcctggggaatactggccatgcctggaga catatcactcaatttctctgaggacacagataggatggggtgtctgtgttatttgtggggtacagagatgaaagaggggtgggatccacactg agagagtggagagtgacatgtgctggacactgtccatgaagcactgagcagaagctggaggcacaacgcaccagacactcacagcaag gatggagctgaaaacataacccactctgtcctggaggcactgggaagcctagagaaggctgtgagccaaggagggagggtcttcctttgg catgggatggggatgaagtaaggagagggactggaccccctggaagctgattcactatggggggaggtgtattgaagtcctccagacaa ccctcagatttgatgatttcctagtagaactcacagaaataaagagctgttatactgtg (SEQ ID No: 24). In another embodiment, the KLK3 protein is encoded by residues 42-758 of SEQ ID No: 24. In another embodiment, the KLK3 protein is encoded by a homologue of SEQ ID No: 24. In another embodiment, the KLK3 protein is encoded by a variant of SEQ ID No: 24. In another embodiment, the KLK3 protein is encoded by an isomer of SEQ ID No: 24. In another embodiment, the KLK3 protein is encoded by a fragment of SEQ ID No: 24. Each possibility represents a separate embodiment of the methods and compositions as disclosed herein.

In another embodiment, the KLK3 protein has the sequence: MWVPVVFLTLSVTWIGAAPLILSRIVGGWECEKHSQPWQVLVASRGRAVCGGVLVHP QWVLTAAHCIRKPGDDSSHDLMLLRLSEPAELTDAVKVMDLPTQEPALGTTCYASGW GSTEPEEFLTPKKLQCVDLHVISNDVCAQVHPQKVTKFMLCAGRWTGGKSTCSGDSGG PLVCNGVLQGITSWGSEPCALPERPSLYTKVVHYRKWIKDTIVANP (SEQ ID No: 25; GenBank Accession No. NP 001025219). In another embodiment, the KLK3 protein is a homologue of SEQ ID No: 25. In another embodiment, the KLK3 protein is a variant of SEQ ID No: 25. In another embodiment, the KLK3 protein is an isomer of SEQ ID No: 25. In another embodiment, the KLK3 protein is a fragment of SEQ ID No: 25. Each possibility represents a separate embodiment of the methods and compositions as disclosed herein.

In another embodiment, the KLK3 protein is encoded by a nucleotide molecule having the sequence: agccccaagcttaccacctgcacccggagagctgtgtcaccatgtgggtcccggttgtcttcctcaccctgtccgtgacgtggattggtgctg cacccctcatcctgtctcggattgtgggaggctgggagtgcgagaagcattcccaaccctggcaggtgcttgtggcctctcgtggcagggc agtctgcggcggtgttctggtgcacccccagtgggtcctcacagctgcccactgcatcaggaagccaggtgatgactccagccacgacct catgctgctccgcctgtcagagcctgccgagctcacggatgctgtgaaggtcatggacctgcccacccaggagccagcactggggacca cctgctacgcctcaggctggggcagcattgaaccagaggagttcttgaccccaaagaaacttcagtgtgtggacctccatgttatttccaatg acgtgtgtgcgcaagttcaccctcagaaggtgaccaagttcatgctgtgtgctggacgctggacagggggcaaaagcacctgctcgggtg attctgggggcccacttgtctgtaatggtgtgcttcaaggtatcacgtcatggggcagtgaaccatgtgccctgcccgaaaggccttccctgt acaccaaggtggtgcattacccaaggacaccatcgtggccaacccctgagcacccctatcaaccccctattgtagtaaacttggaaccttgg aaatgaccaggccaagactcaagcctccccagttctactgacctttgtccttaggtgtgaggtccagggttgctaggaaaagaaatcagcag acacaggtgtagaccagagtgtttcttaaatggtgtaattttgtcctctctgtgtcctggggaatactggccatgcctggagacatatcactcaat actctgaggacacagataggatggggtgtctgtgttatttgtggggtacagagatgaaagaggggtgggatccacactgagagagtggag agtgacatgtgctggacactgtccatgaagcactgagcagaagctggaggcacaacgcaccagacactcacagcaaggatggagctga aaacataacccactctgtcctggaggcactgggaagcctagagaaggctgtgagccaaggagggagggtcttcctttggcatgggatgg ggatgaagtaaggagagggactggaccccctggaagctgattcactatggggggaggtgtattgaagtcctccagacaaccctcagatttg atgatttcctagtagaactcacagaaataaagagctgttatactgtg (SEQ ID No: 26; GenBank Accession No. NM_001030048). In another embodiment, the KLK3 protein is encoded by residues 42-758 of SEQ ID No: 26. In another embodiment, the KLK3 protein is encoded by a homologue of SEQ ID No: 26. In another embodiment, the KLK3 protein is encoded by a variant of SEQ ID No: 26. In another embodiment, the KLK3 protein is encoded by an isomer of SEQ ID No: 26. In another embodiment, the KLK3 protein is encoded by a fragment of SEQ ID No: 26. Each possibility represents a separate embodiment of the methods and compositions as disclosed herein.

In another embodiment, the KLK3 protein has the sequence: MWVPVVFLTLSVTWIGAAPLILSRIVGGWECEKHSQPWQVLVASRGRAVCGGVLVHP QWVLTAAHCIRNKSVILLGRHSLFHPEDTGQVFQVSHSFPHPLYDMSLLKNRFLRPGD DSSHDLMLLRLSEPAELTDAVKVMDLPTQEPALGTTCYASGWGSTEPEEFLTPKKLQC VDLHVISNDVCAQVHPQKVTKFMLCAGRWTGGKSTCSGDSGGPLVCNGVLQGITSWG SEPCALPERPSLYTKVVHYRKWIKDTIVANP (SEQ ID No: 27; GenBank Accession No. NP_001639). In another embodiment, the KLK3 protein is a homologue of SEQ ID No: 27. In another embodiment, the KLK3 protein is a variant of SEQ ID No: 27. In another embodiment, the KLK3 protein is an isomer of SEQ ID No: 27. In another embodiment, the KLK3 protein is a fragment of SEQ ID No: 27.

In another embodiment, the KLK3 protein is encoded by a nucleotide molecule having the sequence: agccccaagcttaccacctgcacccggagagctgtgtcaccatgtgggtcccggttgtcacctcaccctgtccgtgacgtggattggtgctg cacccctcatcctgtctcggattgtgggaggctgggagtgcgagaagcattcccaaccctggcaggtgcttgtggcctctcgtggcagggc agtctgcggcggtgttctggtgcacccccagtgggtcctcacagctgcccactgcatcaggaacaaaagcgtgatcttgctgggtcggcac agcctgtttcatcctgaagacacaggccaggtatttcaggtcagccacagcttcccacacccgctctacgatatgagcctcctgaagaatcg attcctcaggccaggtgatgactccagccacgacctcatgctgctccgcctgtcagagcctgccgagctcacggatgctgtgaaggtcatg gacctgcccacccaggagccagcactggggaccacctgctacgcctcaggctggggcagcattgaaccagaggagttcttgaccccaa agaaacttcagtgtgtggacctccatgttatttccaatgacgtgtgtgcgcaagttcaccctcagaaggtgaccaagttcatgctgtgtgctgg acgctggacagggggcaaaagcacctgctcgggtgattctgggggcccacttgtctgtaatggtgtgcttcaaggtatcacgtcatggggc agtgaaccatgtgccctgcccgaaaggccttccctgtacaccaaggtggtgcattaccggaagtggatcaaggacaccatcgtggccaac ccctgagcacccctatcaaccccctattgtagtaaacttggaaccttggaaatgaccaggccaagactcaagcctccccagttctactgacct ttgtccttaggtgtgaggtccagggttgctaggaaaagaaatcagcagacacaggtgtagaccagagtgtttcttaaatggtgtaattttgtcct ctctgtgtcctggggaatactggccatgcctggagacatatcactcaatttctctgaggacacagataggatggggtgtctgtgttatttgtggg gtacagagatgaaagaggggtgggatccacactgagagagtggagagtgacatgtgctggacactgtccatgaagcactgagcagaag ctggaggcacaacgcaccagacactcacagcaaggatggagctgaaaacataacccactctgtcctggaggcactgggaagcctagag aaggctgtgagccaaggagggagggtcttcctttggcatgggatggggatgaagtaaggagagggactggaccccctggaagctgattc actatggggggaggtgtattgaagtcctccagacaaccctcagatttgatgatttcctagtagaactcacagaaataaagagctgttatactgt g (SEQ ID No: 28; GenBank Accession No. NM_001648). In another embodiment, the KLK3 protein is encoded by residues 42-827 of SEQ ID No: 28. In another embodiment, the KLK3 protein is encoded by a homologue of SEQ ID No: 28. In another embodiment, the KLK3 protein is encoded by a variant of SEQ ID No: 28. In another embodiment, the KLK3 protein is encoded by an isomer of SEQ ID No: 28. In another embodiment, the KLK3 protein is encoded by a fragment of SEQ ID No: 28.

In another embodiment, the KLK3 protein has the sequence: MWVPVVFLTLSVTWIGAAPLILSRIVGGWECEKHSQPWQVLVASRGRAVCGGVLVHP QWVLTAAHCIRNKSVILLGRHSLFHPEDTGQVFQVSHSFPHPLYDMSLLKNRFLRPGD DSSHDLMLLRLSEPAELTDAVKVMDLPTQEPALGTTCYASGWGSTEPEEFLTPKKLQC VDLHVISNDVCAQVHPQKVTKFMLCAGRWTGGKSTCSGDSGGPLVCNGVLQGITSWG SEPCALPERPSLYTKVVHYRKWIKDTIVANP (SEQ ID No: 29 GenBank Accession No. AAX29407.1). In another embodiment, the KLK3 protein is a homologue of SEQ ID No: 29. In another embodiment, the KLK3 protein is a variant of SEQ ID No: 29. In another embodiment, the KLK3 protein is an isomer of SEQ ID No: 29. In another embodiment, the sequence of the KLK3 protein comprises SEQ ID No: 29. In another embodiment, the KLK3 protein is a fragment of SEQ ID No: 29.

In another embodiment, the KLK3 protein is encoded by a nucleotide molecule having the sequence: gggggagccccaagcttaccacctgcacccggagagctgtgtcaccatgtgggtcccggttgtcttcctcaccctgtccgtgacgtggattg gtgctgcacccctcatcctgtctcggattgtgggaggctgggagtgcgagaagcattcccaaccctggcaggtgcttgtggcctctcgtggc agggcagtctgcggcggtgttctggtgcacccccagtgggtcctcacagctgcccactgcatcaggaacaaaagcgtgatcttgctgggtc ggcacagcctgtttcatcctgaagacacaggccaggtatttcaggtcagccacagcttcccacacccgctctacgatatgagcctcctgaag aatcgattcctcaggccaggtgatgactccagccacgacctcatgctgctccgcctgtcagagcctgccgagctcacggatgctgtgaagg tcatggacctgcccacccaggagccagcactggggaccacctgctacgcctcaggctggggcagcattgaaccagaggagttcttgacc ccaaagaaacttcagtgtgtggacctccatgttatttccaatgacgtgtgtgcgcaagttcaccctcagaaggtgaccaagttcatgctgtgtg ctggacgctggacagggggcaaaagcacctgctcgggtgattctgggggcccacttgtctgtaatggtgtgcttcaaggtatcacgtcatg gggcagtgaaccatgtgccctgcccgaaaggccttccctgtacaccaaggtggtgcattaccggaagtggatcaaggacaccatcgtggc caacccctgagcacccctatcaactccctattgtagtaaacttggaaccttggaaatgaccaggccaagactcaggcctccccagttctactg acctttgtccttaggtgtgaggtccagggttgctaggaaaagaaatcagcagacacaggtgtagaccagagtgtttcttaaatggtgtaatttt gtcctctctgtgtcctggggaatactggccatgcctggagacatatcactcaatttctctgaggacacagataggatggggtgtctgtgttattt gtggggtacagagatgaaagaggggtgggatccacactgagagagtggagagtgacatgtgctggacactgtccatgaagcactgagc agaagctggaggcacaacgcaccagacactcacagcaaggatggagctgaaaacataacccactctgtcctggaggcactgggaagcc tagagaaggctgtgagccaaggagggagggtcttcctttggcatgggatggggatgaagtagggagagggactggaccccctggaagc tgattcactatggggggaggtgtattgaagtcctccagacaaccctcagatttgatgatttcctagtagaactcacagaaataaagagctgttat actgcgaaaaaaaaaaaaaaaaaaaaaaaaaa (SEQ ID No: 30; GenBank Accession No. BC056665). In another embodiment, the KLK3 protein is encoded by residues 47-832 of SEQ ID No: 30. In another embodiment, the KLK3 protein is encoded by a homologue of SEQ ID No: 30. In another embodiment, the KLK3 protein is encoded by a variant of SEQ ID No: 30. In another embodiment, the KLK3 protein is encoded by an isomer of SEQ ID No: 30. In another embodiment, the KLK3 protein is encoded by a fragment of SEQ ID No: 30.

In another embodiment, the KLK3 protein has the sequence: MWVPVVFLTLSVTWIGAAPLILSRIVGGWECEKHSQPWQVLVASRGRAVCGGVLVHP QWVLTAAHCIRNKSVILLGRHSLFHPEDTGQVFQVSHSFPHPLYDMSLLKNRFLRPGD DSSIEPEEFLTPKKLQCVDLHVISNDVCAQVHPQKVTKFMLCAGRWTGGKSTCSGDSG GPLVCNGVLQGITSWGSEPCALPERPSLYTKVVHYRKWIKDTIVA (SEQ ID No: 31; GenBank Accession No. AJ459782). In another embodiment, the KLK3 protein is a homologue of SEQ ID No: 31. In another embodiment, the KLK3 protein is a variant of SEQ ID No: 31. In another embodiment, the KLK3 protein is an isomer of SEQ ID No: 31. In another embodiment, the KLK3 protein is a fragment of SEQ ID No: 31.

In another embodiment, the KLK3 protein has the sequence: MWVPVVFLTLSVTWIGAAPLILSRIVGGWECEKHSQPWQVLVASRGRAVCGGVLVHP QWVLTAAHCIRNKSVILLGRHSLFHPEDTGQVFQVSHSFPHPLYDMSLLKNRFLRPGD DSSHDLMLLRLSEPAELTDAVKVMDLPTQEPALGTTCYASGWGSIEPEEFLTPKKLQC VDLHVISNDVCAQVHPQKVTKFMLCAGRWTGGKSTCSVSHPYSQDLEGKGEWGP (SEQ ID No: 32, GenBank Accession No. AJ512346). In another embodiment, the KLK3 protein is a homologue of SEQ ID No: 32. In another embodiment, the KLK3 protein is a variant of SEQ ID No: 32. In another embodiment, the KLK3 protein is an isomer of SEQ ID No: 32. In another embodiment, the sequence of the KLK3 protein comprises SEQ ID No: 32. In another embodiment, the KLK3 protein is a fragment of SEQ ID No: 32.

In another embodiment, the KLK3 protein has the sequence: MWVPVVFLTLSVTWIGERGHGWGDAGEGASPDCQAEALSPPTQHPSPDRELGSFLSLP APLQAHTPSPSILQQSSLPHQVPAPSHLPQNFLPIAQPAPCSQLLY (SEQ ID No: 33; GenBank Accession No. AJ459784). In another embodiment, the KLK3 protein is a homologue of SEQ ID No: 33. In another embodiment, the KLK3 protein is a variant of SEQ ID No: 33. In another embodiment, the sequence of the KLK3 protein comprises SEQ ID No: 33. In another embodiment, the KLK3 protein is an isomer of SEQ ID No: 33. In another embodiment, the KLK3 protein is a fragment of SEQ ID No: 33.

In another embodiment, the KLK3 protein has the sequence: MWVPVVFLTLSVTWIGAAPLILSRIVGGWECEKHSQPWQVLVASRGRAVCGGVLVHP QWVLTAAHCIRNKSVILLGRHSLFHPEDTGQVFQVSHSFPHPLYDMSLLKNRFLRPGD DSSHDLMLLRLSEPAELTDAVKVMDLPTQEPALGTTCYASGWGSIEPEEFLTPKKLQC VDLHVISNDVCAQVHPQKVTKFMLCAGRWTGGKSTCSGDSGGPLVCNGVLQGITSWG SEPCALPERPSLYTKVVHYRKWIKDTIVANP (SEQ ID NO: 34 GenBank Accession No. AJ459783). In another embodiment, the KLK3 protein is a homologue of SEQ ID No: 34. In another embodiment, the KLK3 protein is a variant of SEQ ID No: 34. In another embodiment, the KLK3 protein is an isomer of SEQ ID No: 34. In another embodiment, the KLK3 protein is a fragment of SEQ ID No: 34.

In another embodiment, the KLK3 protein is encoded by a nucleotide molecule having the sequence: aagtttcccttctcccagtccaagaccccaaatcaccacaaaggacccaatccccagactcaagatatggtctgggcgctgtcttgtgtctcct accctgatccctgggttcaactctgctcccagagcatgaagcctctccaccagcaccagccaccaacctgcaaacctagggaagattgaca gaattcccagcctacccagctccccctgcccatgtcccaggactcccagccttggttctctgcccccgtgtcttttcaaacccacatcctaaat ccatctcctatccgagtcccccagttcctcctgtcaaccctgattcccctgatctagcaccccctctgcaggtgctgcacccctcatcctgtctc ggattgtgggaggctgggagtgcgagaagcattcccaaccctggcaggtgcttgtagcctctcgtggcagggcagtctgcggcggtgttct ggtgcacccccagtgggtcctcacagctacccactgcatcaggaacaaaagcgtgatcttgctgggtcggcacagcctgtttcatcctgaa gacacaggccaggtatttcaggtcagccacagcttcccacacccgctctacgatatgagcctcctgaagaatcgattcctcaggccaggtg atgactccagccacgacctcatgctgctccgcctgtcagagcctgccgagctcacggatgctatgaaggtcatggacctgcccacccagg agccagcactggggaccacctgctacgcctcaggctggggcagcattgaaccagaggagttcttgaccccaaagaaacttcagtgtgtgg acctccatgttatttccaatgacgtgtgtgcgcaagttcaccctcagaaggtgaccaagttcatgctgtgtgctggacgctggacagggggc aaaagcacctgctcgggtgattctgggggcccacttgtctgtaatggtgtgcttcaaggtatcacgtcatggggcagtgaaccatgtgccctg cccgaaaggccttccctgtacaccaaggtggtgcattaccggaagtggatcaaggacaccatcgtggccaacccctgagcacccctatca actccctattgtagtaaacttggaaccttggaaatgaccaggccaagactcaggcctccccagttctactgacctttgtccttaggtgtgaggt ccagggttgctaggaaaagaaatcagcagacacaggtgtagaccagagtgtttcttaaatggtgtaattttgtcctctctgtgtcctggggaat actggccatgcctggagacatatcactcaatttctctgaggacacagataggatggggtgtctgtgttatttgtggggtacagagatgaaaga ggggtgggatccacactgagagagtggagagtgacatgtgctggacactgtccatgaagcactgagcagaagctggaggcacaacgca ccagacactcacagcaaggatggagctgaaaacataacccactctgtcctggaggcactgggaagcctagagaaggctgtgaaccaag gagggagggtcttcctttggcatgggatggggatgaagtaaggagagggactgaccccctggaagctgattcactatggggggaggtgt attgaagtcctccagacaaccctcagatttgatgatttcctagtagaactcacagaaataaagagctgttatactgtgaa (SEQ ID No: 35; GenBank Accession No. X07730). In another embodiment, the KLK3 protein is encoded by residues 67-1088 of SEQ ID No: 35. In another embodiment, the KLK3 protein is encoded by a homologue of SEQ ID No: 35. In another embodiment, the KLK3 protein is encoded by a variant of SEQ ID No: 35. In another embodiment, the KLK3 protein is encoded by an isomer of SEQ ID No: 35. In another embodiment, the KLK3 protein is encoded by a fragment of SEQ ID No: 35.

In another embodiment, the KLK3 protein has the sequence:

MWVPVVFLTLSVTWIGAAPLILSRIVGGWECEKHSQPWQVLVASRGRAVCG GVLVHPQWVLTAAHCIRK (SEQ ID No: 36; GenBank Accession No. NM_001030050). In another embodiment, the KLK3 protein is a homologue of SEQ ID No: 36. In another embodiment, the KLK3 protein is a variant of SEQ ID No: 36. In another embodiment, the sequence of the KLK3 protein comprises SEQ ID No: 36. In another embodiment, the KLK3 protein is an isomer of SEQ ID No: 36. In another embodiment, the KLK3 protein is a fragment of SEQ ID No: 36.

In another embodiment, the KLK3 protein that is the source of the KLK3 peptide has the sequence:

MWVPVVFLTLSVTWIGAAPLILSRIVGGWECEKHSQPWQVLVASRGRAVCG GVLVHPQWVLTAAHORNKSVILLGRHSLFHPEDTGQVFQVSHSFPHPLYDMSLLKNR FLRPGDDSSIEPEEFLTPKKLQCVDLHVISNDVCAQVHPQKVTKFMLCAGRWTGGKST CSGDSGGPLVCNGVLQGITSWGSEPCALPERPSLYTKVVHYRKWIKDTIVANP (SEQ ID No: 37; GenBank Accession No. NM_001064049). In another embodiment, the KLK3 protein is a homologue of SEQ ID No: 37. In another embodiment, the KLK3 protein is a variant of SEQ ID No: 37. In another embodiment, the KLK3 protein is an isomer of SEQ ID No: 37. In another embodiment, the KLK3 protein is a fragment of SEQ ID No: 37.

In another embodiment, the KLK3 protein has the sequence:

MWVPVVFLTLSVTWIGAAPLILSRIVGGWECEKHSQPWQVLVASRGRAVCG GVLVHPQWVLTAAHCIRKPGDDSSHDLMLLRLSEPAELTDAVKVMDLPTQEPALGTT CYASGWGSIEPEEFLTPKKLQCVDLHVISNDVCAQVHPQKVTKFMLCAGRWTGGKST CSGDSGGPLVCNGVLQGITSWGSEPCALPERPSLYTKVVHYRKWIKDTIVANP (SEQ ID No: 38; GenBank Accession No. NM_001030048). In another embodiment, the KLK3 protein is a homologue of SEQ ID No: 38. In another embodiment, the KLK3 protein is a variant of SEQ ID No: 38. In another embodiment, the KLK3 protein is an isomer of SEQ ID No: 38. In another embodiment, the KLK3 protein is a fragment of SEQ ID No: 38.

In another embodiment, the KLK3 protein is encoded by a sequence set forth in one of the following GenBank Accession Numbers: BC005307, AJ310938, AJ310937, AF335478, AF335477, M27274, and M26663. In another embodiment, the KLK3 protein is encoded by a sequence set forth in one of the above GenBank Accession Numbers.

In another embodiment, the KLK3 protein is encoded by a sequence set forth in one of the following GenBank Accession Numbers: NM_001030050, NM_001030049, NM_001030048, NM_001030047, NM_001648, AJ459782, AJ512346, or AJ459784. Each possibility represents a separate embodiment of the methods and compositions as disclosed herein. In one embodiment, the KLK3 protein is encoded by a variation of any of the sequences described herein wherein the sequence lacks MWVPVVFLTLSVTWIGAAPLILSR (SEQ ID NO: 39).

In another embodiment, the KLK3 protein has the sequence that comprises a sequence set forth in one of the following GenBank Accession Numbers: X13943, X13942, X13940, X13941, and X13944.

In another embodiment, the KLK3 protein is any other KLK3 protein known in the art.

In another embodiment, the KLK3 peptide is any other KLK3 peptide known in the art. In another embodiment, the KLK3 peptide is a fragment of any other KLK3 peptide known in the art. Each type of KLK3 peptide represents a separate embodiment of the methods and compositions as disclosed herein.

“KLK3 peptide” refers, in another embodiment, to a full-length KLK3 protein. In another embodiment, the term refers to a fragment of a KLK3 protein. In another embodiment, the term refers to a fragment of a KLK3 protein that is lacking the KLK3 signal peptide. In another embodiment, the term refers to a KLK3 protein that contains the entire KLK3 sequence except the KLK3 signal peptide. “KLK3 signal sequence” refers, in another embodiment, to any signal sequence found in nature on a KLK3 protein. In another embodiment, a KLK3 protein of methods and compositions as disclosed herein does not contain any signal sequence. Each possibility represents a separate embodiment of the methods and compositions as disclosed herein.

In another embodiment, the kallikrein-related peptidase 3 (KLK3 protein) that is the source of a KLK3 peptide for use in the methods and compositions as disclosed herein is a PSA protein. In another embodiment, the KLK3 protein is a P-30 antigen protein. In another embodiment, the KLK3 protein is a gamma-seminoprotein protein. In another embodiment, the KLK3 protein is a kallikrein 3 protein. In another embodiment, the KLK3 protein is a semenogelase protein. In another embodiment, the KLK3 protein is a seminin protein. In another embodiment, the KLK3 protein is any other type of KLK3 protein that is known in the art. Each possibility represents a separate embodiment of the methods and compositions as disclosed herein.

In another embodiment, the KLK3 protein is a splice variant 1 KLK3 protein. In another embodiment, the KLK3 protein is a splice variant 2 KLK3 protein. In another embodiment, the KLK3 protein is a splice variant 3 KLK3 protein. In another embodiment, the KLK3 protein is a transcript variant 1 KLK3 protein. In another embodiment, the KLK3 protein is a transcript variant 2 KLK3 protein. In another embodiment, the KLK3 protein is a transcript variant 3 KLK3 protein. In another embodiment, the KLK3 protein is a transcript variant 4 KLK3 protein. In another embodiment, the KLK3 protein is a transcript variant 5 KLK3 protein. In another embodiment, the KLK3 protein is a transcript variant 6 KLK3 protein. In another embodiment, the KLK3 protein is a splice variant RP5 KLK3 protein. In another embodiment, the KLK3 protein is any other splice variant KLK3 protein known in the art. In another embodiment, the KLK3 protein is any other transcript variant KLK3 protein known in the art. Each possibility represents a separate embodiment of the methods and compositions as disclosed herein.

In another embodiment, the KLK3 protein is a mature KLK3 protein. In another embodiment, the KLK3 protein is a pro-KLK3 protein. In another embodiment, the leader sequence has been removed from a mature KLK3 protein of methods and compositions as disclosed herein. Each possibility represents a separate embodiment of the methods and compositions as disclosed herein.

In another embodiment, the KLK3 protein that is the source of a KLK3 peptide of methods and compositions as disclosed herein is a human KLK3 protein. In another embodiment, the KLK3 protein is a primate KLK3 protein. In another embodiment, the KLK3 protein is a KLK3 protein of any other species known in the art. In another embodiment, one of the above KLK3 proteins is referred to in the art as a “KLK3 protein.”

In one embodiment, a recombinant polypeptide disclosed herein comprising a truncated LLO fused to a PSA protein disclosed herein is encoded by a sequence comprising:

ATGAAAAAAATAATGCTAGTTTTTATTACACTTATATTAGTTAGTCTACCA ATTGCGCAACAAACTGAAGCAAAGGATGCATCTGCATTCAATAAAGAAAATTCAA TTTCATCCATGGCACCACCAGCATCTCCGCCTGCAAGTCCTAAGACGCCAATCGAA AAGAAACACGCGGATGAAATCGATAAGTATATACAAGGATTGGATTACAATAAAA ACAATGTATTAGTATACCACGGAGATGCAGTGACAAATGTGCCGCCAAGAAAAGG TTACAAAGATGGAAATGAATATATTGTTGTGGAGAAAAAGAAGAAATCCATCAAT CAAAATAATGCAGACATTCAAGTTGTGAATGCAATTTCGAGCCTAACCTATCCAGG TGCTCTCGTAAAAGCGAATTCGGAATTAGTAGAAAATCAACCAGATGTTCTCCCTG TAAAACGTGATTCATTAACACTCAGCATTGATTTGCCAGGTATGACTAATCAAGAC AATAAAATAGTTGTAAAAAATGCCACTAAATCAAACGTTAACAACGCAGTAAATA CATTAGTGGAAAGATGGAATGAAAAATATGCTCAAGCTTATCCAAATGTAAGTGC AAAAATTGATTATGATGACGAAATGGCTTACAGTGAATCACAATTAATTGCGAAAT TTGGTACAGCATTTAAAGCTGTAAATAATAGCTTGAATGTAAACTTCGGCGCAATC AGTGAAGGGAAAATGCAAGAAGAAGTCATTAGTTTTAAACAAATTTACTATAACG TGAATGTTAATGAACCTACAAGACCTTCCAGATTTTTCGGCAAAGCTGTTACTAAA GAGCAGTTGCAAGCGCTTGGAGTGAATGCAGAAAATCCTCCTGCATATATCTCAAG TGTGGCGTATGGCCGTCAAGTTTATTTGAAATTATCAACTAATTCCCATAGTACTAA AGTAAAAGCTGCTTTTGATGCTGCCGTAAGCGGAAAATCTGTCTCAGGTGATGTAG AACTAACAAATATCATCAAAAATTCTTCCTTCAAAGCCGTAATTTACGGAGGTTCC GCAAAAGATGAAGTTCAAATCATCGACGGCAACCTCGGAGACTTACGCGATATTTT GAAAAAAGGCGCTACTTTTAATCGAGAAACACCAGGAGTTCCCATTGCTTATACAA CAAACTTCCTAAAAGACAATGAATTAGCTGTTATTAAAAACAACTCAGAATATATT GAAACAACTTCAAAAGCTTATACAGATGGAAAAATTAACATCGATCACTCTGGAG GATACGTTGCTCAATTCAACATTTCTTGGGATGAAGTAAATTATGATCTCGAGattgtg ggaggctgggagtgcgagaagcattcccaaccctggcaggtgcttgtggcctctcgtggcagggcagtctgcggcggtgttctggtgc acccccagtgggtcctcacagctgcccactgcatcaggaacaaaagcgtgatcttgctgggtcggcacagcctgtttcatcctgaagac acaggccaggtatttcaggtcagccacagcttcccacacccgctctacgatatgagcctcctgaagaatcgattcctcaggccaggtga tgactccagccacgacctcatgctgctccgcctgtcagagcctgccgagctcacggatgctgtgaaggtcatggacctgcccacccag gagccagcactggggaccacctgctacgcctcaggctggggcagcattgaaccagaggagttcttgaccccaaagaaacttcagtgt gtggacctccatgttatttccaatgacgtgtgtgcgcaagttcaccctcagaaggtgaccaagttcatgctgtgtgctggacgctggaca gggggcaaaagcacctgctcgggtgattctgggggcccacttgtctgttatggtgtgcttcaaggtatcacgtcatggggcagtgaacc atgtgccctgcccgaaaggccttccctgtacaccaaggtggtgcattaccggaagtggatcaaggacaccatcgtggccaacccc (SEQ ID NO: 91). In another embodiment, the fusion protein is encoded by a homologue of SEQ ID No: 91. In another embodiment, the fusion protein is encoded by a variant of SEQ ID No: 91. In another embodiment, the fusion protein is encoded by an isomer of SEQ ID No: 91. In one embodiment, the “ctcgag” sequence within the fusion protein represents a Xho I restriction site used to ligate the tumor antigen to truncated LLO in the plasmid.

In another embodiment, a recombinant polypeptide disclosed herein comprising a truncated LLO fused to a PSA protein disclosed herein comprises the following sequence:

(SEQ ID NO: 92)
MKKIMLVFITLILVSLPIAQQTEAKDASAFNKENSISSMAPPASPPAS
PKTPIEKKHADEIDKYIQGLDYNKNNVLVYHGDAVTNVPPRKGYKDGN
EYIVVEKKKKSINQNNADIQVVNAISSLTYPGALVKANSELVENQPDV
LPVKRDSLTLSIDLPGMTNQDNKIVVKNATKSNVNNAVNTLVERWNEK
YAQAYPNVSAKIDYDDEMAYSESQLIAKFGTAFKAVNNSLNVNFGAIS
EGKMQEEVISFKQIYYNVNVNEPTRPSRFFGKAVTKEQLQALGVNAEN
PPAYISSVAYGRQVYLKLSTNSHSTKVKAAFDAAVSGKSVSGDVELTN
IIKNSSFKAVIYGGSAKDEVTIDGNLGDLRDILKKGATFNRETPGVPI
AYTTNFLKDNELAVIKNNSEYIETTSKAYTDGKINIDHSGGYVAQFNI
SWDEVNYDLEIVGGWECEKHSQPWQVLVASRGRAVCGGVLVHPQWVLT
AAHQRNKSVILLGRHSLFHPEDTGQVFQVSHSFPHPLYDMSLLKNRFL
RPGDDSSHDLMLLRLSEPAELTDAVKVMDLPTQEPALGTTCYASGWGS
IEPEEFLTPKKLQCVDLHVISNDVCAQVHPQKVTKFMLCAGRWTGGKS
TCSGDSGGPLVCYGVLQGITSWGSEPCALPERPSLYTKVVHYRKWIKD
TIVANP.
(PSA sequence is underlined)

In another embodiment, the tLLO-PSA fusion protein is a homologue of SEQ ID NO: 92. In another embodiment, the tLLO-PSA fusion protein is a variant of SEQ ID NO: 92. In another embodiment, the tLLO-PSA fusion protein is an isomer of SEQ ID NO: 92. In another embodiment, the tLLO-PSA fusion protein is a fragment of SEQ ID NO: 92.

In one embodiment, the recombinant Listeria strain as disclosed herein comprises a nucleic acid molecule encoding a tumor associated antigen, wherein the antigen comprises an HPV-E7 protein. In one embodiment, the recombinant Listeria strain as disclosed herein comprises a nucleic acid molecule encoding HPV-E7 protein.

In one embodiment, either a whole E7 protein or a fragment thereof is fused to a LLO protein or truncation or peptide thereof, an ActA protein or truncation or peptide thereof, or a PEST-like sequence-containing peptide to generate a recombinant polypeptide or peptide of the composition and methods of the present invention. The E7 protein that is utilized (either whole or as the source of the fragments) has, in another embodiment, the sequence

MHGDTPTLHEYMLDLQPETTDLYCYEQLNDSSEEEDEIDGPAGQAEPDRAH YNIVTFCCKCDSTLRLCVQSTHVDIRTLEDLLMGTLGIVCPICSQKP (SEQ ID No: 40). In another embodiment, the E7 protein is a homologue of SEQ ID No: 40. In another embodiment, the E7 protein is a variant of SEQ ID No: 40. In another embodiment, the E7 protein is an isomer of SEQ ID No: 40. In another embodiment, the E7 protein is a fragment of SEQ ID No: 40. In another embodiment, the E7 protein is a fragment of a homologue of SEQ ID No: 40. In another embodiment, the E7 protein is a fragment of a variant of SEQ ID No: 40. In another embodiment, the E7 protein is a fragment of an isomer of SEQ ID No: 40. Each possibility represents a separate embodiment of the present invention.

In another embodiment, the sequence of the E7 protein is:

MHGPKATLQDIVLHLEPQNEIPVDLLCHEQLSDSEEENDEIDGVNHQHLPAR RAEPQRHTMLCMCCKCEARIELVVESSADDLRAFQQLFLNTLSFVCPWCASQQ (SEQ ID No: 41). In another embodiment, the E6 protein is a homologue of SEQ ID No: 41. In another embodiment, the E6 protein is a variant of SEQ ID No: 41. In another embodiment, the E6 protein is an isomer of SEQ ID No: 41. In another embodiment, the E6 protein is a fragment of SEQ ID No: 41. In another embodiment, the E6 protein is a fragment of a homologue of SEQ ID No: 41. In another embodiment, the E6 protein is a fragment of a variant of SEQ ID No: 41. In another embodiment, the E6 protein is a fragment of an isomer of SEQ ID No: 41. Each possibility represents a separate embodiment of the present invention.

In another embodiment, the E7 protein has a sequence set forth in one of the following GenBank entries: M24215, NC_004500, V01116, X62843, or M14119. In another embodiment, the E7 protein is a homologue of a sequence from one of the above GenBank entries. In another embodiment, the E7 protein is a variant of a sequence from one of the above GenBank entries. In another embodiment, the E7 protein is an isomer of a sequence from one of the above GenBank entries. In another embodiment, the E7 protein is a fragment of a sequence from one of the above GenBank entries. In another embodiment, the E7 protein is a fragment of a homologue of a sequence from one of the above GenBank entries. In another embodiment, the E7 protein is a fragment of a variant of a sequence from one of the above GenBank entries. In another embodiment, the E7 protein is a fragment of an isomer of a sequence from one of the above GenBank entries. Each possibility represents a separate embodiment of the present invention.

In one embodiment the HPV antigen is an HPV 16. In another embodiment, the HPV is an HPV-18. In another embodiment, the HPV is selected from HPV-16 and HPV-18. In another embodiment, the HPV is an HPV-31. In another embodiment, the HPV is an HPV-35. In another embodiment, the HPV is an HPV-39. In another embodiment, the HPV is an HPV-45. In another embodiment, the HPV is an HPV-51. In another embodiment, the HPV is an HPV-52. In another embodiment, the HPV is an HPV-58. In another embodiment, the HPV is a high-risk HPV type. In another embodiment, the HPV is a mucosal HPV type. Each possibility represents a separate embodiment of the present invention.

In one embodiment, the HPV E6 is from HPV-16. In another embodiment, the HPV E7 is from HPV-16. In another embodiment, the HPV-E6 is from HPV-18. In another embodiment, the HPV-E7 is from HPV-18. In another embodiment, an HPV E6 antigen is utilized instead of or in addition to an E7 antigen in a composition or method of the present invention for treating or ameliorating an HPV-mediated disease, disorder, or symptom. In another embodiment, an HPV-16 E6 and E7 is utilized instead of or in combination with an HPV-18 E6 and E7. In such an embodiment, the recombinant Listeria may express the HPV-16 E6 and E7 from the chromosome and the HPV-18 E6 and E7 from a plasmid, or vice versa. In another embodiment, the HPV-16 E6 and E7 antigens and the HPV-18 E6 and E7 antigens are expressed from a plasmid present in a recombinant Listeria disclosed herein. In another embodiment, the HPV-16 E6 and E7 antigens and the HPV-18 E6 and E7 antigens are expressed from the chromosome of a recombinant Listeria disclosed herein. In another embodiment, the HPV-16 E6 and E7 antigens and the HPV-18 E6 and E7 antigens are expressed in any combination of the above embodiments, including where each E6 and E7 antigen from each HPV strain is expressed from either the plasmid or the chromosome.

In another embodiment, either a whole E7 protein or a fragment thereof is fused to a LLO protein, ActA protein, or PEST amino acid sequence-containing peptide to generate a recombinant polypeptide disclosed herein. In one embodiment, the E7 protein that is utilized (either whole or as the source of the fragments) comprises the amino acid sequence set forth in SEQ ID NO: 93

H G D T P T L H E Y M L D L Q P E T T D L Y C Y E Q L N D S S E E E D E I D G P A G Q A E P D R A H Y N I V T F C C K C D S T L R L C V Q S T H V D I R T L E D L L M G T L G I V C P I C S Q K P (SEQ ID NO: 93). In another embodiment, the E7 protein is a homologue of SEQ ID No: 93. In another embodiment, the E7 protein is a variant of SEQ ID No: 93. In another embodiment, the E7 protein is an isomer of SEQ ID No: 93. In another embodiment, the E7 protein is a fragment of SEQ ID No: 93. In another embodiment, the E7 protein is a fragment of a homologue of SEQ ID No: 93. In another embodiment, the E7 protein is a fragment of a variant of SEQ ID No: 93. In another embodiment, the E7 protein is a fragment of an isomer of SEQ ID No: 93.

In another embodiment, the amino acid sequence of a truncated LLO fused to an E7 protein comprises the following amino acid sequence:

(SEQ ID NO: 94)
MKKINILVFITLILVSLPIAQQTEAKDASAFNKENSISSMAPPASPPA
SPKTPIEKKHADEIDKYIQGLDYNKNNVLVYHGDAVTNVPPRKGYKDG
NEYIVVEKKKKSINQNNADIQVVNAISSLTYPGALVKANSELVENQPD
VLPVKRDSLTLSIDLPGMTNQDNKIVVKNATKSNVNNAVNTLVERWNE
KYAQAYPNVSAKIDYDDEMAYSESQLIAKFGTAFKAVNNSLNVNFGAI
SEGKMQEEVISFKQIYYNVNVNEPTRPSRFFGKAVTKEQLQALGVNAE
NPPAYISSVAYGRQVYLKLSTNSHSTKVKAAFDAAVSGKSVSGDVELT
NIIKNSSFKAVIYGGSAKDEVQIIDGNLGDLRDILKKGATFNRETPGV
PIAYTTNFLKDNELAVIKNNSEYIETTSKAYTDGKINIDHSGGYVAQF
NISWDEVNYDLEHGDTPTLHEYMLDLQPETTDLYCYEQLNDSSEEEDE
IDGPAGQAEPDRAHYNIVTFCCKCDSTLRLCVQSTHVDIRTLEDLLMG
TLGIVCPICSQKP.

In another embodiment, the fusion protein of tLLO-E7 is a homologue of SEQ ID No: 94. In another embodiment, the fusion protein is a variant of SEQ ID No: 94. In another embodiment, the tLLO-E7 fusion protein is an isomer of SEQ ID No: 94. In another embodiment, the tLLO-E7 fusion protein is a fragment of SEQ ID No: 94. In another embodiment, the tLLO-E7 fusion protein is a fragment of a homologue of SEQ ID No: 94. In another embodiment, the tLLO-E7 fusion protein is a fragment of a variant of SEQ ID No: 94. In another embodiment, the tLLO-E7 fusion protein is a fragment of an isomer of SEQ ID No: 94.

In one embodiment, the recombinant Listeria strain as disclosed herein comprises a nucleic acid molecule encoding a tumor associated antigen, wherein the tumor associated antigen comprises an Her-2/neu peptide. In one embodiment, a tumor associated antigen comprises an Her-2/neu antigen. In one embodiment the Her-2/neu peptide comprises a chimeric Her-2/neu antigen (cHer-2).

In one embodiment, the attenuated auxotrophic Listeria strain is based on a Listeria vaccine vector which is attenuated due to the deletion of virulence gene actA and retains the plasmid for Her2/neu expression in vivo and in vitro by complementation of dal gene. In one embodiment, the Listeria strain expresses and secretes a chimeric Her2/neu protein fused to the first 441 amino acids of listeriolysin O (LLO). In another embodiment, the Listeria is a dal/dat/actA Listeria having a mutation in the dal, dat and actA endogenous genes. In another embodiment, the mutation is a deletion, a truncation or an inactivation of the mutated genes. In another embodiment, Listeria strain exerts strong and antigen specific anti-tumor responses with ability to break tolerance toward HER2/neu in transgenic animals. In another embodiment, the dal/dat/actA strain is highly attenuated and has a better safety profile than previous Listeria vaccine generation, as it is more rapidly cleared from the spleens of the immunized mice. In another embodiment, the Listeria strain results in a longer delay of tumor onset in transgenic animals than Lm-LLO-ChHer2, the antibiotic resistant and more virulent version of this vaccine (see US Publication No. 2011/0142791, which is incorporated by reference herein in its entirety). In another embodiment, the Listeria strain causes a significant decrease in intra-tumoral T regulatory cells (Tregs). In another embodiment, the lower frequency of Tregs in tumors treated with LmddA vaccines result in an increased intratumoral CD8/Tregs ratio, suggesting that a more favorable tumor microenvironment can be obtained after immunization with LmddA vaccines. In one embodiment, the present invention provides a recombinant polypeptide comprising an N-terminal fragment of an LLO protein fused to a Her-2 chimeric protein or fused to a fragment thereof. In one embodiment, the present invention provides a recombinant polypeptide consisting of an N-terminal fragment of an LLO protein fused to a Her-2 chimeric protein or fused to a fragment thereof. In the embodiment, the heterologous antigen is a Her-2 chimeric protein or fragment thereof.

In another embodiment, the Her-2 chimeric protein of the methods and compositions of the present invention is a human Her-2 chimeric protein. In another embodiment, the Her-2 protein is a mouse Her-2 chimeric protein. In another embodiment, the Her-2 protein is a rat Her-2 chimeric protein. In another embodiment, the Her-2 protein is a primate Her-2 chimeric protein. In another embodiment, the Her-2 protein is a Her-2 chimeric protein of human or any other animal species or combinations thereof known in the art. Each possibility represents a separate embodiment of the present invention.

In another embodiment, a Her-2 protein is a protein referred to as “HER-2/neu,” “Erbb2,” “v-erb-b2,” “c-erb-b2,” “neu,” or “cNeu.” Each possibility represents a separate embodiment of the present invention.

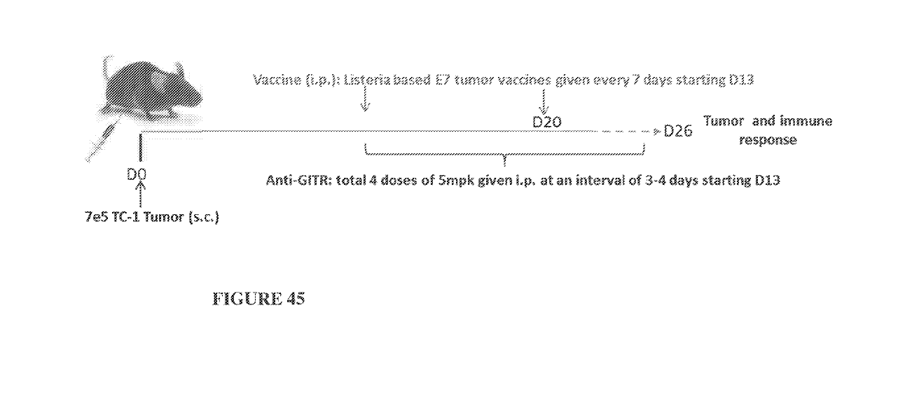

In one embodiment, the Her2-neu chimeric protein, harbors two of the extracellular and one intracellular fragments of Her2/neu antigen showing clusters of MHC-class I epitopes of the oncogene, where, in another embodiment, the chimeric protein harbors 3 H2Dq and at least 17 of the mapped human MHC-class I epitopes of the Her2/neu antigen (fragments EC1, EC2, and IC1) (FIG. 45). In another embodiment, the chimeric protein harbors at least 13 of the mapped human MHC-class I epitopes (fragments EC2 and IC1). In another embodiment, the chimeric protein harbors at least 14 of the mapped human MHC-class I epitopes (fragments EC1 and IC1). In another embodiment, the chimeric protein harbors at least 9 of the mapped human MHC-class I epitopes (fragments EC1 and IC2). In another embodiment, the Her2-neu chimeric protein is fused to a non-hemolytic listeriolysin O (LLO). In another embodiment, the Her2-neu chimeric protein is fused to the first 441 amino acids of the Listeria-monocytogenes listeriolysin O (LLO) protein and expressed and secreted by the Listeria monocytogenes attenuated auxotrophic strain LmddA. In another embodiment, the expression and secretion of the fusion protein tLLO-ChHer2 from the attenuated auxotrophic strain disclosed herein that expresses a chimeric Her2/neu antigen/LLO fusion protein is comparable to that of the Lm-LLO-ChHer2 in TCA precipitated cell culture supernatants after 8 hours of in vitro growth (FIG. 45B).

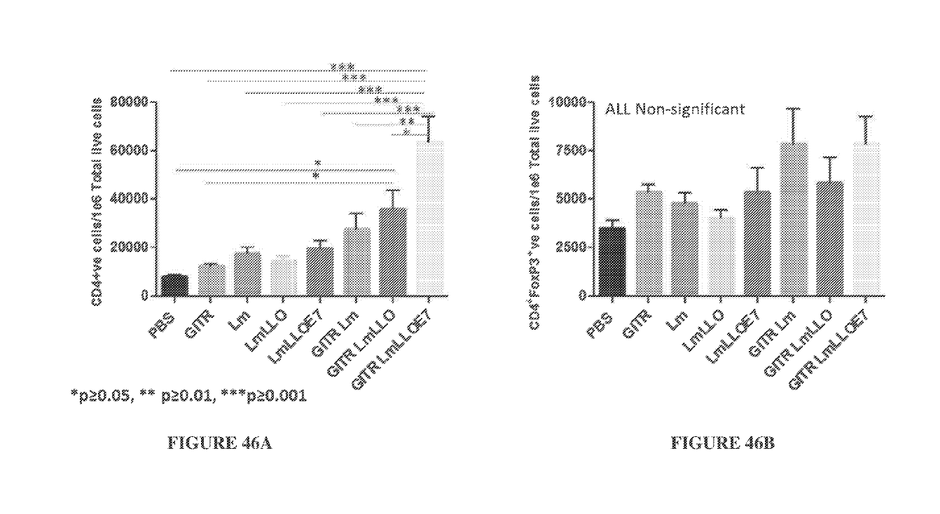

In one embodiment, no CTL activity is detected in naïve animals or mice injected with an irrelevant Listeria vaccine (FIG. 46A). While in another embodiment, the attenuated auxotrophic strain disclosed herein is able to stimulate the secretion of IFN-γ by the splenocytes from wild type FVB/N mice (FIG. 46B).

In another embodiment, the Her-2 chimeric protein is encoded by the following nucleic acid sequence set forth in SEQ ID NO:95

(SEQ ID NO: 95)
gagacccacctggacatgctccgccacctctaccagggctgccaggtg
gtgcagggaaacctggaactcacctacctgcccaccaatgccagcctg
tccttcctgcaggatatccaggaggtgcagggctacgtgctcatcgct
cacaaccaagtgaggcaggtcccactgcagaggctgcggattgtgcga
ggcacccagctctttgaggacaactatgccctggccgtgctagacaat
ggagacccgctgaacaataccacccctgtcacaggggcctccccagga
ggcctgcgggagctgcagcttcgaagcctcacagagatcttgaaagga
ggggtcttgatccagcggaacccccagctctgctaccaggacacgatt
ttgtggaagaatatccaggagtttgctggctgcaagaagatctttggg
agcctggcatttctgccggagagctttgatggggacccagcctccaac
actgccccgctccagccagagcagctccaagtgtttgagactctggaa
gagatcacaggttacctatacatctcagcatggccggacagcctgcct
gacctcagcgtcttccagaacctgcaagtaatccggggacgaattctg
cacaatggcgcctactcgctgaccctgcaagggctgggcatcagctgg
ctggggctgcgctcactgagggaactgggcagtggactggccctcatc
caccataacacccacctctgcttcgtgcacacggtgccctgggaccag
ctctttcggaacccgcaccaagctctgctccacactgccaaccggcca
gaggacgagtgtgtgggcgagggcctggcctgccaccagctgtgcgcc
cgagggcagcagaagatccggaagtacacgatgcggagactgctgcag
gaaacggagctggtggagccgctgacacctagcggagcgatgcccaac
caggcgcagatgcggatcctgaaagagacggagctgaggaaggtgaag
gtgcttggatctggcgcttttggcacagtctacaagggcatctggatc
cctgatggggagaatgtgaaaattccagtggccatcaaagtgttgagg
gaaaacacatcccccaaagccaacaaagaaatcttagacgaagcatac
gtgatggctggtgtgggctccccatatgtctcccgccttctgggcatc
tgcctgacatccacggtgcagctggtgacacagcttatgccctatggc
tgcctcttagactaa.

In another embodiment, the Her-2 chimeric protein comprises the sequence:

(SEQ ID NO: 96)
T H L D M L R H L Y Q G C Q V V Q G N L E L T Y
L P T N A S L S F L Q D I Q E V Q G Y V L I A H
N Q V R Q V P L Q R L R I V R G T Q L F E D N Y
A L A V L D N G D P L N N T T P V T G A S P G G
L R E L Q L R S L T E I L K G G V L I Q R N P Q
L C Y Q D T I L W K N I Q E F A G C K K I F G S
L A F L P E S F D G D P A S N T A P L Q P E Q L
Q V F E T L E E I T G Y L Y I S A W P D S L P D
L S V F Q N L Q V I R G R I L H N G A Y S L T L
Q G L G I S W L G L R S L R E L G S G L A L I H
H N T H L C F V H T V P W D Q L F R N P H Q A L
L H T A N R P E D E C V G E G L A C H Q L C A R
G Q Q K I R K Y T M R R L L Q E T E L V E P L T
P S G A M P N Q A Q M R I L K E T E L R K V K V
L G S G A F G T V Y K G I W I P D G E N V K I P
V A I K V L R E N T S P K A N K E I L D E A Y V
M A G V G S P Y V S R L L G I C L T S T V Q L V
T Q L M P Y G C L L D.

In one embodiment, the Her2 chimeric protein or fragment thereof of the methods and compositions disclosed herein does not include a signal sequence thereof. In another embodiment, omission of the signal sequence enables the Her2 fragment to be successfully expressed in Listeria, due the high hydrophobicity of the signal sequence. Each possibility represents a separate embodiment of the present invention.

In another embodiment, the fragment of a Her2 chimeric protein of methods and compositions of the present invention does not include a transmembrane domain (TM) thereof. In one embodiment, omission of the TM enables the Her-2 fragment to be successfully expressed in Listeria, due the high hydrophobicity of the TM. Each possibility represents a separate embodiment of the present invention.

In one embodiment, LmddA164 comprises a nucleic acid sequence comprising an open reading frame encoding tLLO fused to cHER2, wherein said nucleic acid sequence comprises SEQ ID NO: 97: atgaaaaaaataatgctagtttttattacacttatattagttagtctaccaattgcgcaacaaactgaagcaaaggatgcatctgcattcaata aagaaaattcaatttcatccatggcaccaccagcatctccgcctgcaagtcctaagacgccaatcgaaaagaaacacgcggatgaaat cgataagtatatacaaggattggattacaataaaaacaatgtattagtataccacggagatgcagtgacaaatgtgccgccaagaaaag gttacaaagatggaaatgaatatattgttgtggagaaaaagaagaaatccatcaatcaaaataatgcagacattcaagttgtgaatgcaat ttcgagcctaacctatccaggtgctctcgtaaaagcgaattcggaattagtagaaaatcaaccagatgttctccctgtaaaacgtgattcat taacactcagcattgatttgccaggtatgactaatcaagacaataaaatagttgtaaaaaatgccactaaatcaaacgttaacaacgcagt aaatacattagtggaaagatggaatgaaaaatatgctcaagcttatccaaatgtaagtgcaaaaattgattatgatgacgaaatggcttac agtgaatcacaattaattgcgaaatttggtacagcatttaaagctgtaaataatagcttgaatgtaaacttcggcgcaatcagtgaaggga aaatgcaagaagaagtcattagttttaaacaaatttactataacgtgaatgttaatgaacctacaagaccttccagatttttcggcaaagctg ttactaaagagcagttgcaagcgcttggagtgaatgcagaaaatcctcctgcatatatctcaagtgtggcgtatggccgtcaagtttatttg aaattatcaactaattcccatagtactaaagtaaaagctgcttagatgctgccgtaagcggaaaatctgtctcaggtgatgtagaactaac aaatatcatcaaaaattcaccttcaaagccgtaatttacggaggttccgcaaaagatgaagttcaaatcatcgacggcaacctcggaga cttacgcgatattttgaaaaaaggcgctacttttaatcgagaaacaccaggagttcccattgcttatacaacaaacttcctaaaagacaatg aattagctgttattaaaaacaactcagaatatattgaaacaacttcaaaagcttatacagatggaaaaattaacatcgatcactctggagga tacgttgctcaattcaacatttcttgggatgaagtaaattatgatctcgagACCCACCTGGACATGCTCCGCCACC TCTACCAGGGCTGCCAGGTGGTGCAGGGAAACCTGGAACTCACCTACCTGCCCAC CAATGCCAGCCTGTCCTTCCTGCAGGATATCCAGGAGGTGCAGGGCTACGTGCTC ATCGCTCACAACCAAGTGAGGCAGGTCCCACTGCAGAGGCTGCGGATTGTGCGA GGCACCCAGCTCTTTGAGGACAACTATGCCCTGGCCGTGCTAGACAATGGAGACC CGCTGAACAATACCACCCCTGTCACAGGGGCCTCCCCAGGAGGCCTGCGGGAGCT GCAGCTTCGAAGCCTCACAGAGATCTTGAAAGGAGGGGTCTTGATCCAGCGGAA CCCCCAGCTCTGCTACCAGGACACGATTTTGTGGAAGAATATCCAGGAGTTTGCT GGCTGCAAGAAGATCTTTGGGAGCCTGGCATTTCTGCCGGAGAGCTTTGATGGGG ACCCAGCCTCCAACACTGCCCCGCTCCAGCCAGAGCAGCTCCAAGTGTTTGAGAC TCTGGAAGAGATCACAGGTTACCTATACATCTCAGCATGGCCGGACAGCCTGCCT GACCTCAGCGTCTTCCAGAACCTGCAAGTAATCCGGGGACGAATTCTGCACAATG GCGCCTACTCGCTGACCCTGCAAGGGCTGGGCATCAGCTGGCTGGGGCTGCGCTC ACTGAGGGAACTGGGCAGTGGACTGGCCCTCATCCACCATAACACCCACCTCTGC TTCGTGCACACGGTGCCCTGGGACCAGCTCTTTCGGAACCCGCACCAAGCTCTGC TCCACACTGCCAACCGGCCAGAGGACGAGTGTGTGGGCGAGGGCCTGGCCTGCC ACCAGCTGTGCGCCCGAGGGCAGCAGAAGATCCGGAAGTACACGATGCGGAGAC TGCTGCAGGAAACGGAGCTGGTGGAGCCGCTGACACCTAGCGGAGCGATGCCCA ACCAGGCGCAGATGCGGATCCTGAAAGAGACGGAGCTGAGGAAGGTGAAGGTGC TTGGATCTGGCGCTTTTGGCACAGTCTACAAGGGCATCTGGATCCCTGATGGGGA GAATGTGAAAATTCCAGTGGCCATCAAAGTGTTGAGGGAAAACACATCCCCCAA AGCCAACAAAGAAATCTTAGACGAAGCATACGTGATGGCTGGTGTGGGCTCCCC ATATGTCTCCCGCCTTCTGGGCATCTGCCTGACATCCACGGTGCAGCTGGTGACA CAGCTTATGCCCTATGGCTGCCTCTTAGAC (SEQ ID NO: 97), wherein the UPPERCASE sequences encode cHER2, the lowercase sequences encode tLLO and the underlined “ctcgag” sequence represents the Xho I restriction site used to ligate the tumor antigen to truncated LLO in the plasmid. In another embodiment, plasmid pAdv168 comprises SEQ ID NO: 97. In one embodiment, the truncated LLO-cHER2 fusion is a homolog of SEQ ID NO: 97. In another embodiment, the truncated LLO-cHER2 fusion is a variant of SEQ ID NO: 97. In another embodiment, the truncated LLO-cHER2 fusion is an isomer of SEQ ID NO: 97.

In one embodiment, an amino acid sequence of a recombinant protein comprising tLLO fused to a cHER2 comprises SEQ ID NO: 98: MKKIMLVFITLILVSLPIAQQTEAKDASAFNKENSISSMAPPASPPASPKTPIEKKHADE IDKYIQGLDYNKNNVLVYHGDAVTNVPPRKGYKDGNEYIVVEKKKKSINQNNADIQ VVNAISSLTYPGALVKANSELVENQPDVLPVKRDSLTLSIDLPGMTNQDNKWVKNA TKSNVNNAVNTLVERWNEKYAQAYPNVSAKIDYDDEMAYSESQLIAKFOTAFKAV NNSLNVNFGAISEGKMQEEVISFKQIYYNVNVNEPTRPSRFFGKAVTKEQLQALGVN AENPPAYISSVAYGRQVYLKLSTNSIISTKVKAAFDAAVSGKSVSGDVELTNIIKNSSF KAVIYGGSAKDEVQIIDGNLGDLRDILKKGATFNRETPGVPIAYTTNFLKDNELAVIK NNSEYIETTSKAYTDGKINIDHSGGYVAQFNISWDEVNYDLETHLDMLRHLYQGCQV VQGNLELTYLPTNASLSFLQDIQEVQGYVLIAHNQVRQVPLQRLRIVRGTQLFEDNY ALAVLDNGDPLNNTTPVTGASPGGLRELQLRSLTEILKGGVLIQRNPQLCYQDTILWK NIQEFAGCKKIFGSLAFLPESFDGDPASNTAPLQPEQLQVFETLEEITGYLYISAWPDSL PDLSVFQNLQVIRGRILHNGAYSLTLQGLGISWLGLRSLRELGSGLALIHHNTHLCFV HTVPWDQLFRNPHQALLHTANRPEDECVGEGLACHQLCARGQQKIRKYTMRRLLQE TELVEPLTPSGAMPNQAQMKILKETELRKVKVLGSGAFGTVYKGIWIPDGENVKIPV AIKVLRENTSPKANKEILDEAYVMAGVGSPYVSRLLGICLTSTVQLVTQLMPYGCLL D (SEQ ID NO: 98). In one embodiment, the truncated LLO-cHER2 fusion is a homolog of SEQ ID NO: 98. In another embodiment, the truncated LLO-cHER2 fusion is a variant of SEQ ID NO: 98. In another embodiment, the truncated LLO-cHER2 fusion is an isomer of SEQ ID NO: 98.

Point mutations or amino-acid deletions in the oncogenic protein Her2/neu, have been reported to mediate treatment of resistant tumor cells, when these tumors have been targeted by small fragment Listeria-based vaccines or trastuzumab (a monoclonal antibody against an epitope located at the extracellular domain of the Her2/neu antigen). In one embodiment, disclosed is a chimeric Her2/neu based composition which harbors two of the extracellular and one intracellular fragments of Her2/neu antigen showing clusters of MHC-class I epitopes of the oncogene. This chimeric protein, which harbors 3 H2Dq and at least 17 of the mapped human MHC-class I epitopes of the Her2/neu antigen was fused to the first 441 amino acids of the Listeria-monocytogenes listeriolysin O protein and expressed and secreted by the Listeria monocytogenes attenuated strain LmddA.

In another embodiment, the antigen of interest is a KLK9 polypeptide.

In another embodiment, the tumor-associated antigen is HPV-E7. In another embodiment, the antigen is HPV-E6. In another embodiment, the antigen is Her-2. In another embodiment, the antigen is NY-ESO-1. In another embodiment, the antigen is telomerase. In another embodiment, the antigen is SCCE. In another embodiment, the antigen is WT-1. In another embodiment, the antigen is HW-1 Gag. In another embodiment, the antigen is Proteinase 3. In another embodiment, the antigen is Tyrosinase related protein 2. In another embodiment, the antigen is PSA (prostate-specific antigen). In another embodiment, the antigen is selected from E7, E6, Her-2, NY-ESO-1, telomerase, SCCE, WT-1, HIV-1 Gag, Proteinase 3, Tyrosinase related protein 2, PSA (prostate-specific antigen). In another embodiment, the antigen is a tumor-associated antigen. In another embodiment, the antigen is an infectious disease antigen.

In another embodiment, the tumor-associated antigen is an angiogenic antigen. In another embodiment, the angiogenic antigen is expressed on both activated pericytes and pericytes in tumor angiogenic vasculature, which in another embodiment, is associated with neovascularization in vivo. In another embodiment, the angiogenic antigen is HMW-MAA. In another embodiment, the angiogenic antigen is one known in the art and are disclosed in WO2010/102140, which is incorporated by reference herein.

In other embodiments, the antigen is derived from a fungal pathogen, bacteria, parasite, helminth, or viruses. In other embodiments, the antigen is selected from tetanus toxoid, hemagglutinin molecules from influenza virus, diphtheria toxoid, HIV gp120, HIV gag protein, IgA protease, insulin peptide B, Spongospora subterranea antigen, vibriose antigens, Salmonella antigens, pneumococcus antigens, respiratory syncytial virus antigens, Haemophilus influenza outer membrane proteins, Helicobacter pylori urease, Neisseria meningitidis pilins, N. gonorrhoeae pilins, the melanoma-associated antigens (TRP-2, MAGE-1, MAGE-3, gp-100, tyrosinase, MART-1, HSP-70, beta-HCG), human papilloma virus antigens E1 and E2 from type HPV-16, -18, -31, -33, -35 or -45 human papilloma viruses, the tumor antigens CEA, the ras protein, mutated or otherwise, the p53 protein, mutated or otherwise, Muc1, mesothelin, EGFRVIII or pSA.

In other embodiments, the antigen is associated with one of the following diseases; cholera, diphtheria, Haemophilus, hepatitis A, hepatitis B, influenza, measles, meningitis, mumps, pertussis, small pox, pneumococcal pneumonia, polio, rabies, rubella, tetanus, tuberculosis, typhoid, Varicella-zoster, whooping cough, yellow fever, the immunogens and antigens from Addison's disease, allergies, anaphylaxis, Bruton's syndrome, cancer, including solid and blood borne tumors, eczema, Hashimoto's thyroiditis, polymyositis, dermatomyositis, type 1 diabetes mellitus, acquired immune deficiency syndrome, transplant rejection, such as kidney, heart, pancreas, lung, bone, and liver transplants, Graves' disease, polyendocrine autoimmune disease, hepatitis, microscopic polyarteritis, polyarteritis nodosa, pemphigus, primary biliary cirrhosis, pernicious anemia, coeliac disease, antibody-mediated nephritis, glomerulonephritis, rheumatic diseases, systemic lupus erythematosus, rheumatoid arthritis, seronegative spondylarthritides, rhinitis, sjogren's syndrome, systemic sclerosis, sclerosing cholangitis, Wegener's granulomatosis, dermatitis herpetiformis, psoriasis, vitiligo, multiple sclerosis, encephalomyelitis, Guillain-Barre syndrome, myasthenia gravis, Lambert-Eaton syndrome, sclera, episclera, uveitis, chronic mucocutaneous candidiasis, urticaria, transient hypogammaglobulinemia of infancy, myeloma, X-linked hyper IgM syndrome, Wiskott-Aldrich syndrome, ataxia telangiectasia, autoimmune hemolytic anemia, autoimmune thrombocytopenia, autoimmune neutropenia, Waldenstrom's macroglobulinemia, amyloidosis, chronic lymphocytic leukemia, non-Hodgkin's lymphoma, malarial circumsporozoite protein, microbial antigens, viral antigens, autoantigens, and listeriosis. Each antigen represents a separate embodiment of the methods and compositions as disclosed herein.

In another embodiment, the heterologous antigen disclosed herein is a tumor-associated antigen, which in one embodiment, is one of the following tumor antigens: a MAGE (Melanoma-Associated Antigen E) protein, e.g. MAGE 1, MAGE 2, MAGE 3, MAGE 4, a tyrosinase; a mutant ras protein; a mutant p53 protein; p97 melanoma antigen, a ras peptide or p53 peptide associated with advanced cancers; the HPV 16/18 antigens associated with cervical cancers, KLH antigen associated with breast carcinoma, CEA (carcinoembryonic antigen) associated with colorectal cancer, gp100, a MART1 antigen associated with melanoma, or the PSA antigen associated with prostate cancer. In another embodiment, the antigen for the compositions and methods as disclosed herein are melanoma-associated antigens, which in one embodiment are TRP-2, MAGE-1, MAGE-3, gp-100, tyrosinase, HSP-70, beta-HCG, or a combination thereof. In another embodiment, the tumor associated antigen is an angiogenic antigen.

In one embodiment, the antigen is a chimeric Her2 antigen described in US patent application publication US2011/0142791, which is hereby incorporated by reference herein in its entirety.

In another embodiment, the heterologous antigen is an infectious disease antigen. In one embodiment, the antigen is an auto antigen or a self-antigen.

In another embodiment, the heterologous antigen is derived from a fungal pathogen, bacteria, parasite, helminth, or viruses. In other embodiments, the antigen is selected from tetanus toxoid, hemagglutinin molecules from influenza virus, diphtheria toxoid, HIV gp120, HIV gag protein, IgA protease, insulin peptide B, Spongospora subterranea antigen, vibriose antigens, Salmonella antigens, pneumococcus antigens, respiratory syncytial virus antigens, Haemophilus influenza outer membrane proteins, Helicobacter pylori urease, Neisseria meningitidis pilins, N. gonorrhoeae pilins, human papilloma virus antigens E1 and E2 from type HPV-16, -18, -31, -33, -35 or -45 human papilloma viruses, or a combination thereof.

In another embodiment of the methods and compositions as disclosed herein, “nucleic acids” or “nucleotide” refers to a string of at least two base-sugar-phosphate combinations. The term includes, in one embodiment, DNA and RNA. “Nucleotides” refers, in one embodiment, to the monomeric units of nucleic acid polymers. RNA may be, in one embodiment, in the form of a tRNA (transfer RNA), snRNA (small nuclear RNA), rRNA (ribosomal RNA), mRNA (messenger RNA), anti-sense RNA, small inhibitory RNA (siRNA), micro RNA (miRNA) and ribozymes. The use of siRNA and miRNA has been described (Caudy A A et al, Genes & Devel 16: 2491-96 and references cited therein). DNA may be in form of plasmid DNA, viral DNA, linear DNA, or chromosomal DNA or derivatives of these groups. In addition, these forms of DNA and RNA may be single, double, triple, or quadruple stranded. The term also includes, in another embodiment, artificial nucleic acids that may contain other types of backbones but the same bases. In one embodiment, the artificial nucleic acid is a PNA (peptide nucleic acid). PNA contain peptide backbones and nucleotide bases and are able to bind, in one embodiment, to both DNA and RNA molecules. In another embodiment, the nucleotide is oxetane modified. In another embodiment, the nucleotide is modified by replacement of one or more phosphodiester bonds with a phosphorothioate bond. In another embodiment, the artificial nucleic acid contains any other variant of the phosphate backbone of native nucleic acids known in the art. The use of phosphothioate nucleic acids and PNA are known to those skilled in the art, and are described in, for example, Neilsen P E, Curr Opin Struct Biol 9:353-57; and Raz N K et al Biochem Biophys Res Commun. 297:1075-84. The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G. C. Fareed. Each nucleic acid derivative represents a separate embodiment as disclosed herein.

In one embodiment, the term “oligonucleotide” is interchangeable with the term “nucleic acid”, and may refer to a molecule, which may include, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also refers to sequences that include any of the known base analogs of DNA and RNA.

In another embodiment, the construct or nucleic acid molecule disclosed herein is integrated into the Listerial chromosome using homologous recombination. Techniques for homologous recombination are well known in the art, and are described, for example, in Baloglu S, Boyle S M, et al. (Immune responses of mice to vaccinia virus recombinants expressing either Listeria monocytogenes partial listeriolysin or Brucella abortus ribosomal L7/L12 protein. Vet Microbiol 2005, 109(1-2): 11-7); and Jiang L L, Song H H, et al., (Characterization of a mutant Listeria monocytogenes strain expressing green fluorescent protein. Acta Biochim Biophys Sin (Shanghai) 2005, 37(1): 19-24). In another embodiment, homologous recombination is performed as described in U.S. Pat. No. 6,855,320. In this case, a recombinant Lm strain that expresses E7 was made by chromosomal integration of the E7 gene under the control of the hly promoter and with the inclusion of the hly signal sequence to ensure secretion of the gene product, yielding the recombinant referred to as Lm-AZ/E7. In another embodiment, a temperature sensitive plasmid is used to select the recombinants. Each technique represents a separate embodiment of the present invention.

In another embodiment, the construct or nucleic acid molecule is integrated into the Listerial chromosome using transposon insertion. Techniques for transposon insertion are well known in the art, and are described, inter alia, by Sun et al. (Infection and Immunity 1990, 58: 3770-3778) in the construction of DP-L967. Transposon mutagenesis has the advantage, in another embodiment, that a stable genomic insertion mutant can be formed but the disadvantage that the position in the genome where the foreign gene has been inserted is unknown.

In another embodiment, the construct or nucleic acid molecule is integrated into the Listerial chromosome using phage integration sites (Lauer P, Chow M Y et al, Construction, characterization, and use of two Listeria monocytogenes site-specific phage integration vectors. J Bacteriol 2002; 184(15): 4177-86). In certain embodiments of this method, an integrase gene and attachment site of a bacteriophage (e.g. U153 or PSA listeriophage) is used to insert the heterologous gene into the corresponding attachment site, which may be any appropriate site in the genome (e.g. comK or the 3′ end of the arg tRNA gene). In another embodiment, endogenous prophages are cured from the attachment site utilized prior to integration of the construct or heterologous gene. In another embodiment, this method results in single-copy integrants. In another embodiment, the present invention further comprises a phage based chromosomal integration system for clinical applications, where a host strain that is auxotrophic for essential enzymes, including, but not limited to, d-alanine racemase can be used, for example Lmdal(−)dat(−). In another embodiment, in order to avoid a “phage curing step,” a phage integration system based on PSA is used. This requires, in another embodiment, continuous selection by antibiotics to maintain the integrated gene. Thus, in another embodiment, the current invention enables the establishment of a phage based chromosomal integration system that does not require selection with antibiotics. Instead, an auxotrophic host strain can be complemented. Each possibility represents a separate embodiment of the present invention.

In one embodiment of the methods and compositions as disclosed herein, the term “recombination site” or “site-specific recombination site” refers to a sequence of bases in a nucleic acid molecule that is recognized by a recombinase (along with associated proteins, in some cases) that mediates exchange or excision of the nucleic acid segments flanking the recombination sites. The recombinases and associated proteins are collectively referred to as “recombination proteins” see, e.g., Landy, A., (Current Opinion in Genetics & Development) 3:699-707; 1993).

A “phage expression vector” or “phagemid” refers to any phage-based recombinant expression system for the purpose of expressing a nucleic acid sequence of the methods and compositions as disclosed herein in vitro or in vivo, constitutively or inducibly, in any cell, including prokaryotic, yeast, fungal, plant, insect or mammalian cell. A phage expression vector typically can both reproduce in a bacterial cell and, under proper conditions, produce phage particles. The term includes linear or circular expression systems and encompasses both phage-based expression vectors that remain episomal or integrate into the host cell genome.

In one embodiment, the term “operably linked” as used herein means that the transcriptional and translational regulatory nucleic acid, is positioned relative to any coding sequences in such a manner that transcription is initiated. Generally, this will mean that the promoter and transcriptional initiation or start sequences are positioned 5′ to the coding region.

In one embodiment, an “open reading frame” or “ORF” is a portion of an organism's genome which contains a sequence of bases that could potentially encode a protein. In another embodiment, the start and stop ends of the ORF are not equivalent to the ends of the mRNA, but they are usually contained within the mRNA. In one embodiment, ORFs are located between the start-code sequence (initiation codon) and the stop-codon sequence (termination codon) of a gene. Thus, in one embodiment, a nucleic acid molecule operably integrated into a genome as an open reading frame with an endogenous polypeptide is a nucleic acid molecule that has integrated into a genome in the same open reading frame as an endogenous polypeptide.

In one embodiment, the present invention provides a fusion polypeptide comprising a linker sequence. In one embodiment, a “linker sequence” refers to an amino acid sequence that joins two heterologous polypeptides, or fragments or domains thereof. In general, as used herein, a linker is an amino acid sequence that covalently links the polypeptides to form a fusion polypeptide. A linker typically includes the amino acids translated from the remaining recombination signal after removal of a reporter gene from a display vector to create a fusion protein comprising an amino acid sequence encoded by an open reading frame and the display protein. As appreciated by one of skill in the art, the linker can comprise additional amino acids, such as glycine and other small neutral amino acids.

In one embodiment, “endogenous” refers to an item that has developed or originated within the reference organism or arisen from causes within the reference organism. In another embodiment, endogenous refers to native.

“Stably maintained” refers, in another embodiment, to maintenance of a nucleic acid molecule or plasmid in the absence of selection (e.g. antibiotic selection) for 10 generations, without detectable loss. In another embodiment, the period is 15 generations. In another embodiment, the period is 20 generations. In another embodiment, the period is 25 generations. In another embodiment, the period is 30 generations. In another embodiment, the period is 40 generations. In another embodiment, the period is 50 generations. In another embodiment, the period is 60 generations. In another embodiment, the period is 80 generations. In another embodiment, the period is 100 generations. In another embodiment, the period is 150 generations. In another embodiment, the period is 200 generations. In another embodiment, the period is 300 generations. In another embodiment, the period is 500 generations. In another embodiment, the period is more than generations. In another embodiment, the nucleic acid molecule or plasmid is maintained stably in vitro (e.g. in culture). In another embodiment, the nucleic acid molecule or plasmid is maintained stably in vivo. In another embodiment, the nucleic acid molecule or plasmid is maintained stably both in vitro and in vitro.

In another embodiment, a recombinant Listeria strain of the methods and compositions as disclosed herein comprise a nucleic acid molecule operably integrated into the Listeria genome as an open reading frame with an endogenous ActA sequence. In another embodiment, a recombinant Listeria strain of the methods and compositions as disclosed herein comprise an episomal expression vector comprising a nucleic acid molecule encoding a fusion protein comprising an antigen fused to an ActA or a truncated ActA. In one embodiment, the expression and secretion of the antigen is under the control of an actA promoter and ActA signal sequence and it is expressed as fusion to 1-233 amino acids of ActA (truncated ActA or tActA). In another embodiment, the truncated ActA consists of the first 390 amino acids of the wild type ActA protein as described in U.S. Pat. No. 7,655,238, which is incorporated by reference herein in its entirety. In another embodiment, the truncated ActA is an ActA-N100 or a modified version thereof (referred to as ActA-N100*) in which a PEST motif has been deleted and containing the nonconservative QDNKR substitution as described in US Patent Publication Serial No. 2014/0186387.

In another embodiment, a “functional fragment” is an immunogenic fragment and elicits an immune response when administered to a subject alone or in a vaccine composition disclosed herein. In another embodiment, a functional fragment has biological activity as will be understood by a skilled artisan and as further disclosed herein.

The recombinant Listeria strain of methods and compositions disclosed herein is, in another embodiment, a recombinant Listeria monocytogenes strain. In another embodiment, the Listeria strain is a recombinant Listeria seeligeri strain. In another embodiment, the Listeria strain is a recombinant Listeria grayi strain. In another embodiment, the Listeria strain is a recombinant Listeria ivanovii strain. In another embodiment, the Listeria strain is a recombinant Listeria murrayi strain. In another embodiment, the Listeria strain is a recombinant Listeria welshimeri strain. In another embodiment, the Listeria strain is a recombinant strain of any other Listeria species known in the art.

In another embodiment, a recombinant Listeria strain disclosed herein has been passaged through an animal host. In another embodiment, the passaging maximizes efficacy of the strain as a vaccine vector. In another embodiment, the passaging stabilizes the immunogenicity of the Listeria strain. In another embodiment, the passaging stabilizes the virulence of the Listeria strain. In another embodiment, the passaging increases the immunogenicity of the Listeria strain. In another embodiment, the passaging increases the virulence of the Listeria strain. In another embodiment, the passaging removes unstable sub-strains of the Listeria strain. In another embodiment, the passaging reduces the prevalence of unstable sub-strains of the Listeria strain. In another embodiment, the Listeria strain contains a genomic insertion of the gene encoding the antigen-containing recombinant peptide. In another embodiment, the Listeria strain carries a plasmid comprising the gene encoding the antigen-containing recombinant peptide. In another embodiment, the passaging is performed as described herein. In another embodiment, the passaging is performed by any other method known in the art. In another embodiment, a recombinant Listeria strain disclosed herein has not been passaged through an animal host.

In another embodiment, a recombinant nucleic acid disclosed herein is operably linked to a promoter/regulatory sequence that drives expression of the encoded peptide in the Listeria strain. Promoter/regulatory sequences useful for driving constitutive expression of a gene are well known in the art and include, but are not limited to, for example, the P_{hlyA}, P_{ActA}, and p60 promoters of Listeria, the Streptococcus bac promoter, the Streptomyces griseus sgiA promoter, and the B. thuringiensis phaZ promoter.

In another embodiment, inducible and tissue specific expression of the nucleic acid encoding a peptide disclosed herein is accomplished by placing the nucleic acid encoding the peptide under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In another embodiment, a promoter that is induced in response to inducing agents such as metals, glucocorticoids, and the like, is utilized. Thus, it will be appreciated that the invention includes the use of any promoter/regulatory sequence, which is either known or unknown, and which is capable of driving expression of the desired protein operably linked thereto. It will be appreciated by a skilled artisan that the term “heterologous” encompasses a nucleic acid, amino acid, peptide, polypeptide, or protein derived from a different species than the reference species. Thus, for example, a Listeria strain expressing a heterologous polypeptide, in one embodiment, would express a polypeptide that is not native or endogenous to the Listeria strain, or in another embodiment, a polypeptide that is not normally expressed by the Listeria strain, or in another embodiment, a polypeptide from a source other than the Listeria strain. In another embodiment, heterologous may be used to describe something derived from a different organism within the same species. In another embodiment, the heterologous antigen is expressed by a recombinant strain of Listeria, and is processed and presented to cytotoxic T-cells upon infection of mammalian cells by the recombinant strain. In another embodiment, the heterologous antigen expressed by Listeria species need not precisely match the corresponding unmodified antigen or protein in the tumor cell or infectious agent so long as it results in a T-cell response that recognizes the unmodified antigen or protein which is naturally expressed in the mammal.

It will be appreciated by the skilled artisan that the term “episomal expression vector” encompasses a nucleic acid vector which may be linear or circular, and which is usually double-stranded in form and is extrachromosomal in that it is present in the cytoplasm of a host bacteria or cell as opposed to being integrated into the bacteria's or cell's genome. In one embodiment, an episomal expression vector comprises a gene of interest. In another embodiment, episomal vectors persist in multiple copies in the bacterial cytoplasm, resulting in amplification of the gene of interest, and, in another embodiment, viral trans-acting factors are supplied when necessary. In another embodiment, the episomal expression vector may be referred to as a plasmid herein. In another embodiment, an “integrative plasmid” comprises sequences that target its insertion or the insertion of the gene of interest carried within into a host genome. In another embodiment, an inserted gene of interest is not interrupted or subjected to regulatory constraints which often occur from integration into cellular DNA. In another embodiment, the presence of the inserted heterologous gene does not lead to rearrangement or interruption of the cell's own important regions. In another embodiment, in stable transfection procedures, the use of episomal vectors often results in higher transfection efficiency than the use of chromosome-integrating plasmids (Belt, P.B.G.M., et al (1991) Efficient cDNA cloning by direct phenotypic correction of a mutant human cell line (HPRT2) using an Epstein-Barr virus-derived cDNA expression vector. Nucleic Acids Res. 19, 4861-4866; Mazda, O., et al. (1997) Extremely efficient gene transfection into lympho-hematopoietic cell lines by Epstein-Barr virus-based vectors. J. Immunol. Methods 204, 143-151). In one embodiment, the episomal expression vectors of the methods and compositions as disclosed herein may be delivered to cells in vivo, ex vivo, or in vitro by any of a variety of the methods employed to deliver DNA molecules to cells. The vectors may also be delivered alone or in the form of a pharmaceutical composition that enhances delivery to cells of a subject.

In one embodiment, the term “fused” refers to operable linkage by covalent bonding. In one embodiment, the term includes recombinant fusion (of nucleic acid sequences or open reading frames thereof). In another embodiment, the term includes chemical conjugation.

“Transforming,” in one embodiment, refers to engineering a bacterial cell to take up a plasmid or other heterologous DNA molecule. In another embodiment, “transforming” refers to engineering a bacterial cell to express a gene of a plasmid or other heterologous DNA molecule. Each possibility represents a separate embodiment of the methods and compositions as disclosed herein.

In another embodiment, conjugation is used to introduce genetic material and/or plasmids into bacteria. Methods for conjugation are well known in the art, and are described, for example, in Nikodinovic J. et al (A second generation snp-derived Escherichia coli-Streptomyces shuttle expression vector that is generally transferable by conjugation. Plasmid. 2006 November; 56(3):223-7) and Auchtung J M et al (Regulation of a Bacillus subtilis mobile genetic element by intercellular signaling and the global DNA damage response. Proc Natl Acad Sci USA. 2005 Aug. 30; 102(35):12554-9). Each method represents a separate embodiment of the methods and compositions as disclosed herein.

In one embodiment, the term “attenuation,” refers to a diminution in the ability of the bacterium to cause disease in an animal. In other words, the pathogenic characteristics of the attenuated Listeria strain have been lessened compared with wild-type Listeria, although the attenuated Listeria is capable of growth and maintenance in culture. Using as an example the intravenous inoculation of Balb/c mice with an attenuated Listeria, the lethal dose at which 50% of inoculated animals survive (LD₅₀) is preferably increased above the LD₅₀ of wild-type Listeria by at least about 10-fold, more preferably by at least about 100-fold, more preferably at least about 1,000 fold, even more preferably at least about 10,000 fold, and most preferably at least about 100,000-fold. An attenuated strain of Listeria is thus one which does not kill an animal to which it is administered, or is one which kills the animal only when the number of bacteria administered is vastly greater than the number of wild type non-attenuated bacteria which would be required to kill the same animal. An attenuated bacterium should also be construed to mean one which is incapable of replication in the general environment because the nutrient required for its growth is not present therein. Thus, the bacterium is limited to replication in a controlled environment wherein the required nutrient is provided. The attenuated strains of the present invention are therefore environmentally safe in that they are incapable of uncontrolled replication.

### Compositions

In one embodiment, compositions of the present invention are immunogenic compositions. In one embodiment, compositions of the present invention induce a strong innate stimulation of interferon-gamma, which in one embodiment, has anti-angiogenic properties. In one embodiment, a Listeria disclosed herein induces a strong innate stimulation of interferon-gamma, which in one embodiment, has anti-angiogenic properties (Dominiecki et al., Cancer Immunol Immunother. 2005 May; 54(5):477-88. Epub 2004 Oct. 6, incorporated herein by reference in its entirety; Beatty and Paterson, J. Immunol. 2001 Feb. 15; 166(4):2276-82, incorporated herein by reference in its entirety). In one embodiment, anti-angiogenic properties of Listeria are mediated by CD4⁺ T cells (Beatty and Paterson, 2001). In another embodiment, anti-angiogenic properties of Listeria are mediated by CD8⁺ T cells. In another embodiment, IFN-gamma secretion as a result of Listeria vaccination is mediated by NK cells, NKT cells, Th1 CD4⁺ T cells, TC1 CD8⁺ T cells, or a combination thereof.

In another embodiment, administration of the compositions disclosed herein induce production of one or more anti-angiogenic proteins or factors. In one embodiment, the anti-angiogenic protein is IFN-gamma. In another embodiment, the anti-angiogenic protein is pigment epithelium-derived factor (PEDF); angiostatin; endostatin; fms-like tyrosine kinase (sFlt)-1; or soluble endoglin (sEng). In one embodiment, a Listeria of the present invention is involved in the release of anti-angiogenic factors, and, therefore, in one embodiment, has a therapeutic role in addition to its role as a vector for introducing an antigen to a subject. Each Listeria strain and type thereof represents a separate embodiment of the present invention.

The immune response induced by methods and compositions as disclosed herein is, in another embodiment, a T cell response. In another embodiment, the immune response comprises a T cell response. In another embodiment, the response is a CD8+ T cell response. In another embodiment, the response comprises a CD8⁺ T cell response. Each possibility represents a separate embodiment as disclosed herein.

In another embodiment, administration of the compositions disclosed herein increase the number of antigen-specific T cells. In another embodiment, administration of compositions activates co-stimulatory receptors on T cells. In another embodiment, administration of compositions induces proliferation of memory and/or effector T cells. In another embodiment, administration of compositions increases proliferation of T cells.

As used throughout, the terms “composition” and “immunogenic composition” are interchangeable having all the same meanings and qualities. In one embodiment, an immunogenic composition disclosed herein comprises a recombinant Listeria strain and further comprising an antibody for concomitant or sequential administration of each component is also referred to as a “combination therapy.” In another embodiment, an immunogenic composition disclosed herein comprising a recombinant Listeria strain and further comprising an antibody for concomitant or sequential administration of each component is also referred to as a “combination therapy.” It is to be understood by a skilled artisan that a combination therapy may also comprise additional components, antibodies, therapies, etc. The term “pharmaceutical composition” refers, in some embodiments, to a composition suitable for pharmaceutical use, for example, to administer to a subject in need.

Compositions of this invention may be used in methods of this invention in order to elicit an enhanced anti-tumor T cell response in a subject, in order to inhibit tumor-mediated immunosuppression in a subject, or for increasing the ratio or T effector cells to regulatory T cells (Tregs) in the spleen and tumor of a subject, or any combination thereof.

In another embodiment, a composition comprising a Listeria strain disclosed herein further comprises an adjuvant. In one embodiment, a composition of the present invention further comprises an adjuvant. The adjuvant utilized in methods and compositions of the present invention is, in another embodiment, a granulocyte/macrophage colony-stimulating factor (GM-CSF) protein. In another embodiment, the adjuvant comprises a GM-CSF protein. In another embodiment, the adjuvant is a nucleotide molecule encoding GM-CSF. In another embodiment, the adjuvant comprises a nucleotide molecule encoding GM-CSF. In another embodiment, the adjuvant is saponin QS21. In another embodiment, the adjuvant comprises saponin QS21. In another embodiment, the adjuvant is monophosphoryl lipid A. In another embodiment, the adjuvant comprises monophosphoryl lipid A. In another embodiment, the adjuvant is SBAS2. In another embodiment, the adjuvant comprises SBAS2. In another embodiment, the adjuvant is an unmethylated CpG-containing oligonucleotide. In another embodiment, the adjuvant comprises an unmethylated CpG-containing oligonucleotide. In another embodiment, the adjuvant is an immune-stimulating cytokine. In another embodiment, the adjuvant comprises an immune-stimulating cytokine. In another embodiment, the adjuvant is a nucleotide molecule encoding an immune-stimulating cytokine. In another embodiment, the adjuvant comprises a nucleotide molecule encoding an immune-stimulating cytokine. In another embodiment, the adjuvant is or comprises a quill glycoside. In another embodiment, the adjuvant is or comprises a bacterial mitogen. In another embodiment, the adjuvant is or comprises a bacterial toxin. In another embodiment, the adjuvant is or comprises any other adjuvant known in the art. Each possibility represents a separate embodiment of the present invention.

In one embodiment, an immunogenic composition disclosed herein comprises a recombinant Listeria strain comprising a nucleic acid molecule, said nucleic acid molecule comprising a first open reading frame encoding a fusion polypeptide, wherein said fusion polypeptide comprises a truncated listeriolysin O (LLO) protein, a truncated ActA protein, or a PEST amino acid sequence fused to a heterologous antigen or fragment thereof. In another embodiment, an immunogenic composition disclosed herein comprises a recombinant Listeria strain comprising a nucleic acid molecule, said nucleic acid molecule comprising a first open reading frame encoding a truncated listeriolysin O (LLO) protein, a truncated ActA protein, or a PEST amino acid sequence.

In one embodiment, an immunogenic composition disclosed herein comprises a recombinant Listeria strain comprising a nucleic acid molecule, said nucleic acid molecule comprising a first open reading frame encoding a fusion polypeptide, wherein said fusion polypeptide comprises a truncated listeriolysin O (LLO) protein, a truncated ActA protein, or a PEST amino acid sequence fused to a heterologous antigen or fragment thereof, said composition further comprising an antibody or fragment thereof. In another embodiment said antibody or fragment thereof comprises a polyclonal antibody, a monoclonal antibody, an Fab fragment, an F(ab′)2 fragment, an Fv fragment, a single chain antibody, or any combination thereof.

In another embodiment, an immunogenic composition disclosed herein comprises a recombinant Listeria strain comprising a nucleic acid molecule, said nucleic acid molecule comprising a first open reading frame encoding a fusion polypeptide, wherein said fusion polypeptide comprises a truncated listeriolysin O (LLO) protein, a truncated ActA protein, or a PEST amino acid sequence fused to a heterologous antigen or fragment thereof, said composition further comprising an antibody or fragment thereof. In another embodiment said antibody or fragment thereof comprises a polyclonal antibody, a monoclonal antibody, an Fab fragment, an F(ab′)2 fragment, an Fv fragment, a single chain antibody, or any combination thereof.

In some embodiments, the term “antibody” refers to intact molecules as well as functional fragments thereof, also referred to herein as “antigen binding fragments”, such as Fab, F(ab′)2, and Fv that are capable of specifically interacting with a desired target as described herein, for example, binding to TNF receptor superfamily members, or T-cell receptor co-stimulatory molecules, or an antigen presenting cell receptor binding a co-stimulatory molecule.

In some embodiments, the antibody fragments comprise: (1) Fab, the fragment which contains a monovalent antigen-binding fragment of an antibody molecule, which can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)₂, the fragment of the antibody that can be obtained by treating whole antibody with the enzyme pepsin without subsequent reduction; F(ab′)₂ is a dimer of two Fab′ fragments held together by two disulfide bonds; (4) Fv, a genetically engineered fragment containing the variable region of the light chain and the variable region of the heavy chain expressed as two chains; or (5) Single chain antibody (“SCA”), a genetically engineered molecule containing the variable region of the light chain and the variable region of the heavy chain, linked by a suitable polypeptide linker as a genetically fused single chain molecule. Each possibility represents a separate embodiment of the present invention.

Methods of making these fragments are known in the art. (See for example, Harlow and Lane, Antibodies: A Laboratory Manual, Cold Spring Harbor Laboratory, New York, 1988, incorporated herein by reference).

In some embodiments, the antibody fragments may be prepared by proteolytic hydrolysis of the antibody or by expression in E. coli or mammalian cells (e.g. Chinese hamster ovary cell culture or other protein expression systems) of DNA encoding the fragment.

Antibody fragments can, in some embodiments, be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R., Biochem. J., 73: 119-126, 1959. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al., Proc. Nat'l Acad. Sci. USA 69:2659-62, 1972. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods, 2: 97-105, 1991; Bird et al., Science 242:423-426, 1988; Pack et al., Bio/Technology 11:1271-77, 1993; and Ladner et al., U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry, Methods, 2: 106-10, 1991.

In some embodiments, the antibodies or fragments as described herein may comprise “humanized forms” of antibodies. In some embodiments, the term “humanized forms of antibodies” refers to non-human (e.g. murine) antibodies, which are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human can be made by introducing of human immunoglobulin loci into transgenic animals, e.g. mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

In one embodiment, an antibody or functional fragment thereof binds to an antigen or a portion thereof comprising a T-cell receptor co-stimulatory molecule, an antigen presenting cell receptor binding co-stimulatory molecule or a member of the TNF receptor superfamily. In another embodiment, an antigen or portion thereof comprises a T-cell receptor co-stimulatory molecule comprising CD28, ICOS. In another embodiment, an antigen or portion thereof comprises an antigen presenting cell receptor binding co-stimulatory molecule comprising a CD80 receptor, a CD86 receptor, or a CD46 receptor. In another embodiment, an antigen or portion thereof comprises a TNF receptor superfamily member comprising glucocorticoid-induced TNF receptor (GITR), OX40 (CD134 receptor), 4-1BB (CD137 receptor) or TNFR25.

In one embodiment, an antibody or functional fragment comprises a T-cell receptor co-stimulatory molecule binding region, an antigen presenting cell receptor binding co-stimulatory molecule binding region, or a member of the TNF receptor superfamily binding region. In another embodiment, an antibody disclosed herein is a CD28 antibody, a ICOS antibody, or antibody against a heretofore unnamed co-stimulatory receptor. In another embodiment, the antibody is a CD80 receptor antibody, a CD86 receptor antibody, or a CD46 receptor antibody. In another embodiment, an antibody is a TNF receptor superfamily member-binding antibody which comprise a glucocorticoid-induced TNF receptor (GITR) antibody, an OX40 (CD134 receptor) antibody, a 4-1BB (CD137 receptor) antibody or a TNFR25 antibody. The form of the antibodies can be monoclonal, polyclonal, Human, or Humanized antibody derived from a non-human species of animal. The antibodies can be complete or partial with the variable portion of one or both antibody chains being specific to function as an agonist for the co-stimulatory receptor binding site.

In another embodiment, the antibody disclosed herein is an anti-OX40 antibody or antigen binding fragment thereof. In another embodiment, the antibody is an anti-GITR antibody or antigen binding fragment thereof.

In another embodiment, disclosed is a method of treating cancer or an infectious disease in a subject, the method comprising the steps of obtaining a population of effector T cells, treating the population with a GITR agonist is selected from the group consisting of GITRL, an active fragment of GITRL, a fusion protein containing GITRL, a fusion protein containing an active fragment of GITRL, an agonistic small molecule, and an agonistic anti-antibody. In another embodiment, the subject is afflicted with cancer.

In another embodiment, disclosed is a combination therapy comprising a recombinant Listeria strain and a GITR agonist selected from the group consisting of GITRL, an active fragment of GITRL, a fusion protein containing GITRL, a fusion protein containing an active fragment of GITRL, an agonistic small molecule, and an agonistic anti-antibody, wherein said combination therapy is for use in treating a subject having a tumor or cancer.

In one embodiment, the disclosure provides isolated binding molecules that bind to the human CD134, including anti-CD134 antibodies, and derivatives of the anti-CD134.

In another embodiment of the disclosure provides a binding molecule that binds to human CD134, wherein the binding molecule does not prevent human CD134 (OX40 ligand (OX40L) and wherein said binding molecule further does not impede the immunostimulatory and/or proliferative responses of human OX40L on human CD134 expressing T-effector cells.

In another embodiment, the disclosure provides a binding molecule that binds to human CD134, wherein the effect on binding of OX40L to CD134 on human CD134 expressing T-cells is reduced by not more than about 70%, or about 60%, or about 50%, or about 40%, or about 30%, or about 20%, or about 10% or less, and wherein said binding molecule enhances the immunostimulatory and/or proliferative responses of human OX40L on human CD134 expressing T-effector cells.

In another embodiment, the disclosure provides a binding molecule that binds to human CD134, wherein the binding molecule does not prevent human CD134 (OX40 ligand (OX40L) and wherein said binding molecule enhances the immunostimulatory and/or proliferative responses of human OX40L on human CD134 expressing T-effector cells.

In one embodiment, the disease disclosed herein is a cancer or a tumor. In one embodiment, the cancer treated by a method of the present invention is breast cancer. In another embodiment, the cancer is a cervical cancer. In another embodiment, the cancer is an Her2 containing cancer. In another embodiment, the cancer is a melanoma. In another embodiment, the cancer is pancreatic cancer. In another embodiment, the cancer is ovarian cancer. In another embodiment, the cancer is gastric cancer. In another embodiment, the cancer is a carcinomatous lesion of the pancreas. In another embodiment, the cancer is pulmonary adenocarcinoma. In another embodiment, the cancer is pulmonary adenocarcinoma. In another embodiment, it is a glioblastoma multiforme. In another embodiment, the cancer is colorectal adenocarcinoma. In another embodiment, the cancer is pulmonary squamous adenocarcinoma. In another embodiment, the cancer is gastric adenocarcinoma. In another embodiment, the cancer is an ovarian surface epithelial neoplasm (e.g. a benign, proliferative or malignant variety thereof). In another embodiment, the cancer is an oral squamous cell carcinoma. In another embodiment, the cancer is non-small-cell lung carcinoma. In another embodiment, the cancer is an endometrial carcinoma. In another embodiment, the cancer is a bladder cancer. In another embodiment, the cancer is a head and neck cancer. In another embodiment, the cancer is a prostate carcinoma. In another embodiment, the cancer is oropharyngeal cancer. In another embodiment, the cancer is lung cancer. In another embodiment, the cancer is anal cancer. In another embodiment, the cancer is colorectal cancer. In another embodiment, the cancer is esophageal cancer. In another embodiment, the cancer is mesothelioma.

In one embodiment, a heterologous antigen disclosed herein is HPV-E7. In another embodiment, the antigen is HPV-E6. In another embodiment, the HPV-E7 is from HPV strain 16. In another embodiment, the HPV-E7 is from HPV strain 18. In another embodiment, the HPV-E6 is from HPV strain 16. In another embodiment, the HPV-E7 is from HPV strain 18. In another embodiment, fragments of a heterologous antigen disclosed herein are also encompassed by the present invention.

In another embodiment, the antigen is Her-2/neu. In another embodiment, the antigen is NY-ESO-1. In another embodiment, the antigen is telomerase (TERT). In another embodiment, the antigen is SCCE. In another embodiment, the antigen is CEA. In another embodiment, the antigen is LMP-1. In another embodiment, the antigen is p53. In another embodiment, the antigen is carboxic anhydrase IX (CAIX). In another embodiment, the antigen is PSMA. In another embodiment, the antigen is prostate stem cell antigen (PSCA). In another embodiment, the antigen is HMW-MAA. In another embodiment, the antigen is WT-1. In another embodiment, the antigen is HIV-1 Gag. In another embodiment, the antigen is Proteinase 3. In another embodiment, the antigen is Tyrosinase related protein 2. In another embodiment, the antigen is PSA (prostate-specific antigen). In another embodiment, the antigen is selected from HPV-E7, HPV-E6, Her-2, NY-ESO-1, telomerase (TERT), SCCE, HMW-MAA, EGFR-survivin, baculoviral inhibitor of apoptosis repeat-containing 5 (BIRC5), WT-1, HIV-1 Gag, CEA, LMP-1, p53, PSMA, PSCA, Proteinase 3, Tyrosinase related protein 2, Muc1, PSA (prostate-specific antigen), or a combination thereof.

In another embodiment, an “immunogenic fragment” is one that elicits an immune response when administered to a subject alone or in a vaccine composition disclosed herein. Such a fragment contains, in another embodiment, the necessary epitopes in order to elicit either a humoral immune response, and/or an adaptive immune response.

In one embodiment, compositions disclosed herein comprise an antibody or a functional fragment thereof. In another embodiment, the compositions comprise at least one antibody or functional fragment thereof. In another embodiment, a composition may comprise 2 antibodies, 3 antibodies, 4 antibodies, or more than 4 antibodies. In another embodiment, a composition of this invention comprises an Lm strain and an antibody or a functional fragment thereof. In another embodiment, a composition disclosed herein comprises an Lm strain and at least one antibody or a functional fragment thereof. In another embodiment, a composition disclosed herein comprises an Lm strain and 2 antibodies, 3 antibodies, 4 antibodies, or more than 4 antibodies. In another embodiment, a composition disclosed herein comprises an antibody or a functional fragment thereof. Different antibodies present in the same or different compositions need not have the same form, for example one antibody may be a monoclonal antibody and another may be a FAb fragment.

In one embodiment, compositions disclosed herein comprise an antibody or a functional fragment thereof, which specifically binds GITR or a portion thereof. In another embodiment, compositions disclosed herein comprise an antibody or functional fragment thereof, which specifically binds OX40 or a portion thereof. In another embodiment, a composition may comprise an antibody that specifically bind GITR or a portion thereof, and an antibody that specifically binds OX40. In another embodiment, a composition of this invention comprises an Lm strain and an antibody or a functional fragment thereof that specifically binds GITR. In another embodiment, a composition of this invention comprises an Lm strain and an antibody or a functional fragment thereof that specifically binds OX40. In another embodiment, a composition of this invention comprises an Lm strain and an antibody that specifically binds GITR or a portion thereof, and an antibody that specifically binds OX40 or a portion thereof. In another embodiment, a composition of this invention comprises an antibody or a functional fragment thereof that specifically binds GITR, wherein the composition does not include a Listeria strain disclosed herein. In another embodiment, a composition of this invention comprises an antibody or a functional fragment thereof that specifically binds OX40, wherein the composition does not include a Listeria strain disclosed herein. In another embodiment, a composition of this invention comprises an antibody or a functional fragment thereof that specifically binds GITR, and an antibody that specifically binds GITR, wherein the composition does not include a Listeria strain disclosed herein. Different antibodies present in the same or different compositions need not have the same form, for example one antibody may be a monoclonal antibody and another may be a FAb fragment. Each possibility represents a different embodiment of this invention.

The term “antibody functional fragment” refers to a portion of an intact antibody that is capable of specifically binding to an antigen to cause the biological effect intended by the present invention. Examples of antibody fragments include, but are not limited to, Fab, Fab′, F(ab′)₂, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations, κ and λ light chains refer to the two major antibody light chain isotypes.

A skilled artisan will understand that the term “synthetic antibody” may encompass an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

In one embodiment, an antibody or functional fragment thereof comprises an antigen binding region. In one embodiment, an antigen binding regions is an antibody or an antigen-binding domain thereof. In one embodiment, the antigen-binding domain thereof is a Fab or a scFv.

It will be appreciated by a skilled artisan that the term “binds” or “specifically binds,” with respect to an antibody, encompasses an antibody or functional fragment thereof, which recognizes a specific antigen, but does not substantially recognize or bind other molecules in a sample. For example, an antibody that specifically binds to an antigen from one species may also bind to that antigen from one or more species, but, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than a specific amino acid sequence.

In one embodiment, a composition of this invention comprises a recombinant Listeria monocytogenes (Lm) strain. In another embodiment, a composition disclosed herein comprises an antibody or functional fragment thereof, as described herein.

In one embodiment, an immunogenic composition comprises an antibody or a functional fragment thereof, disclosed herein, and a recombinant attenuated Listeria, disclosed herein. In another embodiment, each component of the immunogenic compositions disclosed herein is administered prior to, concurrently with, or after another component of the immunogenic compositions disclosed herein. In one embodiment, even when administered concurrently, an Lm composition and an antibody or functional fragment thereof may be administered as two separate compositions. Alternately, in another embodiment, an Lm composition may comprise an antibody or a functional fragment thereof.

The compositions disclosed herein, in another embodiment, are administered to a subject by any method known to a person skilled in the art, such as parenterally, paracancerally, transmucosally, transdermally, intramuscularly, intravenously, intra-dermally, subcutaneously, intra-peritonealy, intra-ventricularly, intra-cranially, intra-vaginally or intra-tumorally.

In another embodiment, the compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment of the present invention, the active ingredient is formulated in a capsule. In accordance with this embodiment, the compositions of the present invention comprise, in addition to the active compound and the inert carrier or diluent, a hard gelating capsule.

In another embodiment, compositions are administered by intravenous, intra-arterial, or intra-muscular injection of a liquid preparation. Suitable liquid formulations include solutions, suspensions, dispersions, emulsions, oils and the like. In one embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intramuscularly and are thus formulated in a form suitable for intra-muscular administration.

In some embodiments, when the antibody or functional fragment thereof is administered separately from a composition comprising a recombinant Lm strain, the antibody may be injected intravenously, subcutaneously, or directly into the tumor or tumor bed. In one embodiment, a composition comprising an antibody is injected into the space left after a tumor has been surgically removed, e.g., the space in a prostate gland following removal of a prostate tumor.

In one embodiment, the term “immunogenic composition” may encompass the recombinant Listeria disclosed herein, and an adjuvant, and an antibody or functional fragment thereof, or any combination thereof. In another embodiment, an immunogenic composition comprises a recombinant Listeria disclosed herein. In another embodiment, an immunogenic composition comprises an adjuvant known in the art or as disclosed herein. It is also to be understood that administration of such compositions enhance an immune response, or increase a T effector cell to regulatory T cell ratio or elicit an anti-tumor immune response, as further disclosed herein.

In one embodiment, this invention provides methods of use which comprise administering a composition comprising the described Listeria strains, and further comprising an antibody or functional fragment thereof. In another embodiment, methods of use comprise administering more than one antibody disclosed herein, which may be present in the same or a different composition, and which may be present in the same composition as the Listeria or in a separate composition.

In one embodiment, the term “pharmaceutical composition” encompasses a therapeutically effective amount of the active ingredient or ingredients including the Listeria strain, and at least one antibody or functional fragment thereof, together with a pharmaceutically acceptable carrier or diluent. It is to be understood that the term a “therapeutically effective amount” refers to that amount which provides a therapeutic effect for a given condition and administration regimen.

It will be understood by the skilled artisan that the term “administering” encompasses bringing a subject in contact with a composition of the present invention. In one embodiment, administration can be accomplished in vitro, i.e. in a test tube, or in vivo, i.e. in cells or tissues of living organisms, for example humans. In one embodiment, the present invention encompasses administering the Listeria strains and compositions thereof of the present invention to a subject.

The term “about” as used herein means in quantitative terms plus or minus 5%, or in another embodiment plus or minus 10%, or in another embodiment plus or minus 15%, or in another embodiment plus or minus 20%. It is to be understood by the skilled artisan that the term “subject” can encompass a mammal including an adult human or a human child, teenager or adolescent in need of therapy for, or susceptible to, a condition or its sequalae, and also may include non-human mammals such as dogs, cats, pigs, cows, sheep, goats, horses, rats, and mice. It will also be appreciated that the term may encompass livestock. The term “subject” does not exclude an individual that is normal in all respects.

Following the administration of the immunogenic compositions disclosed herein, the methods disclosed herein induce the expansion of T effector cells in peripheral lymphoid organs leading to an enhanced presence of T effector cells at the tumor site. In another embodiment, the methods disclosed herein induce the expansion of T effector cells in peripheral lymphoid organs leading to an enhanced presence of T effector cells at the periphery. Such expansion of T effector cells leads to an increased ratio of T effector cells to regulatory T cells in the periphery and at the tumor site without affecting the number of Tregs. It will be appreciated by the skilled artisan that peripheral lymphoid organs include, but are not limited to, the spleen, peyer's patches, the lymph nodes, the adenoids, etc. In one embodiment, the increased ratio of T effector cells to regulatory T cells occurs in the periphery without affecting the number of Tregs. In another embodiment, the increased ratio of T effector cells to regulatory T cells occurs in the periphery, the lymphoid organs and at the tumor site without affecting the number of Tregs at these sites. In another embodiment, the increased ratio of T effector cells decrease the frequency of Tregs, but not the total number of Tregs at these sites.

### Combination Therapies and Methods of Use Thereof

In one embodiment, disclosed provides a method of eliciting an enhanced anti-tumor T cell response in a subject, the method comprising the step of administering to the subject an effective amount of an immunogenic composition comprising a recombinant Listeria strain comprising a nucleic acid molecule, the nucleic acid molecule comprising a first open reading frame encoding a fusion polypeptide, wherein the fusion polypeptide comprises a truncated listeriolysin O (LLO) protein, a truncated ActA protein, or a PEST amino acid sequence fused to a heterologous antigen or fragment thereof, wherein said method further comprises a step of administering an effective amount of a composition comprising an antibody or fragment thereof to said subject. In another embodiment, the antibody is an agonist antibody or antigen binding fragment thereof. In another embodiment, the antibody is an anti-TNF receptor antibody or antigen binding fragment thereof. In another embodiment, the antibody is an anti-OX40 antibody or antigen binding fragment thereof. In another embodiment, the antibody is an anti-GITR antibody or antigen binding fragment thereof. In another embodiment, said method further comprises administering additional antibodies, which may be comprise in the composition comprising said recombinant Listeria strain or may be comprised in a separate composition.

In another embodiment, disclosed is a method of eliciting an enhanced anti-tumor T cell response in a subject, the method comprising the step of administering to the subject an effective amount of an immunogenic composition comprising a recombinant Listeria strain comprising a nucleic acid molecule, the nucleic acid molecule comprising a first open reading frame encoding a truncated listeriolysin O (LLO) protein, a truncated ActA protein, or a PEST amino acid sequence, wherein said method further comprises a step of administering an effective amount of a composition comprising an antibody or fragment thereof to said subject. In another embodiment, the antibody is an agonist antibody or antigen binding fragment thereof. In another embodiment, the antibody is an anti-TNF receptor antibody or antigen binding fragment thereof. In another embodiment, the antibody is an anti-OX40 antibody or antigen binding fragment thereof. In another embodiment, the antibody is an anti-GITR antibody or antigen binding fragment thereof. In another embodiment, said method further comprises administering additional antibodies, which may be comprise in the composition comprising said recombinant Listeria strain or may be comprised in a separate composition.

In one embodiment, any composition comprising a Listeria strain described herein may be used in the methods disclosed herein. In one embodiment, any composition comprising a Listeria strain and an antibody or fragment thereof, for example an antibody binding a TNF receptor super family member, or an antibody binding to a T-cell receptor co-stimulatory molecule or an antibody binding to an antigen presenting cell receptor binding a co-stimulatory molecule, as described herein, may be used in the methods of this invention. In one embodiment, any composition comprising an antibody or functional fragment thereof described herein may be used in the methods disclosed herein. Compositions comprising Listeria strains with and without antibodies have been described in detail above. Compositions with antibodies have also been described in detail above. In some embodiment, in a method of this invention a composition comprising an antibody or fragment thereof, for example an antibody binding to a TNF receptor super family member, or an antibody binding to a T-cell receptor co-stimulatory molecule or an antibody binding to an antigen presenting cell receptor binding a co-stimulatory molecule, may be administered prior to, concurrent with or following administration of a composition comprising a Listeria strain.

In one embodiment, repeat administrations (doses) of compositions disclosed herein may be undertaken immediately following the first course of treatment or after an interval of days, weeks or months to achieve tumor regression. In another embodiment, repeat doses may be undertaken immediately following the first course of treatment or after an interval of days, weeks or months to achieve suppression of tumor growth. Assessment may be determined by any of the techniques known in the art, including diagnostic methods such as imaging techniques, analysis of serum tumor markers, biopsy, or the presence, absence or amelioration of tumor associated symptoms.

In one embodiment, disclosed herein are methods and compositions for preventing, treating and vaccinating against a heterologous antigen-expressing tumor and inducing an immune response against sub-dominant epitopes of the heterologous antigen, while preventing an escape mutation of the tumor.

In one embodiment, the methods and compositions for preventing, treating and vaccinating against a heterologous antigen-expressing tumor comprise the use of a truncated Listeriolysin (tLLO) protein. In another embodiment, the methods and compositions disclosed herein comprise a recombinant Listeria overexpressing tLLO. In another embodiment, the tLLO is expressed from a plasmid within the Listeria.

In another embodiment, disclosed herein is a method of preventing or treating a tumor growth or cancer in a subject, the method comprising the step of administering to the subject an immunogenic composition comprising an antibody or functional fragment thereof, as described herein, and a recombinant Listeria strain comprising a nucleic acid molecule, the nucleic acid molecule comprising a first open reading frame encoding a fusion polypeptide, wherein the fusion polypeptide comprises a truncated listeriolysin O (LLO) protein, a truncated ActA protein, or a PEST amino acid sequence fused to a heterologous antigen or fragment thereof. In another embodiment, disclosed herein is a method of preventing or treating a tumor growth or cancer in a subject, the method comprising the step of administering to the subject an immunogenic composition comprising an antibody or functional fragment thereof, as described herein, and a recombinant Listeria strain comprising a nucleic acid molecule, the nucleic acid molecule comprising a first open reading frame encoding a truncated listeriolysin O (LLO) protein, a truncated ActA protein, or a PEST amino acid sequence.

In one embodiment, the term “treating” refers to curing a disease. In another embodiment, “treating” refers to preventing a disease. In another embodiment, “treating” refers to reducing the incidence of a disease. In another embodiment, “treating” refers to ameliorating symptoms of a disease. In another embodiment, “treating” refers to increasing performance free survival or overall survival of a patient. In another embodiment, “treating” refers to stabilizing the progression of a disease. In another embodiment, “treating” refers to inducing remission. In another embodiment, “treating” refers to slowing the progression of a disease. The terms “reducing”, “suppressing” and “inhibiting” refer in another embodiment to lessening or decreasing.

In one embodiment, disclosed herein is a method of increasing a ratio of T effector cells to regulatory T cells (Tregs) in the spleen and tumor microenvironments of a subject, comprising administering the immunogenic composition disclosed herein. In another embodiment, increasing a ratio of T effector cells to regulatory T cells (Tregs) in the spleen and tumor microenvironments in a subject allows for a more profound anti-tumor response in the subject.

In another embodiment, the T effector cells comprise CD4+FoxP3− T cells. In another embodiment, the T effector cells are CD4+FoxP3− T cells. In another embodiment, the T effector cells comprise CD4+FoxP3− T cells and CD8+ T cells. In another embodiment, the T effector cells are CD4+FoxP3− T cells and CD8+ T cells. In another embodiment, the regulatory T cells is a CD4+FoxP3+ T cell.

In one embodiment, the present invention provides methods of treating, protecting against, and inducing an immune response against a tumor or a cancer, comprising the step of administering to a subject the immunogenic composition disclosed herein.

In one embodiment, the present invention provides a method of preventing or treating a tumor or cancer in a human subject, comprising the step of administering to the subject the immunogenic composition strain disclosed herein, the recombinant Listeria strain comprising a recombinant polypeptide comprising an N-terminal fragment of an LLO protein and tumor-associated antigen, whereby the recombinant Listeria strain induces an immune response against the tumor-associated antigen, thereby treating a tumor or cancer in a human subject. In another embodiment, the immune response is a T-cell response. In another embodiment, the T-cell response is a CD4+FoxP3− T cell response. In another embodiment, the T-cell response is a CD8+ T cell response. In another embodiment, the T-cell response is a CD4+FoxP3− and CD8+ T cell response. In another embodiment, the present invention provides a method of protecting a subject against a tumor or cancer, comprising the step of administering to the subject the immunogenic composition disclosed herein. In another embodiment, the present invention provides a method of inducing regression of a tumor in a subject, comprising the step of administering to the subject the immunogenic composition disclosed herein. In another embodiment, disclosed herein is a method of reducing the incidence or relapse of a tumor or cancer, comprising the step of administering to the subject the immunogenic composition disclosed herein. In another embodiment, disclosed herein is a method of suppressing the formation of a tumor in a subject, comprising the step of administering to the subject the immunogenic composition disclosed herein. In another embodiment, disclosed herein is a method of inducing a remission of a cancer in a subject, comprising the step of administering to the subject the immunogenic composition disclosed herein. In one embodiment, the nucleic acid molecule comprising a first open reading frame encoding a fusion polypeptide is integrated into the Listeria genome. In another embodiment, the nucleic acid is in a plasmid in the recombinant Listeria vaccine strain.

In one embodiment, the method comprises the step of co-administering the recombinant Listeria with an additional therapy. In another embodiment, the additional therapy is surgery, chemotherapy, an immunotherapy, a radiation therapy, antibody based immuno therapy, or a combination thereof. In another embodiment, the additional therapy precedes administration of the recombinant Listeria. In another embodiment, the additional therapy follows administration of the recombinant Listeria. In another embodiment, the additional therapy is an antibody therapy. In another embodiment, the recombinant Listeria is administered in increasing doses in order to increase the T-effector cell to regulatory T cell ration and generate a more potent anti-tumor immune response. It will be appreciated by a skilled artisan that the anti-tumor immune response can be further strengthened by providing the subject having a tumor with cytokines including, but not limited to IFN-γ, TNF-α, and other cytokines known in the art to enhance cellular immune response, some of which can be found in U.S. Pat. No. 6,991,785, incorporated by reference herein.

In one embodiment, the methods disclosed herein further comprise the step of co-administering an immunogenic composition disclosed herein with an antibody or functional fragment thereof that enhances an anti-tumor immune response in said subject.

In one embodiment, the methods disclosed herein further comprise the step of co-administering an immunogenic composition disclosed herein with a indoleamine 2,3-dioxygenase (IDO) pathway inhibitor. IDO pathway inhibitors for use in the present invention include any IDO pathway inhibitor known in the art, including but not limited to, 1-methyltryptophan (1MT), 1-methyltryptophan (1MT), Necrostatin-1, Pyridoxal Isonicotinoyl Hydrazone, Ebselen, 5-Methylindole-3-carboxaldehyde, CAY10581, an anti-IDO antibody or a small molecule DO inhibitor. In another embodiment, the compositions and methods disclosed herein are also used in conjunction with, prior to, or following a chemotherapeutic or radiotherapeutic regiment. In another embodiment, DO inhibition enhances the efficiency of chemotherapeutic agents.

In another embodiment, disclosed herein is a method of increasing survival of a subject suffering from cancer or having a tumor, the method comprising the step of administering to the subject an immunogenic composition comprising an antibody or functional fragment thereof, as described herein, and a recombinant Listeria strain comprising a nucleic acid molecule, the nucleic acid molecule comprising a first open reading frame encoding a fusion polypeptide, wherein the fusion polypeptide comprises a truncated listeriolysin O (LLO) protein, a truncated ActA protein, or a PEST amino acid sequence fused to a heterologous antigen or fragment thereof.

In another embodiment, disclosed herein is a method of increasing antigen-specific T cells in a subject suffering from cancer or having a tumor, the method comprising the step of administering to the subject an immunogenic composition comprising an antibody or functional fragment thereof, as described herein, and a recombinant Listeria strain comprising a nucleic acid molecule, the nucleic acid molecule comprising a first open reading frame encoding a fusion polypeptide, wherein the fusion polypeptide comprises a truncated listeriolysin O (LLO) protein, a truncated ActA protein, or a PEST amino acid sequence fused to a heterologous antigen or fragment thereof. In another embodiment, disclosed herein is a method of increasing T cells in a subject suffering from cancer or having a tumor, the method comprising the step of administering to the subject an immunogenic composition comprising an antibody or functional fragment thereof, as described herein, and a recombinant Listeria strain comprising a nucleic acid molecule, the nucleic acid molecule comprising a first open reading frame encoding a truncated listeriolysin O (LLO) protein, a truncated ActA protein, or a PEST amino acid sequence.

In another embodiment, a method of present invention further comprises the step of boosting the subject with a recombinant Listeria strain or an antibody or functional fragment thereof, as disclosed herein. In another embodiment, the recombinant Listeria strain used in the booster inoculation is the same as the strain used in the initial “priming” inoculation. In another embodiment, the booster strain is different from the priming strain. In another embodiment, the antibody used in the booster inoculation binds the same antigen as the antibody used in the initial “priming” inoculation. In another embodiment, the booster antibody is different from the priming antibody. In another embodiment, the same doses are used in the priming and boosting inoculations. In another embodiment, a larger dose is used in the booster. In another embodiment, a smaller dose is used in the booster. In another embodiment, the methods of the present invention further comprise the step of administering to the subject a booster vaccination. In one embodiment, the booster vaccination follows a single priming vaccination. In another embodiment, a single booster vaccination is administered after the priming vaccinations. In another embodiment, two booster vaccinations are administered after the priming vaccinations. In another embodiment, three booster vaccinations are administered after the priming vaccinations. In one embodiment, the period between a prime and a boost strain is experimentally determined by the skilled artisan. In another embodiment, the period between a prime and a boost strain is 1 week, in another embodiment it is 2 weeks, in another embodiment, it is 3 weeks, in another embodiment, it is 4 weeks, in another embodiment, it is 5 weeks, in another embodiment it is 6-8 weeks, in yet another embodiment, the boost strain is administered 8-10 weeks after the prime strain.

In another embodiment, a method of the present invention further comprises boosting the subject with a immunogenic composition comprising an attenuated Listeria strain disclosed herein. In another embodiment, a method of the present invention comprises the step of administering a booster dose of the immunogenic composition comprising the attenuated Listeria strain disclosed herein. In another embodiment, the booster dose is an alternate form of said immunogenic composition. In another embodiment, the methods of the present invention further comprise the step of administering to the subject a booster immunogenic composition. In one embodiment, the booster dose follows a single priming dose of said immunogenic composition. In another embodiment, a single booster dose is administered after the priming dose. In another embodiment, two booster doses are administered after the priming dose. In another embodiment, three booster doses are administered after the priming dose. In one embodiment, the period between a prime and a boost dose of an immunogenic composition comprising the attenuated Listeria disclosed herein is experimentally determined by the skilled artisan. In another embodiment, the dose is experimentally determined by a skilled artisan. In another embodiment, the period between a prime and a boost dose is 1 week, in another embodiment it is 2 weeks, in another embodiment, it is 3 weeks, in another embodiment, it is 4 weeks, in another embodiment, it is 5 weeks, in another embodiment it is 6-8 weeks, in yet another embodiment, the boost dose is administered 8-10 weeks after the prime dose of the immunogenic composition.

Heterologous “prime boost” strategies have been effective for enhancing immune responses and protection against numerous pathogens. Schneider et al., Immunol. Rev. 170:29-38 (1999); Robinson, H. L., Nat. Rev. Immunol. 2:239-50 (2002); Gonzalo, R. M. et al., Strain 20:1226-31 (2002); Tanghe, A., Infect. Immun. 69:3041-7 (2001). Providing antigen in different forms in the prime and the boost injections appears to maximize the immune response to the antigen. DNA strain priming followed by boosting with protein in adjuvant or by viral vector delivery of DNA encoding antigen appears to be the most effective way of improving antigen specific antibody and CD4+ T-cell responses or CD8+ T-cell responses respectively. Shiver J. W. et al., Nature 415: 331-5 (2002); Gilbert, S. C. et al., Strain 20:1039-45 (2002); Billaut-Mulot, O. et al., Strain 19:95-102 (2000); Sin, J. I. et al., DNA Cell Biol. 18:771-9 (1999). Recent data from monkey vaccination studies suggests that adding CRL1005 poloxamer (12 kDa, 5% POE), to DNA encoding the HIV gag antigen enhances T-cell responses when monkeys are vaccinated with an HIV gag DNA prime followed by a boost with an adenoviral vector expressing HIV gag (Ad5-gag). The cellular immune responses for a DNA/poloxamer prime followed by an Ad5-gag boost were greater than the responses induced with a DNA (without poloxamer) prime followed by Ad5-gag boost or for Ad5-gag only. Shiver, J. W. et al. Nature 415:331-5 (2002). U.S. Patent Appl. Publication No. US 2002/0165172 A1 describes simultaneous administration of a vector construct encoding an immunogenic portion of an antigen and a protein comprising the immunogenic portion of an antigen such that an immune response is generated. The document is limited to hepatitis B antigens and HIV antigens. Moreover, U.S. Pat. No. 6,500,432 is directed to methods of enhancing an immune response of nucleic acid vaccination by simultaneous administration of a polynucleotide and polypeptide of interest. According to the patent, simultaneous administration means administration of the polynucleotide and the polypeptide during the same immune response, preferably within 0-10 or 3-7 days of each other. The antigens contemplated by the patent include, among others, those of Hepatitis (all forms), HSV, HIV, CMV, EBV, RSV, VZV, HPV, polio, influenza, parasites (e.g., from the genus Plasmodium), and pathogenic bacteria (including but not limited to M. tuberculosis, M. leprae, Chlamydia, Shigella, B. burgdorferi, enterotoxigenic E. coli, S. typhosa, H. pylori, V. cholerae, B. pertussis, etc.). All of the above references are herein incorporated by reference in their entireties.

In one embodiment, a treatment protocol of the present invention is therapeutic. In another embodiment, the protocol is prophylactic. In another embodiment, the compositions of the present invention are used to protect people at risk for cancer such as breast cancer or other types of tumors because of familial genetics or other circumstances that predispose them to these types of ailments as will be understood by a skilled artisan. In another embodiment, the vaccines are used as a cancer immunotherapy after debulking of tumor growth by surgery, conventional chemotherapy or radiation treatment. Following such treatments, the vaccines of the present invention are administered so that the CTL response to the tumor antigen of the vaccine destroys remaining metastases and prolongs remission from the cancer. In another embodiment, vaccines of the present invention are used to effect the growth of previously established tumors and to kill existing tumor cells.

In some embodiments, the term “comprise” or grammatical forms thereof, refers to the inclusion of the indicated active agent, such as the Lm strains of this invention, as well as inclusion of other active agents, such as an antibody or functional fragment thereof, and pharmaceutically acceptable carriers, excipients, emollients, stabilizers, etc., as are known in the pharmaceutical industry. In some embodiments, the term “consisting essentially of” refers to a composition, whose only active ingredient is the indicated active ingredient, however, other compounds may be included which are for stabilizing, preserving, etc. the formulation, but are not involved directly in the therapeutic effect of the indicated active ingredient. In some embodiments, the term “consisting essentially of” may refer to components, which exert a therapeutic effect via a mechanism distinct from that of the indicated active ingredient. In some embodiments, the term “consisting essentially of” may refer to components, which exert a therapeutic effect and belong to a class of compounds distinct from that of the indicated active ingredient. In some embodiments, the term “consisting essentially of” may refer to components, which exert a therapeutic effect and may be distinct from that of the indicated active ingredient, by acting via a different mechanism of action, for example. In some embodiments, the term “consisting essentially of” may refer to components which facilitate the release of the active ingredient. In some embodiments, the term “consisting” refers to a composition, which contains the active ingredient and a pharmaceutically acceptable carrier or excipient.

As used herein, the singular form “a,” “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

In the following examples, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

### EXAMPLES

### Materials and Experimental Methods (Examples 1-2)

Cell Lines

The C57BL/6 syngeneic TC-1 tumor was immortalized with HPV-16 E6 and E7 and transformed with the c-Ha-ras oncogene. TC-1, provided by T. C. Wu (Johns Hopkins University School of Medicine, Baltimore, Md.) is a highly tumorigenic lung epithelial cell expressing low levels of with HPV-16 E6 and E7 and transformed with the c-Ha-ras oncogene. TC-1 was grown in RPMI 1640, 10% FCS, 2 mM L-glutamine, 100 Um′ penicillin, 100 μg/ml streptomycin, 100 μM nonessential amino acids, 1 mM sodium pyruvate, 50 micromolar (mcM) 2-ME, 400 microgram (mcg)/ml G418, and 10% National Collection Type Culture-109 medium at 37° with 10% CO₂. C3 is a mouse embryo cell from C57BL/6 mice immortalized with the complete genome of HPV 16 and transformed with pEJ-ras. EL-4/E7 is the thymoma EL-4 retrovirally transduced with E7.

L. monocytogenes Strains and Propagation

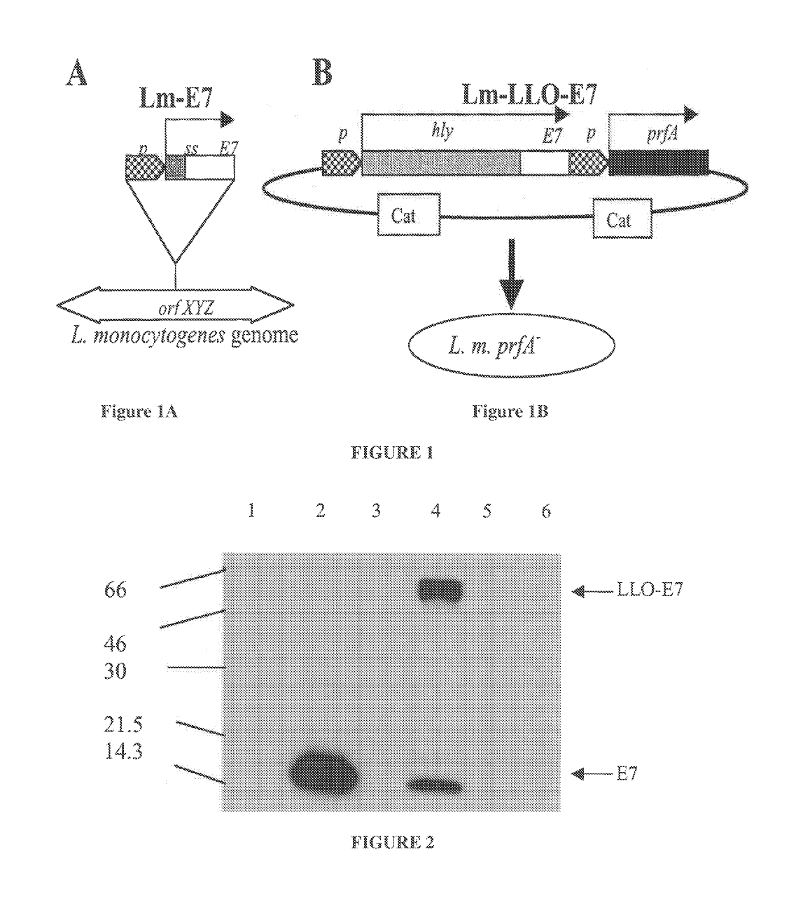

Listeria strains used were Lm-LLO-E7, also referred to herein as ADXS11-001, (hly-E7 fusion gene in an episomal expression system; FIG. 1A), Lm-E7 (single-copy E7 gene cassette integrated into Listeria genome), Lm-LLO-NP (“DP-L2028”; hly-NP fusion gene in an episomal expression system), and Lm-Gag (“ZY-18”; single-copy HIV-1 Gag gene cassette integrated into the chromosome). E7 was amplified by PCR using the primers 5′-GGCTCGAGCATGGAGATACACC-3′ (SEQ ID No: 51; XhoI site is underlined) and 5′-GGGGACTAGTTTATGGTTTCTGAGAACA-3′ (SEQ ID No: 52; SpeI site is underlined) and ligated into pCR2.1 (Invitrogen, San Diego, Calif.). E7 was excised from pCR2.1 by XhoI/SpeI digestion and ligated into pGG-55. The hly-E7 fusion gene and the pluripotential transcription factor prfA were cloned into pAM401, a multicopy shuttle plasmid (Wirth R et al, J Bacterial, 165: 831, 1986), generating pGG-55. The hly promoter drives the expression of the first 441 AA of the hly gene product, (lacking the hemolytic C-terminus, referred to below as “ΔLLO,” and having the sequence set forth in SEQ ID No: 3), which is joined by the XhoI site to the E7 gene, yielding a hly-E7 fusion gene that is transcribed and secreted as LLO-E7. Transformation of a prfA negative strain of Listeria, XFL-7 (provided by Dr. Hao Shen, University of Pennsylvania), with pGG-55 selected for the retention of the plasmid in vivo (FIGS. 1A-B). The hly promoter and gene fragment were generated using primers 5′-GGGGGCTAGCCCTCCTTTGATTAGTATATTC-3′ (SEQ ID No: 53; NheI site is underlined) and 5′-CTCCCTCGAGATCATAATTTACTTCATC-3′ (SEQ ID No: 54; XhoI site is underlined). The prfA gene was PCR amplified using primers 5′-GACTACAAGGACGATGACCGACAAGTGATAACCCGGGATCTAAATAAATCCGTTT-3′ (SEQ ID No: 55; XbaI site is underlined) and 5′-CCCGTCGACCAGCTCTTCTTGGTGAAG-3′ (SEQ ID No: 56; SalI site is underlined). Lm-E7 was generated by introducing an expression cassette containing the hly promoter and signal sequence driving the expression and secretion of E7 into the orfZ domain of the LM genome. E7 was amplified by PCR using the primers 5′-GCGGATCCCATGGAGATACACCTAC-3′ (SEQ ID No: 57; BamHI site is underlined) and 5′-GCTCTAGATTATGGTTTCTGAG-3′ (SEQ ID No: 58; XbaI site is underlined). E7 was then ligated into the pZY-21 shuttle vector. LM strain 10403S was transformed with the resulting plasmid, pZY-21-E7, which includes an expression cassette inserted in the middle of a 1.6-kb sequence that corresponds to the orfX, Y, Z domain of the LM genome. The homology domain allows for insertion of the E7 gene cassette into the orfZ domain by homologous recombination. Clones were screened for integration of the E7 gene cassette into the orfZ domain. Bacteria were grown in brain heart infusion medium with (Lm-LLO-E7 and Lm-LLO-NP) or without (Lm-E7 and ZY-18) chloramphenicol (20 μg/ml). Bacteria were frozen in aliquots at −80° C. Expression was verified by Western blotting (FIG. 2).

Western Blotting

Listeria strains were grown in Luria-Bertoni medium at 37° C. and were harvested at the same optical density measured at 600 nm. The supernatants were TCA precipitated and resuspended in 1× sample buffer supplemented with 0.1 N NaOH. Identical amounts of each cell pellet or each TCA-precipitated supernatant were loaded on 4-20% Tris-glycine SDS-PAGE gels (NOVEX, San Diego, Calif.). The gels were transferred to polyvinylidene difluoride and probed with an anti-E7 monoclonal antibody (mAb) (Zymed Laboratories, South San Francisco, Calif.), then incubated with HRP-conjugated anti-mouse secondary Ab (Amersham Pharmacia Biotech, Little Chalfont, U.K.), developed with Amersham ECL detection reagents, and exposed to Hyperfilm (Amersham Pharmacia Biotech).

Measurement of Tumor Growth

Tumors were measured every other day with calipers spanning the shortest and longest surface diameters. The mean of these two measurements was plotted as the mean tumor diameter in millimeters against various time points. Mice were sacrificed when the tumor diameter reached 20 mm. Tumor measurements for each time point are shown only for surviving mice.

Effects of Listeria Recombinants on Established Tumor Growth

Six- to 8-wk-old C57BL/6 mice (Charles River) received 2×10⁵ TC-1 cells s.c. on the left flank. One week following tumor inoculation, the tumors had reached a palpable size of 4-5 mm in diameter. Groups of eight mice were then treated with 0.1 LD₅₀ i.p. Lm-LLO-E7 (10⁷ CFU), Lm-E7 (10⁶ CFU), Lm-LLO-NP (10⁷ CFU), or Lm-Gag (5×10⁵ CFU) on days 7 and 14.

⁵¹Cr Release Assay

C57BL/6 mice, 6-8 wk old, were immunized i.p. with 0.1 LD₅₀ Lm-LLO-E7, Lm-E7, Lm-LLO-NP, or Lm-Gag. Ten days post-immunization, spleens were harvested. Splenocytes were established in culture with irradiated TC-1 cells (100:1, splenocytes:TC-1) as feeder cells; stimulated in vitro for 5 days, then used in a standard ⁵¹Cr release assay, using the following targets: EL-4, EL-4/E7, or EL-4 pulsed with E7 H-2b peptide (RAHYNIVTF). E:T cell ratios, performed in triplicate, were 80:1, 40:1, 20:1, 10:1, 5:1, and 2.5:1. Following a 4-h incubation at 37° C., cells were pelleted, and 50 μl supernatant was removed from each well. Samples were assayed with a Wallac 1450 scintillation counter (Gaithersburg, Md.). The percent specific lysis was determined as [(experimental counts per minute (cpm)−spontaneous cpm)/(total cpm−spontaneous cpm)]×100.

TC-1-Specific Proliferation

C57BL/6 mice were immunized with 0.1 LD₅₀ and boosted by i.p. injection 20 days later with 1 LD₅₀ Lm-LLO-E7, Lm-E7, Lm-LLO-NP, or Lm-Gag. Six days after boosting, spleens were harvested from immunized and naive mice. Splenocytes were established in culture at 5×10⁵/well in flat-bottom 96-well plates with 2.5×10⁴, 1.25×10⁴, 6×10³, or 3×10³ irradiated TC-1 cells/well as a source of E7 Ag, or without TC-1 cells or with 10 μg/ml Con A. Cells were pulsed 45 h later with 0.5 μCi [³H]thymidine/well. Plates were harvested 18 h later using a Tomtec harvester 96 (Orange, Conn.), and proliferation was assessed with a Wallac 1450 scintillation counter. The change in cpm was calculated as experimental cpm−no Ag cpm.

Flow Cytometric Analysis

C57BL/6 mice were immunized intravenously (i.v.) with 0.1 LD₅₀ Lm-LLO-E7 or Lm-E7 and boosted 30 days later. Three-color flow cytometry for CD8 (53-6.7, PE conjugated), CD62 ligand (CD62L; MEL-14, APC conjugated), and E7 H-2Db tetramer was performed using a FACSCalibur® flow cytometer with CellQuest® software (Becton Dickinson, Mountain View, Calif.). Splenocytes harvested 5 days after the boost were stained at room temperature (rt) with H-2Db tetramers loaded with the E7 peptide (RAHYNIVTF) or a control (HIV-Gag) peptide. Tetramers were used at a 1/200 dilution and were provided by Dr. Larry R. Pease (Mayo Clinic, Rochester, Minn.) and by the NIAID Tetramer Core Facility and the NIH AIDS Research and Reference Reagent Program. Tetramer⁺, CD8⁺, CD62L^{low} cells were analyzed.

B16F0-Ova Experiment

24 C57BL/6 mice were inoculated with 5×10⁵ B16F0-Ova cells. On days 3, 10 and 17, groups of 8 mice were immunized with 0.1 LD₅₀ Lm-OVA (10⁶ cfu), Lm-LLO-OVA (10⁸ cfu) and eight animals were left untreated.

Statistics

For comparisons of tumor diameters, mean and SD of tumor size for each group were determined, and statistical significance was determined by Student's t test. p≦0.05 was considered significant.

### Example 1: LLO-Antigen Fusions Induce Anti-Tumor Immunity

### Results

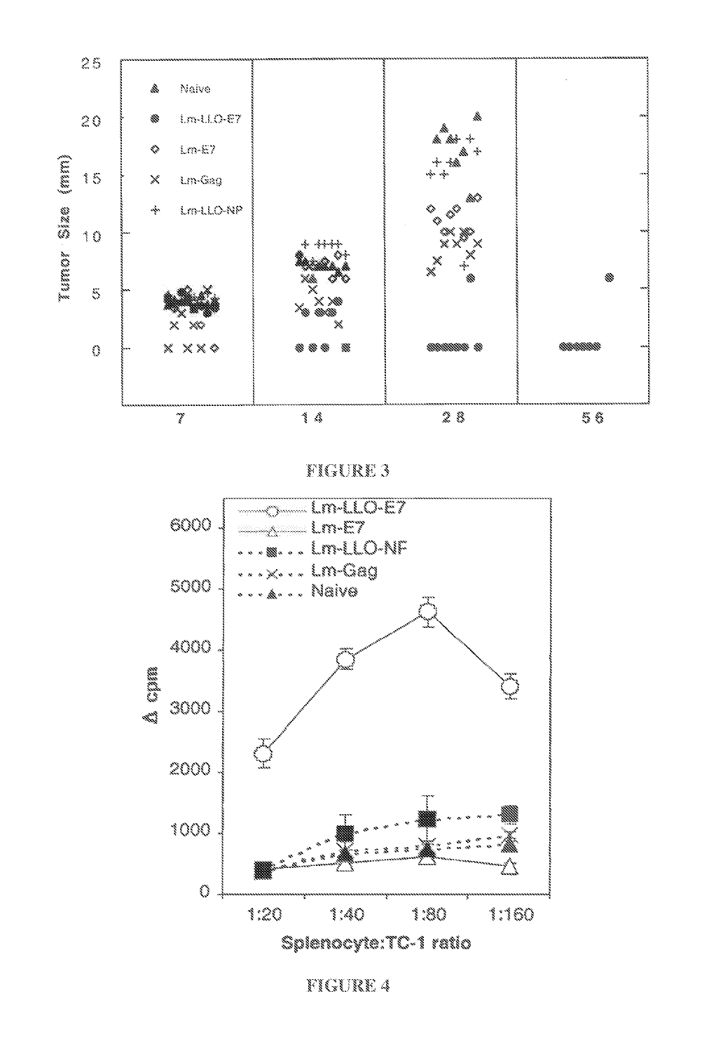

Lm-E7 and Lm-LLO-E7 were compared for their abilities to impact on TC-1 growth. Subcutaneous tumors were established on the left flank of C57BL/6 mice. Seven days later tumors had reached a palpable size (4-5 mm). Mice were vaccinated on days 7 and 14 with 0.1 LD₅₀ Lm-E7, Lm-LLO-E7, or, as controls, Lm-Gag and Lm-LLO-NP. Lm-LLO-E7 induced complete regression of 75% of established TC-1 tumors, while tumor growth was controlled in the other 2 mice in the group (FIG. 3). By contrast, immunization with Lm-E7 and Lm-Gag did not induce tumor regression. This experiment was repeated multiple times, always with very similar results. In addition, similar results were achieved for Lm-LLO-E7 under different immunization protocols. In another experiment, a single immunization was able to cure mice of established 5 mm TC-1 tumors.

In other experiments, similar results were obtained with 2 other E7-expressing tumor cell lines: C3 and EL-4/E7. To confirm the efficacy of vaccination with Lm-LLO-E7, animals that had eliminated their tumors were re-challenged with TC-1 or EL-4/E7 tumor cells on day 60 or day 40, respectively. Animals immunized with Lm-LLO-E7 remained tumor free until termination of the experiment (day 124 in the case of TC-1 and day 54 for EL-4/E7).

Thus, expression of an antigen as a fusion protein with ΔLLO enhances the immunogenicity of the antigen.

### Example 2: LM-LLO-E7 Treatment Elicits TC-1 Specific Splenocyte Proliferation

To measure induction of T cells by Lm-E7 with Lm-LLO-E7, TC-1-specific proliferative responses, a measure of antigen-specific immunocompetence, were measured in immunized mice. Splenocytes from Lm-LLO-E7-immunized mice proliferated when exposed to irradiated TC-1 cells as a source of E7, at splenocyte: TC-1 ratios of 20:1, 40:1, 80:1, and 160:1 (FIG. 4). Conversely, splenocytes from Lm-E7 and rLm control-immunized mice exhibited only background levels of proliferation.

### Example 3: ActA-E7 and PEST-E7 Fusions Confer Anti-Tumor Immunity

### Materials and Methods

### Construction of Lm-ActA-E7

Lm-ActA-E7 is a recombinant strain of LM, comprising a plasmid that expresses the E7 protein fused to a truncated version of the actA protein. Lm-actA-E7 was generated by introducing a plasmid vector pDD-1, constructed by modifying pDP-2028, into Listeria. pDD-1 comprises an expression cassette expressing a copy of the 310 bp hly promoter and the hly signal sequence (ss), which drives the expression and secretion of ActA-E7; 1170 bp of the actA gene that comprises four PEST sequences (SEQ ID NO: 14) (the truncated ActA polypeptide consists of the first 390 AA of the molecule, SEQ ID NO: 12); the 300 bp HPV E7 gene; the 1019 bp prfA gene (controls expression of the virulence genes); and the CAT gene (chloramphenicol resistance gene) for selection of transformed bacteria clones (Sewell et al. (2004), Arch. Otolaryngol. Head Neck Surg., 130: 92-97).

The hly promoter (pHly) and gene fragment were PCR amplified from pGG55 (Example 1) using primer 5′-GGGGTCTAGACCTCCTTTGATTAGTATATTC-3′ (Xba I site is underlined; SEQ ID NO: 59) and primer 5′-ATCTTCGCTATCTGTCGCCGCGGCGCGTGCTTCAGTTTGTTGCGC-′3 (Not I site is underlined. The first 18 nucleotides are the ActA gene overlap; SEQ ID NO: 60). The actA gene was PCR amplified from the LM 10403s wildtype genome using primer 5′-GCGCAACAAACTGAAGCAGCGGCCGCGGCGACAGATAGCGAAGAT-3′ (NotI site is underlined; SEQ ID NO: 61) and primer 5′-TGTAGGTGTATCTCCATGCTCGAGAGCTAGGCGATCAATTTC-3′ (XhoI site is underlined; SEQ ID NO: 62). The E7 gene was PCR amplified from pGG55 (pLLO-E7) using primer 5′-GGAATTGATCGCCTAGCTCTCGAGCATGGAGATACACCTACA-3′ (XhoI site is underlined; SEQ ID NO: 63) and primer 5′-AAACGGATTTATTTAGATCCCGGGTTATGGTTTCTGAGAACA-3′ (XmaI site is underlined; SEQ ID NO: 64). The prfA gene was PCR amplified from the LM 10403s wild-type genome using primer 5′-TGTTCTCAGAAACCATAACCCGGGATCTAAATAAATCCGTTT-3′ (XmaI site is underlined; SEQ ID NO: 65) and primer 5′-GGGGGTCGACCAGCTCTTCTTGGTGAAG-3′ (SalI site is underlined; SEQ ID NO: 66). The hly promoter-actA gene fusion (pHly-actA) was PCR generated and amplified from purified pHly DNA and purified actA DNA using the upstream pHly primer (SEQ ID NO: 59) and downstream actA primer (SEQ ID NO: 62).

The E7 gene fused to the prfA gene (E7-prfA) was PCR generated and amplified from purified E7 DNA and purified prfA DNA using the upstream E7 primer (SEQ ID NO: 63) and downstream prfA gene primer (SEQ ID NO: 66).

The pHly-actA fusion product fused to the E7-prfA fusion product was PCR generated and amplified from purified fused pHly-actA DNA product and purified fused E7-prfA DNA product using the upstream pHly primer (SEQ ID NO: 59) and downstream prfA gene primer (SEQ ID NO: 66) and ligated into pCRII (Invitrogen, La Jolla, Calif.). Competent E. coli (TOP10′F, Invitrogen, La Jolla, Calif.) were transformed with pCRII-ActAE7. After lysis and isolation, the plasmid was screened by restriction analysis using BamHI (expected fragment sizes 770 bp and 6400 bp (or when the insert was reversed into the vector: 2500 bp and 4100 bp)) and BstXI (expected fragment sizes 2800 bp and 3900 bp) and also screened with PCR analysis using the upstream pHly primer (SEQ ID NO:59) and the downstream prfA gene primer (SEQ ID NO: 66).

The pHly-actA-E7-prfA DNA insert was excised from pCRII by double digestion with Xba I and Sal I and ligated into pDP-2028 also digested with Xba I and Sal I. After transforming TOP10′F competent E. coli (Invitrogen, La Jolla, Calif.) with expression system pActAE7, chloramphenicol resistant clones were screened by PCR analysis using the upstream pHly primer (SEQ ID NO: 59) and the downstream PrfA gene primer (SEQ ID NO: 66). A clone comprising pActAE7 was grown in brain heart infusion medium (with chloramphenicol (20 mcg (microgram)/ml (milliliter), Difco, Detroit, Mich.) and pActAE7 was isolated from the bacteria cell using a midiprep DNA purification system kit (Promega, Madison, Wis.). A prfA-negative strain of penicillin-treated Listeria (strain XFL-7) was transformed with expression system pActAE7, as described in Ikonomidis et al. (1994, J. Exp. Med. 180: 2209-2218) and clones were selected for the retention of the plasmid in vivo. Clones were grown in brain heart infusion with chloramphenicol (20 mcg/ml) at 37° C. Bacteria were frozen in aliquots at −80° C.

### Immunoblot Verification of Antigen Expression

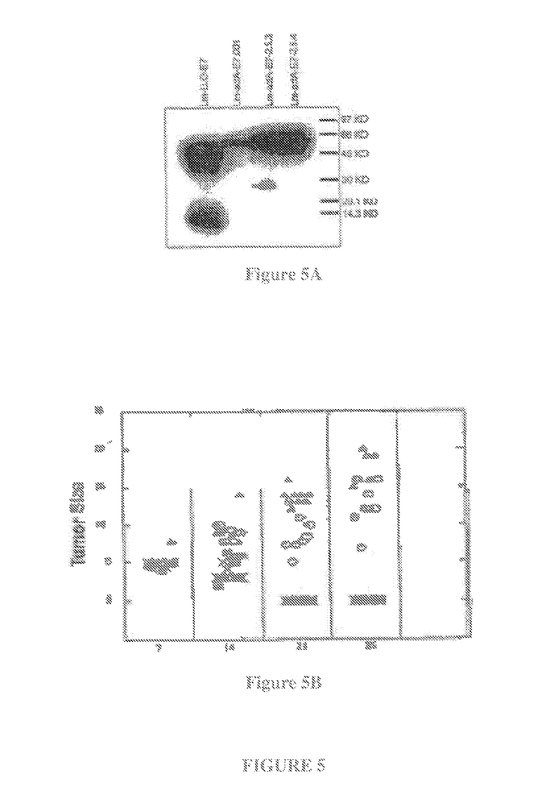

To verify that Lm-ActA-E7 secretes ActA-E7, (about 64 kD), Listeria strains were grown in Luria-Bertoni (LB) medium at 37° C. Protein was precipitated from the culture supernatant with trichloroacetic acid (TCA) and resuspended in 1× sample buffer with 0.1N sodium hydroxide. Identical amounts of each TCA precipitated supernatant were loaded on 4% to 20% Tris-glycine sodium dodecyl sulfate-polyacrylamide gels (NOVEX, San Diego, Calif.). Gels were transferred to polyvinylidene difluoride membranes and probed with 1:2500 anti-E7 monoclonal antibody (Zymed Laboratories, South San Francisco, Calif.), then with 1:5000 horseradish peroxidase-conjugated anti-mouse IgG (Amersham Pharmacia Biotech, Little Chalfont, England). Blots were developed with Amersham enhanced chemiluminescence detection reagents and exposed to autoradiography film (Amersham) (FIG. 5A).

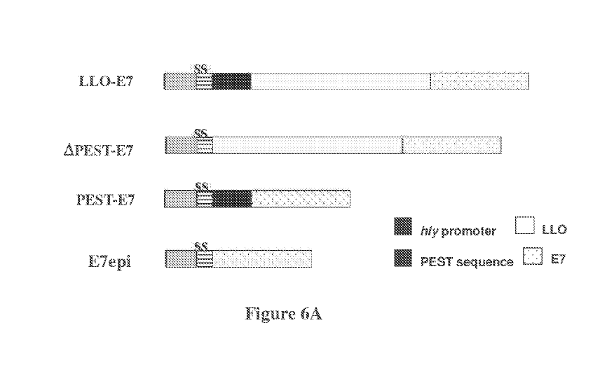

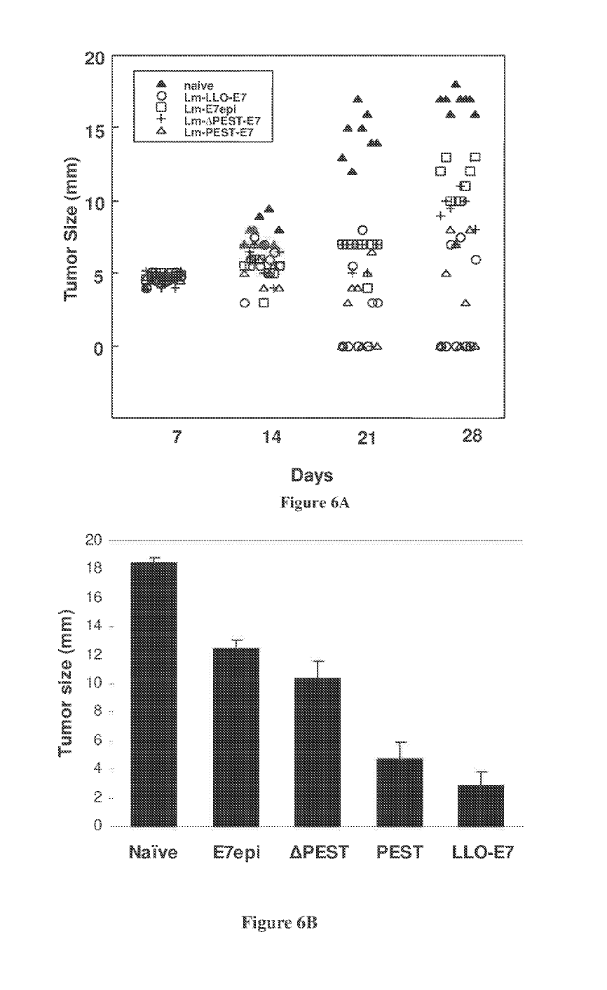

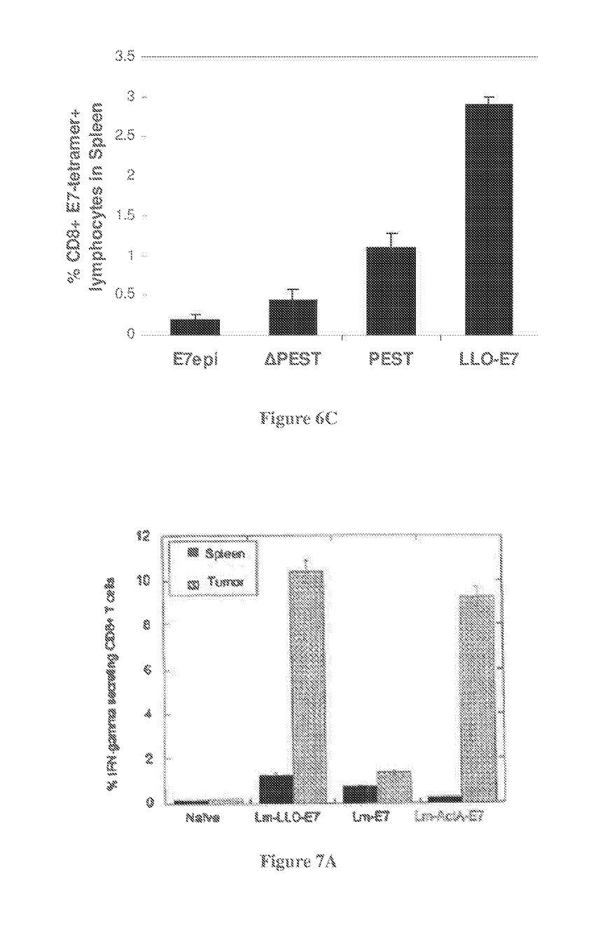

### Construction of Lm-PEST-E7, Lm-ΔPEST-E7, and Lm-E7epi (FIG. 6A)

Lm-PEST-E7 is identical to Lm-LLO-E7, except that it contains only the promoter and PEST sequence of the hly gene, specifically the first 50 AA of LLO. To construct Lm-PEST-E7, the hly promoter and PEST regions were fused to the full-length E7 gene using the SOE (gene splicing by overlap extension) PCR technique. The E7 gene and the hly-PEST gene fragment were amplified from the plasmid pGG-55, which contains the first 441 AA of LLO, and spliced together by conventional PCR techniques. To create a final plasmid, pVS16.5, the hly-PEST-E7 fragment and the prfA gene were subcloned into the plasmid pAM401, which includes a chloramphenicol resistance gene for selection in vitro, and the resultant plasmid was used to transform XFL-7.

Lm-ΔPEST-E7 is a recombinant Listeria strain that is identical to Lm-LLO-E7 except that it lacks the PEST sequence. It was made essentially as described for Lm-PEST-E7, except that the episomal expression system was constructed using primers designed to remove the PEST-containing region (bp 333-387) from the hly-E7 fusion gene. Lm-E7epi is a recombinant strain that secretes E7 without the PEST region or LLO. The plasmid used to transform this strain contains a gene fragment of the hly promoter and signal sequence fused to the E7 gene. This construct differs from the original Lm-E7, which expressed a single copy of the E7 gene integrated into the chromosome. Lm-E7epi is completely isogenic to Lm-LLO-E7, Lm-PEST-E7, and Lm-ΔPEST-E7 except for the form of the E7 antigen expressed.

### Results

To compare the anti-tumor immunity induced by Lm-ActA-E7 versus Lm-LLO-E7, 2×10⁵ TC-1 tumor cells were implanted subcutaneously in mice and allowed to grow to a palpable size (approximately 5 millimeters [mm]). Mice were immunized i.p. with one LD₅₀ of either Lm-ActA-E7 (5×10⁸ CFU), (crosses) Lm-LLO-E7 (10⁸ CFU) (squares) or Lm-E7 (10⁶ CFU) (circles) on days 7 and 14. By day 26, all of the animals in the Lm-LLO-E7 and Lm-ActA-E7 were tumor free and remained so, whereas all of the naive animals (triangles) and the animals immunized with Lm-E7 grew large tumors (FIG. 5B). Thus, vaccination with ActA-E7 fusions causes tumor regression.

In addition, Lm-LLO-E7, Lm-PEST-E7, Lm-ΔPEST-E7, and Lm-E7epi were compared for their ability to cause regression of E7-expressing tumors. s.c. TC-1 tumors were established on the left flank of 40 C57BL/6 mice. After tumors had reached 4-5 mm, mice were divided into 5 groups of 8 mice. Each groups was treated with 1 of 4 recombinant LM vaccines, and 1 group was left untreated. Lm-LLO-E7 and Lm-PEST-E7 induced regression of established tumors in ⅝ and ⅜ cases, respectively. There was no statistical difference between the average tumor size of mice treated with Lm-PEST-E7 or Lm-LLO-E7 at any time point. However, the vaccines that expressed E7 without the PEST sequences, Lm-ΔPEST-E7 and Lm-E7epi, failed to cause tumor regression in all mice except one (FIG. 6B, top panel). This was representative of 2 experiments, wherein a statistically significant difference in mean tumor sizes at day 28 was observed between tumors treated with Lm-LLO-E7 or Lm-PEST-E7 and those treated with Lm-E7epi or Lm-ΔPEST-E7; P<0.001, Student's t test; FIG. 6B, bottom panel). In addition, increased percentages of tetramer-positive splenocytes were seen reproducibly over 3 experiments in the spleens of mice vaccinated with PEST-containing vaccines (FIG. 6C). Thus, vaccination with PEST-E7 fusions causes tumor regression.

### Example 4: Fusion of E7 to LLO, Acta, or a Pest-Like Sequence Enhances E7-Specific Immunity and Generates Tumor-Infiltrating E7-Specific CD8⁺ Cells

### Materials and Experimental Methods

500 mcl (microliter) of MATRIGEL®, comprising 100 mcl of 2×10⁵ TC-1 tumor cells in phosphate buffered saline (PBS) plus 400 mcl of MATRIGEL® (BD Biosciences, Franklin Lakes, N.J.) were implanted subcutaneously on the left flank of 12 C57BL/6 mice (n=3). Mice were immunized intraperitoneally on day 7, 14 and 21, and spleens and tumors were harvested on day 28. Tumor MATRIGELs were removed from the mice and incubated at 4° C. overnight in tubes containing 2 milliliters (ml) of RP 10 medium on ice. Tumors were minced with forceps, cut into 2 mm blocks, and incubated at 37° C. for 1 hour with 3 ml of enzyme mixture (0.2 mg/ml collagenase-P, 1 mg/ml DNAse-1 in PBS). The tissue suspension was filtered through nylon mesh and washed with 5% fetal bovine serum+0.05% of NaN₃ in PBS for tetramer and TN-gamma staining.

Splenocytes and tumor cells were incubated with 1 micromole (mcm) E7 peptide for 5 hours in the presence of brefeldin A at 10⁷ cells/ml. Cells were washed twice and incubated in 50 mcl of anti-mouse Fc receptor supernatant (2.4 G2) for 1 hour or overnight at 4° C. Cells were stained for surface molecules CD8 and CD62L, permeabilized, fixed using the permeabilization kit Golgi-Stop® or Golgi-Plug® (Pharmingen, San Diego, Calif.), and stained for TN-gamma. 500,000 events were acquired using two-laser flow cytometer FACSCalibur and analyzed using Cellquest Software (Becton Dickinson, Franklin Lakes, N.J.). Percentages of IFN-gamma secreting cells within the activated (CD62L^{low}) CD8⁺ T cells were calculated.

For tetramer staining, H-2D^{b} tetramer was loaded with phycoerythrin (PE)-conjugated E7 peptide (RAHYNIVTF, SEQ ID NO: 67), stained at rt for 1 hour, and stained with anti-allophycocyanin (APC) conjugated MEL-14 (CD62L) and FITC-conjugated CD8□ at 4° C. for 30 min. Cells were analyzed comparing tetramer⁺CD8⁺ CD62L^{low} cells in the spleen and in the tumor.

### Results

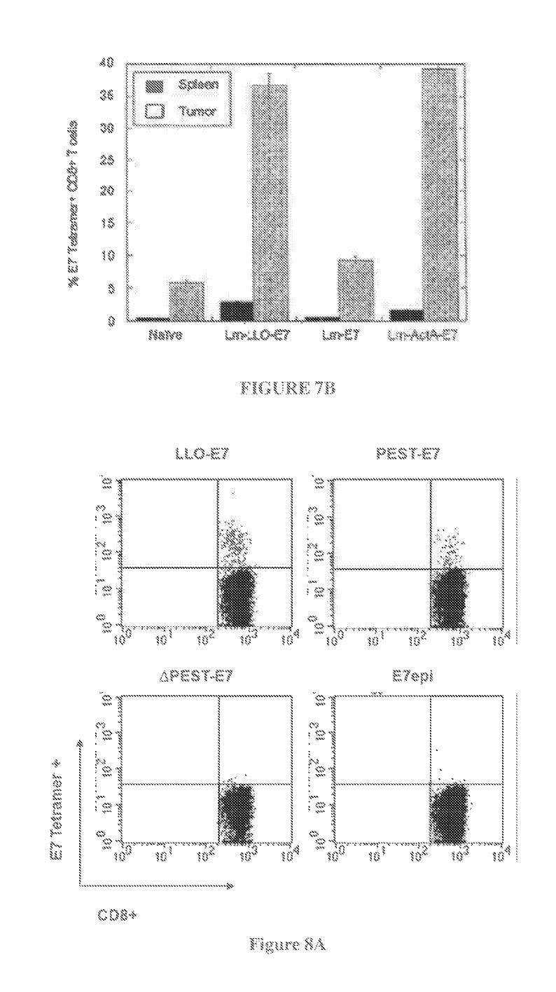

To analyze the ability of Lm-ActA-E7 to enhance antigen specific immunity, mice were implanted with TC-1 tumor cells and immunized with either Lm-LLO-E7 (1×10⁷ CFU), Lm-E7 (1×10⁶ CFU), or Lm-ActA-E7 (2×10⁸ CFU), or were untreated (naïve). Tumors of mice from the Lm-LLO-E7 and Lm-ActA-E7 groups contained a higher percentage of IFN-gamma-secreting CD8⁺ T cells (FIG. 7A) and tetramer-specific CD8⁺ cells (FIG. 7B) than in Lm-E7 or naive mice.

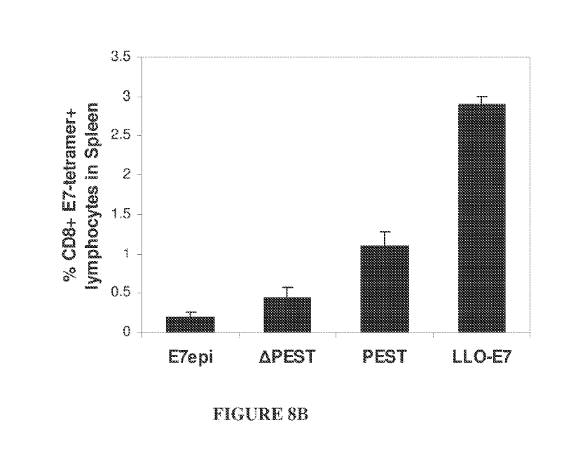

In another experiment, tumor-bearing mice were administered Lm-LLO-E7, Lm-PEST-E7, Lm-ΔPEST-E7, or Lm-E7epi, and levels of E7-specific lymphocytes within the tumor were measured. Mice were treated on days 7 and 14 with 0.1 LD₅₀ of the 4 vaccines. Tumors were harvested on day 21 and stained with antibodies to CD62L, CD8, and with the E7/Db tetramer. An increased percentage of tetramer-positive lymphocytes within the tumor were seen in mice vaccinated with Lm-LLO-E7 and Lm-PEST-E7 (FIG. 8A). This result was reproducible over three experiments (FIG. 8B).

Thus, Lm-LLO-E7, Lm-ActA-E7, and Lm-PEST-E7 are each efficacious at induction of tumor-infiltrating CD8⁺ T cells and tumor regression.

### Example 5: LLO and ActA Fusions Reduce Autochthonous (Spontaneous) Tumors in E6/E7 Transgenic Mice

To determine the impact of the Lm-LLO-E7 and Lm-ActA-E7 vaccines on autochthonous tumors in the E6/E7 transgenic mouse, 6 to 8 week old mice were immunized with 1×10⁸ Lm-LLO-E7 or 2.5×10⁸ Lm-ActA-E7 once per month for 8 months. Mice were sacrificed 20 days after the last immunization and their thyroids removed and weighed. This experiment was performed twice (Table 1).

TABLE 1
Thyroid weight (mg) in unvaccinated and vaccinated
transgenic mice at 8 months of age (mg)*.

| | ± | Lm- | ± | Lm- | ± | Lm- | ± |
|---|---|---|---|---|---|---|---|
| Untreated | S.D. | LLO-NP | S.D. | LLO-E7 | S.D. | ActA-E7 | S.D. |
| Expt. 1 | | | | | | | |
| 408 | 123 | 385 | 130 | 225 | 54 | 305 | 92 |
| Expt. 2 | | | | | | | |
| 588 | 94 | 503 | 86 | 239 | 68 | 275 | 84 |

*Statistical analyses performed using Student's t test showed that the difference in thyroid weight between Lm-LLO-NP treated mice and untreated mice was not significant but that the difference between Lm-LLO-E7 and Lm-ActA-E7 treated mice was highly significant
(p < 0.001)

The difference in thyroid weight between Lm-LLO-E7 treated mice and untreated mice and between Lm-LLO-ActA treated mice and untreated mice was significant (p<0.001 and p<0.05, respectively) for both experiments, while the difference between Lm-LLO-NP treated mice (irrelevant antigen control) and untreated mice was not significant (Student's t test), showing that Lm-LLO-E7 and Lm-ActA-E7 controlled spontaneous tumor growth. Thus, vaccines of the present invention prevent formation of new E7-expressing tumors.

To summarize the findings in the above Examples, LLO-antigen and ActA-antigen fusions (a) induce tumor-specific immune response that include tumor-infiltrating antigen-specific T cells; and are capable of inducing tumor regression and controlling tumor growth of both normal and particularly aggressive tumors; (b) overcome tolerance to self antigens; and (c) prevent spontaneous tumor growth. These findings are generalizable to a large number of antigens, PEST-like sequences, and tumor types, as evidenced by their successful implementation with a variety of different antigens, PEST-like sequences, and tumor types.

### Example 6: LM-LLO-E7 Vaccines are Safe and Improve Clinical Indicators in Cervical Cancer Patients

### Materials and Experimental Methods

Inclusion Criteria.

All patients in the trial were diagnosed with “advanced, progressive or recurrent cervical cancer,” and an assessment at the time of entry indicated that all were staged as having IVB disease. All patients manifested a positive immune response to an anergy panel containing 3 memory antigens selected from candidin, mumps, tetanus, or Tuberculin Purified Protein Derivative (PPD); were not pregnant or HIV positive, had taken no investigational drugs within 4 weeks, and were not receiving steroids.

Protocol:

Patients were administered 2 vaccinations at a 3-week interval as a 30-minute intravenous (IV) infusion in 250 ml of normal saline to inpatients. After 5 days, patients received a single course of IV ampicillin and were released with an additional 10 days of oral ampicillin Karnofsky Performance Index, which is a measurement of overall vitality and quality of life such as appetite, ability to complete daily tasks, restful sleep, etc, was used to determine overall well-being. In addition, the following indicators of safety and general wellbeing were determined: alkaline phosphatase; bilirubin, both direct and total; gamma glutamyl transpeptidase (ggt); cholesterol; systole, diastole, and heart rate; Eastern Collaborative Oncology Group's (ECOG)'s criteria for assessing disease progression—a Karnofsky like—quality of life indicator; hematocrit; hemoglobin; platelet levels; lymphocytes levels; AST (aspartate aminotransferase); ALT (alanine aminotransferase); and LDH (lactate dehydrogenase). Patients were followed at 3 weeks and 3 months subsequent to the second dosing, at which time Response Evaluation Criteria in Solid Tumors (RECIST) scores of the patients were determined, scans were performed to determine tumor size, and blood samples were collected for immunological analysis at the end of the trial, which includes the evaluation of IFN-γ, IL-4, CD4⁺ and CD8⁺ cell populations.

Listeria Strains:

The creation of LM-LLO-E7 is described in Example 1.

### Results

Prior to the clinical trial, a preclinical experiment was performed to determine the anti-tumor efficacy of intravenous (i.v.) vs. i.p. administration of LM-LLO-E7. A tumor containing 1×10⁴ TC-1 cells was established sub-cutaneously. On days 7 and 14, mice were immunized with either 10⁸ LM-LLO-E7 i.p. or LM-LLO-E7 i.v. at doses of 10⁸, 10⁷, 10⁶, or 10⁵. At day 35, ⅝ of the mice that received 10⁸ LM-LLO-E7 by either route or 10⁷ LM-LLO-E7 i.v, and 4/8 of the mice that received 10⁶ LM-LLO-E7 i.v, were cured. By contrast, doses of less than 10⁷ or in some cases even 10⁸ LM-LLO-E7 administered i.p. were ineffective at controlling tumor growth. Thus, i.v. administration of LM-LLO-E7 is more effective than i.p. administration.

### Clinical Trial

A phase I/II clinical trial was conducted to assess safety and efficacy of LM-LLO-E7 vaccines in patients with advanced, progressive, or recurrent cervical cancer. 5 patients each were assigned to cohorts 1-2, which received 1×10⁹ or 3.3×10⁹ CFU, respectfully. An additional 5 patients each will be assigned to cohorts 3-4, which will receive 1×10¹⁰ or 3.31×10¹⁰ CFU, respectfully.

### Safety Data

### First Cohort

All patients in the first cohort reported onset of mild-to-moderate fever and chills within 1-2 hours after onset of the infusion. Some patients exhibited vomiting, with or without nausea. With 1 exception (described below), a single dose of a non-steroidal agent such as paracetamol was sufficient to resolve these symptoms. Modest, transient cardiovascular effects were observed, consistent with, and sharing the time course of, the fever. No other adverse effects were reported.

At this late stage of cervical cancer, 1 year survival is typically 10-15% of patients and no tumor therapy has ever been effective. Indeed, Patient 2 was a young patient with very aggressive disease who passed away shortly after completing the trial.

Quantitative blood cultures were assessed on days 2, 3, and 5 post-administration. Of the 5 evaluable patients in this cohort, 4 exhibited no serum Listeria at any time and 1 had a very small amount (35 cfu) of circulating Listeria on day 2, with no detectable Listeria on day 3 or 5.

Patient 5 responded to initial vaccination with mild fever over the 48 hours subsequent to administration, and was treated with anti-inflammatory agents. On 1 occasion, the fever rose to moderate severity (at no time above 38.4° C.), after which she was given a course of ampicillin, which resolved the fever. During the antibiotic administration she experienced mild urticaria, which ended after antibiotic administration. Blood cultures were all sterile, cardiovascular data were within the range observed for other patients, and serum chemistry values were normal, showing that this patient had no listerial disease. Further, the anergy panel indicated a robust response to ⅓ memory antigens, indicating the presence of functional immunity (similar to the other patients). Patient 5 subsequently evidenced a response similar to all other patients upon receiving the boost.

### Second Cohort and Overall Safety Observations

In both cohorts, minor and transient changes in liver function tests were observed following infusion. These changes were determined by the attending physician monitoring the trial to have no clinical significance, and were expected for a short-lived infection of bacteria that are rapidly removed from the systemic circulation to the liver and spleen. In general, all the safety indicators described in the Methods section above displayed little or no net change, indicative of an excellent safety profile. The side effect profile in this cohort was virtually identical to that seen in the in the initial cohort and appeared to be a dose independent series of symptoms related to the consequences of cytokines and similar agents that occur consequent to the induction of an iatrogenic infection. No serum Listeria was observed at any time and no dose limiting toxicity was observed in either cohort.

### Efficacy—First Cohort

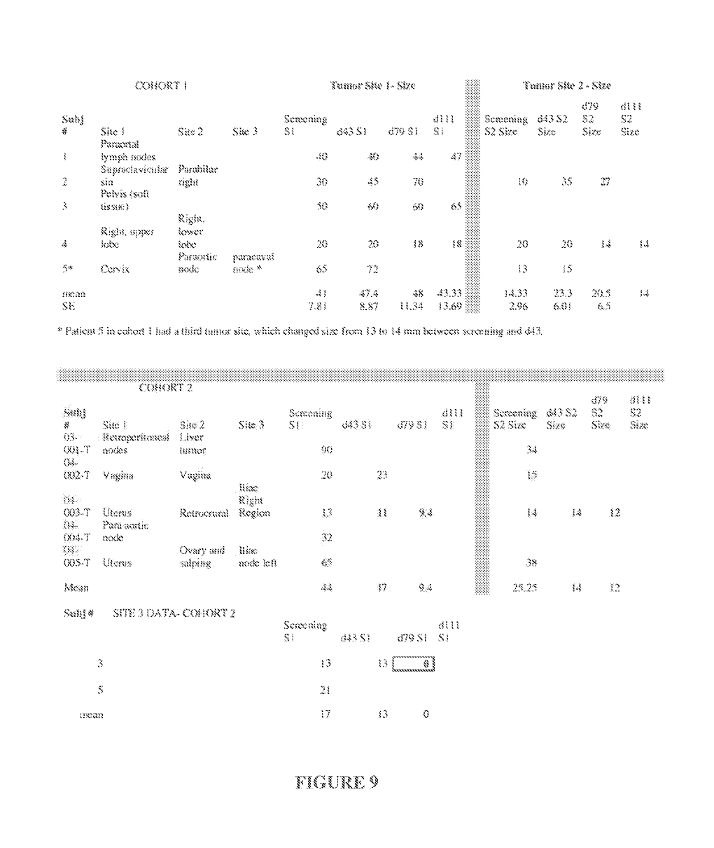

The following indications of efficacy were observed in the 3 patients in the first cohort that finished the trial: (FIG. 9).

Patient 1 entered the trial with 2 tumors of 20 mm each, which shrunk to 18 and 14 aim over the course of the trial, indicating therapeutic efficacy of the vaccine. In addition, patient 1 entered the trial with a Karnofsky Performance Index of 70, which rose to 90 after dosing. In the Safety Review Panel meeting, Sini\check{s}a Radulovic, the chairman of the Department of Oncology, Institute for Oncology and Radiology, Belgrade, Serbia presented the results to a representative of the entity conducting the trials; Michael Kurman, an independent oncologist who works as a consultant for the entity; Kevin Ault, an academic gynecologic oncologist at Emory University who conducted the phase III Gardasil trials for Merck and the Cervarix trials for Glaxo SmithKline; and Tate Thigpen, a founder of the Gynecologic Oncology Group at NCI and professor of gynecologic oncology at the University of Mississippi. In the opinion of Dr. Radulovic, patient 1 exhibited a clinical benefit from treatment with the vaccine.

Before passing away, Patient 2 exhibited a mixed response, with ½ tumors shrinking.

Patient 3 enrolled with paraneoplastic disease, (an epiphenomenon of cancer wherein the overall debilitated state of the patient has other sequalae that are secondary to the cancer), including an elevation of platelet count to 936×10⁹/ml. The count decreased to 405×10⁹/ml, approximately a normal level, following the first dose.

Patient 4 entered the trial with 2 tumors of 20 mm each, which shrunk to 18 and 14 mm over the course of the trial, indicating therapeutic efficacy of the vaccine. Patient 4 exhibited a weight gain of 1.6 Kg and an increased hemoglobin count of approximately 10% between the first and second doses.

### Efficacy—Second Cohort and General Observations

In the lowest dose cohort, 2 patients demonstrated the shrinkage of tumors. The timing of this effect was consistent with that observed in immunological responses, in that it followed chronologically development of the immune response. One of the 2 patients in the second cohort evaluated so far for tumor burden exhibited a dramatic tumor load reduction at a post-vaccination time point. At the start of the trial, this patient had 3 tumors of 13, 13, and 14 mm. After the 2 doses of the vaccine, 2 of the tumor had shrunk to 9.4 and 12 mm, and the third was no longer detectable.

Tumors loads for the 2 cohorts are depicted in FIG. 13B. In summary, even relatively low doses of LM-LLO-E7, administered in a therapeutic regimen containing a priming injection and a single boost, achieved 3 objective responses out of 6 patients for whom data has been collected.

### Discussion

At this late stage of cervical cancer, 1 year survival is typically 10-15% of patients and no tumor therapy has ever been effective. No treatment has shown to be effective in reversing stage IVB cervical cancer. Despite the difficulty of treating cervical cancer at this stage, an anti-tumor effect was observed in 2/6 patients. In addition, other indications of efficacy were observed in patients that finished the trial, as described hereinabove.

Thus, LM-LLO-E7 is safe in human subjects and improves clinical indicators of cervical cancer patients, even when administered at relatively low doses. Additional positive results are likely to be observed when the dose and number of booster vaccinations is increased; and/or when antibiotics are administered in smaller doses or at a later time point after infusion. Pre-clinical studies have shown that a dose increase of a single order of magnitude can cause dramatic changes in response rate (e.g. a change from 0% response rate to 50-100% complete remission rate. Additional booster doses are also very likely to further enhance the immune responses obtained. Moreover, the positive effects of the therapeutic immune response observed are likely to continue with the passage of additional time, as the immune system continues to attack the cancer.

### Example 7: Construction of Attenuated Listeria Strain-LmddΔactA and Insertion of the Human Klk3 Gene in Frame to the Lily Gene in the Lmdd and Lmdda Strains

### Materials and Methods

A recombinant Lm was developed that secretes PSA fused to tLLO (Lm-LLO-PSA), which elicits a potent PSA-specific immune response associated with regression of tumors in a mouse model for prostate cancer, wherein the expression of tLLO-PSA is derived from a plasmid based on pGG55 (Table 2), which confers antibiotic resistance to the vector. We recently developed a new strain for the PSA vaccine based on the pADV142 plasmid, which has no antibiotic resistance markers, and referred as LmddA-142 (Table 3). This new strain is 10 times more attenuated than Lm-LLO-PSA. In addition, LmddA-142 was slightly more immunogenic and significantly more efficacious in regressing PSA expressing tumors than the Lm-LLO-PSA.

TABLE 2
Plasmids and strains

| Plasmids | Features |
|---|---|
| pGG55 | pAM401/pGB354 shuttle plasmid with gram(−) and |
| | gram(+) cm resistance, LLO-E7 expression cassette and a |
| | copy of Lm prfA gene |
| pTV3 | Derived from pGG55 by deleting cm genes and |
| | inserting the Lm dal gene |
| pADV119 | Derived from pTV3 by deleting the prfA gene |
| pADV134 | Derived from pADV119 by replacing the Lm dal gene by |
| | the Bacillus dal gene |
| pADV142 | Derived from pADV134 by replacing HPV16 e7 with klk3 |
| pADV168 | Derived from pADV134 by replacing HPV16 e7 with |
| | hmw-maa₂₁₆₀₋₂₂₅₈ |
| Strains | Genotype |
| 10403S | Wild-type Listeria monocytogenes:: str |
| XFL-7 | 10403S prfA⁽⁻⁾ |
| Lmdd | 10403S dal⁽⁻⁾ dat⁽⁻⁾ |
| LmddA | 10403S dal⁽⁻⁾ dat⁽⁻⁾ actA⁽⁻⁾ |
| LmddA-134 | 10403S dal⁽⁻⁾ dat⁽⁻⁾ actA⁽⁻⁾ pADV134 |
| LmddA-142 | 10403S dal⁽⁻⁾ dat⁽⁻⁾ actA⁽⁻⁾ pADV142 |
| LmddA-143 | 10403S dal⁽⁻⁾ dat⁽⁻⁾ with klk3 fused to the hly |
| | gene in the chromosome |
| LmddA-143 | 10403S dal⁽⁻⁾ dat⁽⁻⁾ actA⁽⁻⁾ with klk3 |
| | fused to the hly gene in the chromosome |
| LmddA-168 | 10403S dal⁽⁻⁾ dat⁽⁻⁾ actA⁽⁻⁾ pADV168 |
| Lmdd- | Lmdd-143 pADV134 |
| 143/134 | |
| LmddA- | LmddA-143 pADV134 |
| 143/134 | |
| Lmdd- | Lmdd-143 pADV168 |
| 143/168 | |
| LmddA- | LmddA-143 pADV168 |
| 143/168 | |

The sequence of the plasmid pAdv142 (6523 bp) was as follows:

(SEQ ID NO: 68)
cggagtgtatactggcttactatgttggcactgatgagggtgtcagtg
aagtgcttcatgtggcaggagaaaaaaggctgcaccggtgcgtcagca
gaatatgtgatacaggatatattccgcttcctcgctcactgactcgct
acgctcggtcgttcgactgcggcgagcggaaatggcttacgaacgggg
cggagatttcctggaagatgccaggaagatacttaacagggaagtgag
agggccgcggcaaagccgtttttccataggctccgcccccctgacaag
catcacgaaatctgacgctcaaatcagtggtggcgaaacccgacagga
ctataaagataccaggcgtttccccctggcggctccctcgtgcgctct
cctgttcctgcctttcggtttaccggtgtcattccgctgttatggccg
cgtttgtctcattccacgcctgacactcagttccgggtaggcagttcg
ctccaagctggactgtatgcacgaaccccccgttcagtccgaccgctg
cgccttatccggtaactatcgtcttgagtccaacccggaaagacatgc
aaaagcaccactggcagcagccactggtaattgatttagaggagttag
tcttgaagtcatgcgccggttaaggctaaactgaaaggacaagttttg
gtgactgcgctcctccaagccagttacctcggttcaaagagttggtag
ctcagagaaccttcgaaaaaccgccctgcaaggcggttttttcgtttt
cagagcaagagattacgcgcagaccaaaacgatctcaagaagatcatc
ttattaatcagataaaatatttctagccctcctttgattagtatattc
ctatcttaaagttacttttatgtggaggcattaacatttgttaatgac
gtcaaaaggatagcaagactagaataaagctataaagcaagcatataa
tattgcgtttcatctttagaagcgaatttcgccaatattataattatc
aaaagagaggggtggcaaacggtatttggcattattaggttaaaaaat
gtagaaggagagtgaaacccatgaaaaaaataatgctagtttttatta
cacttatattagttagtctaccaattgcgcaacaaactgaagcaaagg
atgcatctgcattcaataaagaaaattcaatttcatccatggcaccac
cagcatctccgcctgcaagtcctaagacgccaatcgaaaagaaacacg
cggatgaaatcgataagtatatacaaggattggattacaataaaaaca
atgtattagtataccacggagatgcagtgacaaatgtgccgccaagaa
aaggttacaaagatggaaatgaatatattgttgtggagaaaaagaaga
aatccatcaatcaaaataatgcagacattcaagttgtgaatgcaattt
cgagcctaacctatccaggtgctctcgtaaaagcgaattcggaattag
tagaaaatcaaccagatgttctccctgtaaaacgtgattcattaacac
tcagcattgatttgccaggtatgactaatcaagacaataaaatagttg
taaaaaatgccactaaatcaaacgttaacaacgcagtaaatacattag
tggaaagatggaatgaaaaatatgctcaagcttatccaaatgtaagtg
caaaaattgattatgatgacgaaatggcttacagtgaatcacaattaa
ttgcgaaatttggtacagcatttaaagctgtaaataatagcttgaatg
taaacttcggcgcaatcagtgaagggaaaatgcaagaagaagtcatta
gttttaaacaaatttactataacgtgaatgttaatgaacctacaagac
cttccagatttttcggcaaagctgttactaaagagcagttgcaagcgc
ttggagtgaatgcagaaaatcctcctgcatatatctcaagtgtggcgt
atggccgtcaagtttatttgaaattatcaactaattcccatagtacta
aagtaaaagctgcttttgatgctgccgtaagcggaaaatctgtctcag
gtgatgtagaactaacaaatatcatcaaaaattcttccttcaaagccg
taatttacggaggttccgcaaaagatgaagttcaaatcatcgacggca
acctcggagacttacgcgatatatgaaaaaaggcgctactataatcga
gaaacaccaggagttcccattgatatacaacaaacttcctaaaagaca
atgaattagctgttattaaaaacaactcagaatatattgaaacaactt
caaaagcttatacagatggaaaaattaacatcgatcactctggaggat
acgttgctcaattcaacatttcttgggatgaagtaaattatgatctcg
agattgtgggaggctgggagtgcgagaagcattcccaaccctggcagg
tgcttgtggcctctcgtggcagggcagtctgcggcggtgttctggtgc
acccccagtgggtcctcacagctgcccactgcatcaggaacaaaagcg
tgatcttgctgggtcggcacagcctgtttcatcctgaagacacaggcc
aggtatttcaggtcagccacagcttcccacacccgctctacgatatga
gcctcctgaagaatcgattcctcaggccaggtgatgactccagccacg
acctcatgctgctccgcctgtcagagcctgccgagctcacggatgctg
tgaaggtcatggacctgcccacccaggagccagcactggggaccacct
gctacgcctcaggctggggcagcattgaaccagaggagttcttgaccc
caaagaaacttcagtgtgtggacctccatgttatttccaatgacgtgt
gtgcgcaagttcaccctcagaaggtgaccaagttcatgctgtgtgctg
gacgctggacagggggcaaaagcacctgctcgggtgattctgggggcc
cacttgtctgttatggtgtgcttcaaggtatcacgtcatggggcagtg
aaccatgtgccctgcccgaaaggccttccctgtacaccaaggtggtgc
attaccggaagtggatcaaggacaccatcgtggccaaccccTAAcccg
ggccactaactcaacgctagtagtggatttaatcccaaatgagccaac
agaaccagaaccagaaacagaacaagtaacattggagttagaaatgga
agaagaaaaaagcaatgatttcgtgtgaataatgcacgaaatcattgc
ttattatttaaaaagcgatatactagatataacgaaacaacgaactga
ataaagaatacaaaaaaagagccacgaccagttaaagcctgagaaact
ttaactgcgagccttaattgattaccaccaatcaattaaagaagtcga
gacccaaaatttggtaaagtatttaattactttattaatcagatactt
aaatatctgtaaacccattatatcgggtttttgaggggatttcaagtc
tttaagaagataccaggcaatcaattaagaaaaacttagttgattgcc
ttattgttgtgattcaactttgatcgtagcttctaactaattaatttt
cgtaagaaaggagaacagctgaatgaatatcccttttgttgtagaaac
tgtgcttcatgacggcttgttaaagtacaaatttaaaaatagtaaaat
tcgctcaatcactaccaagccaggtaaaagtaaaggggctatttttgc
gtatcgctcaaaaaaaagcatgattggcggacgtggcgttgttctgac
ttccgaagaagcgattcacgaaaatcaagatacatttacgcattggac
accaaacgtttatcgttatggtacgtatgcagacgaaaaccgttcata
cactaaaggacattctgaaaacaatttaagacaaatcaataccttatt
attgattttgatattcacacggaaaaagaaactatttcagcaagcgat
attttaacaacagctattgatttaggttttatgcctacgttaattatc
aaatctgataaaggttatcaagcatattttgttttagaaacgccagtc
tatgtgacttcaaaatcagaatttaaatctgtcaaagcagccaaaata
atctcgcaaaatatccgagaatattttggaaagtctttgccagttgat
ctaacgtgcaatcattagggattgctcgtataccaagaacggacaatg
tagaattttttgatcccaattaccgttattctttcaaagaatggcaag
attggtctacaaacaaacagataataagggctttactcgttcaagtct
aacggttttaagcggtacagaaggcaaaaaacaagtagatgaaccctg
gtttaatctcttattgcacgaaacgaaattttcaggagaaaagggttt
agtagggcgcaatagcgttatgtttaccctctctttagcctactttag
ttcaggctattcaatcgaaacgtgcgaatataatatgtttgagtttaa
taatcgattagatcaacccttagaagaaaaagaagtaatcaaaattgt
tagaagtgcctattcagaaaactatcaaggggctaatagggaatacat
taccattctttgcaaagcttgggtatcaagtgatttaaccagtaaaga
tttatttgtccgtcaagggtggtttaaattcaagaaaaaaagaagcga
acgtcaacgtgttcatttgtcagaatggaaagaagatttaatggctta
tattagcgaaaaaagcgatgtatacaagccttatttagcgacgaccaa
aaaagagattagagaagtgctaggcattcctgaacggacattagataa
attgctgaaggtactgaaggcgaatcaggaaattttctttaagattaa
accaggaagaaatggtggcattcaacttgctagtgttaaatcattgtt
gctatcgatcattaaattaaaaaaagaagaacgagaaagctatataaa
ggcgctgacagcttcgtttaatttagaacgtacatttattcaagaaac
tctaaacaaattggcagaacgccccaaaacggacccacaactcgattt
gtttagctacgatacaggctgaaaataaaacccgcactatgccattac
atttatatctatgatacgtgtttgtttttctttgctggctagcttaat
tgcttatatttacctgcaataaaggatttcttacttccattatactcc
cattttccaaaaacatacggggaacacgggaacttattgtacaggcca
cctcatagttaatggtttcgagccttcctgcaatctcatccatggaaa
tatattcatccccctgccggcctattaatgtgacttttgtgcccggcg
gatattcctgatccagctccaccataaattggtccatgcaaattcggc
cggcaattttcaggcgttttcccttcacaaggatgtcggtccctttca
attttcggagccagccgtccgcatagcctacaggcaccgtcccgatcc
atgtgtctttttccgctgtgtactcggctccgtagctgacgctctcgc
cttttctgatcagtttgacatgtgacagtgtcgaatgcagggtaaatg
ccggacgcagctgaaacggtatctcgtccgacatgtcagcagacgggc
gaaggccatacatgccgatgccgaatctgactgcattaaaaaagcctt
ttttcagccggagtccagcggcgctgttcgcgcagtggaccattagat
tattaacggcagcggagcaatcagctattaaagcgctcaaactgcatt
aagaaatagcctctttctttttcatccgctgtcgcaaaatgggtaaat
acccctttgcactttaaacgagggttgcggtcaagaattgccatcacg
ttctgaacttcttcctctgtttttacaccaagtctgttcatccccgta
tcgaccttcagatgaaaatgaagagaaccttttttcgtgtggcgggct
gcctcctgaagccattcaacagaataacctgttaaggtcacgtcatac
tcagcagcgattgccacatactccgggggaaccgcgccaagcaccaat
ataggcgccttcaatccctttttgcgcagtgaaatcgcttcatccaaa
atggccacggccaagcatgaagcacctgcgtcaagagcagcctttgct
gtttctgcatcaccatgcccgtaggcgtttgctttcacaactgccatc
aagtggacatgttcaccgatatgttttttcatattgctgacattttcc
tttatcgcggacaagtcaatttccgcccacgtatctctgtaaaaaggt
tttgtgctcatggaaaactcctctcattttcagaaaatcccagtacgt
aattaagtatttgagaattaattttatattgattaatactaagtttac
ccagttttcacctaaaaaacaaatgatgagataatagctccaaaggct
aaagaggactataccaactatttgttaattaa.

This plasmid was sequenced at Genewiz facility from the E. coli strain on 2-20-08.

The strain Lm dal dat (Lmdd) was attenuated by the irreversible deletion of the virulence factor, ActA. An in-frame deletion of actA in the Lmdaldat (Lmdd) background was constructed to avoid any polar effects on the expression of downstream genes. The Lm dal dat ΔactA contains the first 19 amino acids at the N-terminal and 28 amino acid residues of the C-terminal with a deletion of 591 amino acids of ActA.

The actA deletion mutant was produced by amplifying the chromosomal region corresponding to the upstream (657 bp-oligo's Adv 271/272) and downstream (625 bp-oligo's Adv 273/274) portions of actA and joining by PCR. The sequence of the primers used for this amplification is given in the Table 1. The upstream and downstream DNA regions of actA were cloned in the pNEB193 at the EcoRI/PstI restriction site and from this plasmid, the EcoRI/PstI was further cloned in the temperature sensitive plasmid pKSV7, resulting in ΔactA/pKSV7 (pAdv120).

TABLE 1
Sequence of primers that was used
for the amplification of DNA
sequences upstream and downstream of actA

| | | SEQ |
|---|---|---|
| Primer | Sequence | ID NO: |
| Adv271-actAF1 | cgGAATTCGGATCCgcgcca | 69 |
| | aatcattggttgattg | |
| Adv272-actAR1 | gcgaGTCGACgtcggggtta | 70 |
| | atcgtaatgcaattggc | |
| Adv273-actAF2 | gcgaGTCGACccatacgacg | 71 |
| | ttaattcttgcaatg | |
| Adv274-actAR2 | gataCTGCAGGGATCCttcc | 72 |
| | cttctcggtaatcagtcac | |

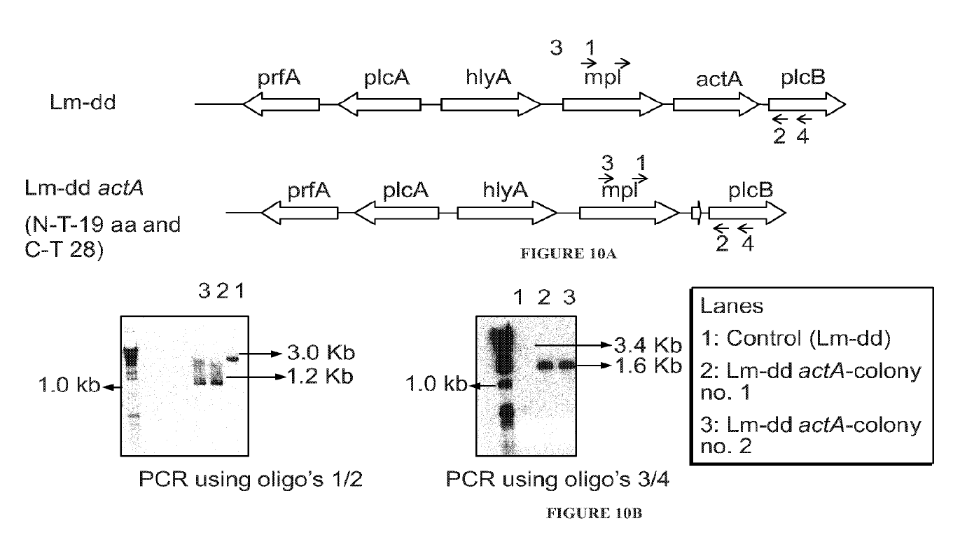

The deletion of the gene from its chromosomal location was verified using primers that bind externally to the actA deletion region, which are shown in FIGS. 10(A and B) as primer 3 (Adv 305-tgggatggccaagaaattc, SEQ ID NO: 73) and primer 4 (Adv304-ctaccatgtcttccgttgcttg; SEQ ID NO: 74). The PCR analysis was performed on the chromosomal DNA isolated from Lmdd and LmddΔactA. The sizes of the DNA fragments after amplification with two different sets of primer pairs ½ and ¾ in Lmdd chromosomal DNA was expected to be 3.0 Kb and 3.4 Kb. On the other hand, the expected sizes of PCR using the primer pairs ½ and ¾ for the LmddΔactA was 1.2 Kb and 1.6 Kb. Thus, PCR analysis in FIGS. 10(A and B) confirms that the 1.8 kb region of actA was deleted in the LmddΔactA strain. DNA sequencing was also performed on PCR products to confirm the deletion of actA containing region in the strain, LmddΔactA.

### Example 8: Construction of the Antibiotic-Independent Episomal Expression System for Antigen Delivery by Lm Vectors

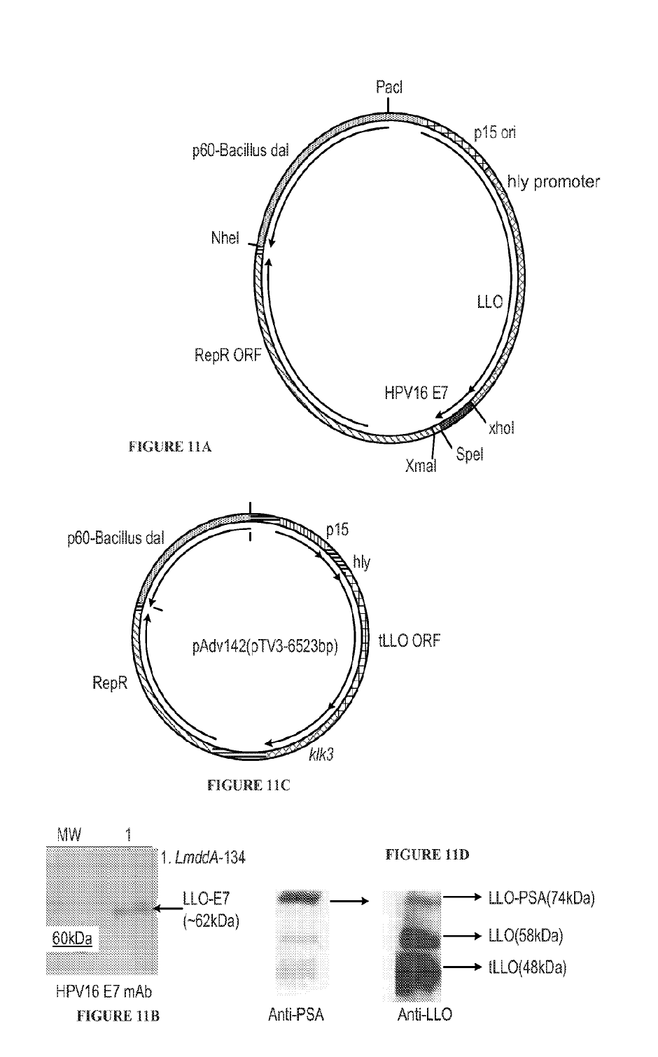

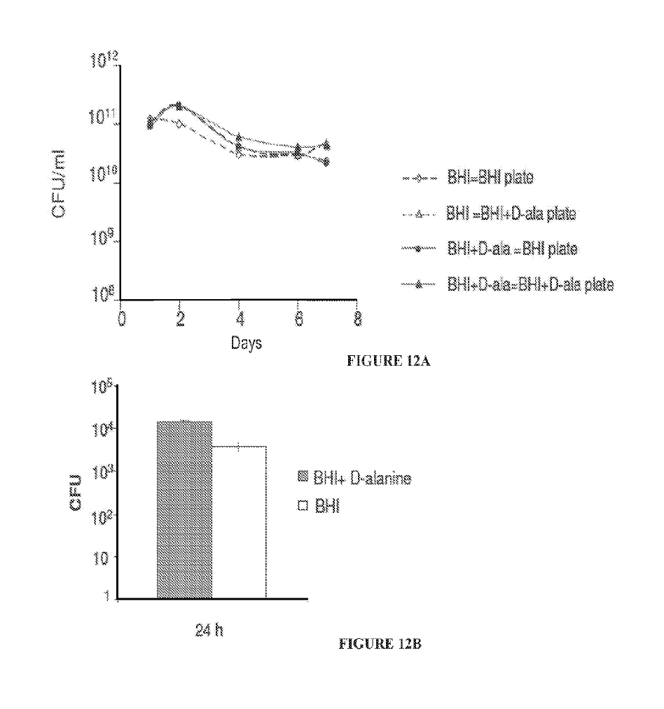

The antibiotic-independent episomal expression system for antigen delivery by Lm vectors (pAdv142) is the next generation of the antibiotic-free plasmid pTV3 (Verch et al., Infect Immun, 2004. 72(11):6418-25, incorporated herein by reference). The gene for virulence gene transcription activator, prfA was deleted from pTV3 since Listeria strain Lmdd contains a copy of prfA gene in the chromosome. Additionally, the cassette for p60-Listeria dal at the NheI/PacI restriction site was replaced by p60-Bacillus subtilis dal resulting in plasmid pAdv134 (FIG. 11A). The similarity of the Listeria and Bacillus dal genes is ˜30%, virtually eliminating the chance of recombination between the plasmid and the remaining fragment of the dal gene in the Lmdd chromosome. The plasmid pAdv134 contained the antigen expression cassette tLLO-E7. The LmddA strain was transformed with the pADV134 plasmid and expression of the LLO-E7 protein from selected clones confirmed by Western blot (FIG. 11B). The Lmdd system derived from the 10403S wild-type strain lacks antibiotic resistance markers, except for the Lmdd streptomycin resistance.

Further, pAdv134 was restricted with XhoI/XmaI to clone human PSA, klk3 resulting in the plasmid, pAdv142. The new plasmid, pAdv142 (FIG. 11C, Table 2) contains Bacillus dal (B-Dal) under the control of Listeria p60 promoter. The shuttle plasmid, pAdv142 complemented the growth of both E. coli ala drx MB2159 as well as Listeria monocytogenes strain Lmdd in the absence of exogenous D-alanine. The antigen expression cassette in the plasmid pAdv142 consists of hly promoter and LLO-PSA fusion protein (FIG. 11C).

The plasmid pAdv142 was transformed to the Listeria background strains, LmddactA strain resulting in Lm-ddA-LLO-PSA. The expression and secretion of LLO-PSA fusion protein by the strain, Lm-ddA-LLO-PSA was confirmed by Western Blot using anti-LLO and anti-PSA antibody (FIG. 11D). There was stable expression and secretion of LLO-PSA fusion protein by the strain, Lm-ddA-LLO-PSA after two in vivo passages.

### Example 9: In Vitro and In Vivo Stability of the Strain LmddA-LLO-PSA

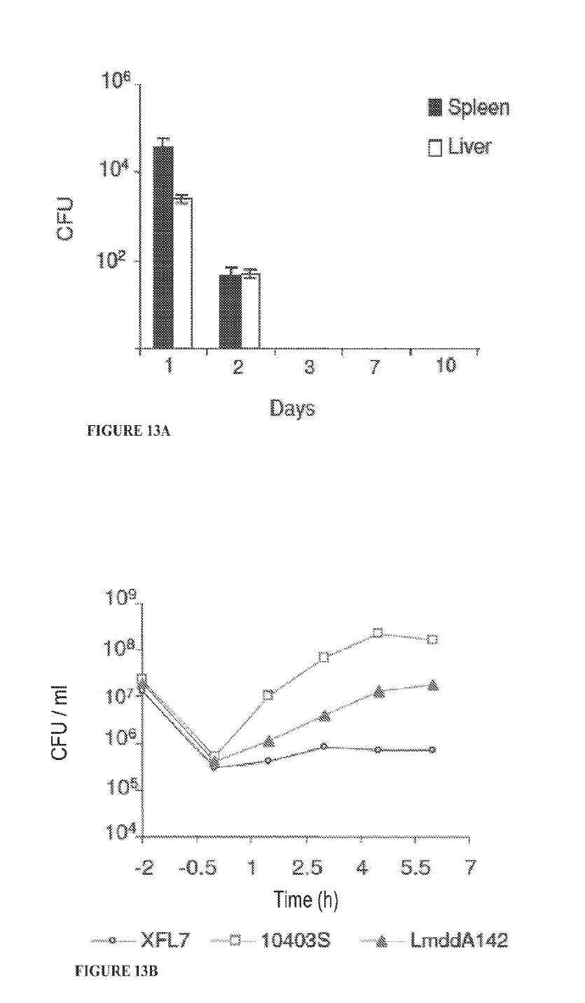

The in vitro stability of the plasmid was examined by culturing the LmddA-LLO-PSA Listeria strain in the presence or absence of selective pressure for eight days. The selective pressure for the strain LmddA-LLO-PSA is D-alanine. Therefore, the strain LmddA-LLO-PSA was passaged in Brain-Heart Infusion (BHI) and BHI+ 100 μg/ml D-alanine. CFUs were determined for each day after plating on selective (BHI) and non-selective (BHI-FD-alanine) medium. It was expected that a loss of plasmid will result in higher CFU after plating on non-selective medium (BHI-FD-alanine). As depicted in FIG. 12A, there was no difference between the number of CFU in selective and non-selective medium. This suggests that the plasmid pAdv142 was stable for at least 50 generations, when the experiment was terminated.

Plasmid maintenance in vivo was determined by intravenous injection of 5×10⁷ CFU LmddA-LLO-PSA, in C57BL/6 mice. Viable bacteria were isolated from spleens homogenized in PBS at 24 h and 48 h. CFUs for each sample were determined at each time point on BHI plates and BHI+100 mg/ml D-alanine. After plating the splenocytes on selective and non-selective medium, the colonies were recovered after 24 h. Since this strain is highly attenuated, the bacterial load is cleared in vivo in 24 h. No significant differences of CFUs were detected on selective and non-selective plates, indicating the stable presence of the recombinant plasmid in all isolated bacteria (FIG. 12B).

### Example 10: In Vivo Passaging, Virulence and Clearance of the Strain LmddA-142 (LmddA-LLO-PSA)

LmddA-142 is a recombinant Listeria strain that secretes the episomally expressed tLLO-PSA fusion protein. To determine a safe dose, mice were immunized with LmddA-LLO-PSA at various doses and toxic effects were determined. LmddA-LLO-PSA caused minimum toxic effects (data not shown). The results suggested that a dose of 10⁸ CFU of LmddA-LLO-PSA was well tolerated by mice. Virulence studies indicate that the strain LmddA-LLO-PSA was highly attenuated.

The in vivo clearance of LmddA-LLO-PSA after administration of the safe dose, 10⁸ CFU intraperitoneally in C57BL/6 mice, was determined. There were no detectable colonies in the liver and spleen of mice immunized with LmddA-LLO-PSA after day 2. Since this strain is highly attenuated, it was completely cleared in vivo at 48 h (FIG. 13A).

To determine if the attenuation of LmddA-LLO-PSA attenuated the ability of the strain LmddA-LLO-PSA to infect macrophages and grow intracellularly, a cell infection assay was performed. Mouse macrophage-like cell line such as J774A.1, were infected in vitro with Listeria constructs and intracellular growth was quantified. The positive control strain, wild type Listeria strain 10403S grows intracellularly, and the negative control XFL7, a prfA mutant, cannot escape the phagolysosome and thus does not grow in J774 cells. The intracytoplasmic growth of LmddA-LLO-PSA was slower than 10403S due to the loss of the ability of this strain to spread from cell to cell (FIG. 13B). The results indicate that LmddA-LLO-PSA has the ability to infect macrophages and grow intracytoplasmically.

### Example 11: Immunogenicity of the Strain-LmddA-LLO-PSA in C57BL/6 Mice

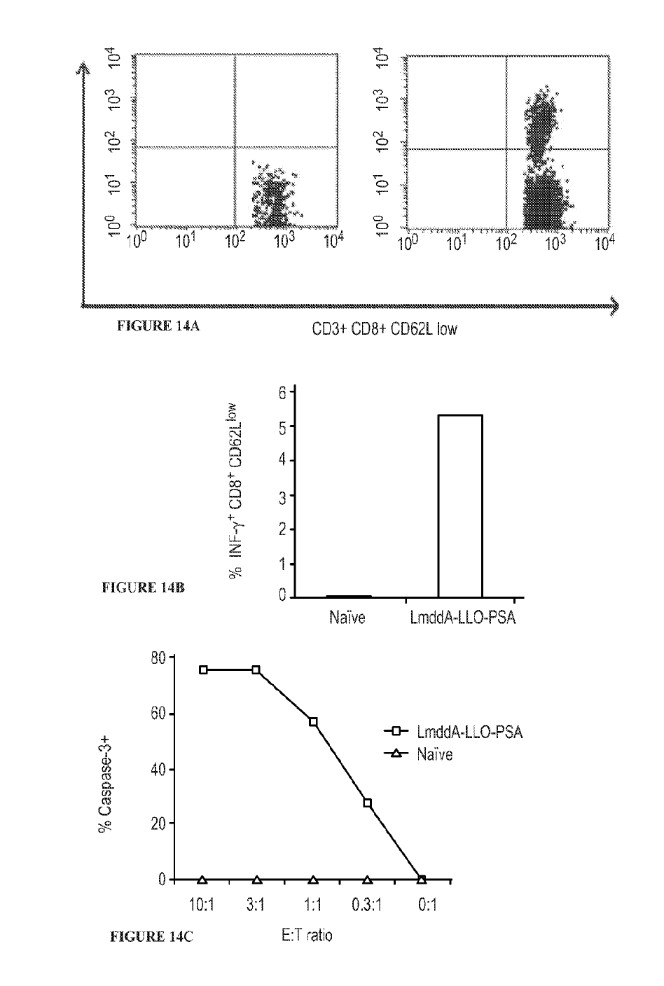

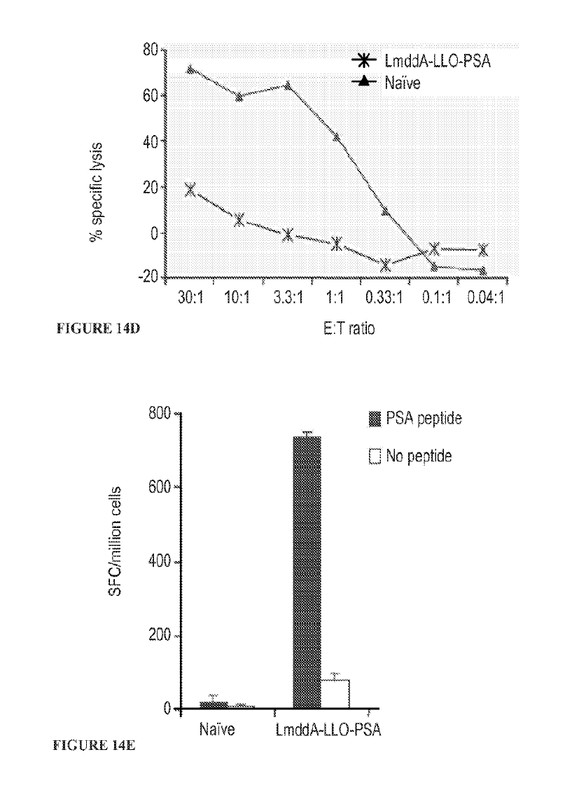

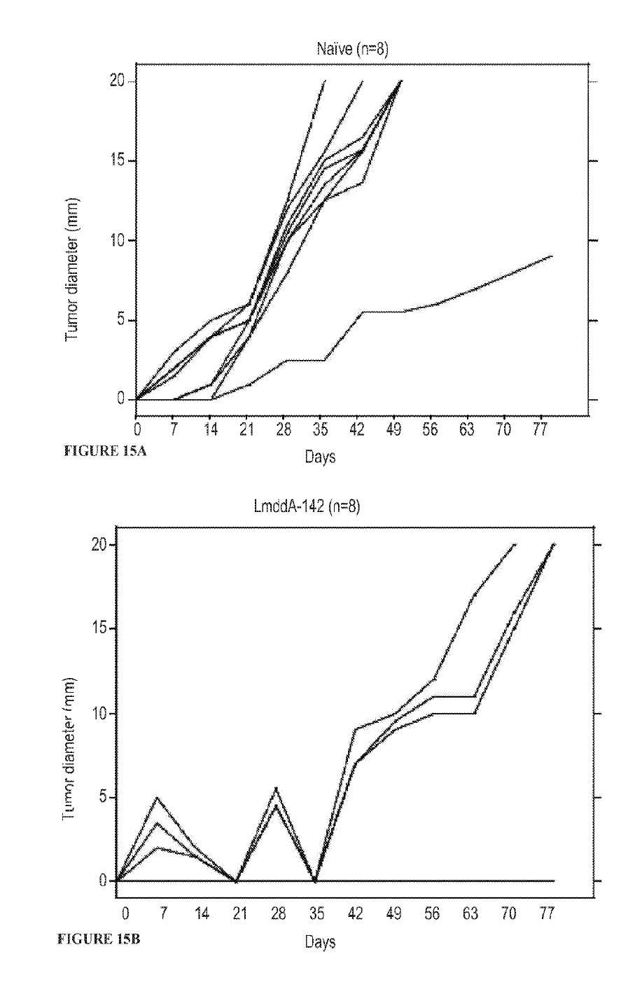

The PSA-specific immune responses elicited by the construct LmddA-LLO-PSA in C57BL/6 mice were determined using PSA tetramer staining. Mice were immunized twice with LmddA-LLO-PSA at one week intervals and the splenocytes were stained for PSA tetramer on day 6 after the boost. Staining of splenocytes with the PSA-specific tetramer showed that LmddA-LLO-PSA elicited 23% of PSA tetramer⁺CD8⁺CD62L^{low} cells (FIG. 14A). The functional ability of the PSA-specific T cells to secrete IFN-γ after stimulation with PSA peptide for 5 h was examined using intracellular cytokine staining. There was a 200-fold increase in the percentage of CD8⁺CD62L^{low}IFN-γ secreting cells stimulated with PSA peptide in the LmddA-LLO-PSA group compared to the naïve mice (FIG. 14B), indicating that the LmddA-LLO-PSA strain is very immunogenic and primes high levels of functionally active PSA CD8⁺ T cell responses against PSA in the spleen.

To determine the functional activity of cytotoxic T cells generated against PSA after immunizing mice with LmddA-LLO-PSA, we tested the ability of PSA-specific CTLs to lyse cells EL4 cells pulsed with H-2D^{b} peptide in an in vitro assay. A FACS-based caspase assay (FIG. 14C) and Europium release (FIG. 14D) were used to measure cell lysis. Splenocytes of mice immunized with LmddA-LLO-PSA contained CTLs with high cytolytic activity for the cells that display PSA peptide as a target antigen.

Elispot was performed to determine the functional ability of effector T cells to secrete IFN-γ after 24 h stimulation with antigen. Using ELISpot, a 20-fold increase in the number of spots for IFN-γ in splenocytes from mice immunized with LmddA-LLO-PSA stimulated with specific peptide when compared to the splenocytes of the naïve mice was observed (FIG. 14E).

### Example 12: Immunization with the LmddA-142 Strains Induces Regression of a Tumor Expressing PSA and Infiltration of the Tumor by PSA-Specific CTLs

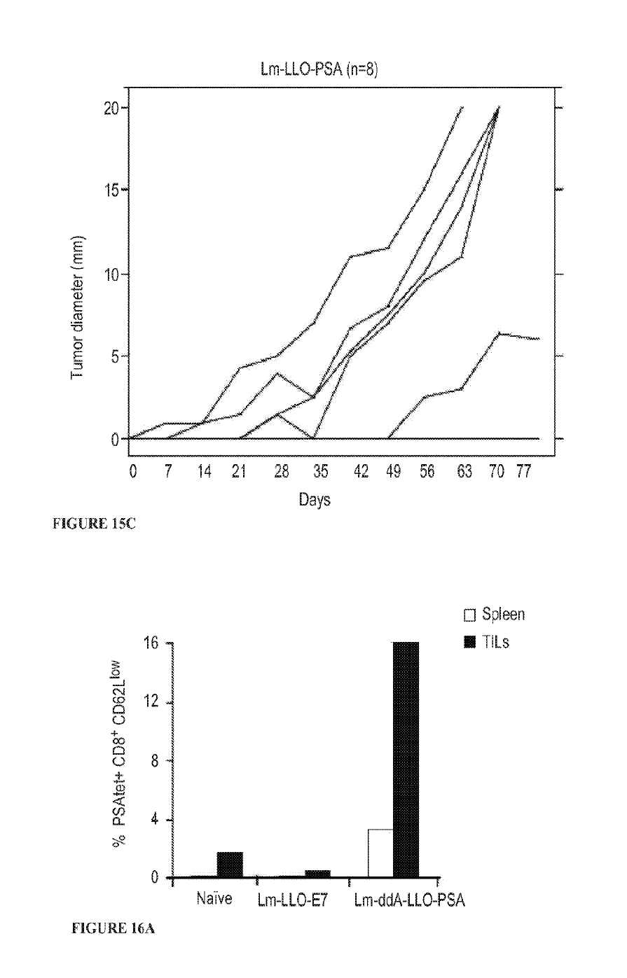

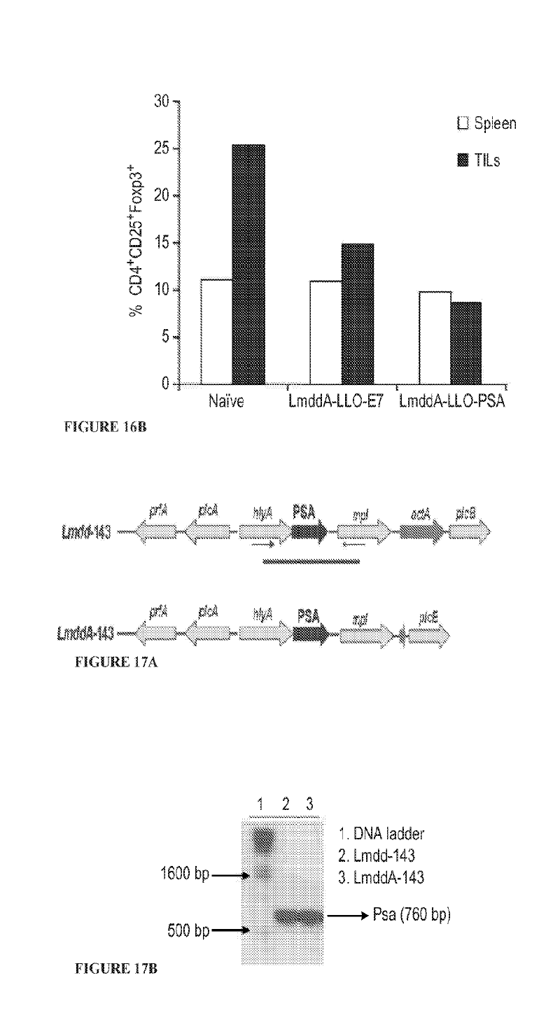

The therapeutic efficacy of the construct LmddA-142 (LmddA-LLO-PSA) was determined using a prostrate adenocarcinoma cell line engineered to express PSA (Tramp-C1-PSA (TPSA); Shahabi et al., 2008). Mice were subcutaneously implanted with 2×10⁶ TPSA cells. When tumors reached the palpable size of 4-6 mm, on day 6 after tumor inoculation, mice were immunized three times at one week intervals with 10⁸ CFU LmddA-142, 10⁷ CFU Lm-LLO-PSA (positive control) or left untreated. The naïve mice developed tumors gradually (FIG. 15A). The mice immunized with LmddA-142 were all tumor-free until day 35 and gradually 3 out of 8 mice developed tumors, which grew at a much slower rate as compared to the naïve mice (FIG. 15B). Five out of eight mice remained tumor free through day 70. As expected, Lm-LLO-PSA-vaccinated mice had fewer tumors than naïve controls and tumors developed more slowly than in controls (FIG. 15C). Thus, the construct LmddA-LLO-PSA could regress 60% of the tumors established by TPSA cell line and slow the growth of tumors in other mice. Cured mice that remained tumor free were rechallenged with TPSA tumors on day 68.

Immunization of mice with the LmddA-142 can control the growth and induce regression of 7-day established Tramp-C1 tumors that were engineered to express PSA in more than 60% of the experimental animals (FIG. 15B), compared to none in the untreated group (FIG. 15A). The LmddA-142 was constructed using a highly attenuated vector (LmddA) and the plasmid pADV142 (Table 2).

Further, the ability of PSA-specific CD8 lymphocytes generated by the LmddA-LLO-PSA construct to infiltrate tumors was investigated. Mice were subcutaneously implanted with a mixture of tumors and matrigel followed by two immunizations at seven day intervals with naïve or control (Lm-LLO-E7) Listeria, or with LmddA-LLO-PSA. Tumors were excised on day 21 and were analyzed for the population of CD8⁺CD62L^{low} PSA^{tetramer+} and CD4⁺ CD25⁺FoxP3⁺ regulatory T cells infiltrating in the tumors.

A very low number of CD8⁺CD62L^{low} PSA^{tetramer+} tumor infiltrating lymphocytes (TILs) specific for PSA that were present in the both naïve and Lm-LLO-E7 control immunized mice was observed. However, there was a 10-30-fold increase in the percentage of PSA-specific CD8⁺CD62L^{low} PSA^{tetramer+} TILs in the mice immunized with LmddA-LLO-PSA (FIG. 7A). Interestingly, the population of CD8⁺CD62L^{low} PSA^{tetramer+} cells in spleen was 7.5 fold less than in tumor (FIG. 16A).

In addition, the presence of CD4⁺/CD25⁺/Foxp3⁺ T regulatory cells (Tregs) in the tumors of untreated mice and Listeria immunized mice was determined. Interestingly, immunization with Listeria resulted in a considerable decrease in the number of CD4⁺ CD25⁺FoxP3⁺ T-regs in tumor but not in spleen (FIG. 16B). However, the construct LmddA-LLO-PSA had a stronger impact in decreasing the frequency of CD4⁺ CD25⁺FoxP3⁺ T-regs in tumors when compared to the naïve and Lm-LLO-E7 immunized group (FIG. 16B).

Thus, the LmddA-142 vaccine can induce PSA-specific CD8⁺ T cells that are able to infiltrate the tumor site (FIG. 16A). Interestingly, immunization with LmddA-142 was associated with a decreased number of regulatory T cells in the tumor (FIG. 16B), probably creating a more favorable environment for an efficient anti-tumor CTL activity.

### Example 13: Lmdd-143 and LmddA-143 Secretes a Functional LLO Despite the PSA Fusion

The Lmdd-143 and LmddA-143 contain the full-length human klk3 gene, which encodes the PSA protein, inserted by homologous recombination downstream and in frame with the hly gene in the chromosome. These constructs were made by homologous recombination using the pKSV7 plasmid (Smith and Youngman, Biochimie. 1992; 74 (7-8) p 705-711), which has a temperature-sensitive replicon, carrying the hly-klk3-mpl recombination cassette. Because of the plasmid excision after the second recombination event, the antibiotic resistance marker used for integration selection is lost. Additionally, the actA gene is deleted in the LmddA-143 strain (FIG. 17A). The insertion of klk3 in frame with hly into the chromosome was verified by PCR (FIG. 17B) and sequencing (data not shown) in both constructs.

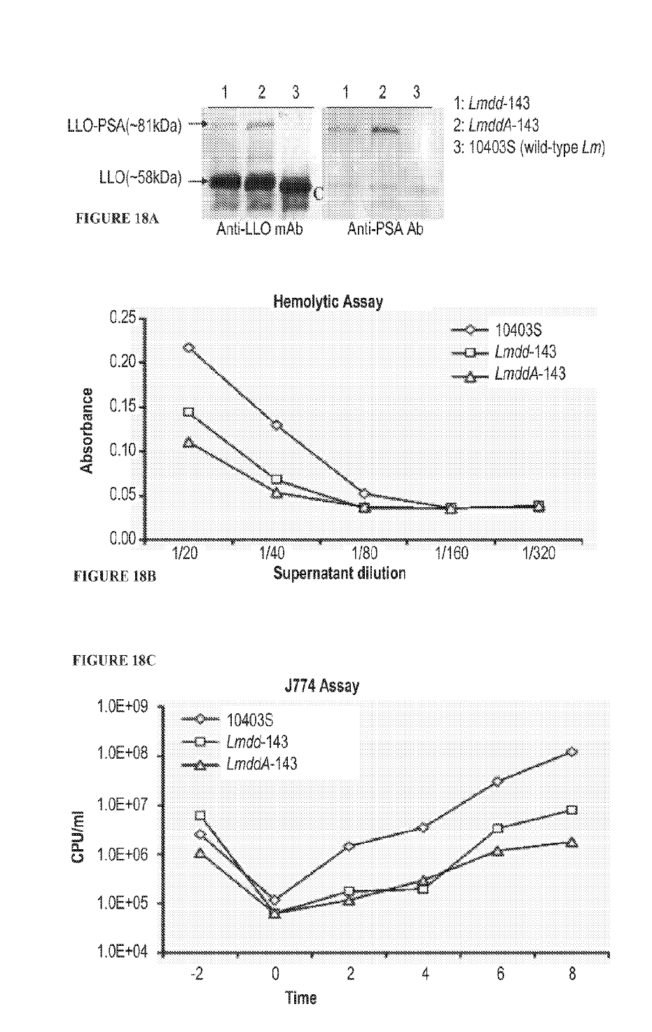

One important aspect of these chromosomal constructs is that the production of LLO-PSA would not completely abolish the function of LLO, which is required for escape of Listeria from the phagosome, cytosol invasion and efficient immunity generated by L. monocytogenes. Western-blot analysis of secreted proteins from Lmdd-143 and LmddA-143 culture supernatants revealed an ˜81 kDa band corresponding to the LLO-PSA fusion protein and an ˜60 kDa band, which is the expected size of LLO (FIG. 18A), indicating that LLO is either cleaved from the LLO-PSA fusion or still produced as a single protein by L. monocytogenes, despite the fusion gene in the chromosome. The LLO secreted by Lmdd-143 and LmddA-143 retained 50% of the hemolytic activity, as compared to the wild-type L. monocytogenes 10403S (FIG. 18B). In agreement with these results, both Lmdd-143 and LmddA-143 were able to replicate intracellularly in the macrophage-like J774 cell line (FIG. 18C).

### Example 14: Both Lmdd-143 and LmddA-143 Elicit Cell-Mediated Immune Responses Against the PSA Antigen

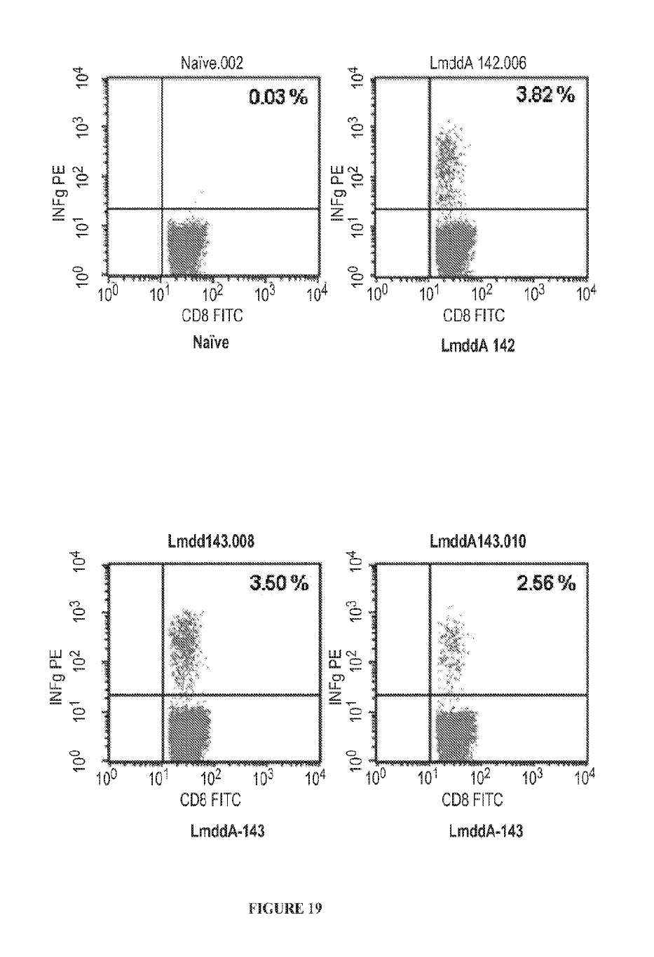

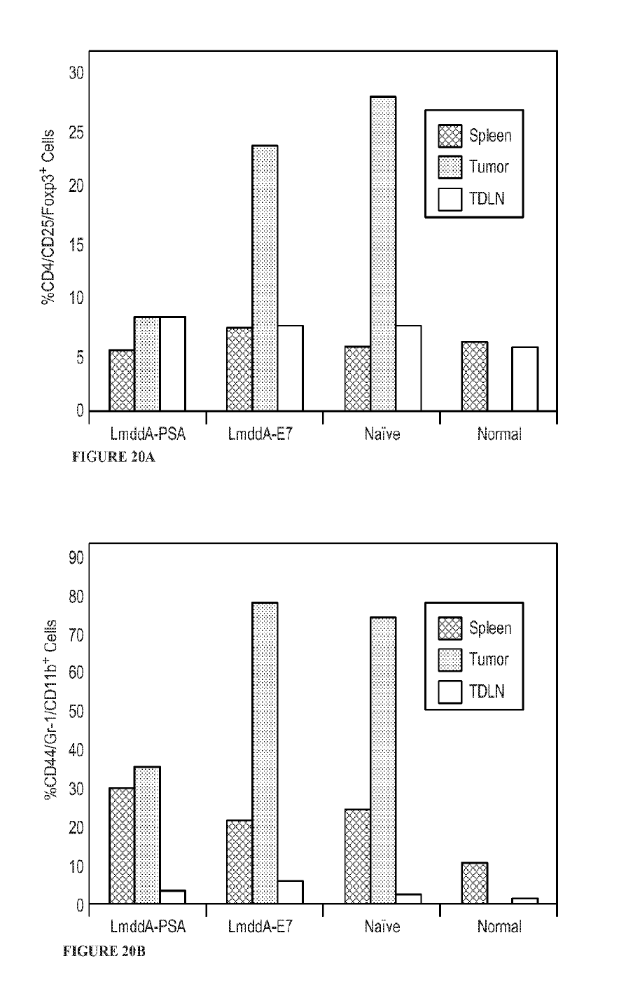

After showing that both Lmdd-143 and LmddA-143 were able to secrete PSA fused to LLO, the question of if these strains could elicit PSA-specific immune responses in vivo was investigated. C57Bl/6 mice were either left untreated or immunized twice with the Lmdd-143, LmddA-143 or LmddA-142. PSA-specific CD8⁺ T cell responses were measured by stimulating splenocytes with the PSA₆₅₋₇₄ peptide and intracellular staining for IFN-γ. As shown in FIG. 19, the immune response induced by the chromosomal and the plasmid-based vectors is similar.

### Materials and Methods (Examples 15-19)

### MDSC and Treg Function

Tumors were implanted in mice on the flank or a physiological site depending on the tumor model. After 7 days, mice were then vaccinated, the initial vaccination day depends on the tumor model being used. The mice were then administered a booster vaccine one week after the vaccine was given.

Mice were then sacrificed and tumors and spleen were harvested 1 week after the boost or, in the case of an aggressive tumor model, 3-4 days after the boost. Five days before harvesting the tumor, non-tumor bearing mice were vaccinated to use for responder T cells. Splenocytes were prepared using standard methodology.

Briefly, single cell suspensions of both the tumors and the spleens were prepared.

Spleens were crushed manually and red blood cells were lysed. Tumors were minced and incubated with collagenase/DNase. Alternatively, the GENTLEMACS™ dissociator was used with the tumor dissociation kit.

MDSCs or Tregs were purified from tumors and spleens using a Miltenyi kit and columns or the autoMACs separator. Cells were then counted.

Single cell suspension was prepared and the red blood cells were lysed. Responder T cells were then labeled with CFSE.

Cells were plated together at a 2:1 ratio of responder T cells (from all division cycle stages) to MDSCs or Tregs at a density of 1×10⁵ T cells per well in 96 well plates. Responder T cells were then stimulated with either the appropriate peptide (PSA OR CA9) or non-specifically with PMA/ionomycin. Cells were incubated in the dark for 2 days at 37° C. with 5% CO₂. Two days later, the cells were stained for FACS and analyzed on a FACS machine.

### Analysis of T-Cell Responses

For cytokine analysis by ELISA, splenocytes were harvested and plated at 1.5 million cells per well in 48-well plates in the presence of media, SEA or conA (as a positive control). After incubation for 72 hours, supernatants were harvested and analyzed for cytokine level by ELISA (BD). For antigen-specific IFN-γ ELISpot, splenocytes were harvested and plated at 300K and 150K cells per well in IFN-γ ELISpot plates in the presence of media, specific CTL peptide, irrelevant peptide, specific helper peptide or conA (as a positive control). After incubation for 20 hours, ELISpots (BD) were performed and spots counted by the Immunospot analyzer (C.T.L.). Number of spots per million splenocytes were graphed.

Splenocytes were counted using a Coulter Counter, Z1. The frequency of IFN-γ producing CD8+ T cells after re-stimulation with gag-CTL, gag-helper, medium, an irrelevant antigen, and con A (positive control) was determined using a standard IFN-γ-based ELISPOT assay.

Briefly, IFN-γ was detected using the mAb R40-A2 at 5 mg/ml and polyclonal rabbit anti-IFN-γ used at an optimal dilution (kindly provided by Dr. Phillip Scott, University of Pennsylvania, Philadelphia, Pa.). The levels of IFN-γ were calculated by comparison with a standard curve using murine rIFN-γ (Life Technologies, Gaithersburg, Md.). Plates were developed using a peroxidase-conjugated goat anti-rabbit IgG Ab (IFN-γ). Plates were then read at 405 nm. The lower limit of detection for the assays was 30 pg/ml.

### Example 15: Suppressor Cell Function after Listeria Vaccine Treatment

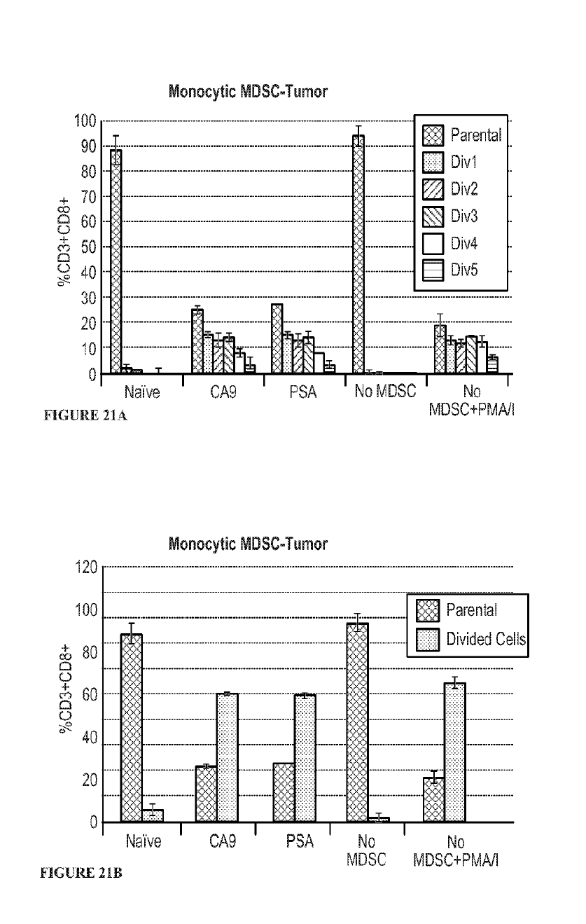

At day 0 tumors were implanted in mice. At day 7 mice were vaccinated with Lmdda-E7 or LmddA-PSA. At day 14 tumors were harvested and the number and percentages of infiltrating MDSCs and Treg were measured for vaccinated and naïve groups. It was found that there is a decrease in the percentages of both MDSC and Tregs in the tumors of Listeria-treated mice, and the absolute number of MDSC, whereas the same effect is not observed in the spleens or the draining lymph nodes (TLDN) (FIG. 20).

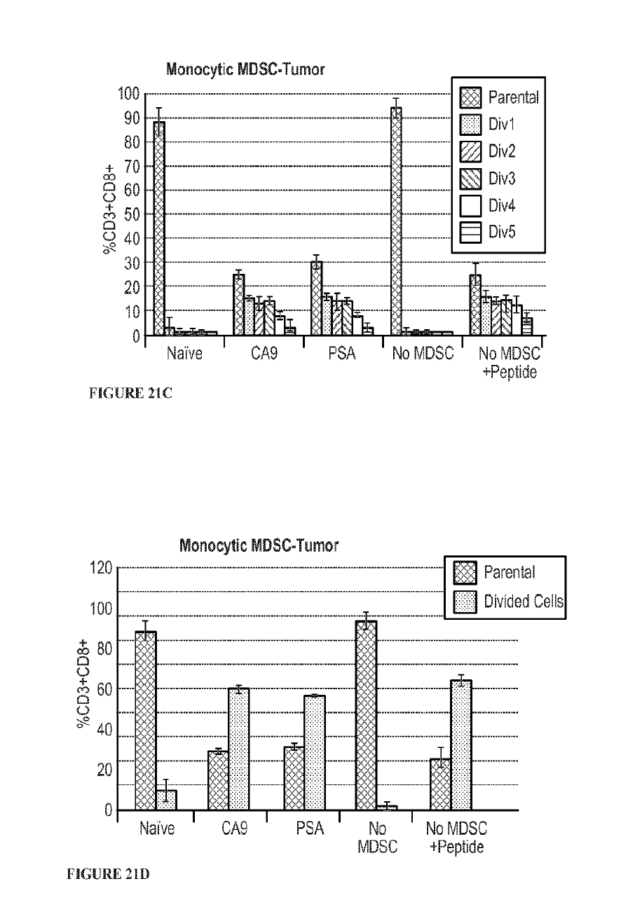

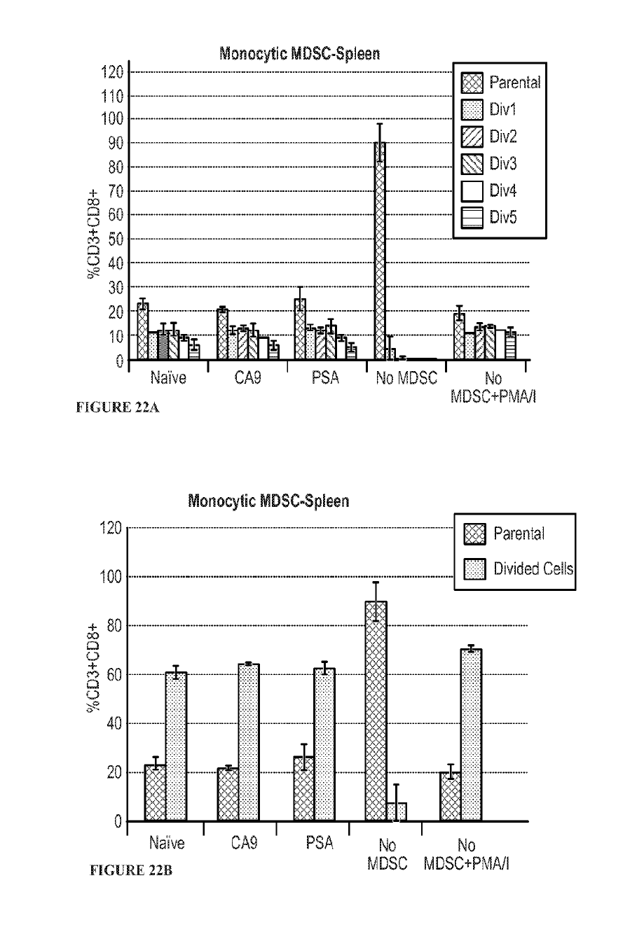

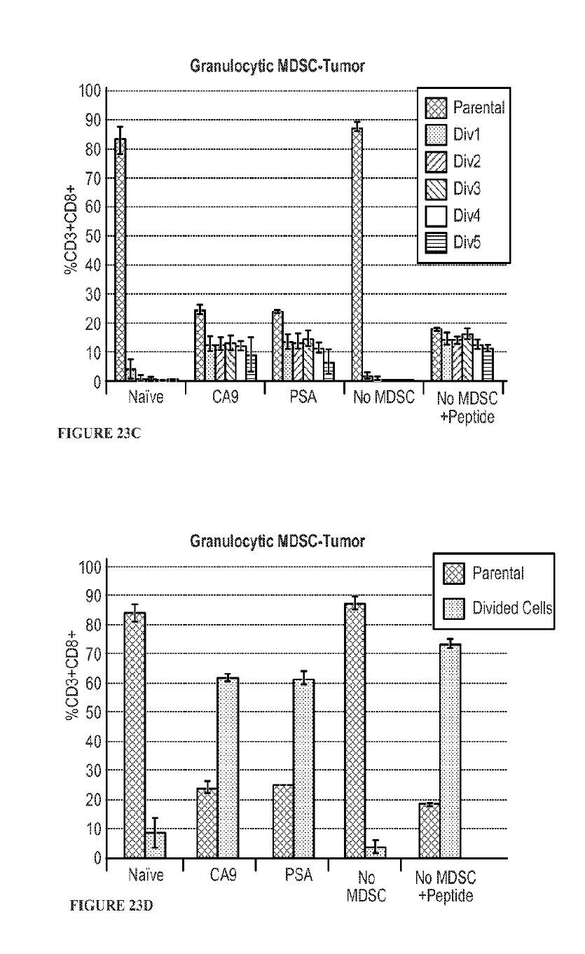

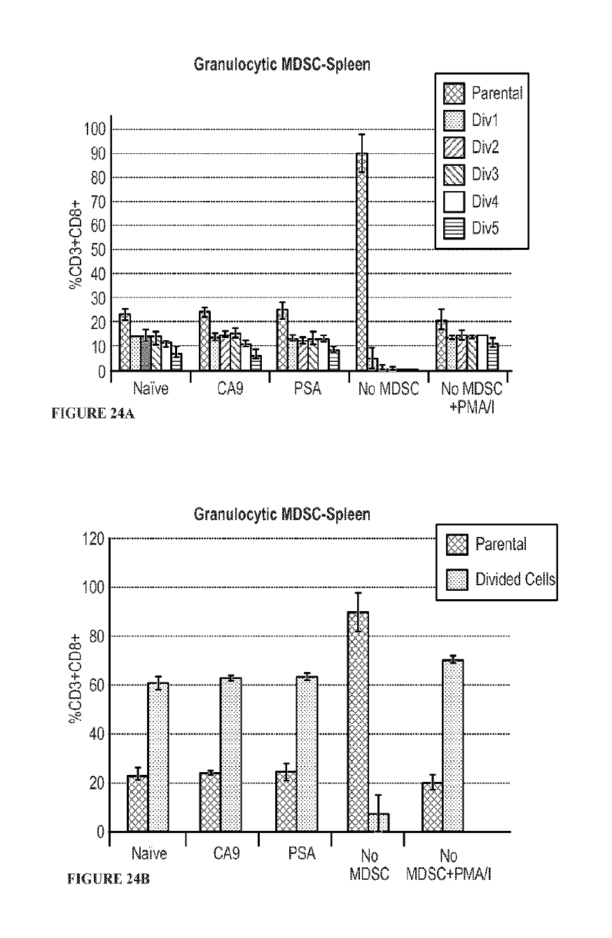

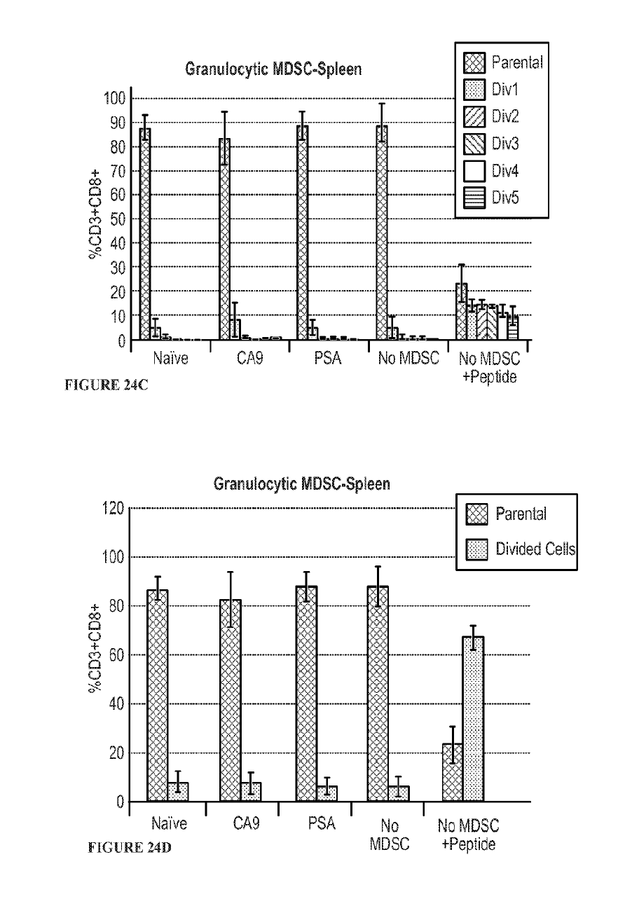

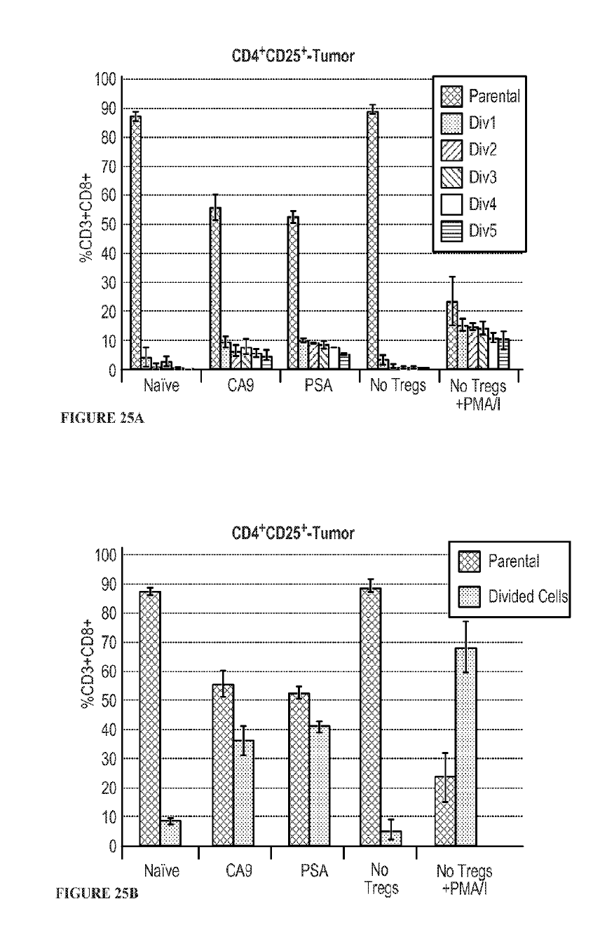

Isolated splenocytes and tumor-infiltrating lymphocytes (TILs) extracted from tumor bearing mice in the above experiment were pooled and stained for CD3, and CD8 to elucidate the effect of immunization with Lm-LLO-E7, Lm-LLO-PSA and Lm-LLO-CA9, Lm-LLO-Her2 (FIGS. 21-23) on the presence of MDSCs and Tregs (both splenic and tumoral MDSCs and Tregs) in the tumor. Each column represents the % of T cell population at a particular cell division stage and is subgrouped under a particular treatment group (naïve, peptide—CA9 or PSA—treated, no MDSC/Treg, and no MDSC+PMA/ionomycin) (FIGS. 21-23).

Blood from tumor-bearing mice was analyzed for the percentages of Tregs and MDSCs present. There is a decrease in both MDSC and Tregs in the blood of mice after Lm vaccination.

### Example 16: MDSCS from TPSA23 Tumors but not Spleen are Less Suppressive after Listeria Vaccination

Suppressor assays were carried out using monocytic and granulocytic MDSCs isolated from TPSA23 tumors with non-specifically activated naïve murine cells, and specifically activated cells (PSA, CA9, PMA/ionomycin). Results demonstrated that the MDSCs isolated from tumors from the Lm vaccinated groups have a diminished capacity to suppress the division of activated T cells as compared to MDSC from the tumors of naïve mice. (see Lm-LLO-PSA and Lm-LLO-treated Groups in FIGS. 21&23, right-hand panel in figures represents pooled cell division data from left-hand panel). In addition, T responder cells from untreated mice where no MDSCs were present and where the cells were unstimulated/activated, remained in their parental (resting) state (FIGS. 21&23), whereas T cells stimulated with PMA or ionomycin were observed to replicate (FIGS. 21&23). Further, it was observed that both, the Gr+Ly6G+ and the GrdimLy6G-MDSCs are less suppressive after treatment with Listeria vaccines. This applies to their decreased abilities to suppress both the division of activated PSA-specific T cells and non-specific (PMA/Ionomycin stimulated) T cells.

Moreover, suppressor assays carried out using MDSCs isolated from TPSA23 tumors with non-specifically activated naïve murine cells demonstrated that the MDSCs isolated from tumors from the Lm vaccinated groups have a diminished capacity to suppress the division of activated T cells as compared to MDSC from the tumors of naïve mice (FIGS. 21&23).

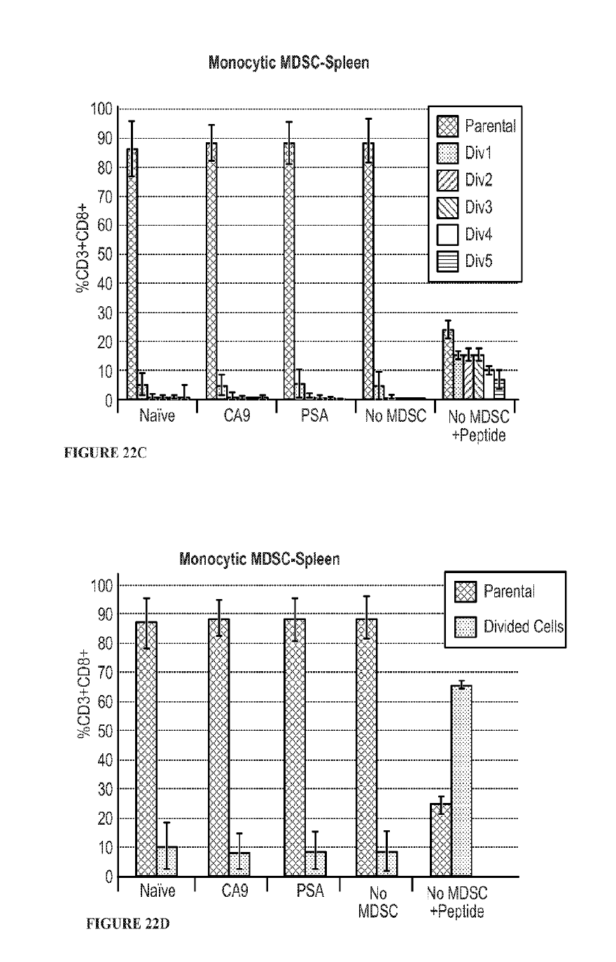

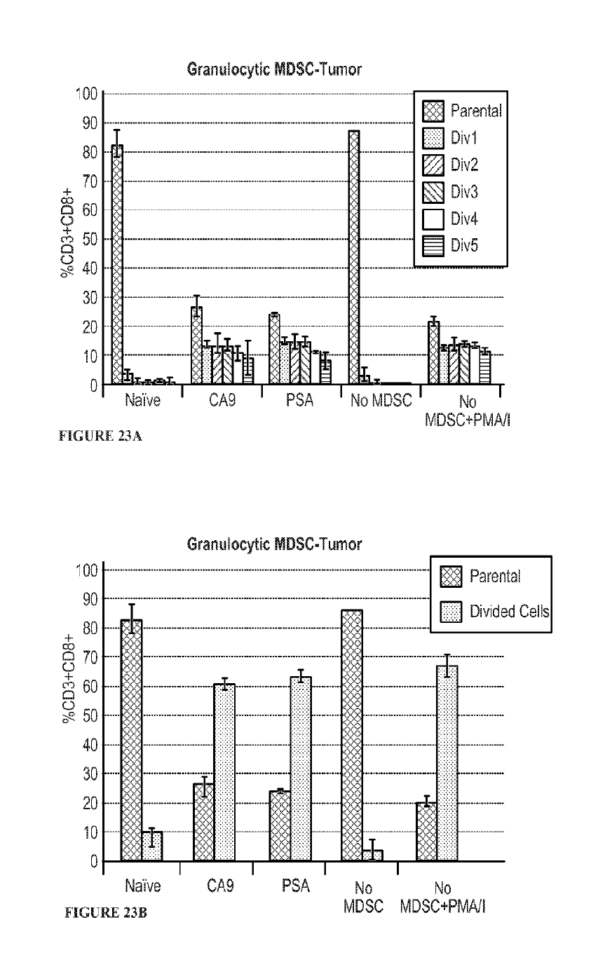

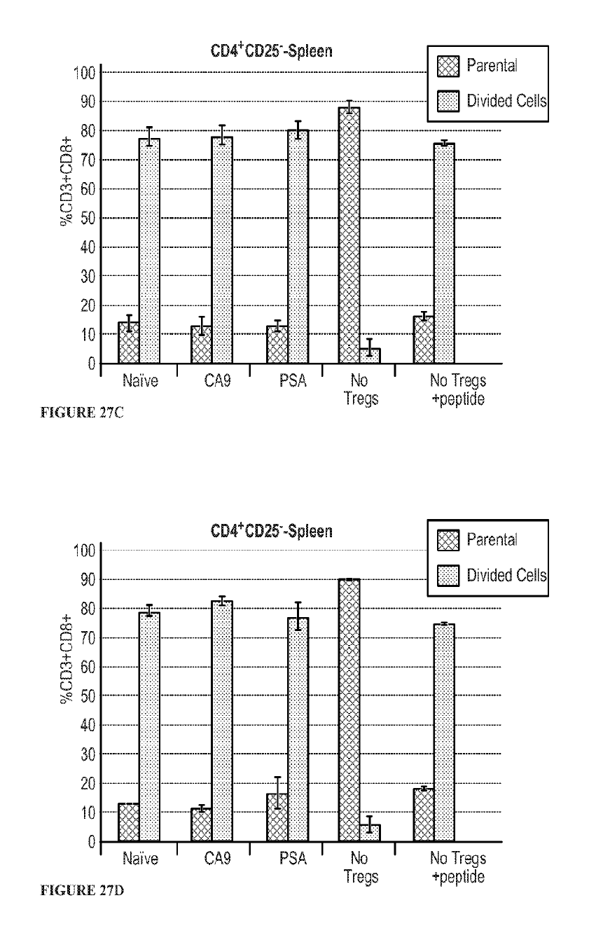

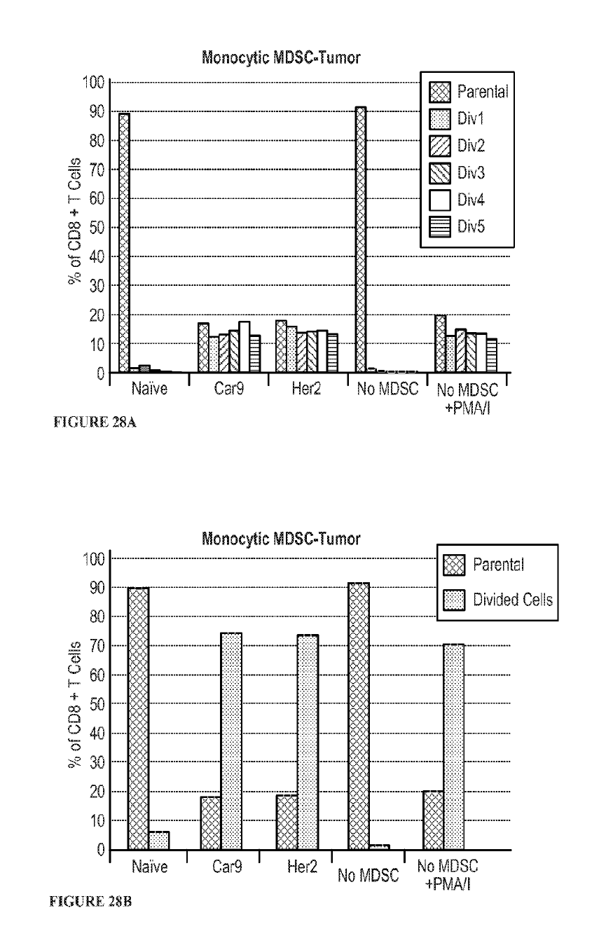

In addition, the observations discussed immediately above relating to FIGS. 21 and 27 were not observed when using splenic MDSCs. In the latter, splenocytes/T cells from the naïve group, the Listeria-treated group (PSA, CA9), and the PMA/ionomycin stimulated group (positive control) all demonstrated the same level of replication (FIGS. 22&24). Hence, these results show that Listeria-mediated inhibition of suppressor cells in tumors worked in an antigen-specific and non-specific manner, whereas Listeria has no effect on splenic granulocytic MDSCs as they are only suppressive in an antigen-specific manner.

### Example 17: Tumor T Regulatory Cells' Reduced Suppression

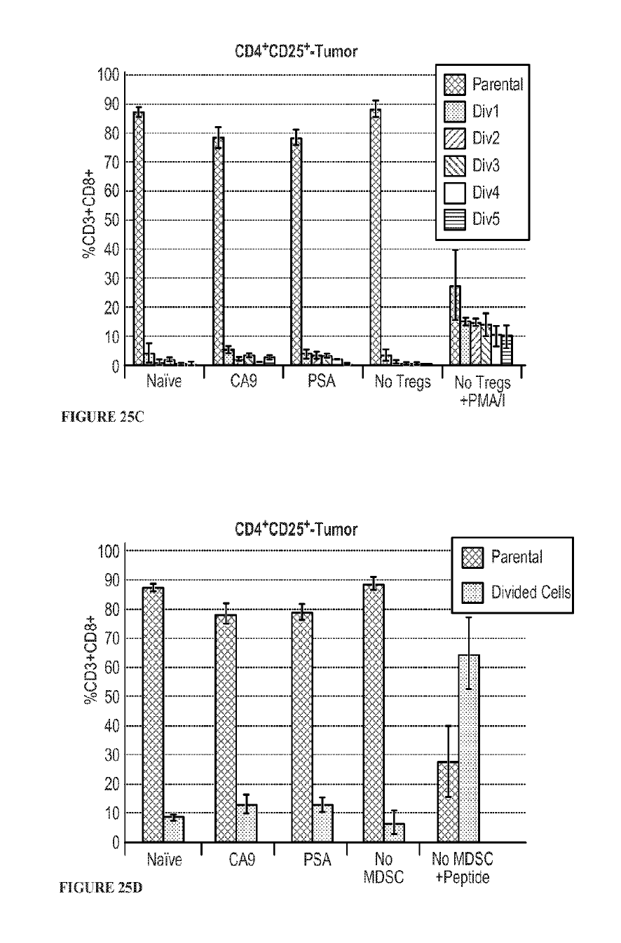

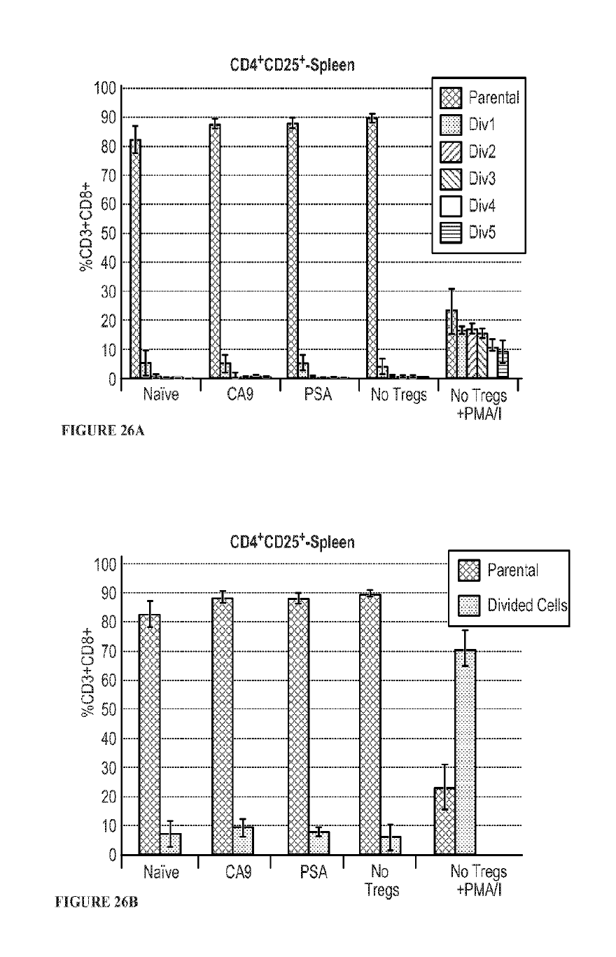

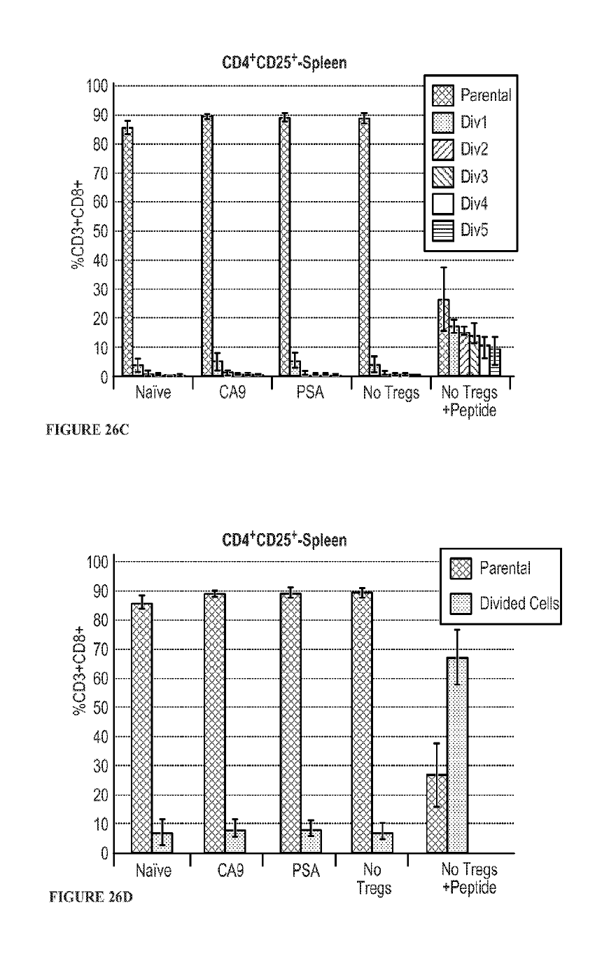

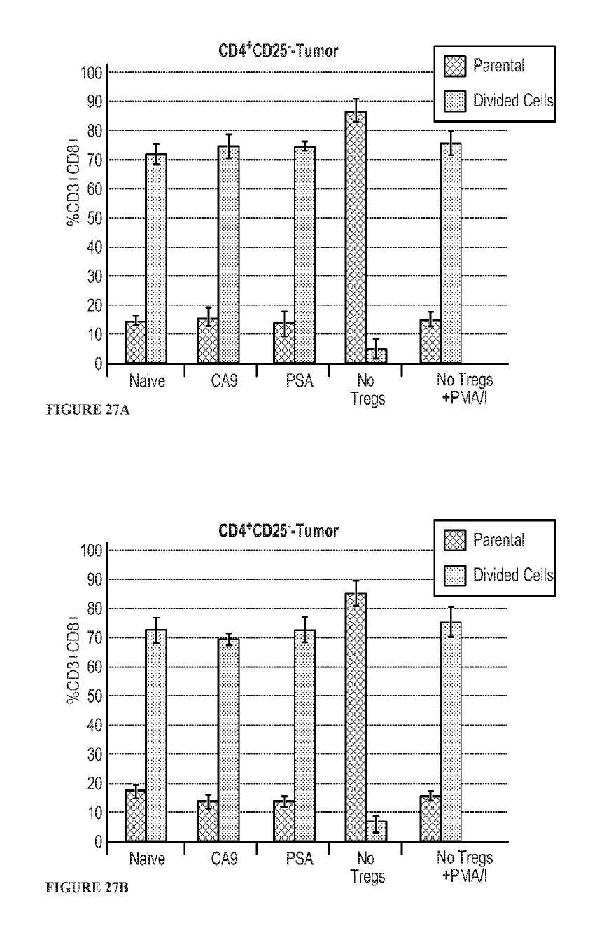

Suppressor assays were carried out using Tregs isolated from TPSA23 tumors after Listeria treatment. It was observed that after treatment with Listeria there is a reduction of the suppressive ability of Tregs from tumors (FIG. 25), however, it was found that splenic Tregs are still suppressive (FIG. 26).

As a control conventional CD4+ T cells were used in place of MDSCs or Tregs and were found not to have an effect on cell division (FIG. 27).

### Example 18: MDSCS and Tregs from 4t1 Tumors but not Spleen are Less Suppressive after Listeria Vaccination

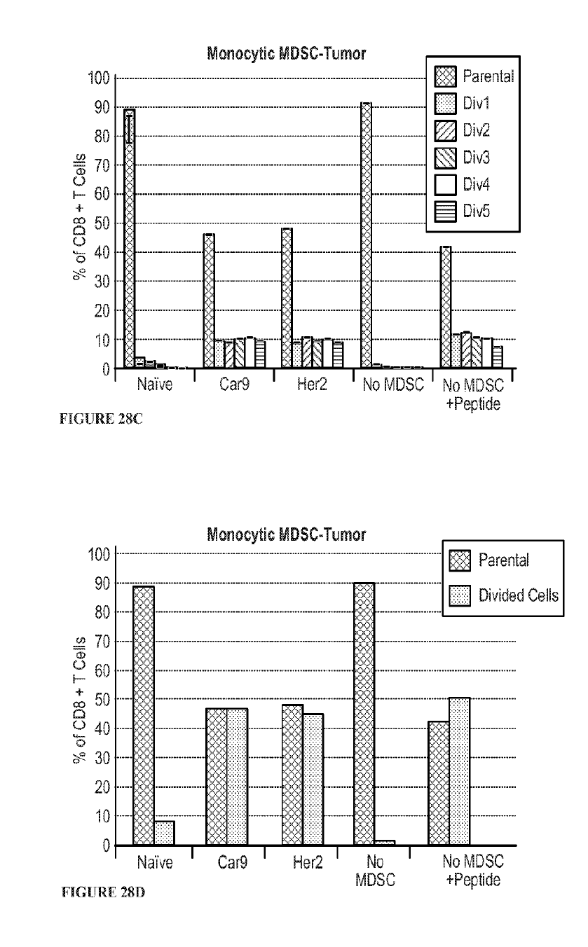

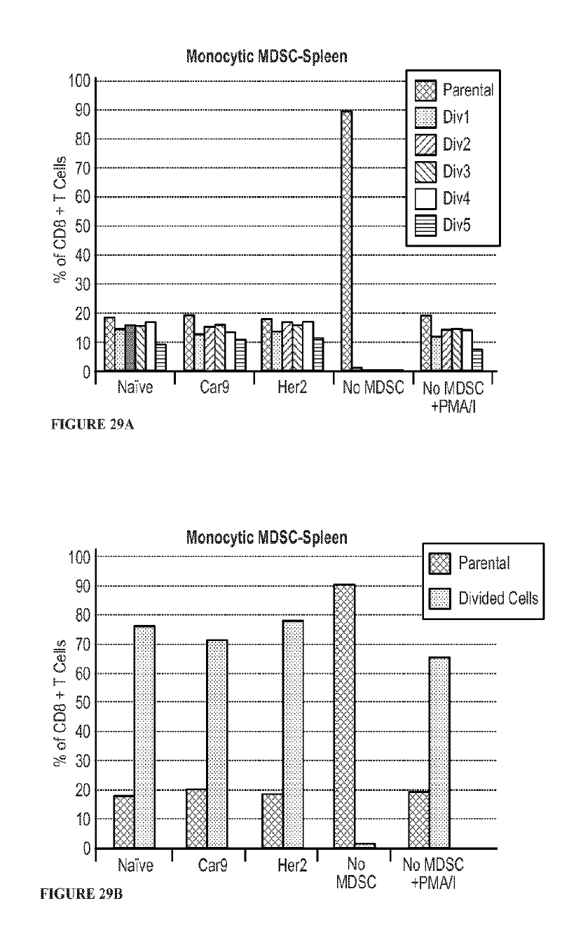

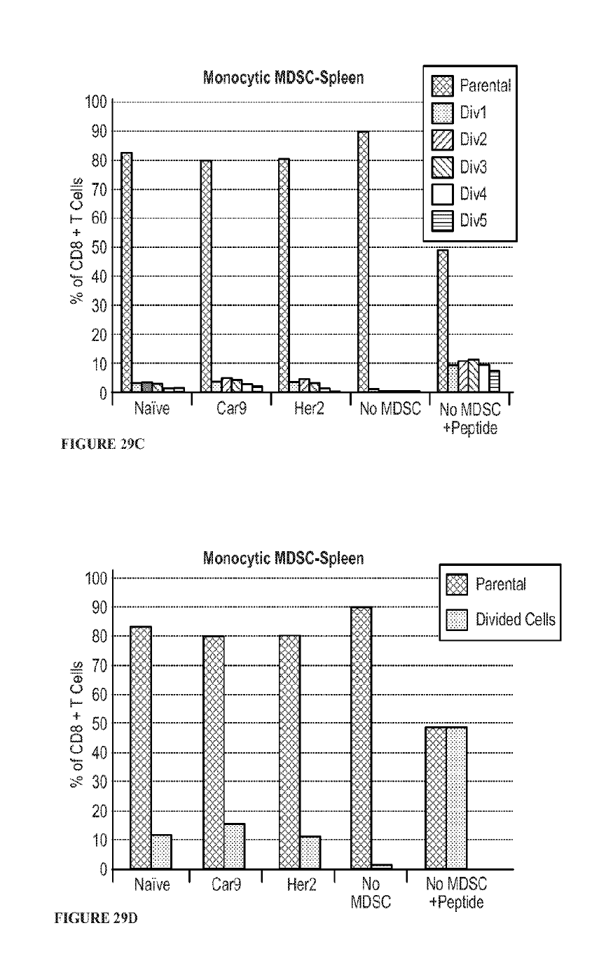

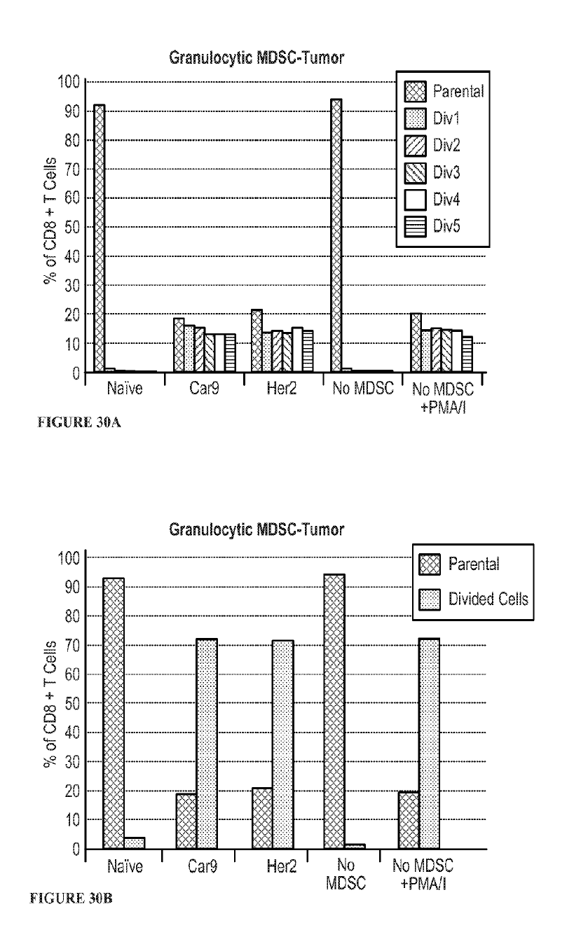

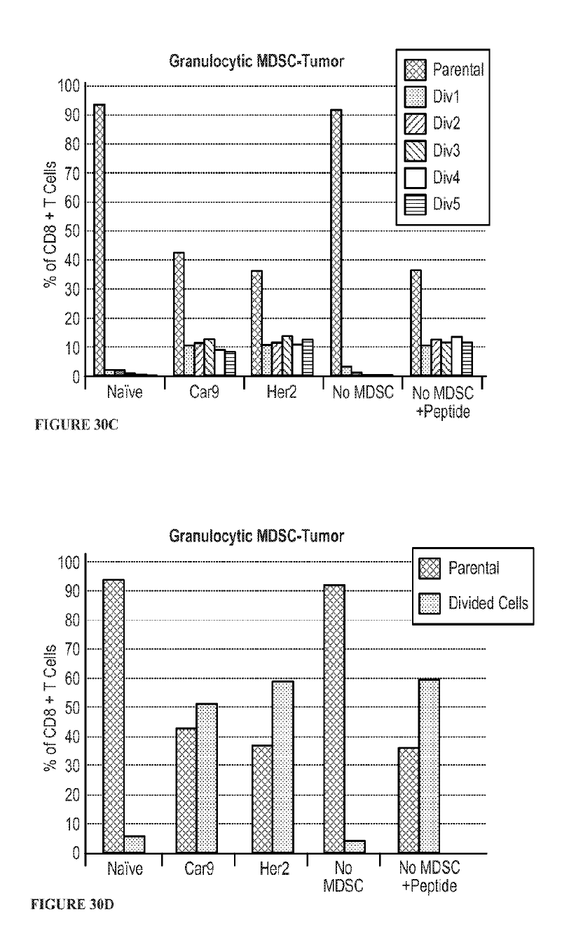

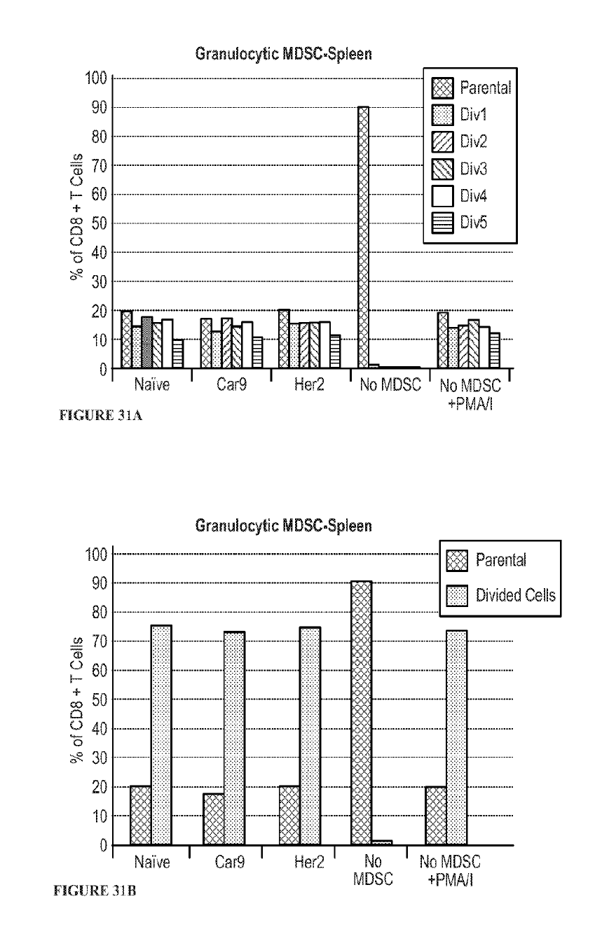

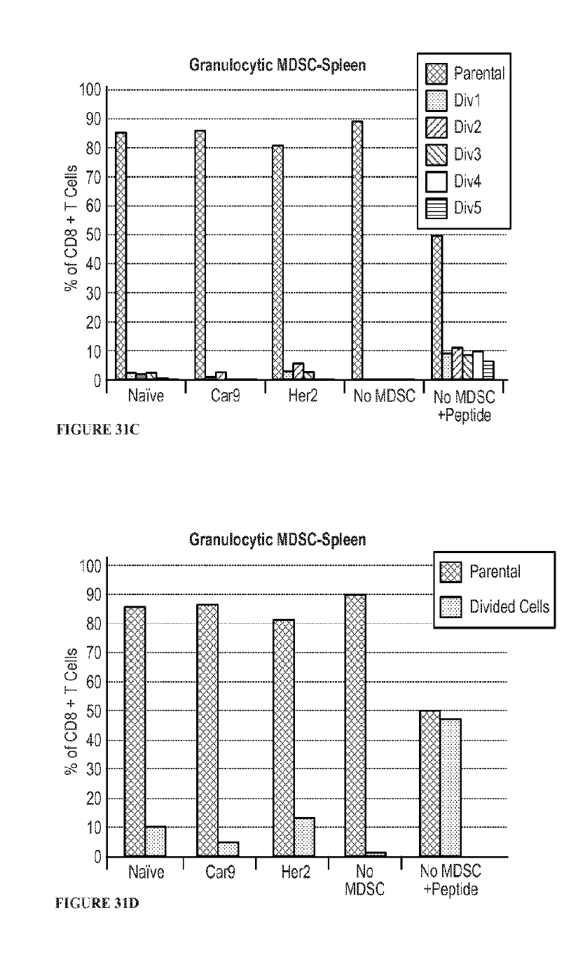

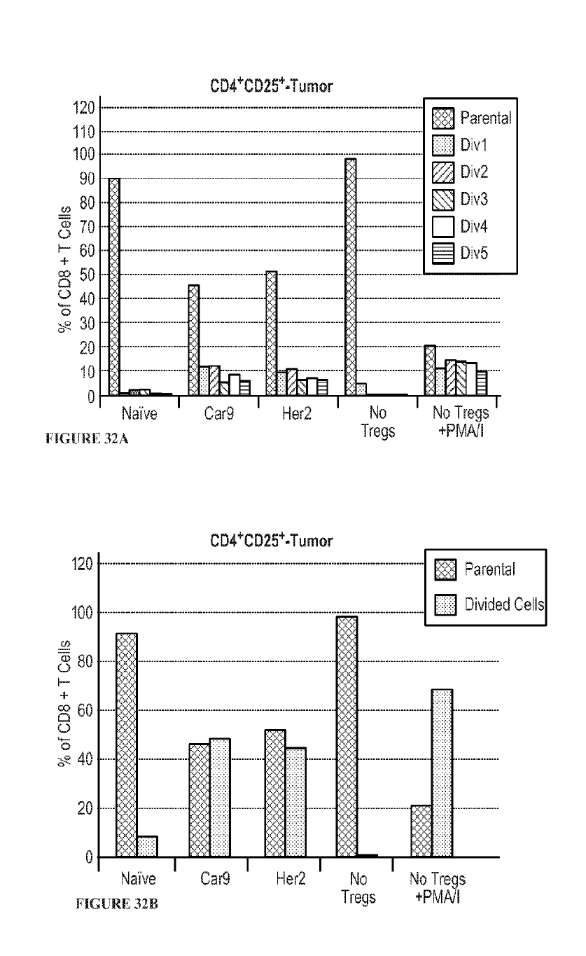

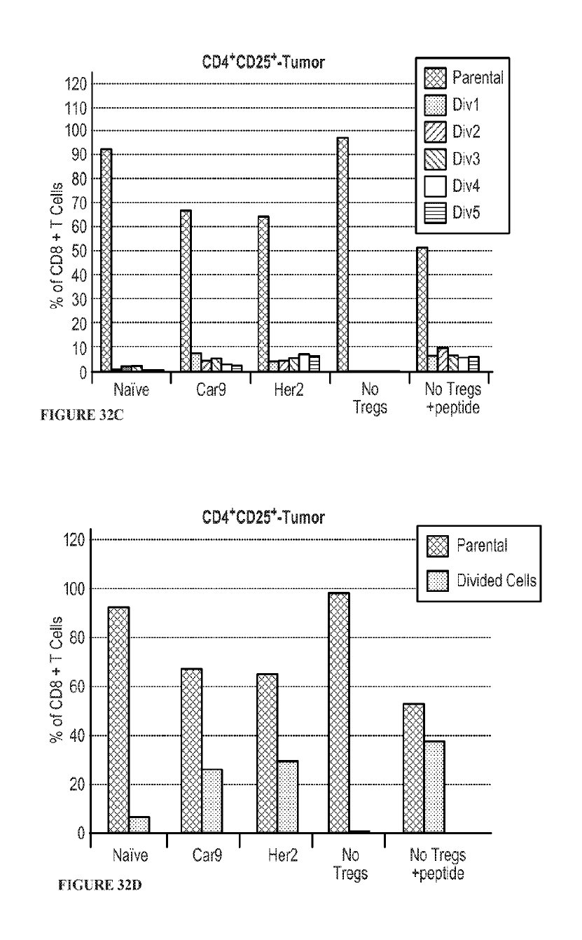

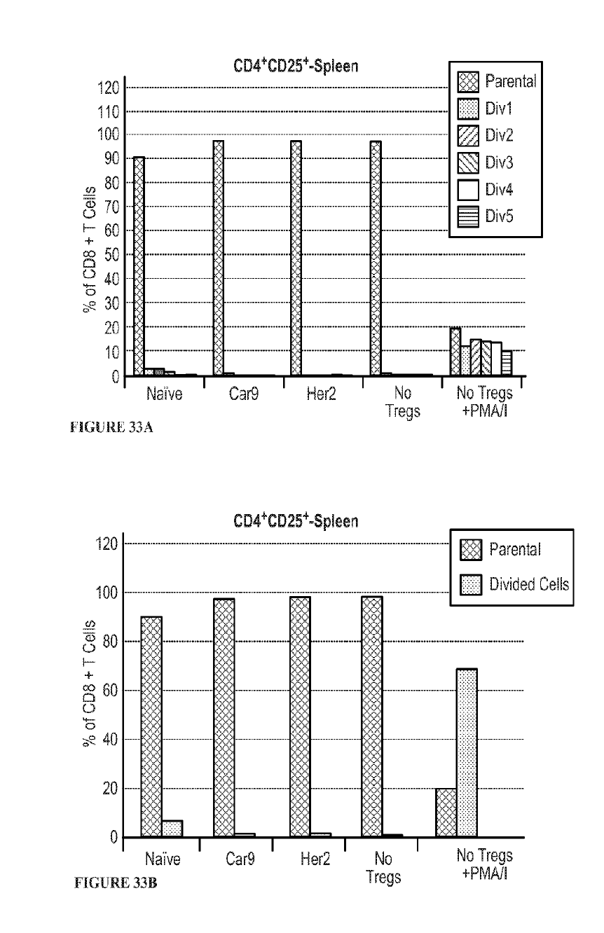

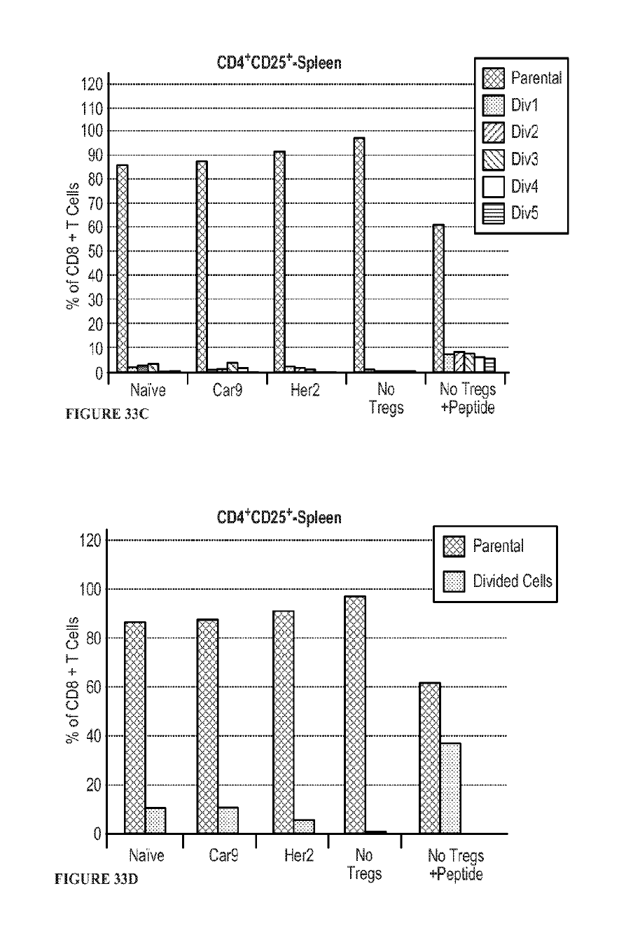

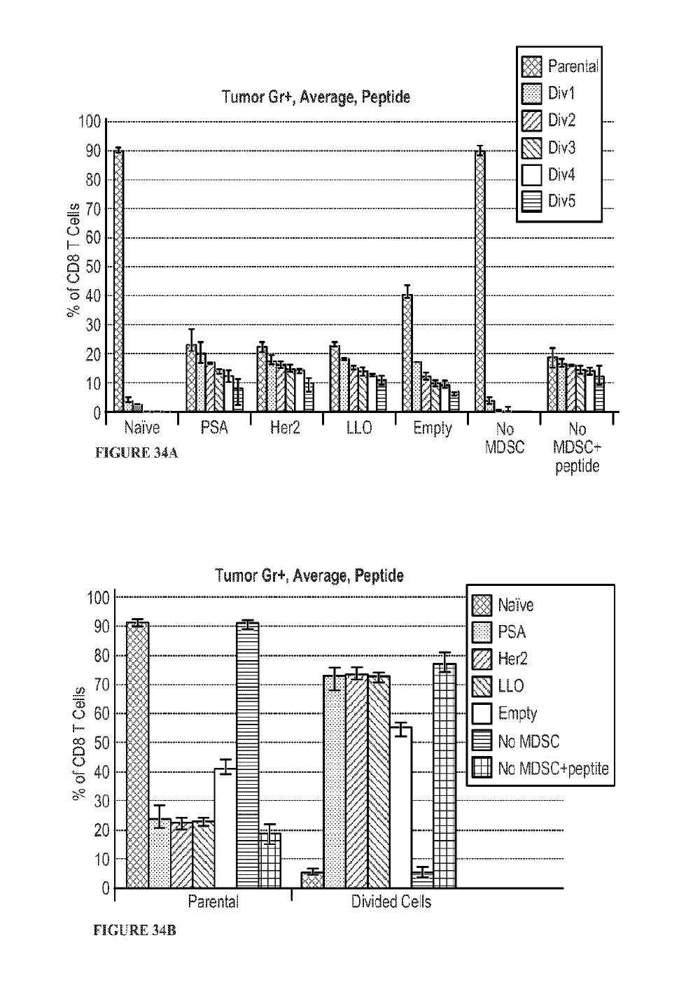

As in the above, the same experiments were carried out using 4T1 tumors and the same observations were made, namely, that MDSCs are less suppressive after Listeria vaccination (FIGS. 28 & 30), that Listeria has no specific effect on splenic monocytic MDSCs (FIGS. 29 & 31), that there is a decrease in the suppressive ability of Tregs from 4T1 tumors after Listeria vaccination (FIG. 32), and that Listeria has no effect on the suppressive ability of splenic Tregs (FIG. 33).

Finally, it was observed that Listeria has no effect on the suppressive ability of splenic Tregs.

### Example 19: Change in the Suppressive Ability of the Granulocity and Monocytic MDSC is Due to the Overexpression of tLLO

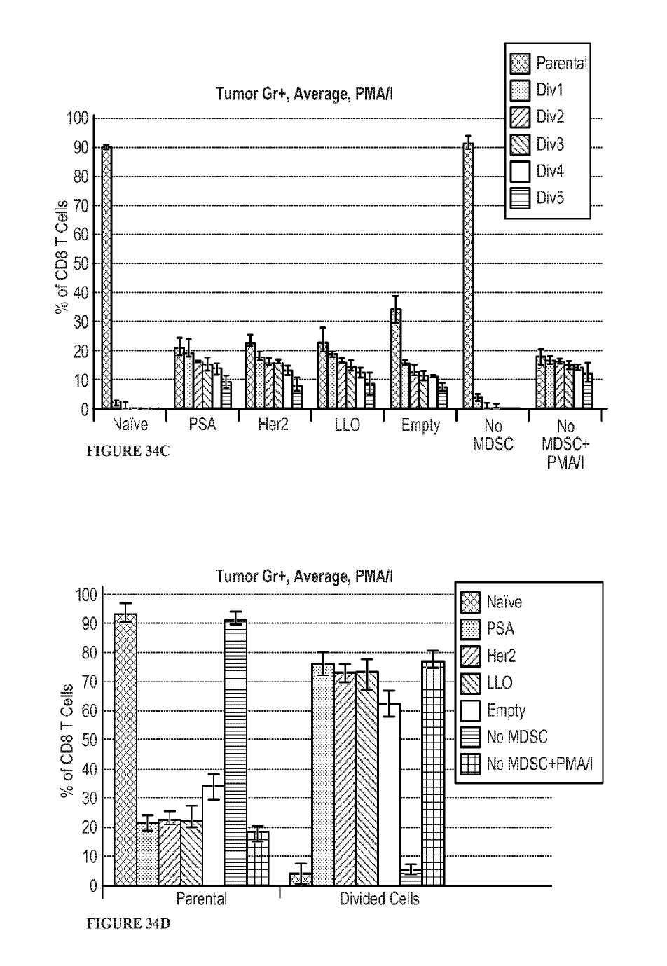

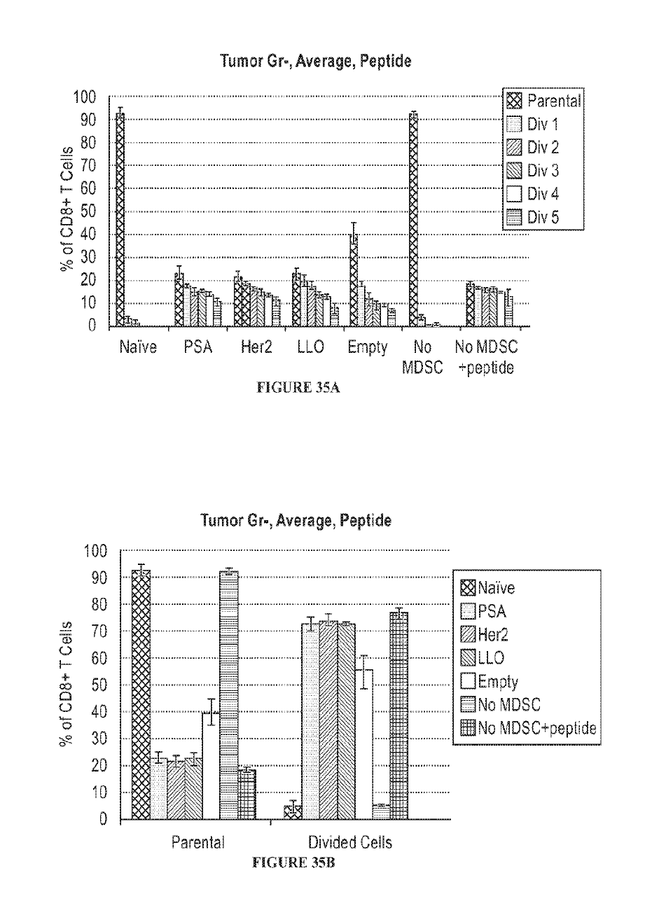

The LLO plasmid shows similar results as the Listeria vaccines with either the TAA or an irrelevant antigen (FIG. 34). This means that the change in the suppressive ability of the granulocytic MDSC is due to the overexpression of tLLO and is independent of the partnering fusion antigen. The empty plasmid construct alone also led to a change in the suppressive ability of the MDSC, although not to exactly the same level as any of the vaccines that contain the truncated LLO on the plasmid. The average of the 3 independent experiments show that the difference in suppression between the empty plasmid and the other plasmids with tLLO (with and without a tumor antigen) are significant. Reduction in MDSC suppressive ability was identical regardless of the fact if antigen specific or non-specific stimulated responder T cells were used.

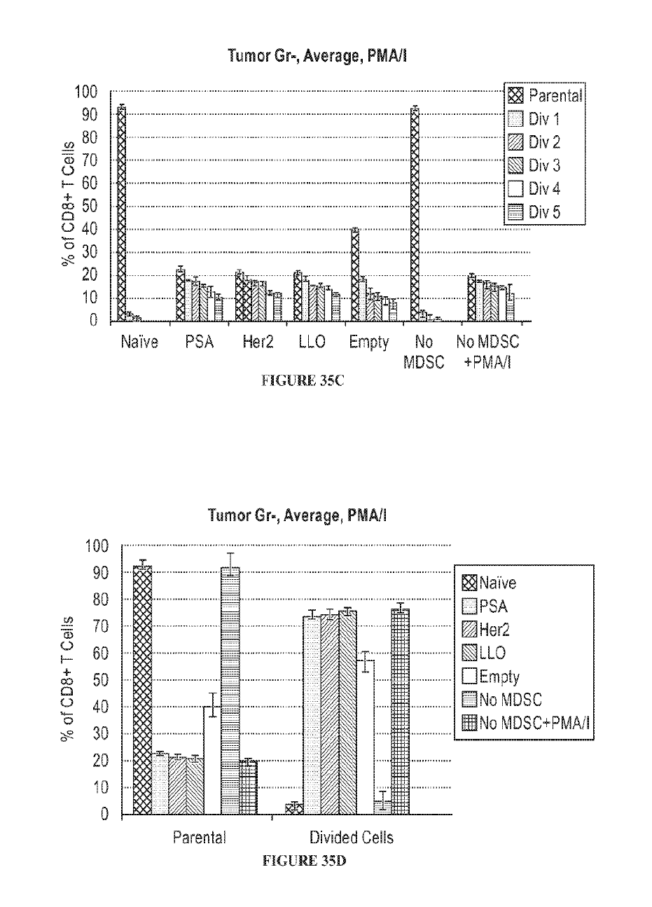

Similar to the granulocytic MDSC, the average of the 3 independent experiments shows that the differences observed in the suppressive ability of the monocytic MDSCs purified from the tumors after vaccination with the Lm-empty plasmid vaccine are significant when compared to the other vaccine constructs (FIG. 35).

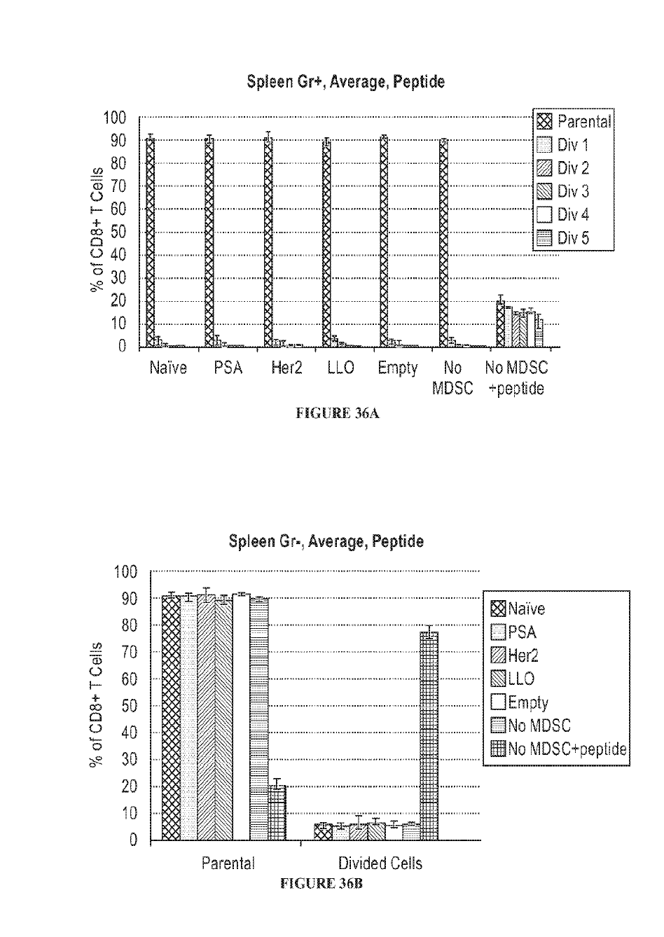

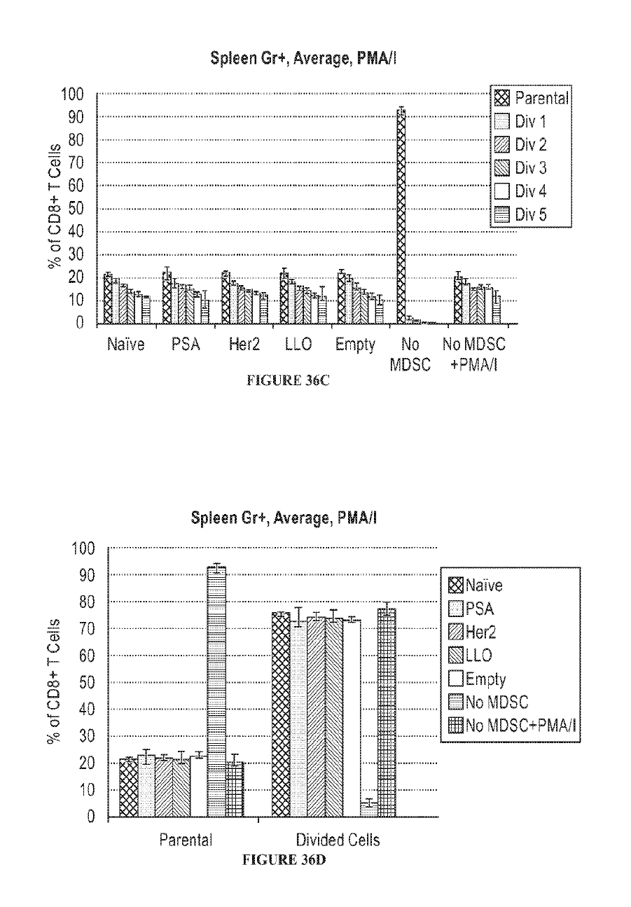

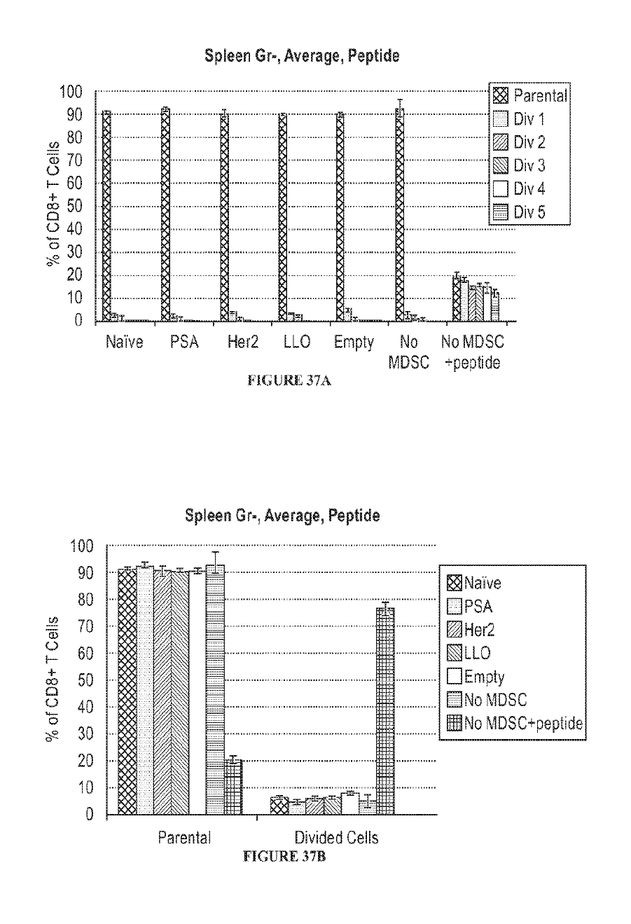

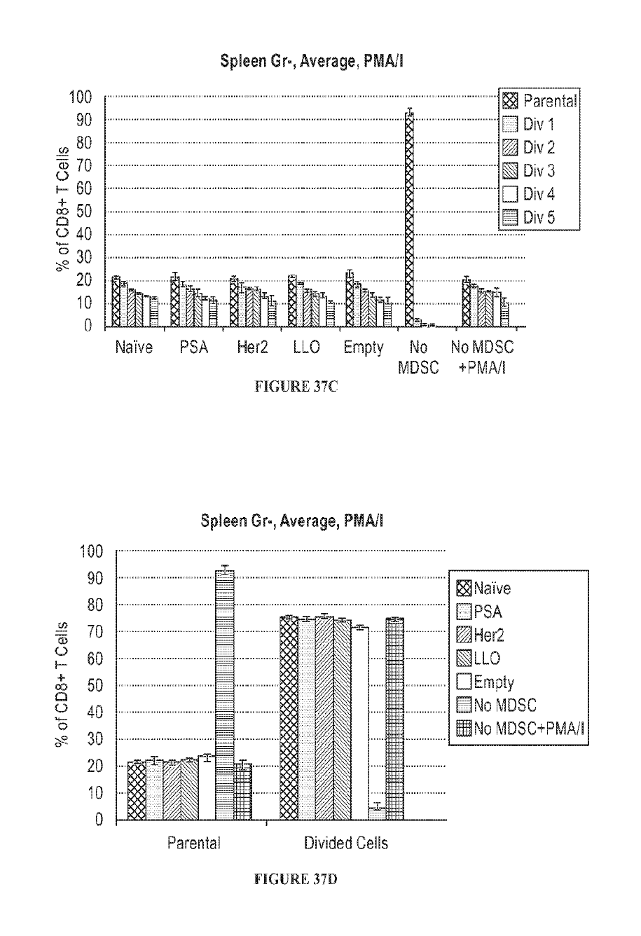

Similar to the above observations, granulocytic MDSC purified from the spleen retain their ability to suppress the division of the antigen-specific responder T cells after Lm vaccination (FIG. 36). However, after non-specific stimulation, activated T cells (with PMA/ionomycin) are still capable of dividing. None of these results are altered with the use of the LLO only or the empty plasmid vaccines showing that the Lm-based vaccines are not affecting the splenic granulocytic MDSC (FIG. 35).

Similarly, monocytic MDSC purified from the spleen retain their ability to suppress the division of the antigen-specific responder T cells after Lm vaccination. However, after non-specific activation (stimulated by PMA/ionomycin), T cells are still capable of dividing. None of these results are altered with the use of the LLO only or the empty plasmid vaccines showing that the Lm vaccines are not affecting the splenic monocytic MDSC (FIG. 37).

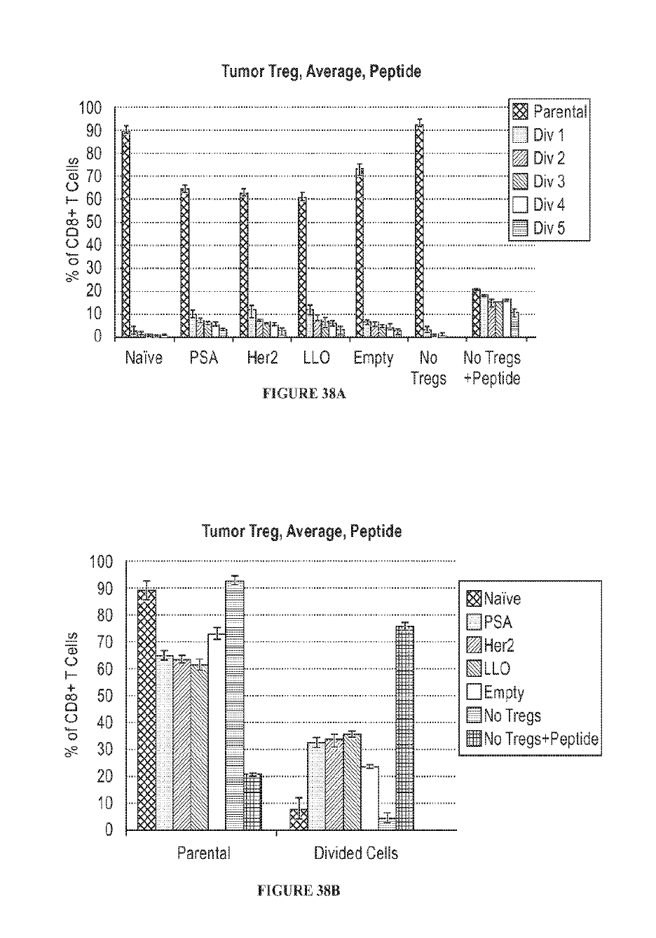

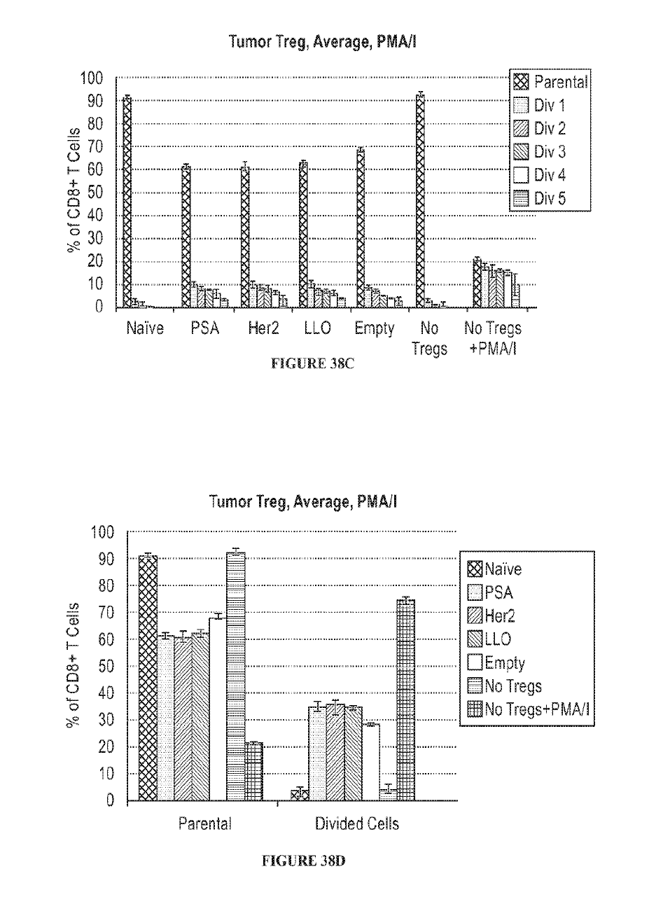

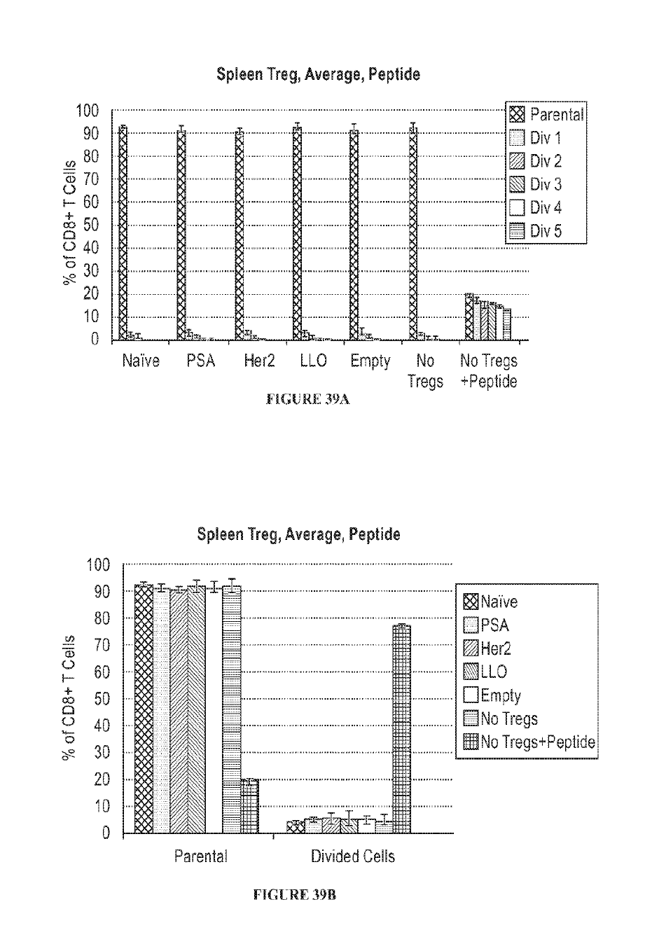

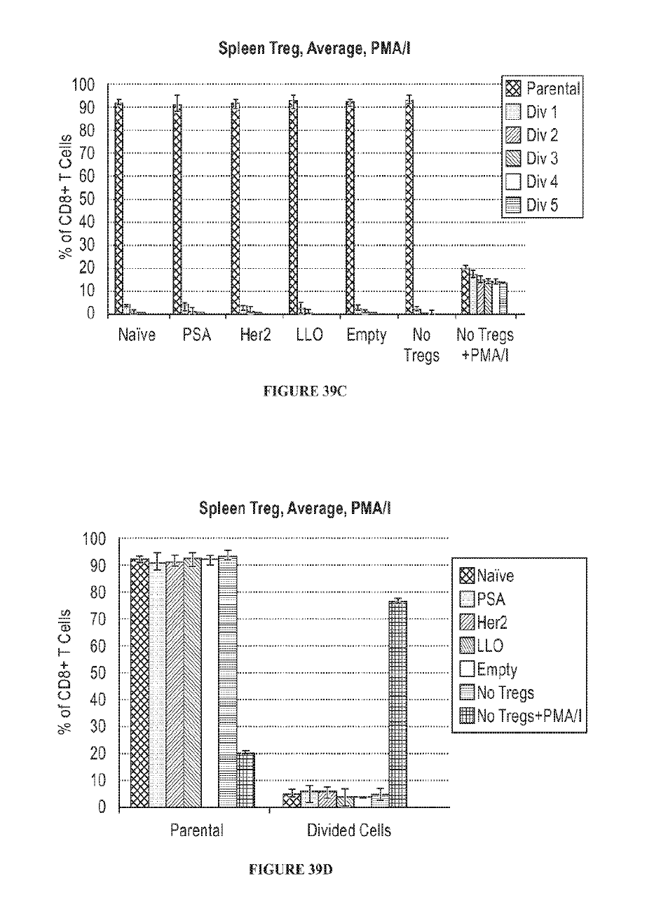

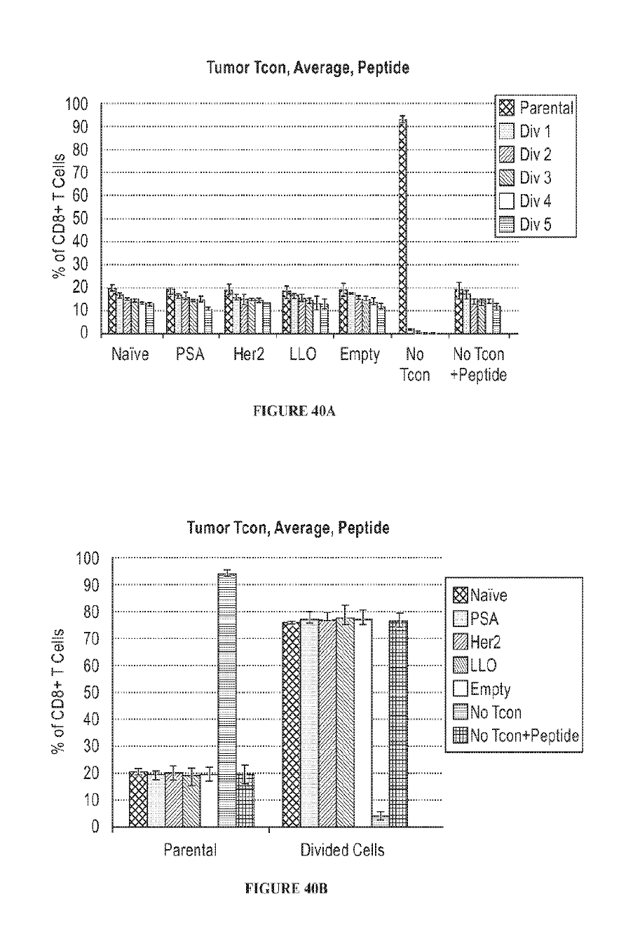

Tregs purified from the tumors of any of the Lm-treated groups have a slightly diminished ability to suppress the division of the responder T cells, regardless of whether the responder cells are antigen specific or non-specifically activated. Especially for the non-specifically activated responder T cells, it looks as though the vaccine with the empty plasmid shows the same results as all the vaccines that contain LLO on the plasmid. Averaging this experiment with the others shows that the differences are not significant (FIG. 38).

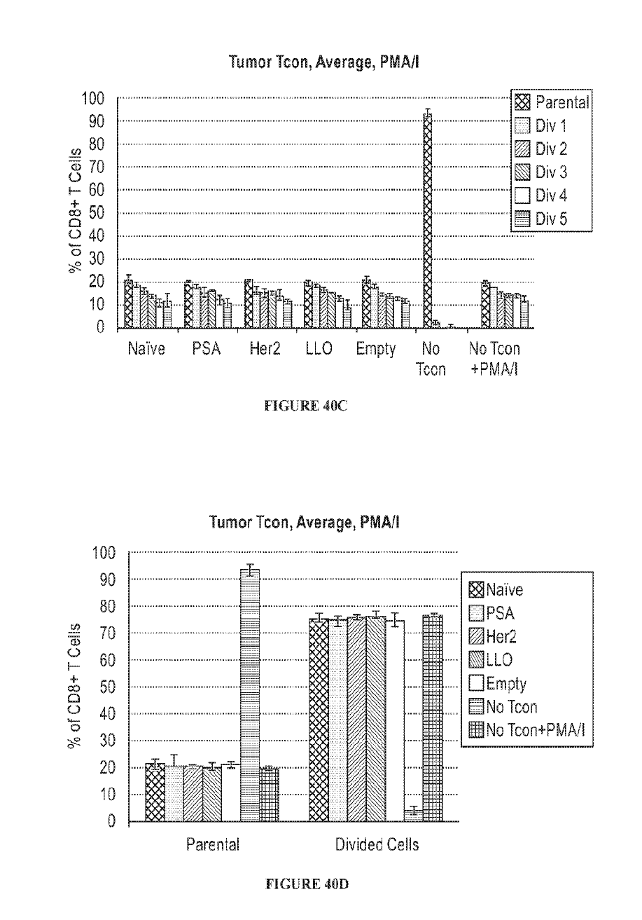

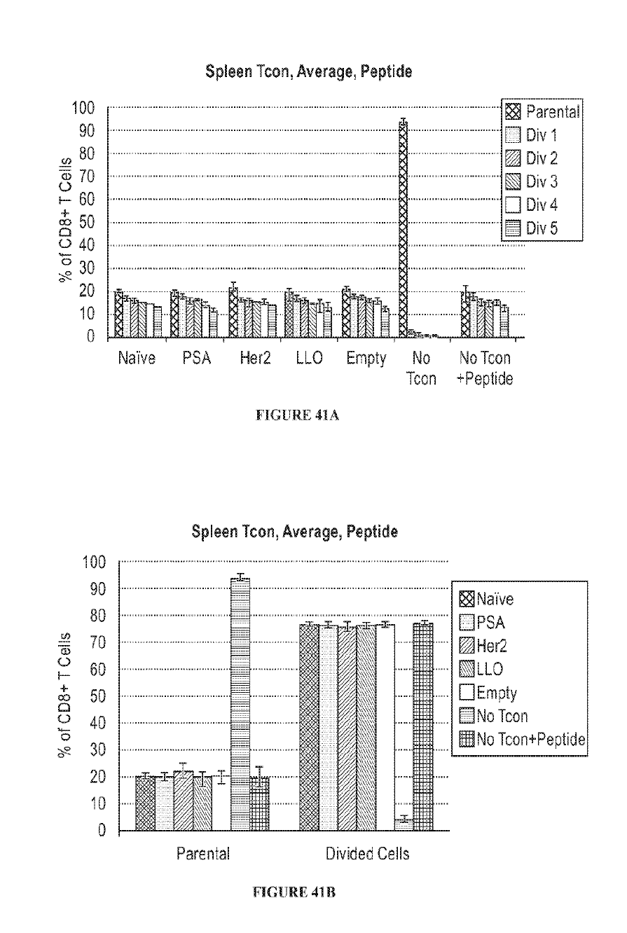

Tregs purified from the spleen are still capable of suppressing the division of both antigen specific and non-specifically activated responder T cells. There is no effect of Lm treatment on the suppressive ability of splenic Tregs (FIG. 39).

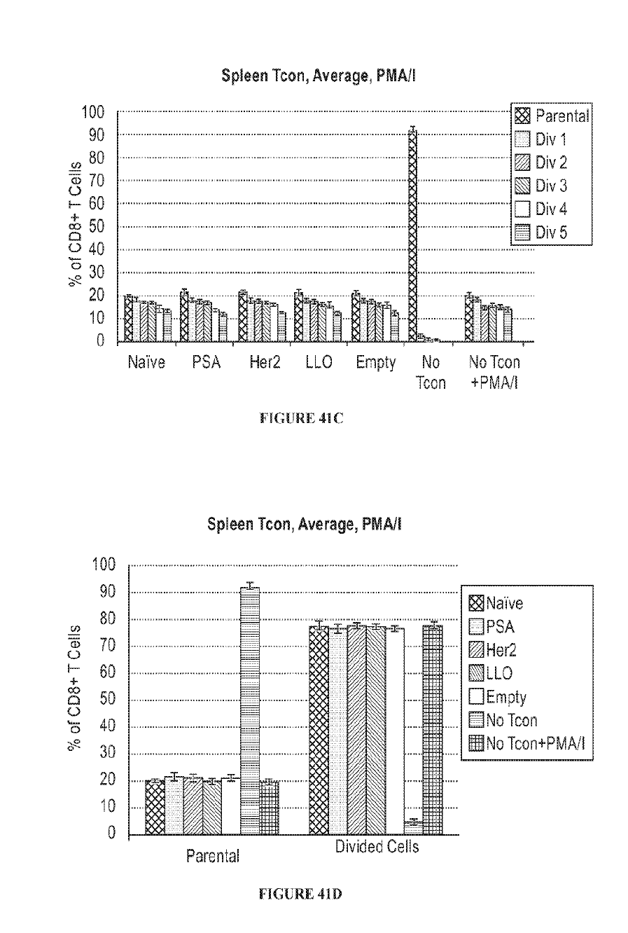

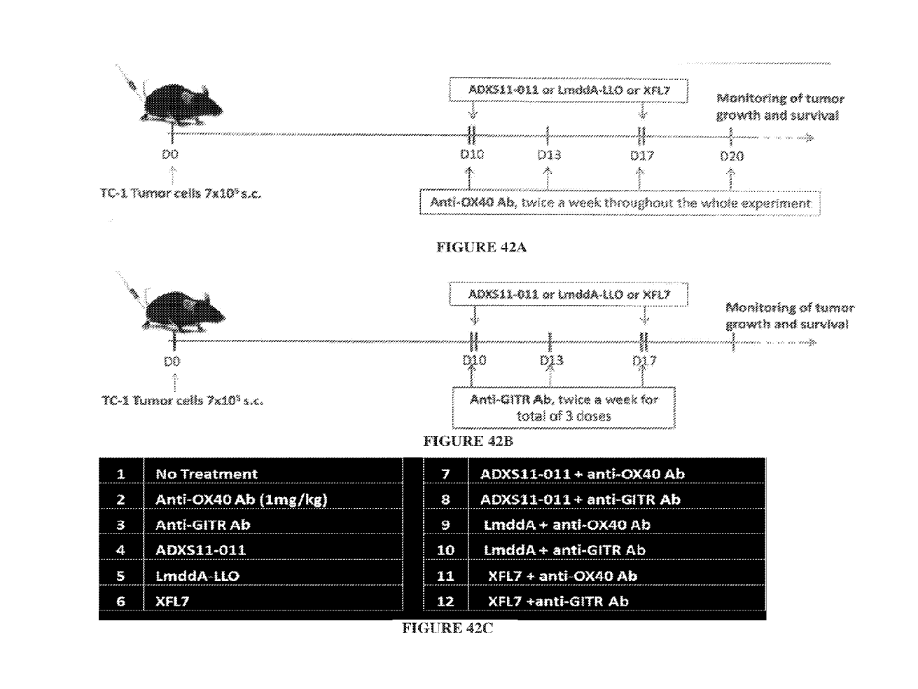

Tcon cells are not capable of suppressing the division of T cells regardless of whether the responder cells are antigens specific or non-specifically activated, which is consistent with the fact that these cells are non-suppressive. Lm has no effect on these cells and there was no difference if the cells were purified from the tumors or the spleen of mice (FIGS. 40-41).

### Example 20: Increased Survival in Mice Administered Combination Listeria-Based Vaccine with Anti-OX40 or Anti-GITR Abs

### Materials and Methods

### Animals, Cells Lines, Vaccine and Other Reagents

Six to eight weeks old female C57BL6 mice were purchased from Jackson Laboratories and kept under pathogen-free conditions. Mice were cared for under protocols approved by the GRU Animal Care and Use Committee according to NIH guidelines. TC-1 cells that were derived by co-transfection of human papillomavirus strain 16 (HPV16) early proteins 6 and 7 (E6 and E7) and activated ras oncogene to primary C57BL/6 mouse lung epithelial cells were obtained from ATCC (Manassas, Va.), and cells were grown in RPMI 1640 supplemented with 10% FBS, penicillin and streptomycin (100 U/ml each) and L-glutamine (2 mM) at 37° C. with 5% CO2. Listeria vaccine vectors with or without human papilloma virus-16 (HPV-16) E7 (Lm-LLO-E7, LmddA-LLO and XFL7) provided by Advaxis Inc. were generated as described above in Example 1, and as disclosed above in the Detailed Description.

Lm-LLO-E7, LmddA-LLO and XFL7 were injected intraperitonealy (i.p.) at 1×10⁸ CFU/mouse dose. The GITR and OX40 antibodies were obtained from Astra Zeneca/Medimmune and were injected as intravenously (i.v.) at a dose of 50 μg/mouse (for anti-OX40Ab) and 250 μg/mouse (for anti-GITR Ab), as shown in FIG. 42A and FIG. 42B.

### Tumor Implantation and Treatment

The therapeutic experiments aimed to analyze tumor growth and survival were performed as follows. Briefly, mice were subcutaneously (s.c.) implanted with 70,000 TC-1 tumor cells/mouse in the right flank on day 0. On day 10 (tumor size ˜4-5 mm in diameter), animals from appropriate groups (10 mice per group) were injected i.p. with Lm-LLO-E7, LmddA-LLO and XFL7 with or without anti-GITR Ab or anti-OX40 Ab or left non-treated (NT). (FIG. 42C) Mice receiving anti-OX40 Ab were treated with vaccine and anti-OX40 Ab twice a week throughout the length of the experiment (FIG. 42, day 10, day 13, day 17, day 20 etc). Mice receiving anti-GITR Ab were treated twice a week for total of 3 doses (FIG. 42B, day 10, day 13 and day 17). Another group of mice remained not treated.

### Results

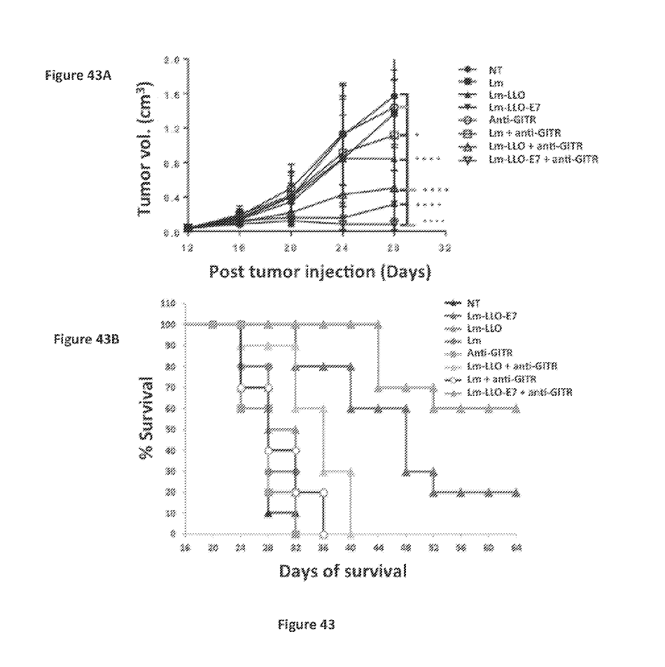

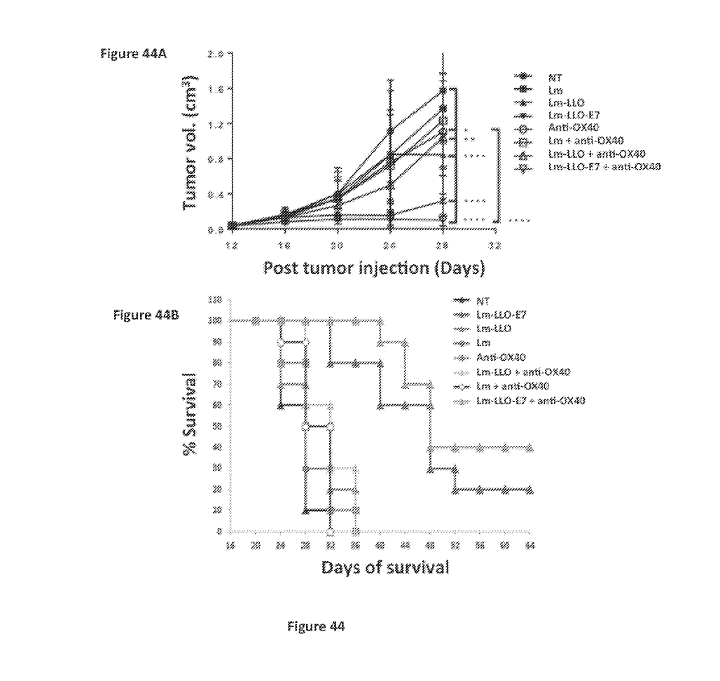

FIG. 43 A-B shows that while administration of Listeria-based vaccine ADXS11-001 alone, extended the survival of treated mice at least twice as long as non-treated or control treated mice, the combination of treatment with ADXS11-001 and administration of anti-GITR Abs increased not only the time of survival but the percent survival within the population. The percent increase was almost 40%. The combination of ADXS11-001 with anti-GITR Abs led to complete regression of established tumors in 60% of treated mice (FIG. 43A). Interestingly, anti-GITR antibodies also showed an increase in survival time in mice treated with LmddA-LLO/anti-GITR compared with mice receiving only LmddA-LLO (FIG. 43B).

FIG. 44 A-B shows that while administration of Listeria-based vaccine ADXS11-001 alone, extended the survival of treated mice at least twice as long as non-treated or control treated mice, the combination of treatment with ADXS11-001 and administration of anti-OX40 Abs increased not only the time of survival but the percent survival within the population (FIG. 44 B). The percent increase was almost 20%. Thus, the combination of ADXS11-001 with anti-OX40 Abs led to complete regression of established tumors in 40% of treated mice (FIG. 44A).

These results show that use of anti-OX40 and anti-GITR antibodies enhanced the therapeutic potency of Listeria-based vaccines.

### Example 21: Use of Agonistic Antibodies Against Co-Stimulatory Molecules GITR and OX40 Significantly Enhance the Anti-Tumor Efficacy of Listeria-Based Immunotherapy

Following the results presented in Example 20 showing that agonistic antibodies, anti-OX40 and anti-GITR, enhanced the therapeutic potency of Listeria-based vaccines, the immune response for this enhanced survival was analyzed.

### Materials and Methods

### Animals, Cells Lines, Vaccine and Other Reagents

Six to eight weeks old female C57BL6 mice were purchased from Jackson Laboratories and kept under pathogen-free conditions. Mice were cared for under protocols approved by the GRU Animal Care and Use Committee according to NIH guidelines. TC-1 cells that were derived by co-transfection of human papillomavirus strain 16 (HPV16) early proteins 6 and 7 (E6 and E7) and activated ras oncogene to primary C57BL/6 mouse lung epithelial cells were obtained from ATCC (Manassas, Va.), and cells were grown in RPMI 1640 supplemented with 10% FBS, penicillin and streptomycin (100 U/ml each) and L-glutamine (2 mM) at 37° C. with 5% CO2. Listeria vaccine vectors with or without human papilloma virus-16 (HPV-16) E7, Lm [XFL7], Lm-LLO [LmddA-LLO], Lm-LLO-E7 [ADXS11-001]) provided by Advaxis Inc. were generated as described above in Example 1, and as disclosed above in the Detailed Description. Listeria-based therapies are shown in Table 3 along with control.

TABLE 3

| Nomenclature | Description of Listeria strain |
|---|---|
| LM | XFL7 |
| Lm-LLO | LmddA-LLO |
| Lm-LLO-E7 | ADXS11-001 |

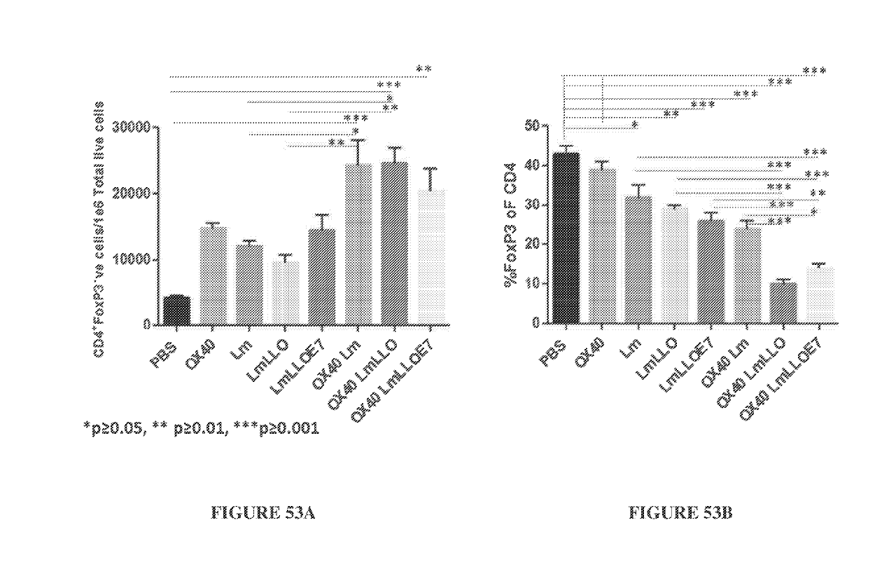

Lm, LM-LLO and Lm-LLO-E7, were injected intraperitonealy (i.p.) at 1×10⁸ CFU/mouse dose every 7 days starting at day D13 (FIG. 45 and FIG. 51A). The GITR and OX40 antibodies were obtained from Astra Zeneca/Medimmune and were injected as intravenously (i.v.), as shown in FIG. 45, FIG. 51.

### Tumor Implantation and Treatment

The therapeutic experiments analyzed tumor growth and immune response and were performed as follows. Briefly, mice were subcutaneously (s.c.) implanted with 70,000 TC-1 tumor cells/mouse in the right flank on day 0. On day 13 (tumor size ˜4-5 mm in diameter), animals from appropriate groups (5 mice per group) were injected i.p. with, LM, LM-LLO or Lm-LLO-E7 with or without anti-GITR Ab (FIG. 45; Table 4) or anti-OX40 Ab (FIG. 51; Table 5) or left non-treated (NT). Mice receiving anti-OX40 Ab were treated with vaccine and anti-OX40 Ab for a total of four doses of 1 mg/Kg mouse weight (mpk) at intervals of 3-4 days starting at day 13 (D13) (FIG. 51). Mice receiving anti-GITR Ab were treated with vaccine and anti-GITR Ab for a total of four doses of 5 mg/Kg mouse weight (mpk) at intervals of 3-4 days starting at day 13 (D13) (FIG. 45). Another group of mice remained not treated with the agonist antibody (PBS row, as described in FIG. 42C).

In a subset of mice, tumors are measured every 3-4 days using digital calipers, and tumor volume will be calculated using the formula V=(W2×L)/2, whereby V is volume, L is length (longer diameter) and W is width (shorter diameter). In these experiments mice will be sacrificed when mice become moribund, tumors ulcerate or tumor volume reaches 1.5 cm³.

All together there were 8 test groups. On day 26 (D26) all animals were terminated spleens and tumors harvested and screened for infiltrating total CD4, Tregs (CD4+FoxP3+), non Tregs (CD4+FOXP3⁻), CD8+, CD8+E7+, myeloid derived suppressor cells (MDSCs), CD8+/Treg, CD8+E7+/Treg, CD8+/MDSC, CD8+E7+/MDSC.

### Analysis of Antigen-Specific Cellular Immune Responses (ASIR), Tregs, MDSC in Periphery and Tumors

ELISPOT is used to detect IFNγ production in E7-restimulated (10 μg/ml) splenocytes cultures from treated and control mice, as suggested by the manufacturer (BD Biosciences, San Jose, Calif.). A CTL Immunospot Analyzer (Cellular Technology Ltd., Shaker Heights, Ohio) will be used to analyze spots. The number of spots from irrelevant peptide (hgp 10025-33-KVPRNQDWL-Celtek Bioscience, Nashville, Tenn.) re-stimulated splenocytes will be subtracted from E7-restimulated cultures. In addition, ASIR within the tumor is demonstrated in FIGS. 48B and 54B as the number of antigen-specific tumor-infiltrating CD8+ T cells (CD8+E7+ cells).

Tumor samples were processed using GentleMACS Dissociator and the solid tumor homogenization protocol, as suggested by the manufacturer (Miltenyi Biotec, Auburn, Calif.). The number of tumor-infiltrated CD8+, CD4+Foxp3+(Treg) and CD11b+Gr-1+(MDSC) cells were analyzed within the CD45+ hematopoietic cell population using flow cytometry assay. The level of Treg cells and MDSC were evaluated in spleens of tumor-bearing treated and control mice using the same flow cytometry assay.

### Statistical Analysis

All statistical parameters were calculated using GraphPad Prism Software (San Diego, Calif.). Statistical significance between groups were determined by one-way ANOVA with Tukey's multiple comparison post-test (P<0.05 was considered statistically significant).

### Results

Combination with GITR Agonist Antibodies

The total number of infiltrating CD4+ T cells was enhanced following combination therapy. FIG. 46A shows that administration of Lm-LLO-E7 in combination with GITR agonist antibody significantly enhanced tumor infiltrating total CD4+ T cells even compared to single therapy alone. Importantly, administration of LM-LLO-E7 in combination with GITR agonist antibody had no significant effect on total number of Treg cells (CD4+Foxp3+) (FIG. 46B).

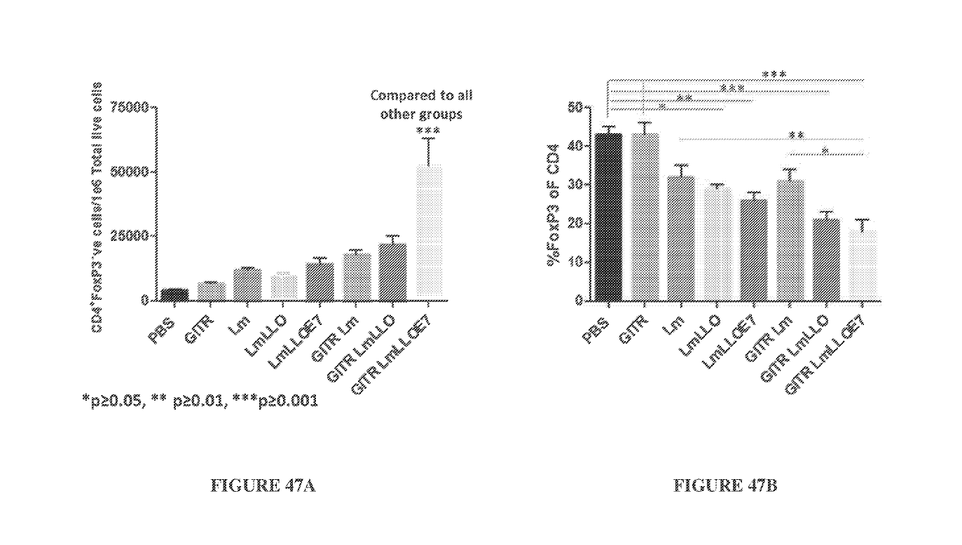

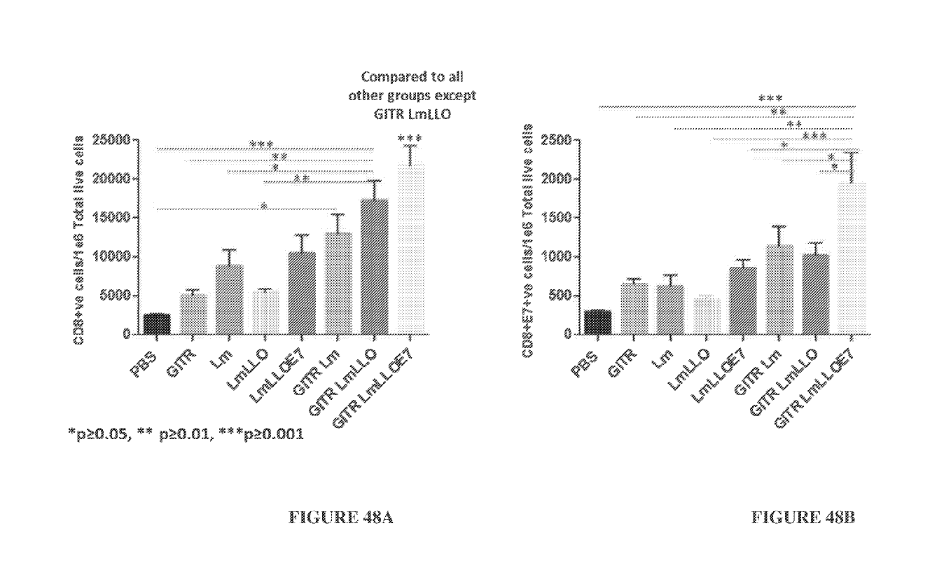

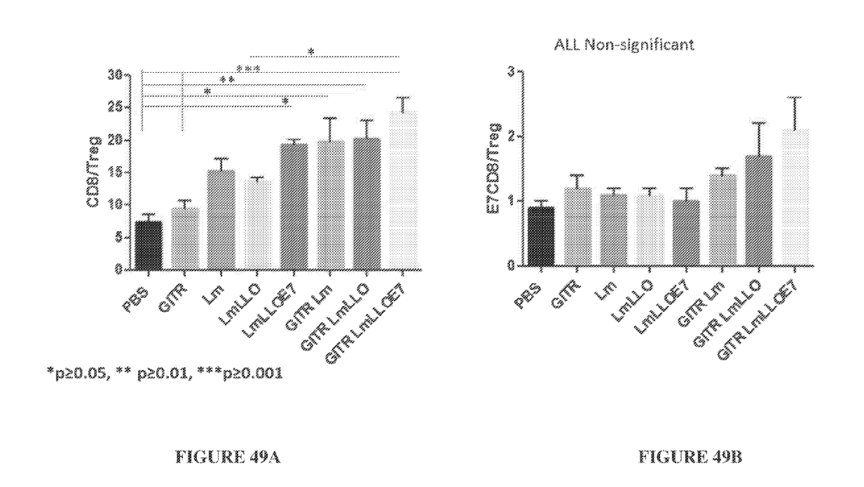

The total number of non-Treg CD4+ T cells was enhanced following combination therapy. FIG. 47A shows that administration of Lm-LLO-E7 in combination with GITR agonist antibody significantly enhanced the total number of non-Treg (CD4+Foxp3⁻) CD4+ T cells. Interestingly, administration of Listeria based vaccine by itself significantly reduced the overall percent of Foxp3 cells in total CD4 and in combination with FUR agonist antibody, the reduction is even significantly higher compared to PBS or agonist group alone (FIG. 47B)

Combination therapy also resulted in enhanced tumor infiltration of total CD8+ T cells. Administration of LM-LLO and LM-LLO-E7 in combination with anti GITR agonist antibodies (Ab) was observed to significantly enhance tumor infiltrating CD8+ T cells. (FIG. 48A) Interestingly, LM-LLO-E7 was observed to significantly enhance tumor infiltrating antigen specific CD8⁺E7⁺ T cells with anti-GITR Ab. (FIG. 48B)

Combination therapy enhanced CD8/Treg ratio in tumors. The CD8/Treg ratio in tumors was found to be significantly enhanced in combination GITR Ab group compared to PBS or antibody group alone. (FIG. 49A) The E7-CD8/Treg ratio was observed to non-significantly increase in the LM-LLO-E7 and anti-GITR combination group. (FIG. 49B)

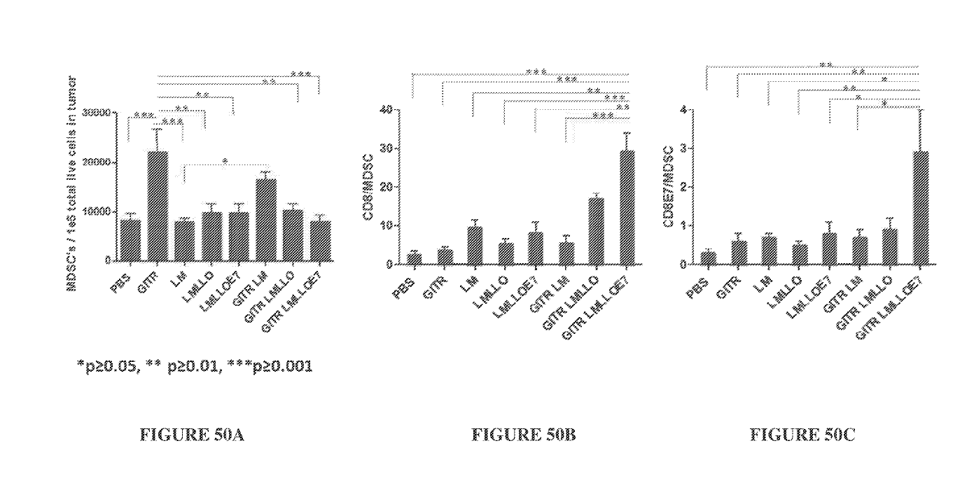

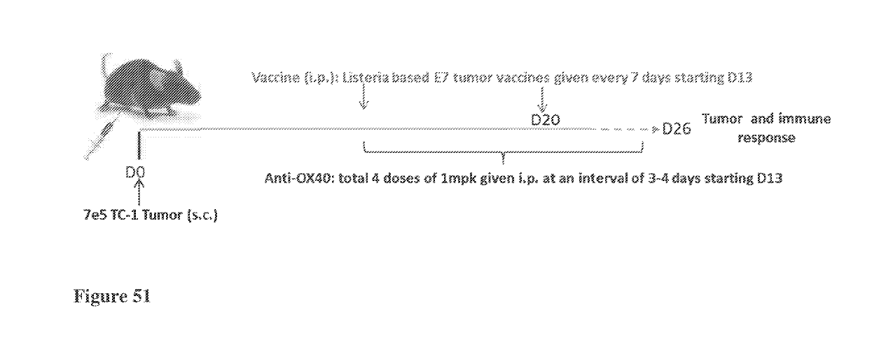

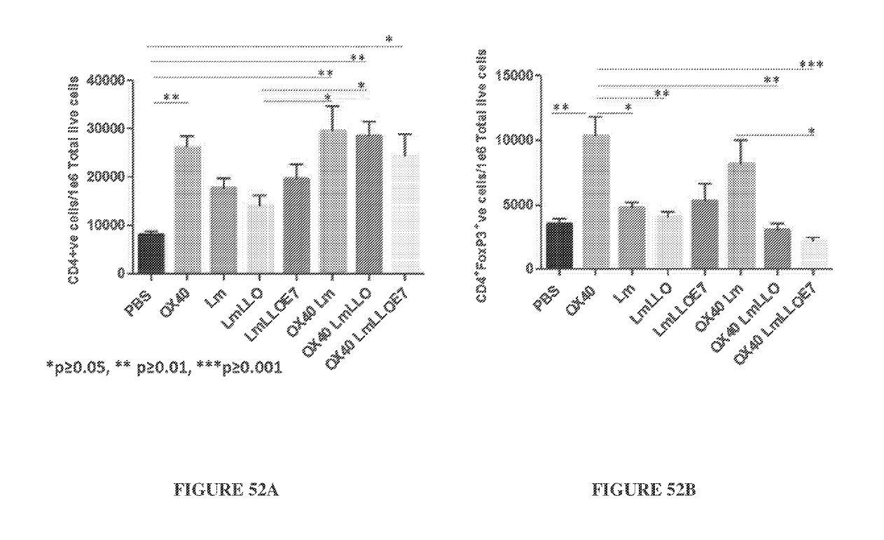

Induction of MDSCs by agonist GITR antibody. Agonist Ab against GITR was observed to induce MDSCs significantly compared to PBS group. (FIG. 50A) In addition, the CD8/MDSC ratio was significantly increased with anti-GITR Ab in combination with LM-LLO-E7. (FIG. 50B) Interestingly E7⁺CD8⁺/MDSC ratio was significantly increased with anti-GITR Ab in combination with LM-LLO-E7. (FIG. 50C)

Combination with OX40 Agonist Antibodies

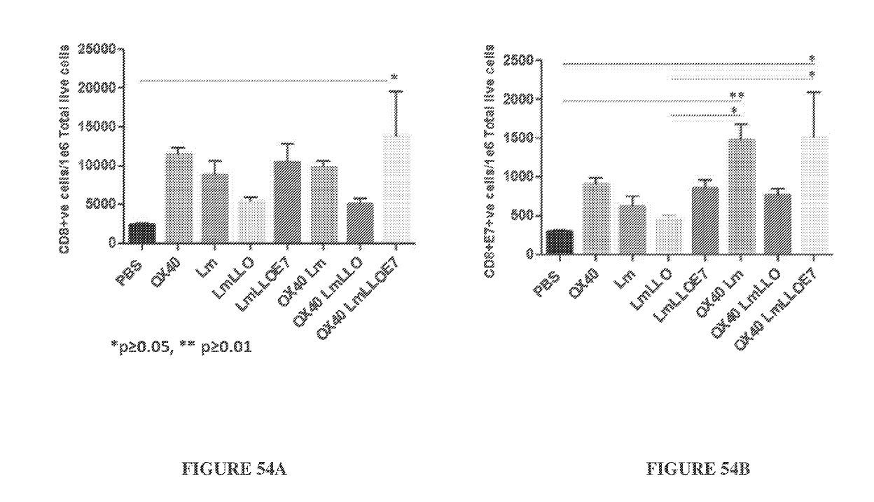

Interestingly, OX40 agonist Ab by itself significantly enhanced total CD4⁺ T cells compared to PBS group. Listeria based LM-LLO-E7 in combination with OX40 agonist Ab, significantly enhanced tumor infiltrating total CD4⁺T cells only compared to PBS group but not to single therapy alone. (FIG. 52A) OX40 Ab induced Treg cells (CD4⁺Foxp3⁺) were significantly reduced in combination with Listeria based LM-LLO and LM-LLO-E7. (FIG. 52B)

The number of tumor-infiltrating total non Treg (CD4+FoxP3⁻) and the percent Treg of the total CD4+ T cells was analyzed. Listeria based E7 vaccine in combination with OX40 agonist Ab significantly enhanced the total number of non Treg (CD4⁺Foxp3⁻) T cells. (FIG. 53A) Listeria based vaccine by itself significantly reduces the overall % of Foxp3 cells in total CD4 and in combination with OX40 agonist Ab the reduction was even significantly higher compared to all groups. (FIG. 53B)

The number of total CD8+ T cells as well as antigen specific CD8+E7+ cells was increased following combination therapy. Combination of LM-LLO-E7 with anti-OX40 Ab lead to a significant increase in the total number of CD8⁺ T cells compared to PBS group. (FIG. 54A) In addition, LM-LLO-E7 was observed to significantly enhance tumor infiltrating antigen specific CD8⁺E7⁺ T cells when combined with administration of anti-OX40 agonist Ab. (FIG. 54B)

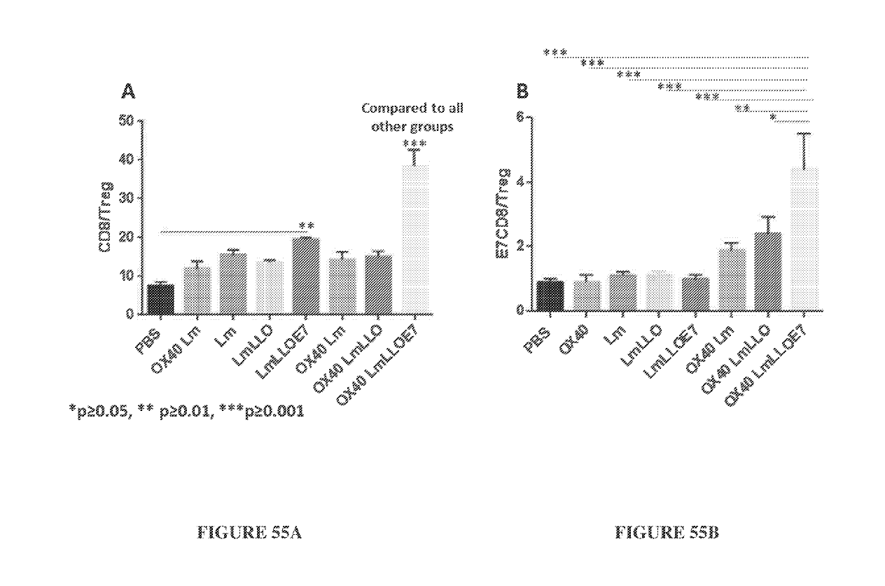

The ratios of CD8/Treg and E7CD8/Treg were enhanced following combination therapy. The CD8/Treg ratio in tumor was found to be significantly enhanced in anti-OX40 agonist Ab and LM-LLO-E7 combination group compared to all groups. (FIG. 55A) The E7CD8/Treg ratio in tumor was found to be significantly enhanced in anti-OX40 agonist Ab and LM-LLO-E7 combination group compared to all groups. (FIG. 55B)

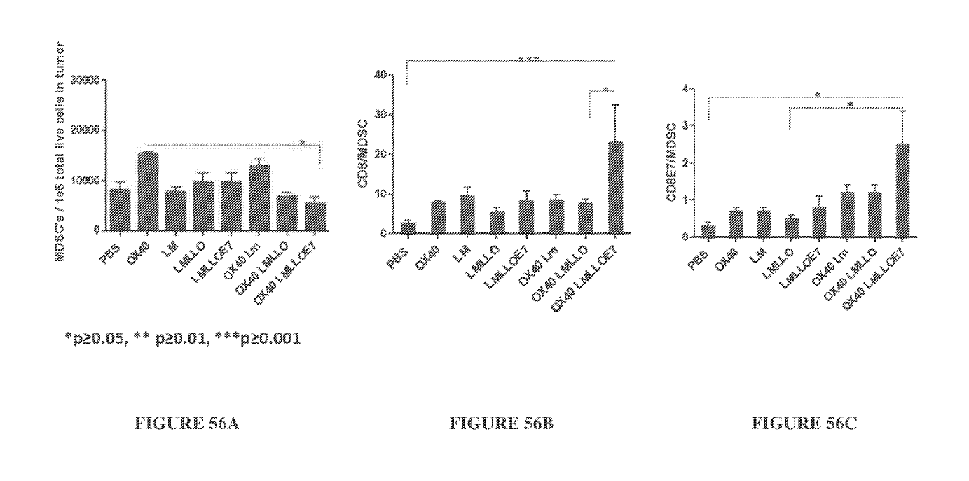

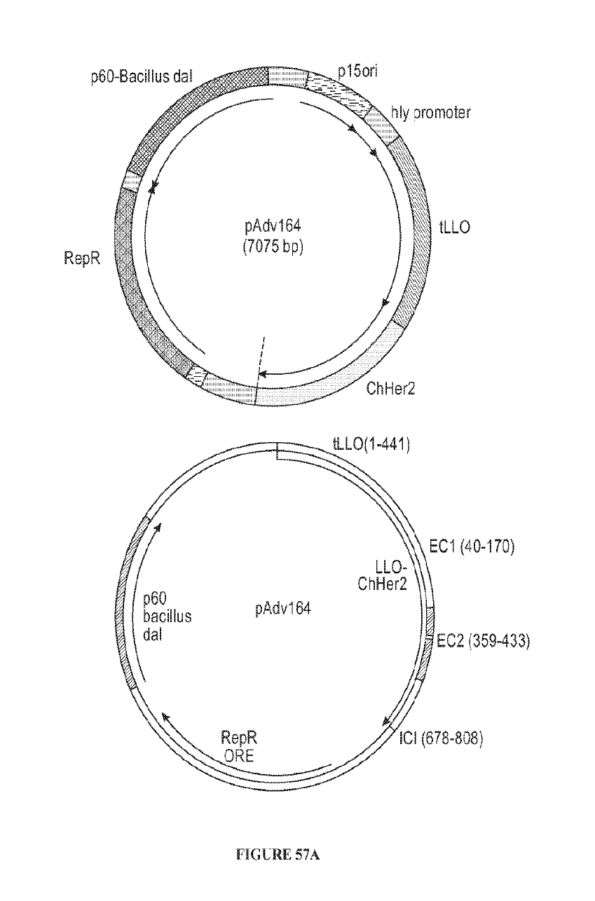

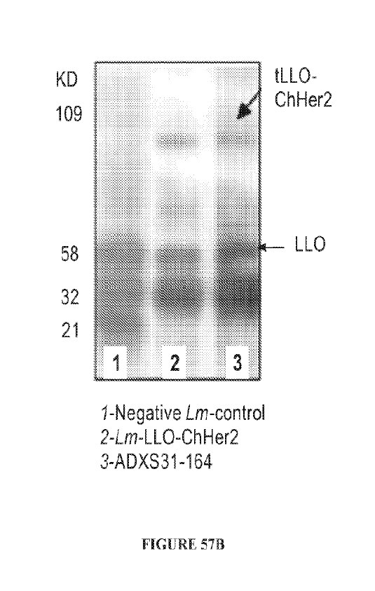

Induction of MDSCs following combination therapy. Agonist OX40 Ab was observed to non-significantly increase MDSC and combination with LM-LLO-E7 significantly decreased this immunosuppressive MDSCs. (FIG. 56A) the CD8/MDSC ratio was significantly increased with anti-OX40 Ab in combination with LM-LLO-E7. (FIG. 56B) And, the E7⁺CD8⁺/MDSC ratio was significantly increased with anti-OX40 Ab in combination with LM-LLO-E7. (FIG. 56C)

### Conclusion

The results presented herein show the anti-tumor and immune response of inhibiting the co-stimulating GITR and OX40 pathways in combination with Listeria based E7 vaccines. Co-stimulation of GITR or OX40 pathway's in presence of LM-LLO-E7 tumor vaccine exhibited enhanced anti-tumor activity and enhanced survival (Example 20). Though the combination of anti-GITR or anti-OX40 with peptide based E7 vaccine significantly increased total as well as antigen specific CD8, they had no effect on Treg or MDSC populations. Rather therapy with these agonist antibodies was found to increase immune suppressive cells in tumor by themselves or in combination with peptide based E7 vaccine in the TC1 tumor model.

Listeria based vaccine are known to decrease immune suppressive cells including Tregs and MDSC's. It was observed here that co-stimulation of GITR or OX40 pathway in presence of Listeria based vaccines increased the ratio of CD8 T cell to MDSC population and augment CD8 and antigen specific CD8, thus overall enhancing the effector cell/immunosuppressive cell ratio correlating with improved anti-tumor activity and survival.

### Materials and Methods (Examples 22-27)

Oligonucleotides were synthesized by Invitrogen (Carlsbad, Calif.) and DNA sequencing was done by Genewiz Inc, South Plainfield, N.J. Flow cytometry reagents were purchased from Becton Dickinson Biosciences (BD, San Diego, Calif.). Cell culture media, supplements and all other reagents, unless indicated, were from Sigma (St. Louise, Mo.). Her2/neu HLA-A2 peptides were synthesized by EZbiolabs (Westfield, Ind.). Complete RPMI 1640 (C-RPMI) medium contained 2 mM glutamine, 0.1 mM non-essential amino acids, and 1 mM sodium pyruvate, 10% fetal bovine serum, penicillin/streptomycin, Hepes (25 mM). The polyclonal anti-LLO antibody was described previously and anti-Her2/neu antibody was purchased from Sigma.

### Mice and Cell Lines

All animal experiments were performed according to approved protocols by IACUC at the University of Pennsylvania or Rutgers University. FVB/N mice were purchased from Jackson laboratories (Bar Harbor, Me.). The FVB/N Her2/neu transgenic mice, which overexpress the rat Her2/neu onco-protein were housed and bred at the animal core facility at the University of Pennsylvania. The NT-2 tumor cell line expresses high levels of rat Her2/neu protein, was derived from a spontaneous mammary tumor in these mice and grown as described previously. DHFR-G8 (3T3/neu) cells were obtained from ATCC and were grown according to the ATCC recommendations. The EMT6-Luc cell line was a generous gift from Dr. John Ohlfest (University of Minnesota, Minn.) and was grown in complete C-RPMI medium. Bioluminescent work was conducted under guidance by the Small Animal Imaging Facility (SAIF) at the University of Pennsylvania (Philadelphia, Pa.).

### Listeria Constructs and Antigen Expression

Her2/neu-pGEM7Z was kindly provided by Dr. Mark Greene at the University of Pennsylvania and contained the full-length human Her2/neu (hHer2) gene cloned into the pGEM7Z plasmid (Promega, Madison Wis.). This plasmid was used as a template to amplify three segments of hHer-2/neu, namely, EC1, EC2, and IC1, by PCR using pfx DNA polymerase (Invitrogen) and the oligos indicated in Table 6.

TABLE 6
Primers for cloning of Human her-2-Chimera

| | | Base | Amino acid |
|---|---|---|---|
| | | pair | region or |
| | DNA sequence | region | junctions |
| Her-2- | TGATCTCGAGACCCA | 120-510 | 40-170 |
| Chimera | CCTGGACATGCTC | | |
| (F) | (SEQ ID NO: 75) | | |
| HerEC1- | CTACCAGGACACGAT | 510/1077 | 170/359 |
| EC2F | TTTGTGGAAGAATAT | | |
| (Junction) | CCAGGAGTTTGCTGG | | |
| | CTGC | | |
| | (SEQ ID NO: 76) | | |
| HerEC1- | GCAGCCAGCAAACTC | | |
| EC2R | CTGGATATTCTTCCA | | |
| (Junction) | CAAAATCGTGTCCTG | | |
| | GTAG | | |
| | (SEQ ID NO: 77) | | |
| HerEC2- | CTGCCACCAGCTGTG | 1554/2034 | 518/679 |
| ICIF | CGCCCGAGGGCAGCA | | |
| (Junction) | GAAGATCCGGAAGTA | | |
| | CACGA | | |
| | (SEQ ID NO: 78) | | |
| HerEC2- | TCGTGTACTTCCGGA | | |
| ICIR | TCTTCTGCTGCCCTC | | |
| (Junction) | GGGCGCACAGCTGGT | | |
| | GGCAG | | |
| | (SEQ ID NO: 79) | | |
| Her-2- | GTGGCCCGGGTCTAG | 2034-2424 | 679-808 |
| Chimera | ATTAGTCTAAGAGGC | | |
| (R) | AGCCATAGG | | |
| | (SEQ ID NO: 80) | | |

The Her-2/neu chimera construct was generated by direct fusion by the SOEing PCR method and each separate hHer-2/neu segment as templates. Primers are shown in Table 7.

TABLE 7

| | | Base | Amino |
|---|---|---|---|
| | | pair | acid |
| | DNA sequence | region | region |
| Her-2- | CCGCCTCGAGGCCGC | 58-979 | 20-326 |
| EC1(F) | GAGCACCCAAGTG | | |
| | (SEQ ID NO: 81) | | |
| Her-2- | CGCGACTAGTTTAAT | | |
| EC1(R) | CCTCTGCTGTCACCT | | |
| | C | | |
| | (SEQ ID NO: 82) | | |
| Her-2- | CCGCCTCGAGTACCT | 907-1504 | 303-501 |
| EC2(F) | TTCTACGGACGTG | | |
| | (SEQ ID NO: 83) | | |
| Her-2- | CGCGACTAGTTTACT | | |
| EC2(R) | CTGGCCGGTTGGCAG | | |
| | (SEQ ID NO: 84) | | |
| Her-2- | CCGCCTCGAGCAGCA | 2034-3243 | 679-1081 |
| Her-2- | GAAGATCCGGAAGTA | | |
| IC1(F) | C | | |
| | (SEQ ID NO: 85) | | |
| Her-2- | CGCGACTAGTTTAAG | | |
| IC1(R) | CCCCTTCGGAGGGTG | | |
| | (SEQ ID NO: 86) | | |

Sequence of Primers for Amplification of Different Segments Human Her2 Regions

ChHer2 gene was excised from pAdv138 using XhoI and SpeI restriction enzymes, and cloned in frame with a truncated, non-hemolytic fragment of LLO in the Lmdd shuttle vector, pAdv134. The sequences of the insert, LLO and hly promoter were confirmed by DNA sequencing analysis. This plasmid was electroporated into electro-competent actA, dal, dat mutant Listeria monocytogenes strain, LmddA and positive clones were selected on Brain Heart infusion (BHI) agar plates containing streptomycin (254 μg/ml). In some experiments similar Listeria strains expressing hHer2/neu (Lm-hHer2) fragments were used for comparative purposes. In all studies, an irrelevant Listeria construct (Lm-control) was included to account for the antigen independent effects of Listeria on the immune system. Lm-controls were based on the same Listeria platform as ADXS31-164 (LmddA-ChHer2), but expressed a different antigen such as HPV16-E7 or NY-ESO-1. Expression and secretion of fusion proteins from Listeria were tested. Each construct was passaged twice in vivo.

### Cytotoxicity Assay

Groups of 3-5 FVB/N mice were immunized three times with one week intervals with 1×10⁸ colony forming units (CFU) of Lm-LLO-ChHer2, ADXS31-164, Lm-hHer2 ICI or Lm-control (expressing an irrelevant antigen) or were left naïve. NT-2 cells were grown in vitro, detached by trypsin and treated with mitomycin C (250 μg/ml in serum free C-RPMI medium) at 37° C. for 45 minutes. After 5 washes, they were co-incubated with splenocytes harvested from immunized or naïve animals at a ratio of 1:5 (Stimulator: Responder) for 5 days at 37° C. and 5% CO₂. A standard cytotoxicity assay was performed using europium labeled 3T3/neu (DHFR-G8) cells as targets according to the method previously described. Released europium from killed target cells was measured after 4 hour incubation using a spectrophotometer (Perkin Elmer, Victor²) at 590 nm. Percent specific lysis was defined as (lysis in experimental group-spontaneous lysis)/(Maximum lysis-spontaneous lysis).

Interferon-γ Secretion by Splenocytes from Immunized Mice

Groups of 3-5 FVB/N or HLA-A2 transgenic mice were immunized three times with one week intervals with 1×10⁸ CFU of ADXS31-164, a negative Listeria control (expressing an irrelevant antigen) or were left naïve. Splenocytes from FVB/N mice were isolated one week after the last immunization and co-cultured in 24 well plates at 5×10⁶ cells/well in the presence of mitomycin C treated NT-2 cells in C-RPMI medium. Splenocytes from the HLA-A2 transgenic mice were incubated in the presence of 1 μM of HLA-A2 specific peptides or 1 μg/ml of a recombinant His-tagged ChHer2 protein, produced in E. coli and purified by a nickel based affinity chromatography system. Samples from supernatants were obtained 24 or 72 hours later and tested for the presence of interferon-γ (IFN-γ) using mouse IFN-γ Enzyme-linked immunosorbent assay (ELISA) kit according to manufacturer's recommendations.

### Tumor Studies in Her2 Transgenic Animals

Six weeks old FVB/N rat Her2/neu transgenic mice (9-14/group) were immunized 6 times with 5×10⁸ CFU of Lm-LLO-ChHer2, ADXS31-164 or Lm-control. They were observed twice a week for the emergence of spontaneous mammary tumors, which were measured using an electronic caliper, for up to 52 weeks. Escaped tumors were excised when they reached a size 1 cm² in average diameter and preserved in RNAlater at −20° C. In order to determine the effect of mutations in the Her2/neu protein on the escape of these tumors, genomic DNA was extracted using a genomic DNA isolation kit, and sequenced.

### Effect of ADXS31-164 on Regulatory T Cells in Spleens and Tumors

Mice were implanted subcutaneously (s.c.) with 1×10⁶ NT-2 cells. On days 7, 14 and 21, they were immunized with 1×10⁸ CFUs of ADXS31-164, LmddA-control or left naïve. Tumors and spleens were extracted on day 28 and tested for the presence of CD3⁺/CD4⁺/FoxP3⁺ Tregs by FACS analysis. Briefly, splenocytes were isolated by homogenizing the spleens between two glass slides in C-RPMI medium. Tumors were minced using a sterile razor blade and digested with a buffer containing DNase (12 U/ml), and collagenase (2 mg/ml) in PBS. After 60 min incubation at RT with agitation, cells were separated by vigorous pipetting. Red blood cells were lysed by RBC lysis buffer followed by several washes with complete RPMI-1640 medium containing 10% FBS. After filtration through a nylon mesh, tumor cells and splenocytes were resuspended in FACS buffer (2% FBS/PBS) and stained with anti-CD3-PerCP-Cy5.5, CD4-FITC, CD25-APC antibodies followed by permeabilization and staining with anti-Foxp3-PE. Flow cytometry analysis was performed using 4-color FACS calibur (BD) and data were analyzed using cell quest software (BD).

### Statistical Analysis

The log-rank Chi-Squared test was used for survival data and student's t-test for the CTL and ELISA assays, which were done in triplicates. A p-value of less than 0.05 (marked as *) was considered statistically significant in these analyzes. All statistical analysis was done with either Prism software, V.4.0a (2006) or SPSS software, V.15.0 (2006). For all FVB/N rat Her2/neu transgenic studies we used 8-14 mice per group, for all wild-type FVB/N studies we used at least 8 mice per group unless otherwise stated. All studies were repeated at least once except for the long term tumor study in Her2/neu transgenic mouse model.

### Example 21: Generation of L. Monocytogenes Strains that Secrete LLO Fragments Fused to Her-2 Fragments: Construction of ADXS31-164

Construction of the chimeric Her2/neu gene (ChHer2) was as follows. Briefly, ChHer2 gene was generated by direct fusion of two extracellular (aa 40-170 and aa 359-433) and one intracellular fragment (aa 678-808) of the Her2/neu protein by SOEing PCR method. The chimeric protein harbors most of the known human MHC class I epitopes of the protein. ChHer2 gene was excised from the plasmid, pAdv138 (which was used to construct Lm-LLO-ChHer2) and cloned into LmddA shuttle plasmid, resulting in the plasmid pAdv164 (FIG. 57A). There are two major differences between these two plasmid backbones. 1) Whereas pAdv138 uses the chloramphenicol resistance marker (cat) for in vitro selection of recombinant bacteria, pAdv164 harbors the D-alanine racemase gene (dal) from bacillus subtilis, which uses a metabolic complementation pathway for in vitro selection and in vivo plasmid retention in LmddA strain which lacks the dal-dat genes. This vaccine platform was designed and developed to address FDA concerns about the antibiotic resistance of the engineered Listeria vaccine strains. 2) Unlike pAdv138, pAdv164 does not harbor a copy of the prfA gene in the plasmid (see sequence below and FIG. 57A), as this is not necessary for in vivo complementation of the Lmdd strain. The LmddA vaccine strain also lacks the actA gene (responsible for the intracellular movement and cell-to-cell spread of Listeria) so the recombinant vaccine strains derived from this backbone are 100 times less virulent than those derived from the Lmdd, its parent strain. LmddA-based vaccines are also cleared much faster (in less than 48 hours) than the Lmdd-based vaccines from the spleens of the immunized mice. The expression and secretion of the fusion protein tLLO-ChHer2 from this strain was comparable to that of the Lm-LLO-ChHer2 in TCA precipitated cell culture supernatants after 8 hours of in vitro growth (FIG. 57B) as a band of ˜104 KD was detected by an anti-LLO antibody using Western Blot analysis. The Listeria backbone strain expressing only tLLO was used as negative control.

pAdv164 Sequence (7075 Base Pairs) (See FIGS. 57A and 57B):

(SED ID NO: 87)
cggagtgtatactggcttactatgttggcactgatgagggtgtcagtga
agtgcttcatgtggcaggagaaaaaaggctgcaccggtgcgtcagcaga
atatgtgatacaggatatattccgcttcctcgctcactgactcgctacg
ctcggtcgttcgactgcggcgagcggaaatggcttacgaacggggcgga
gatttcctggaagatgccaggaagatacttaacagggaagtgagagggc
cgcggcaaagccgtttttccataggctccgcccccctgacaagcatcac
gaaatctgacgctcaaatcagtggtggcgaaacccgacaggactataaa
gataccaggcgtttccccctggcggctccctcgtgcgctctcctgttcc
tgcctttcggtttaccggtgtcattccgctgttatggccgcgtttgtct
cattccacgcctgacactcagttccgggtaggcagttcgctccaagctg
gactgtatgcacgaaccccccgttcagtccgaccgctgcgccttatccg
gtaactatcgtcttgagtccaacccggaaagacatgcaaaagcaccact
ggcagcagccactggtaattgatttagaggagttagtcttgaagtcatg
cgccggttaaggctaaactgaaaggacaagttttggtgactgcgctcct
ccaagccagttacctcggttcaaagagttggtagctcagagaaccttcg
aaaaaccgccctgcaaggcggttttttcgttttcagagcaagagattac
gcgcagaccaaaacgatctcaagaagatcatcttattaatcagataaaa
tatttctagccctcctttgattagtatattcctatcttaaagttacttt
tatgtggaggcattaacatttgttaatgacgtcaaaaggatagcaagac
tagaataaagctataaagcaagcatataatattgcgtttcatctttaga
agcgaatttcgccaatattataattatcaaaagagaggggtggcaaacg
gtatttggcattattaggttaaaaaatgtagaaggagagtgaaacccat
gaaaaaaataatgctagtttttattacacttatattagttagtctacca
attgcgcaacaaactgaagcaaaggatgcatctgcattcaataaagaaa
attcaatttcatccatggcaccaccagcatctccgcctgcaagtcctaa
gacgccaatcgaaaagaaacacgcggatgaaatcgataagtatatacaa
ggattggattacaataaaaacaatgtattagtataccacggagatgcag
tgacaaatgtgccgccaagaaaaggttacaaagatggaaatgaatatat
tgttgtggagaaaaagaagaaatccatcaatcaaaataatgcagacatt
caagttgtgaatgcaatttcgagcctaacctatccaggtgctctcgtaa
aagcgaattcggaattagtagaaaatcaaccagatgttctccctgtaaa
acgtgattcattaacactcagcattgatttgccaggtatgactaatcaa
gacaataaaatagttgtaaaaaatgccactaaatcaaacgttaacaacg
cagtaaatacattagtggaaagatggaatgaaaaatatgctcaagctta
tccaaatgtaagtgcaaaaattgattatgatgacgaaatggcttacagt
gaatcacaattaattgcgaaatttggtacagcatttaaagctgtaaata
atagcttgaatgtaaacttcggcgcaatcagtgaagggaaaatgcaaga
agaagtcattagttttaaacaaatttactataacgtgaatgttaatgaa
cctacaagaccttccagatattcggcaaagctgttactaaagagcagtt
gcaagcgcttggagtgaatgcagaaaatcctcctgcatatatctcaagt
gtggcgtatggccgtcaagtttatttgaaattatcaactaattcccata
gtactaaagtaaaagctgcttttgatgctgccgtaagcggaaaatctgt
ctcaggtgatgtagaactaacaaatatcatcaaaaattcttccttcaaa
gccgtaatttacggaggttccgcaaaagatgaagttcaaatcatcgacg
gcaacctcggagacttacgcgatattttgaaaaaaggcgctacttttaa
tcgagaaacaccaggagttcccattgcttatacaacaaacttcctaaaa
gacaatgaattagctgttattaaaaacaactcagaatatattgaaacaa
cttcaaaagcttatacagatggaaaaattaacatcgatcactctggagg
atacgttgctcaattcaacatttcttgggatgaagtaaattatgatctc
gagacccacctggacatgctccgccacctctaccagggctgccaggtgg
tgcagggaaacctggaactcacctacctgcccaccaatgccagcctgtc
cttcctgcaggatatccaggaggtgcagggctacgtgctcatcgctcac
aaccaagtgaggcaggtcccactgcagaggctgcggattgtgcgaggca
cccagctattgaggacaactatgccctggccgtgctagacaatggagac
ccgctgaacaataccacccctgtcacaggggcctccccaggaggcctgc
gggagctgcagcttcgaagcctcacagagatcttgaaaggaggggtctt
gatccagcggaacccccagctctgctaccaggacacgattttgtggaag
aatatccaggagtttgctggctgcaagaagatctttgggagcctggcat
ttctgccggagagctttgatggggacccagcctccaacactgccccgct
ccagccagagcagctccaagtgtttgagactctggaagagatcacaggt
tacctatacatctcagcatggccggacagcctgcctgacctcagcgtct
tccagaacctgcaagtaatccggggacgaattctgcacaatggcgccta
ctcgctgaccctgcaagggctgggcatcagctggctggggctgcgctca
ctgagggaactgggcagtggactggccctcatccaccataacacccacc
tctgcttcgtgcacacggtgccctgggaccagctctttcggaacccgca
ccaagctctgctccacactgccaaccggccagaggacgagtgtgtgggc
gagggcctggcctgccaccagctgtgcgcccgagggcagcagaagatcc
ggaagtacacgatgcggagactgctgcaggaaacggagctggtggagcc
gctgacacctagcggagcgatgcccaaccaggcgcagatgcggatcctg
aaagagacggagctgaggaaggtgaaggtgcttggatctggcgcttttg
gcacagtctacaagggcatctggatccctgatggggagaatgtgaaaat
tccagtggccatcaaagtgttgagggaaaacacatcccccaaagccaac
aaagaaatcttagacgaagcatacgtgatggctggtgtgggctccccat
atgtctcccgccttctgggcatctgcctgacatccacggtgcagctggt
gacacagcttatgccctatggctgcctcttagactaatctagacccggg
ccactaactcaacgctagtagtggatttaatcccaaatgagccaacaga
accagaaccagaaacagaacaagtaacattggagttagaaatggaagaa
gaaaaaagcaatgatttcgtgtgaataatgcacgaaatcattgcttatt
tttttaaaaagcgatatactagatataacgaaacaacgaactgaataaa
gaatacaaaaaaagagccacgaccagttaaagcctgagaaactttaact
gcgagccttaattgattaccaccaatcaattaaagaagtcgagacccaa
aatttggtaaagtatttaattactttattaatcagatacttaaatatct
gtaaacccattatatcgggtttttgaggggatttcaagtctttaagaag
ataccaggcaatcaattaagaaaaacttagttgattgccattttgttgt
gattcaactttgatcgtagcttctaactaattaattttcgtaagaaagg
agaacagctgaatgaatatcccttttgttgtagaaactgtgcttcatga
cggcttgttaaagtacaaatttaaaaatagtaaaattcgctcaatcact
accaagccaggtaaaagtaaaggggctatttttgcgtatcgctcaaaaa
aaagcatgattggcggacgtggcgttgttctgacttccgaagaagcgat
tcacgaaaatcaagatacatttacgcattggacaccaaacgtttatcgt
tatggtacgtatgcagacgaaaaccgttcatacactaaaggacattctg
aaaacaatttaagacaaatcaataccttctttattgattttgatattca
cacggaaaaagaaactatttcagcaagcgatattttaacaacagctatt
gatttaggattatgcctacgttaattatcaaatctgataaaggttatca
agcatattttgattagaaacgccagtctatgtgacttcaaaatcagaat
ttaaatctgtcaaagcagccaaaataatctcgcaaaatatccgagaata
ttttggaaagtctttgccagttgatctaacgtgcaatcattttgggatt
gctcgtataccaagaacggacaatgtagaatttatgatcccaattaccg
ttattctttcaaagaatggcaagattggtctttcaaacaaacagataat
aagggctttactcgttcaagtctaacggtataagcggtacagaaggcaa
aaaacaagtagatgaaccctggtttaatctcttattgcacgaaacgaaa
ttttcaggagaaaagggtttagtagggcgcaatagcgttatgtttaccc
tctctttagcctactttagttcaggctattcaatcgaaacgtgcgaata
taatatgtttgagtttaataatcgattagatcaacccttagaagaaaaa
gaagtaatcaaaattgttagaagtgcctattcagaaaactatcaagggg
ctaatagggaatacattaccattattgcaaagcttgggtatcaagtgat
ttaaccagtaaagatttatttgtccgtcaagggtggtttaaattcaaga
aaaaaagaagcgaacgtcaacgtgttcatttgtcagaatggaaagaaga
tttaatggcttatattagcgaaaaaagcgatgtatacaagccttattta
gcgacgaccaaaaaagagattagagaagtgctaggcattcctgaacgga
cattagataaattgctgaaggtactgaaggcgaatcaggaaattttctt
taagattaaaccaggaagaaatggtggcattcaacttgctagtgttaaa
tcattgttgctatcgatcattaaattaaaaaaagaagaacgagaaagct
atataaaggcgctgacagcttcgtttaatttagaacgtacatttattca
agaaactctaaacaaattggcagaacgccccaaaacggacccacaactc
gatttgatagctacgatacaggctgaaaataaaacccgcactatgccat
tacatttatatctatgatacgtgtttgtttttctttgctggctagctta
attgcttatatttacctgcaataaaggatttcttacttccattatactc
ccattttccaaaaacatacggggaacacgggaacttattgtacaggcca
cctcatagttaatggtttcgagccttcctgcaatctcatccatggaaat
atattcatccccctgccggcctattaatgtgacttttgtgcccggcgga
tattcctgatccagctccaccataaattggtccatgcaaattcggccgg
caatatcaggcgttttcccttcacaaggatgtcggtccattcaattttc
ggagccagccgtccgcatagcctacaggcaccgtcccgatccatgtgtc
tttttccgctgtgtactcggctccgtagctgacgctctcgccttttctg
atcagtttgacatgtgacagtgtcgaatgcagggtaaatgccggacgca
gctgaaacggtatctcgtccgacatgtcagcagacgggcgaaggccata
catgccgatgccgaatctgactgcattaaaaaagccttttttcagccgg
agtccagcggcgctgttcgcgcagtggaccattagattctttaacggca
gcggagcaatcagctctttaaagcgctcaaactgcattaagaaatagcc
tctttctttttcatccgctgtcgcaaaatgggtaaatacccctttgcac
tttaaacgagggttgcggtcaagaattgccatcacgttctgaacttctt
cctctgtttttacaccaagtctgttcatccccgtatcgaccttcagatg
aaaatgaagagaaccttttttcgtgtggcgggctgcctcctgaagccat
tcaacagaataacctgttaaggtcacgtcatactcagcagcgattgcca
catactccgggggaaccgcgccaagcaccaatataggcgccttcaatcc
ctttttgcgcagtgaaatcgcttcatccaaaatggccacggccaagcat
gaagcacctgcgtcaagagcagcctttgctgtttctgcatcaccatgcc
cgtaggcgtttgctttcacaactgccatcaagtggacatgttcaccgat
atgattttcatattgctgacattttcctttatcgcggacaagtcaattt
ccgcccacgtatctctgtaaaaaggttttgtgctcatggaaaactcctc
tcttattcagaaaatcccagtacgtaattaagtatttgagaattaattt
tatattgattaatactaagtttacccagttttcacctaaaaaacaaatg
atgagataatagctccaaaggctaaagaggactataccaactatttgtt
aattaa

### Example 22: ADXS31-164 is as Immunogenic as Lm-LLO-ChHER2

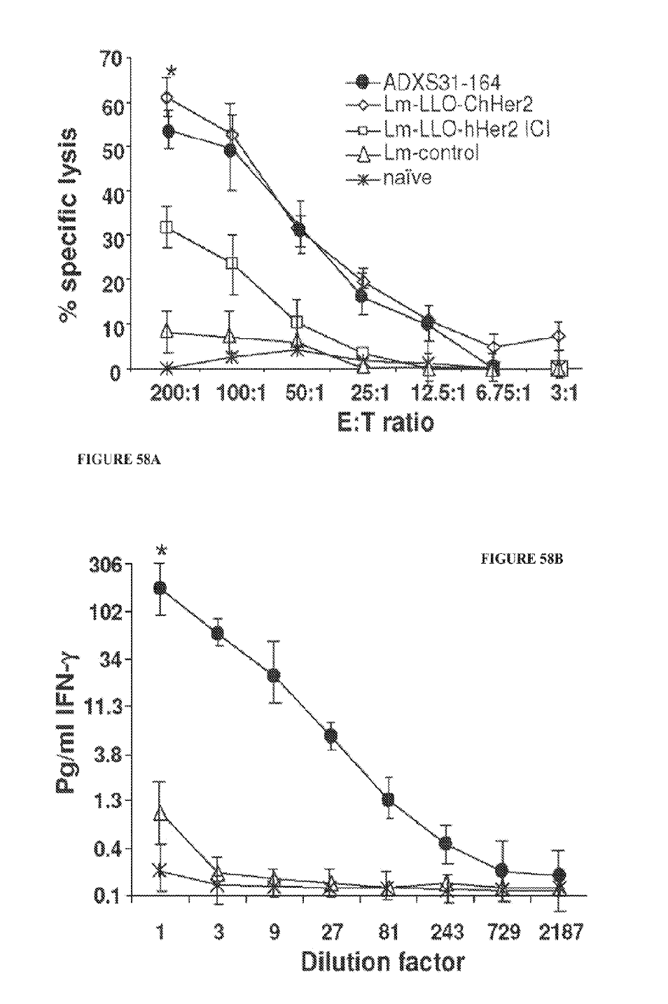

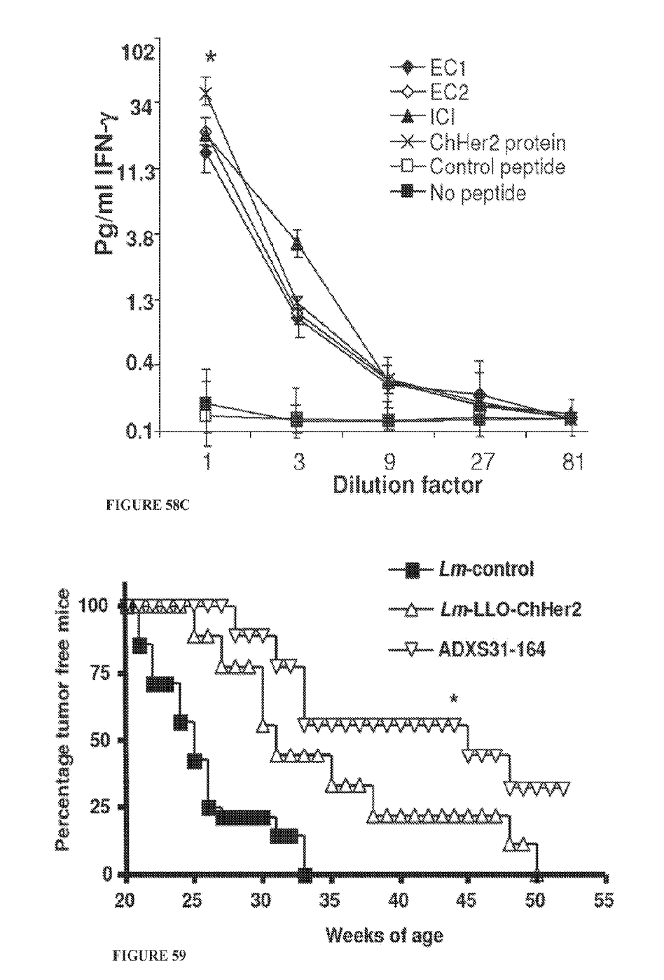

Immunogenic properties of ADXS31-164 in generating anti-Her2/neu specific cytotoxic T cells were compared to those of the Lm-LLO-ChHer2 vaccine in a standard CTL assay. Both vaccines elicited strong but comparable cytotoxic T cell responses toward Her2/neu antigen expressed by 3T3/neu target cells. Accordingly, mice immunized with a Listeria expressing only an intracellular fragment of Her2-fused to LLO showed lower lytic activity than the chimeras which contain more MHC class I epitopes. No CTL activity was detected in naïve animals or mice injected with the irrelevant Listeria vaccine (FIG. 58A). ADXS31-164 was also able to stimulate the secretion of IFN-γ by the splenocytes from wild type FVB/N mice (FIG. 58B). This was detected in the culture supernatants of these cells that were co-cultured with mitomycin C treated NT-2 cells, which express high levels of Her2/neu antigen (FIG. 58C).

Proper processing and presentation of the human MHC class I epitopes after immunizations with ADXS31-164 was tested in HLA-A2 mice. Splenocytes from immunized HLA-A2 transgenics were co-incubated for 72 hours with peptides corresponding to mapped HLA-A2 restricted epitopes located at the extracellular (HLYQGCQVV SEQ ID NO: 88 or KIFGSLAFL SEQ ID NO: 89) or intracellular (RLLQETELV SEQ ID NO: 90) domains of the Her2/neu molecule (FIG. 58C). A recombinant ChHer2 protein was used as positive control and an irrelevant peptide or no peptide as negative controls. The data from this experiment show that ADXS31-164 is able to elicit anti-Her2/neu specific immune responses to human epitopes that are located at different domains of the targeted antigen.

### Example 23: ADXS31-164 was More Efficacious than Lm-LLO-ChHER2 in Preventing the Onset of Spontaneous Mammary Tumors

Anti-tumor effects of ADXS31-164 were compared to those of Lm-LLO-ChHer2 in Her2/neu transgenic animals which develop slow growing, spontaneous mammary tumors at 20-25 weeks of age. All animals immunized with the irrelevant Listeria-control vaccine developed breast tumors within weeks 21-25 and were sacrificed before week 33. In contrast, Listeria-Her2/neu recombinant vaccines caused a significant delay in the formation of the mammary tumors. On week 45, more than 50% of ADXS31-164 vaccinated mice (5 out of 9) were still tumor free, as compared to 25% of mice immunized with Lm-LLO-ChHer2. At week 52, 2 out of 8 mice immunized with ADXS31-164 still remained tumor free, whereas all mice from other experimental groups had already succumbed to their disease (FIG. 59). These results indicate that despite being more attenuated, ADXS31-164 is more efficacious than Lm-LLO-ChHer2 in preventing the onset of spontaneous mammary tumors in Her2/neu transgenic animals.

### Example 24: Mutations in HER2/Neu Gene Upon Immunization with ADXS31-164

Mutations in the MHC class I epitopes of Her2/neu have been considered responsible for tumor escape upon immunization with small fragment vaccines or trastuzumab (Herceptin), a monoclonal antibody that targets an epitope in the extracellular domain of Her2/neu. To assess this, genomic material was extracted from the escaped tumors in the transgenic animals and sequenced the corresponding fragments of the neu gene in tumors immunized with the chimeric or control vaccines. Mutations were not observed within the Her-2/neu gene of any vaccinated tumor samples suggesting alternative escape mechanisms (data not shown).

### Example 25: ADXS31-164 Causes a Significant Decrease in Intra-Tumoral T Regulatory Cells

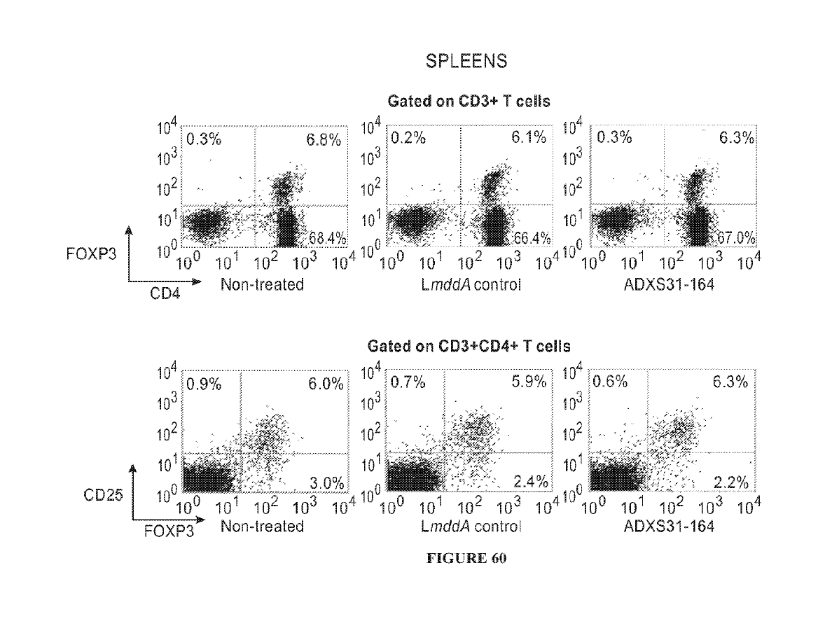

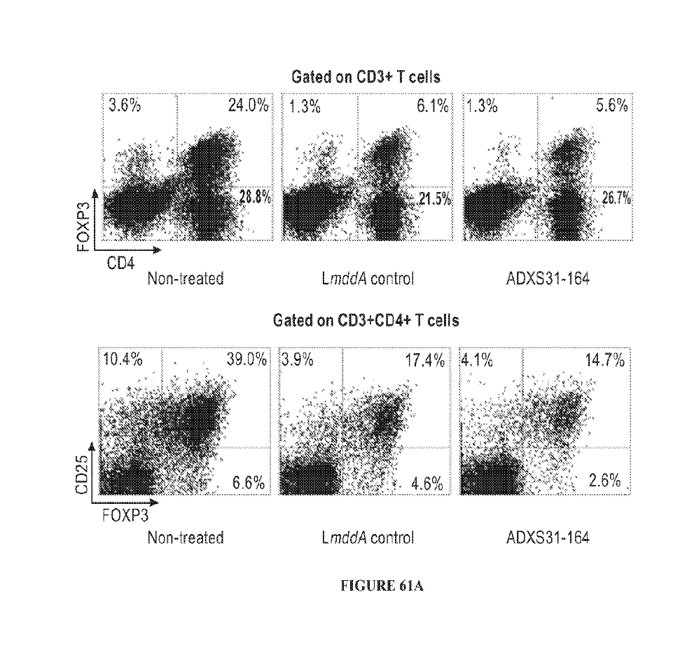

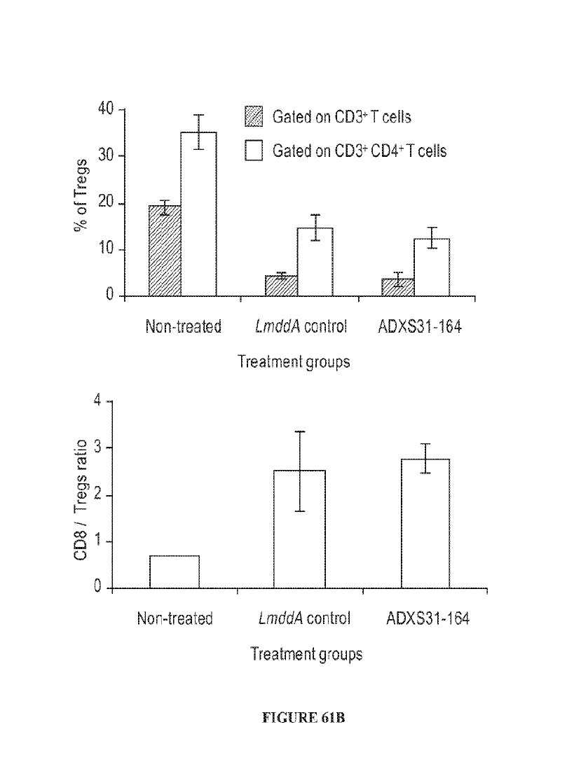

To elucidate the effect of ADXS31-164 on the frequency of regulatory T cells in spleens and tumors, mice were implanted with NT-2 tumor cells. Splenocytes and intra-tumoral lymphocytes were isolated after three immunizations and stained for Tregs, which were defined as CD3⁺/CD4⁺/CD25⁺/FoxP3⁺ cells, although comparable results were obtained with either FoxP3 or CD25 markers when analyzed separately. The results indicated that immunization with ADXS31-164 had no effect on the frequency of Tregs in the spleens, as compared to an irrelevant Listeria vaccine or the naïve animals (FIG. 60). In contrast, immunization with the Listeria vaccines caused a considerable impact on the presence of Tregs in the tumors (FIG. 61A). Whereas in average 19.0% of all CD3⁺ T cells in untreated tumors were Tregs, this frequency was reduced to 4.2% for the irrelevant vaccine and 3.4% for ADXS31-164, a 5-fold reduction in the frequency of intra-tumoral Tregs (FIG. 61B). The decrease in the frequency of intra-tumoral Tregs in mice treated with either of the LmddA vaccines could not be attributed to differences in the sizes of the tumors. In a representative experiment, the tumors from mice immunized with ADXS31-164 were significantly smaller [mean diameter (mm)±SD, 6.71±0.43, n=5] than the tumors from untreated mice (8.69±0.98, n=5, p<0.01) or treated with the irrelevant vaccine (8.41±1.47, n=5, p=0.04), whereas comparison of these last two groups showed no statistically significant difference in tumor size (p=0.73). The lower frequency of Tregs in tumors treated with LmddA vaccines resulted in an increased intratumoral CD8/Tregs ratio, suggesting that a more favorable tumor microenvironment can be obtained after immunization with LmddA vaccines. However, only the vaccine expressing the target antigen HER2/neu (ADXS31-164) was able to reduce tumor growth, indicating that the decrease in Tregs has an effect only in the presence on antigen-specific responses in the tumor.

### Example 26: Peripheral Immunization with ADXS31-164 can Delay the Growth of A Metastatic Breast Cancer Cell Line in the Brain

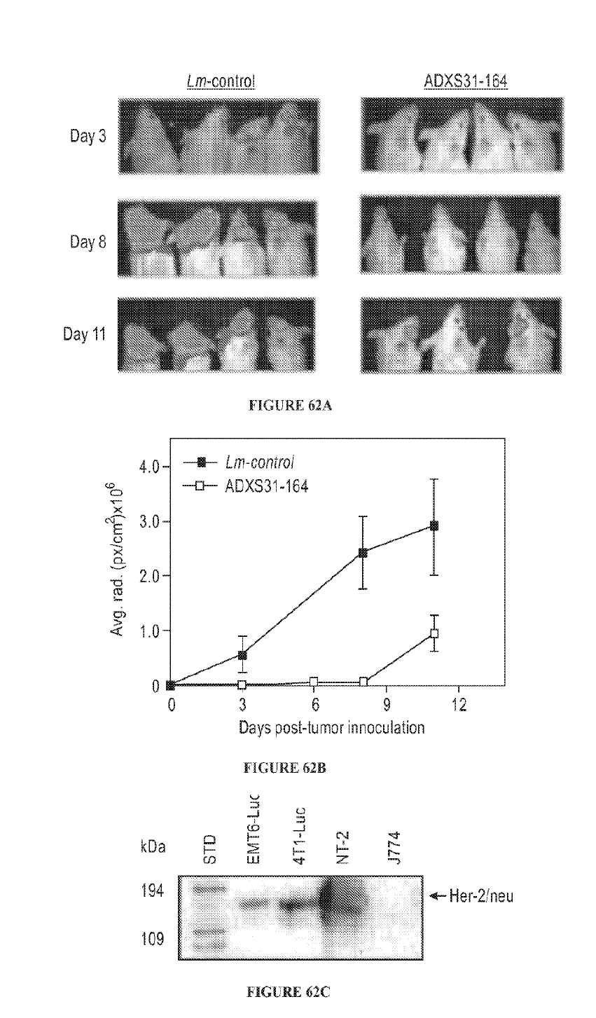

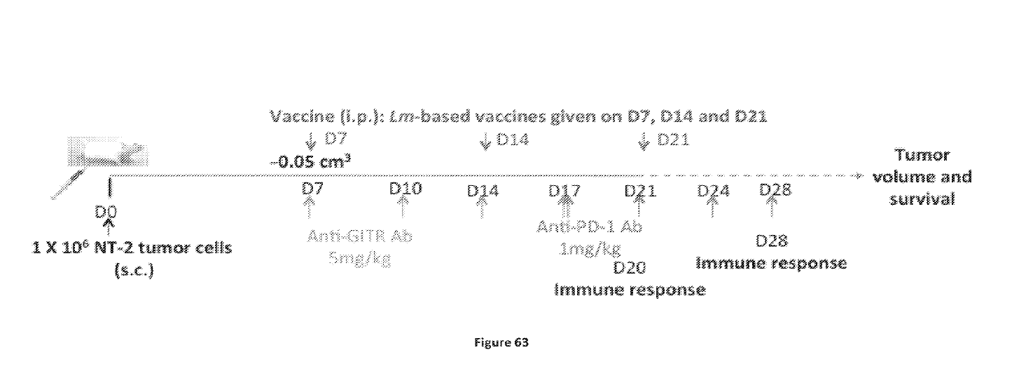

Mice were immunized IP with ADXS31-164 or irrelevant Lm-control vaccines and then implanted intra-cranially with 5,000 EMT6-Luc tumor cells, expressing luciferase and low levels of Her2/neu (FIG. 62A). Tumors were monitored at different times post-inoculation by ex vivo imaging of anesthetized mice. On day 8 post-tumor inoculation tumors were detected in all control animals, but none of the mice in ADXS31-164 group showed any detectable tumors (FIGS. 62A and 62B). ADXS31-164 could clearly delay the onset of these tumors, as on day 11 post-tumor inoculation all mice in negative control group had already succumbed to their tumors, but all mice in ADXS31-164 group were still alive and only showed small signs of tumor growth. These results strongly suggest that the immune responses obtained with the peripheral administration of ADXS31-164 could possibly reach the central nervous system and that LmddA-based vaccines might have a potential use for treatment of CNS tumors.

### Example 27: Therapeutic Efficacy and Immune Modulatory Effects of the Triple Combination of Lm-Based HER2/Neu Vaccine, GITR Agonist Antibodies and Checkpoint Inhibitor, PD-1 Ab in Her2/Neu Positive BC Mouse Models

### Experimental Design:

Mouse Tumor Models:

Two mouse tumor models are used: a rat Her-2/FVB/N mouse model and a FVB/N Her-2/neu transgenic mouse model.

Antibodies:

Anti-PD1 (RMP-14 clone, Rat IgG2a) and anti-GITR (DTA-1 clone). Both the antibodies are injected i.p twice a week. Anti-PD-1 Ab is given throughout the experiment at a dose of 1 mg/Kg b.wt. For agonist GITR Ab, 4 total doses are given at a dose of 5 mg/Kg b.wt.

Experiments with Rat Her-2/FVB/N Mouse Model:

Because the rat and human Her-2/neu proteins are highly homologous, the rat Her-2/FVB/N mouse model is used to test for the therapeutic antitumor effects of the Lm-based Her-2/neu vaccine in combination with GITR agonist and anti-PD-1 Ab. Tumors are implanted s.c. in female FVB/N mice (8-10 weeks old; 5/group) on the right flank by injecting 1×10⁶ NT-2 tumor cells that expresses high levels of rat HER2/neu protein. Once the tumor volume reaches about 0.5 cm³, mice are randomly distributed in 16 groups (Table 8) and treated with highly attenuated Lm-based vaccine vectors (i.p.; 1 to 5×10⁸ colony forming units determined by an in vivo toxicity assay) with or without LLO and HER2/neu (Lm, Lm-LLO and ADXS31-164), anti-PD1 Ab, and agonist anti-GITR Ab. The prime dose of the vaccines is followed by two boosts at 7-d intervals.

TABLE 8
Distribution of mice in 16 groups for therapeutic, immune
response, and tumor prevention studies.

| | +GITR agonist | | +GITR agonist + |
|---|---|---|---|
| Single treatments | Ab | +anti-PD-1 Ab | anti-PD-1 Ab |
| 1. PBS | 5. PBS | 9. PBS | 13. PBS |
| 2. Lm | 6. Lm | 10. Lm | 14. Lm |
| 3. Lm-LLO | 7. Lm-LLO | 11. Lm-LLO | 15. Lm-LLO |
| 4. Lm-LLO- | 8. ADXS31-164 | 12. ADXS31- | 16. ADXS31-164 |
| Her2/neu | | 164 | |
| (ADXS31-164) | | | |

Agonist GITR Ab is administered beginning at the same day with the vaccine for a total of 4 doses. Since PD1 plays a role in both early activation and T cell exhaustion, administration of anti-PD-1 after the vaccine may reinvigorate the exhausting T cells. Therefore, anti-PD1 Ab is injected 3 days after the second vaccination to determine if antigen specific response can further be enhanced. In the control groups, mice receive PBS. Tumor growth and survival is measured. Tumors is measured twice weekly using digital calipers, and tumor volumes is calculated using the formula V=(W²*L)/2, where V is the volume, L is the length (longer diameter) and W is width (shorter diameter). Mice are sacrificed when moribund or if tumor volume reached 1.5 cm³. A general treatment schedule is shown in FIG. 63. Experiments are repeated twice.

Experiments with FVB/N Her-2/Neu Transgenic Mouse Model:

The transplantable tumor murine model using the NT-2 cell line is a fast-growing tumor model with little tolerance toward the Her-2/neu antigen. A more challenging tumor model, where tolerance toward the Her-2/neu antigen might play a significant role in attenuating the immunotherapeutic efficacy of a vaccine, is the rat Her-2/neu transgenic mouse model. In this model, mice develop spontaneous, slow-growing mammary tumors between 20-25 weeks of age. By using this mouse strain, therefore we are able to test whether the Lm-based Her2/neu vaccine is able to overcome tolerance toward the Her-2/neu self-antigen. Breeding pairs of these mice are kindly provided by Advaxis, Inc.

The mice are distributed in 16 groups as shown in Table 8. The mice are immunized for a total of six doses (1 to 5×10⁸ colony forming units) starting from week 6 at an interval of 3 weeks. Compared to the fast growing model explained above, more immunizations with the vaccine are possible in this model since this is a prophylactic model and the spontaneous tumors do not start appearing until about week 20. Agonist GITR Ab is administered beginning at the same day with the vaccine for a total of 6 doses and is given with each dose of vaccine. Treatment with anti-PD1 Ab is started 3-4 days after the last vaccination and continued throughout the experiment. Mice are observed twice a week for the emergence and growth of spontaneous mammary tumors for up to 52 weeks. Spontaneous tumor formation is detected by palpation of the upper and lower mouse mammary glands, which will identify tumors as small as 1 to 2 mm in diameter. A general treatment schedule is shown in FIG. 5.

Immune Response and Modulation Studies:

Further, detailed mechanisms of immune modulation responsible for the effects on tumor growth and survival are investigated in the tumor, spleen, and the tumor draining lymph nodes (TDLN). For these experiments, mice are grouped (4 mice/group) and treated similarly as above and are sacrificed at six days after the second immunization and a week after the third immunization. Tumors, spleen, TDLNs are harvested and the following assays are performed:

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. An immunogenic composition comprising a recombinant Listeria strain comprising a nucleic acid molecule, said nucleic acid molecule comprising a first open reading frame encoding a fusion polypeptide, wherein said fusion polypeptide comprises a truncated listeriolysin O (LLO) protein, a truncated ActA protein, or a PEST amino acid sequence fused to a heterologous antigen or fragment thereof, said composition further comprising an antibody or a functional fragment thereof.

2. The composition of claim 1, wherein said antibody or functional fragment thereof comprises a polyclonal antibody, a monoclonal antibody, an Fab fragment, an F(ab′)2 fragment, an Fv fragment, a single chain antibody (SCA), or any combination thereof.

3. The composition of any one of claims 1-2, wherein said antibody or functional fragment thereof binds to said heterologous antigen or a portion thereof comprising a T-cell receptor co-stimulatory molecule, an antigen presenting cell receptor binding co-stimulatory molecule, or a member of the TNF receptor superfamily.

4. The composition of claim 3, wherein said member of the TNF receptor superfamily is selected from the group consisting of a glucocorticoid-induced TNF receptor (GITR), a OX40 (a CD134 receptor), a 4-1BB (a CD137 receptor) and a TNFR25.

5. The composition of claim 4, wherein said antigen presenting cell receptor binding co-stimulatory molecule is selected from the group consisting of a CD80 receptor, a CD86 receptor and CD40 receptor.

6. The composition of any one of claims 1-5, wherein said nucleic acid molecule comprising a first open reading frame is integrated into the Listeria genome.

7. The composition of any one of claims 1-5, wherein said nucleic acid molecule comprising a first open reading frame is in a plasmid in said recombinant Listeria strain.

8. The composition of claim 7, wherein said plasmid is stably maintained in said recombinant Listeria strain in the absence of antibiotic selection.

9. The composition of claim 7, wherein said plasmid does not confer antibiotic resistance upon said recombinant Listeria.

10. The composition of any of claims 1-9, wherein said heterologous antigen is a tumor-associated antigen.

11. The composition of claim 10, wherein said tumor-associated antigen is a prostate specific antigen (PSA), a human papilloma virus (HPV) antigen or a chimeric Her2/neu antigen.

12. The composition according to any of the claims 1-11, wherein said recombinant Listeria strain is attenuated.

13. The composition of claim 12, wherein said attenuated Listeria comprises a mutation, deletion, disruption, inactivation, replacement, or truncation in an endogenous gene.

14. The composition of claim 13, wherein said endogenous gene comprises an actA virulence gene, a prfA virulence gene, a dal gene, an inlB gene, a dat gene or a combination thereof.

15. The composition of any one of claims 13-14, wherein said endogenous gene is a prfA gene.

16. The composition of any one of claims 13-14, wherein said endogenous genes are the dal/dat and actA genes.

17. The composition of any one of claims 1-15, wherein said nucleic acid comprising a first open reading frame, further comprises a second open reading frame.

18. The composition of claim 17, wherein said second open reading frame encodes a PrfA protein comprising a D133V mutation, and wherein said PrfA protein complements said mutation, deletion, disruption, inactivation, replacement, or truncation in said prfA gene.

19. The composition of any one of claim 1-14, 16 or 17, wherein said second open reading frame encodes a metabolic enzyme and wherein said metabolic enzyme complements said mutation, deletion, disruption, inactivation, replacement, or truncation in said dal and dat genes.

20. The composition according to claim 19, wherein said metabolic enzyme encoded by said second open reading frame is an alanine racemase enzyme or a D-amino acid transferase enzyme.

21. The composition according to any of claims 1-20, further comprising an adjuvant.

22. The composition of claim 21, wherein said adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

23. The composition of any one of claims 1-22, wherein said Listeria strain is Listeria monocytogenes.

24. A method of eliciting an enhanced anti-tumor T cell response in a subject, said method comprising the step of administering to said subject an effective amount of an immunogenic composition comprising a recombinant Listeria strain comprising a nucleic acid molecule, said nucleic acid molecule comprising a first open reading frame encoding a fusion polypeptide, wherein said fusion polypeptide comprises a truncated listeriolysin O (LLO) protein, a truncated ActA protein, or a PEST amino acid sequence fused to a heterologous antigen or fragment thereof, wherein said method further comprises a step of administering an effective amount of a composition comprising an antibody or a fragment thereof to said subject, and wherein said administration enhances the anti-tumor T cell response in said subject.

25. The method of claim 24, wherein said antibody or functional fragment thereof comprises a polyclonal antibody, a monoclonal antibody, an Fab fragment, an F(ab′)2 fragment, an Fv fragment, a single chain antibody (SCA), or any combination thereof.

26. The method of any one of claims 24-25, wherein said antibody or functional fragment thereof binds to a heterologous antigen or a portion thereof comprising a T-cell receptor co-stimulatory molecule, an antigen presenting cell receptor binding co-stimulatory molecules, or a member of the TNF receptor superfamily.

27. The method of claim 26, wherein said member of the TNF receptor superfamily is selected from the group consisting of a glucocorticoid-induced TNF receptor (GITR), a OX40 (a CD134 receptor), a 4-1BB (a CD137 receptor) and a TNFR25.

28. The method of claim 27, wherein said antigen presenting cell receptor binding co-stimulatory molecule is selected from the group consisting of a CD80 receptor, a CD86 receptor and CD40 receptor.

29. The method of claims 24-28, wherein said nucleic acid molecule comprising a first open reading frame, is integrated into the Listeria genome.

30. The method of claims 24-28, wherein said nucleic acid molecule comprising a first open reading frame, is in a plasmid in said recombinant Listeria vaccine strain.

31. The method of claim 30, wherein said plasmid is stably maintained in said recombinant Listeria strain in the absence of antibiotic selection.

32. The method of claim 30, wherein said plasmid does not confer antibiotic resistance upon said recombinant Listeria.

33. The method according to any of the claims 24-32, wherein said heterologous antigen is a tumor-associated antigen.

34. The method according to claim 33, wherein said tumor-associated antigen is a prostate specific antigen (PSA), a human papilloma virus (HPV) antigen or a Her2/neu chimeric antigen.

35. The method according to any of the claims 24-34, wherein said recombinant Listeria strain is attenuated.

36. The method of claim 35, wherein said attenuated Listeria comprises a mutation, deletion, disruption, inactivation, replacement, or truncation in an endogenous gene.

37. The method of claim 36, wherein said endogenous gene comprises an actA virulence gene, a prfA virulence gene, a dal gene, an inlB gene, a dat gene or a combination thereof.

38. The composition of any one of claims 36-37, wherein said endogenous gene is a prfA gene.

39. The composition of any one of claims 36-37, wherein said endogenous genes are the dal/dat and actA genes.

40. The composition of any one of claims 24-37, wherein said nucleic acid comprising a first open reading frame, further comprises a second open reading frame.

41. The composition of claim 40, wherein said second open reading frame encodes a PrfA protein comprising a D133V mutation, and wherein said PrfA protein complements said mutation, deletion, disruption, inactivation, replacement, or truncation in said prfA gene.

42. The composition of any one of claim 24-37, or 39-40, wherein said second open reading frame encodes a metabolic enzyme and wherein said metabolic enzyme complements said mutation, deletion, disruption, inactivation, replacement, or truncation in said dal and dat genes.

43. The composition according to claim 42, wherein said metabolic enzyme encoded by said second open reading frame is an alanine racemase enzyme or a D-amino acid transferase enzyme.

44. The composition according to any of claims 24-43, further comprising an adjuvant.

45. The composition of claim 44, wherein said adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

46. The composition of any one of claims 1-22, wherein said Listeria strain is Listeria monocytogenes.

47. The method of any one of claims 24-46, wherein said composition comprising an antibody or fragment thereof is administered prior to, concurrent with or following the administration of said composition comprising said recombinant attenuated Listeria strain

48. The method of any one of claims 24-47, wherein said anti-tumor T cell response comprises increasing a level of Interferon-gamma (INF-γ) producing cells.

49. The method of any one of claims 24-48, wherein said anti-tumor T cell response comprises an increase of tumor infiltration by T effector cells.

50. The method of claim 49, wherein said T effector cells are CD45+CD8+T cells or CD4+Fox3P− T cells.

51. The method of any one of claims 24-50, wherein said anti-tumor T cell response comprises a decrease in the frequency of T regulatory cells (Tregs) in the spleen and the tumor microenvironment.

52. The method of any one of claims 24-51, wherein said anti-tumor T cell response comprises a decrease in the frequency of myeloid derived suppressor cells (MDSCs) in the spleen and the tumor microenvironment.

53. The method of any one of claims 24-52, wherein said method comprises increasing antigen-specific T-cells in said subject.

54. The method of any one of claims 24-54, wherein said method comprises treating a tumor or cancer in a subject.

55. The method of any one of claims 24-53, wherein said method comprises increasing survival time of a subject suffering from a cancer or a tumor.

56. The method of any one of claims 55-55, wherein said tumor is a breast tumor, a head and neck tumor, a cervical tumor, a prostate tumor.

57. The method of any one of claim 55-55, wherein said cancer is a breast cancer, a head and neck cancer, a cervical cancer, a prostate cancer, an anal cancer, an esophageal cancer, a lung cancer, a melanoma, an osteosarcoma, or an ovarian cancer.


SEQUENCE LISTING (89 sequences)

SEQ ID NO: 1 - protein, 32 aa, Artificial Sequence
  PEST sequence
KENSISSMAPPASPPASPKTPIEKKHADEIDK

SEQ ID NO: 2 - protein, 529 aa, Artificial Sequence
  GenBank Accession No. P13128
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 3 - protein, 441 aa, Artificial Sequence
  N-terminal fragment of an LLO protein
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 4 - protein, 416 aa, Artificial Sequence
  LLO fragment
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTD

SEQ ID NO: 5 - protein, 14 aa, Artificial Sequence
  PEST sequence
KTEEQPSEVNTGPR

SEQ ID NO: 6 - protein, 28 aa, Artificial Sequence
  PEST sequence
KASVTDTSEGDLDSSMQSADESTPQPLK

SEQ ID NO: 7 - protein, 20 aa, Artificial Sequence
  PEST sequence
KNEEVNASDFPPPPTDEELR

SEQ ID NO: 8 - protein, 33 aa, Artificial Sequence
  PEST sequence
RGGIPTSEEFSSLNSGDFTDDENSETTEEEIDR

SEQ ID NO: 9 - protein, 17 aa, Artificial Sequence
  PEST sequence
KQNTASTETTTTNEQPK

SEQ ID NO: 10 - protein, 17 aa, Artificial Sequence
  PEST sequence
KQNTANTETTTTNEQPK

SEQ ID NO: 11 - protein, 639 aa, Artificial Sequence
  ActA protein
MGLNRFMRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRPTSEEFSSLNSGDFTDDENSETTEEEIDRLADLRDRGTGKHSRNAGFLPLNPFASSPVPSLSPKVSKISDRALISDITKKTPFKNPSQPLNVFNKKTTTKTVTKKPTPVKTAPKLAELPATKPQETVLRENKTPFIEKQAETNKQSINMPSLPVIQKEATESDKEEMKPQTEEKMVEESESANNANGKNRSAGIEEGKLIAKSAEDEKAKEEPGNHTTLILAMLAIGVFSLGAFIKIIQLRKNN

SEQ ID NO: 12 - protein, 390 aa, Artificial Sequence
  a truncated ActA protein
MRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRP

SEQ ID NO: 13 - protein, 100 aa, Artificial Sequence
  truncated ActA protein
MGLNRFMRAMMVVFITANCITINPDIIFAATDSEDSSLNTDEWEEEKTEEQPSEVNTGPRYETAREVSSRDIKELEKSNKVRNTNKADLIAMLKEKAEKG

SEQ ID NO: 14 - protein, 1170 aa, Artificial Sequence
  recombinant nucleotide encoding a truncated ActA protein
ATGCGTGCGATGATGGTGGTTTTCATTACTGCCAATTGCATTACGATTAACCCCGACATAATATTTGCAGCGACAGATAGCGAAGATTCTAGTCTAAACACAGATGAATGGGAAGAAGAAAAAACAGAAGAGCAACCAAGCGAGGTAAATACGGGACCAAGATACGAAACTGCACGTGAAGTAAGTTCACGTGATATTAAAGAACTAGAAAAATCGAATAAAGTGAGAAATACGAACAAAGCAGACCTAATAGCAATGTTGAAAGAAAAAGCAGAAAAAGGTCCAAATATCAATAATAACAACAGTGAACAAACTGAGAATGCGGCTATAAATGAAGAGGCTTCAGGAGCCGACCGACCAGCTATACAAGTGGAGCGTCGTCATCCAGGATTGCCATCGGATAGCGCAGCGGAAATTAAAAAAAGAAGGAAAGCCATAGCATCATCGGATAGTGAGCTTGAAAGCCTTACTTATCCGGATAAACCAACAAAAGTAAATAAGAAAAAAGTGGCGAAAGAGTCAGTTGCGGATGCTTCTGAAAGTGACTTAGATTCTAGCATGCAGTCAGCAGATGAGTCTTCACCACAACCTTTAAAAGCAAACCAACAACCATTTTTCCCTAAAGTATTTAAAAAAATAAAAGATGCGGGGAAATGGGTACGTGATAAAATCGACGAAAATCCTGAAGTAAAGAAAGCGATTGTTGATAAAAGTGCAGGGTTAATTGACCAATTATTAACCAAAAAGAAAAGTGAAGAGGTAAATGCTTCGGACTTCCCGCCACCACCTACGGATGAAGAGTTAAGACTTGCTTTGCCAGAGACACCAATGCTTCTTGGTTTTAATGCTCCTGCTACATCAGAACCGAGCTCATTCGAATTTCCACCACCACCTACGGATGAAGAGTTAAGACTTGCTTTGCCAGAGACGCCAATGCTTCTTGGTTTTAATGCTCCTGCTACATCGGAACCGAGCTCGTTCGAATTTCCACCGCCTCCAACAGAAGATGAACTAGAAATCATCCGGGAAACAGCATCCTCGCTAGATTCTAGTTTTACAAGAGGGGATTTAGCTAGTTTGAGAAATGCTATTAATCGCCATAGTCAAAATTTCTCTGATTTCCCACCAATCCCAACAGAAGAAGAGTTGAACGGGAGAGGCGGTAGACCA

SEQ ID NO: 15 - protein, 261 aa, Artificial Sequence
  KLK3 protein
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 16 - protein, 237 aa, Artificial Sequence
  KLK3 protein
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANP

SEQ ID NO: 17 - protein, 237 aa, Artificial Sequence
  KLK3 protein
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 18 - DNA, 5873 bp, Artificial Sequence
  nucleotide encoding the KLK3 protein
ggtgtcttaggcacactggtcttggagtgcaaaggatctaggcacgtgaggctttgtatgaagaatcggggatcgtacccaccccctgtttctgtttcatcctgggcatgtctcctctgcctttgtcccctagatgaagtctccatgagctacaagggcctggtgcatccagggtgatctagtaattgcagaacagcaagtgctagctctccctccccttccacagctctgggtgtgggagggggttgtccagcctccagcagcatggggagggccttggtcagcctctgggtgccagcagggcaggggcggagtcctggggaatgaaggttttatagggctcctgggggaggctccccagccccaagcttaccacctgcacccggagagctgtgtcaccatgtgggtcccggttgtcttcctcaccctgtccgtgacgtggattggtgagaggggccatggttggggggatgcaggagagggagccagccctgactgtcaagctgaggctctttcccccccaacccagcaccccagcccagacagggagctgggctcttttctgtctctcccagccccacttcaagcccatacccccagtcccctccatattgcaacagtcctcactcccacaccaggtccccgctccctcccacttaccccagaactttcttcccatttgcccagccagctccctgctcccagctgctttactaaaggggaagttcctgggcatctccgtgtttctctttgtggggctcaaaacctccaaggacctctctcaatgccattggttccttggaccgtatcactggtccatctcctgagcccctcaatcctatcacagtctactgacttttcccattcagctgtgagtgtccaaccctatcccagagaccttgatgcttggcctcccaatcttgccctaggatacccagatgccaaccagacacctccttctttcctagccaggctatctggcctgagacaacaaatgggtccctcagtctggcaatgggactctgagaactcctcattccctgactcttagccccagactcttcattcagtggcccacattttccttaggaaaaacatgagcatccccagccacaactgccagctctctgagtccccaaatctgcatccttttcaaaacctaaaaacaaaaagaaaaacaaataaaacaaaaccaactcagaccagaactgttttctcaacctgggacttcctaaactttccaaaaccttcctcttccagcaactgaacctcgccataaggcacttatccctggttcctagcaccccttatcccctcagaatccacaacttgtaccaagtttcccttctcccagtccaagaccccaaatcaccacaaaggacccaatccccagactcaagatatggtctgggcgctgtcttgtgtctcctaccctgatccctgggttcaactctgctcccagagcatgaagcctctccaccagcaccagccaccaacctgcaaacctagggaagattgacagaattcccagcctttcccagctccccctgcccatgtcccaggactcccagccttggttctctgcccccgtgtcttttcaaacccacatcctaaatccatctcctatccgagtcccccagttccccctgtcaaccctgattcccctgatctagcaccccctctgcaggcgctgcgcccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaagtgagtaggggcctggggtctggggagcaggtgtctgtgtcccagaggaataacagctgggcattttccccaggataacctctaaggccagccttgggactgggggagagagggaaagttctggttcaggtcacatggggaggcagggttggggctggaccaccctccccatggctgcctgggtctccatctgtgtccctctatgtctctttgtgtcgctttcattatgtctcttggtaactggcttcggttgtgtctctccgtgtgactattttgttctctctctccctctcttctctgtcttcagtctccatatctccccctctctctgtccttctctggtccctctctagccagtgtgtctcaccctgtatctctctgccaggctctgtctctcggtctctgtctcacctgtgccttctccctactgaacacacgcacgggatgggcctgggggaccctgagaaaaggaagggctttggctgggcgcggtggctcacacctgtaatcccagcactttgggaggccaaggcaggtagatcacctgaggtcaggagttcgagaccagcctggccaactggtgaaaccccatctctactaaaaatacaaaaaattagccaggcgtggtggcgcatgcctgtagtcccagctactcaggagctgagggaggagaattgcattgaacctggaggttgaggttgcagtgagccgagaccgtgccactgcactccagcctgggtgacagagtgagactccgcctcaaaaaaaaaaaaaaaaaaaaaaaaaaaaaagaaaagaaaagaaaagaaaaggaagtgttttatccctgatgtgtgtgggtatgagggtatgagagggcccctctcactccattccttctccaggacatccctccactcttgggagacacagagaagggctggttccagctggagctgggaggggcaattgagggaggaggaaggagaagggggaaggaaaacagggtatgggggaaaggaccctggggagcgaagtggaggatacaaccttgggcctgcaggcaggctacctacccacttggaaacccacgccaaagccgcatctacagctgagccactctgaggcctcccctccccggcggtccccactcagctccaaagtctctctcccttttctctcccacactttatcatcccccggattcctctctacttggttctcattcttcctttgacttcctgcttccctttctcattcatctgtttctcactttctgcctggttttgttcttctctctctctttctctggcccatgtctgtttctctatgtttctgtcttttctttctcatcctgtgtattttcggctcaccttgtttgtcactgttctcccctctgccctttcattctctctgcccttttaccctcttccttttcccttggttctctcagttctgtatctgcccttcaccctctcacactgctgtttcccaactcgttgtctgtattttggcctgaactgtgtcttcccaaccctgtgttttctcactgtttctttttctcttttggagcctcctccttgctcctctgtcccttctctctttccttatcatcctcgctcctcattcctgcgtctgcttcctccccagcaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagtgtacgcctgggccagatggtgcagccgggagcccagatgcctgggtctgagggaggaggggacaggactcctgggtctgagggaggagggccaaggaaccaggtggggtccagcccacaacagtgtttttgcctggcccgtagtcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcggtgagtcatccctactcccaagatcttgagggaaaggtgagtgggaccttaattctgggctggggtctagaagccaacaaggcgtctgcctcccctgctccccagctgtagccatgccacctccccgtgtctcatctcattccctccttccctcttctttgactccctcaaggcaataggttattcttacagcacaactcatctgttcctgcgttcagcacacggttactaggcacctgctatgcacccagcactgccctagagcctgggacatagcagtgaacagacagagagcagcccctcccttctgtagcccccaagccagtgaggggcacaggcaggaacagggaccacaacacagaaaagctggagggtgtcaggaggtgatcaggctctcggggagggagaaggggtggggagtgtgactgggaggagacatcctgcagaaggtgggagtgagcaaacacctgcgcaggggaggggagggcctgcggcacctgggggagcagagggaacagcatctggccaggcctgggaggaggggcctagagggcgtcaggagcagagaggaggttgcctggctggagtgaaggatcggggcagggtgcgagagggaacaaaggacccctcctgcagggcctcacctgggccacaggaggacactgcttttcctctgaggagtcaggaactgtggatggtgctggacagaagcaggacagggcctggctcaggtgtccagaggctgcgctggcctcctatgggatcagactgcagggagggagggcagcagggatgtggagggagtgatgatggggctgacctgggggtggctccaggcattgtccccacctgggcccttacccagcctccctcacaggctcctggccctcagtctctcccctccactccattctccacctacccacagtgggtcattctgatcaccgaactgaccatgccagccctgccgatggtcctccatggctccctagtgccctggagaggaggtgtctagtcagagagtagtcctggaaggtggcctctgtgaggagccacggggacagcatcctgcagatggtcctggcccttgtcccaccgacctgtctacaaggactgtcctcgtggaccctcccctctgcacaggagctggaccctgaagtcccttcctaccggccaggactggagcccctacccctctgttggaatccctgcccaccttcttctggaagtcggctctggagacatttctctcttcttccaaagctgggaactgctatctgttatctgcctgtccaggtctgaaagataggattgcccaggcagaaactgggactgacctatctcactctctccctgcttttacccttagggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaagtccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagttaggatggggtgtctgtgttatttgtgggatacagagatgaaagaggggtgggatcc

SEQ ID NO: 19 - protein, 238 aa, Artificial Sequence
  KLK3 protein
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSWVILITELTMPALPMVLHGSLVPWRGGV

SEQ ID NO: 20 - protein, 1906 aa, Artificial Sequence
  nucleotide encoding the KLK3 protein
AGCCCCAAGCTTACCACCTGCACCCGGAGAGCTGTGTCACCATGTGGGTCCCGGTTGTCTTCCTCACCCTGTCCGTGACGTGGATTGGTGCTGCACCCCTCATCCTGTCTCGGATTGTGGGAGGCTGGGAGTGCGAGAAGCATTCCCAACCCTGGCAGGTGCTTGTGGCCTCTCGTGGCAGGGCAGTCTGCGGCGGTGTTCTGGTGCACCCCCAGTGGGTCCTCACAGCTGCCCACTGCATCAGGAACAAAAGCGTGATCTTGCTGGGTCGGCACAGCCTGTTTCATCCTGAAGACACAGGCCAGGTATTTCAGGTCAGCCACAGCTTCCCACACCCGCTCTACGATATGAGCCTCCTGAAGAATCGATTCCTCAGGCCAGGTGATGACTCCAGCCACGACCTCATGCTGCTCCGCCTGTCAGAGCCTGCCGAGCTCACGGATGCTGTGAAGGTCATGGACCTGCCCACCCAGGAGCCAGCACTGGGGACCACCTGCTACGCCTCAGGCTGGGGCAGCATTGAACCAGAGGAGTTCTTGACCCCAAAGAAACTTCAGTGTGTGGACCTCCATGTTATTTCCAATGACGTGTGTGCGCAAGTTCACCCTCAGAAGGTGACCAAGTTCATGCTGTGTGCTGGACGCTGGACAGGGGGCAAAAGCACCTGCTCGTGGGTCATTCTGATCACCGAACTGACCATGCCAGCCCTGCCGATGGTCCTCCATGGCTCCCTAGTGCCCTGGAGAGGAGGTGTCTAGTCAGAGAGTAGTCCTGGAAGGTGGCCTCTGTGAGGAGCCACGGGGACAGCATCCTGCAGATGGTCCTGGCCCTTGTCCCACCGACCTGTCTACAAGGACTGTCCTCGTGGACCCTCCCCTCTGCACAGGAGCTGGACCCTGAAGTCCCTTCCCCACCGGCCAGGACTGGAGCCCCTACCCCTCTGTTGGAATCCCTGCCCACCTTCTTCTGGAAGTCGGCTCTGGAGACATTTCTCTCTTCTTCCAAAGCTGGGAACTGCTATCTGTTATCTGCCTGTCCAGGTCTGAAAGATAGGATTGCCCAGGCAGAAACTGGGACTGACCTATCTCACTCTCTCCCTGCTTTTACCCTTAGGGTGATTCTGGGGGCCCACTTGTCTGTAATGGTGTGCTTCAAGGTATCACGTCATGGGGCAGTGAACCATGTGCCCTGCCCGAAAGGCCTTCCCTGTACACCAAGGTGGTGCATTACCGGAAGTGGATCAAGGACACCATCGTGGCCAACCCCTGAGCACCCCTATCAACCCCCTATTGTAGTAAACTTGGAACCTTGGAAATGACCAGGCCAAGACTCAAGCCTCCCCAGTTCTACTGACCTTTGTCCTTAGGTGTGAGGTCCAGGGTTGCTAGGAAAAGAAATCAGCAGACACAGGTGTAGACCAGAGTGTTTCTTAAATGGTGTAATTTTGTCCTCTCTGTGTCCTGGGGAATACTGGCCATGCCTGGAGACATATCACTCAATTTCTCTGAGGACACAGATAGGATGGGGTGTCTGTGTTATTTGTGGGGTACAGAGATGAAAGAGGGGTGGGATCCACACTGAGAGAGTGGAGAGTGACATGTGCTGGACACTGTCCATGAAGCACTGAGCAGAAGCTGGAGGCACAACGCACCAGACACTCACAGCAAGGATGGAGCTGAAAACATAACCCACTCTGTCCTGGAGGCACTGGGAAGCCTAGAGAAGGCTGTGAGCCAAGGAGGGAGGGTCTTCCTTTGGCATGGGATGGGGATGAAGTAAGGAGAGGGACTGGACCCCCTGGAAGCTGATTCACTATGGGGGGAGGTGTATTGAAGTCCTCCAGACAACCCTCAGATTTGATGATTTCCTAGTAGAACTCACAGAAATAAAGAGCTGTTATACTGTG

SEQ ID NO: 21 - protein, 69 aa, Artificial Sequence
  KLK3 protein
MWVPVVFLTLSVTWIGAAPLILSRIVGGWECEKHSQPWQVLVASRGRAVCGGVLVHPQWVLTAAHCIRK

SEQ ID NO: 22 - DNA, 554 bp, Artificial Sequence
  nucleotide encoding the KLK3 protein
agccccaagcttaccacctgcacccggagagctgtgtcaccatgtgggtcccggttgtcttcctcacccttccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaagtgagtaggggcctggggtctggggagcaggtgtctgtgtcccagaggaataacagctgggcattttccccaggataacctctaaggccagccttgggactgggggagagagggaaagttctggttcaggtcacatggggaggcagggttggggctggaccaccctccccatggctgcctgggtctccatctgtgttcctctatgtctctttgtgtcgctttcattatgtctcttggtaactggcttcggttgtgtctctccgtgtgactattttgttctctctctccctctcttctctgtcttcagt

SEQ ID NO: 23 - protein, 220 aa, Artificial Sequence
  KLK3 protein that is the source of the KLK3 peptide
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 24 - DNA, 1341 bp, Artificial Sequence
  nucleotide encoding the KLK3 protein
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 25 - protein, 218 aa, Artificial Sequence
  KLK3 protein
MWVPVVFLTLSVTWIGAAPLILSRIVGGWECEKHSQPWQVLVASRGRAVCGGVLVHPQWVLTAAHCIRKPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 26 - DNA, 1325 bp, Artificial Sequence
  nucleotide encoding the KLK3 protein
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaagccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattacccaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 27 - protein, 261 aa, Artificial Sequence; identical to SEQ ID NO: 15
  KLK3 protein

SEQ ID NO: 28 - DNA, 1464 bp, Artificial Sequence
  nucleotide encoding the KLK3 protein
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 29 - protein, 261 aa, Artificial Sequence; identical to SEQ ID NO: 15
  the KLK3 protein

SEQ ID NO: 30 - DNA, 1495 bp, Artificial Sequence
  nucleotide encoding the KLK3 protein
gggggagccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaactccctattgtagtaaacttggaaccttggaaatgaccaggccaagactcaggcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtagggagagggactggaccccctggaagctgattcactatggggggaggtgtattgaagtcctccagacaaccctcagatttgatgatttcctagtagaactcacagaaataaagagctgttatactgcgaaaaaaaaaaaaaaaaaaaaaaaaaa

SEQ ID NO: 31 - protein, 218 aa, Artificial Sequence
  KLK3 protein
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVA

SEQ ID NO: 32 - protein, 227 aa, Artificial Sequence
  KLK3 protein
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSVSHPYSQDLEGKGEWGP

SEQ ID NO: 33 - protein, 104 aa, Artificial Sequence
  KLK3 protein
MWVPVVFLTLSVTWIGERGHGWGDAGEGASPDCQAEALSPPTQHPSPDRELGSFLSLPAPLQAHTPSPSILQQSSLPHQVPAPSHLPQNFLPIAQPAPCSQLLY

SEQ ID NO: 34 - protein, 261 aa, Artificial Sequence; identical to SEQ ID NO: 15
  KLK3 protein

SEQ ID NO: 35 - DNA, 1729 bp, Artificial Sequence
  nucleotide encoding the KLK3 protein
aagtttcccttctcccagtccaagaccccaaatcaccacaaaggacccaatccccagactcaagatatggtctgggcgctgtcttgtgtctcctaccctgatccctgggttcaactctgctcccagagcatgaagcctctccaccagcaccagccaccaacctgcaaacctagggaagattgacagaattcccagcctttcccagctccccctgcccatgtcccaggactcccagccttggttctctgcccccgtgtcttttcaaacccacatcctaaatccatctcctatccgagtcccccagttcctcctgtcaaccctgattcccctgatctagcaccccctctgcaggtgctgcacccctcatcctgtctcggattgtgggaggctgggagtgcgagaagcattcccaaccctggcaggtgcttgtagcctctcgtggcagggcagtctgcggcggtgttctggtgcacccccagtgggtcctcacagctacccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctatgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaactccctattgtagtaaacttggaaccttggaaatgaccaggccaagactcaggcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgaaccaaggagggagggtcttcctttggcatgggatggggatgaagtaaggagagggactgaccccctggaagctgattcactatggggggaggtgtattgaagtcctccagacaaccctcagatttgatgatttcctagtagaactcacagaaataaagagctgttatactgtgaa

SEQ ID NO: 36 - protein, 69 aa, Artificial Sequence; identical to SEQ ID NO: 21
  KLK3 protein

SEQ ID NO: 37 - protein, 220 aa, Artificial Sequence; identical to SEQ ID NO: 23
  KLK3 protein that is the source of the KLK3 peptide

SEQ ID NO: 38 - protein, 218 aa, Artificial Sequence; identical to SEQ ID NO: 25
  KLK3 protein

SEQ ID NO: 39 - protein, 24 aa, Artificial Sequence
  The sequence lacking in KLK3 protein
MWVPVVFLTLSVTWIGAAPLILSR

SEQ ID NO: 40 - protein, 98 aa, Artificial Sequence
  E7 protein
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 41 - protein, 105 aa, Artificial Sequence
  E7 protein
MHGPKATLQDIVLHLEPQNEIPVDLLCHEQLSDSEEENDEIDGVNHQHLPARRAEPQRHTMLCMCCKCEARIELVVESSADDLRAFQQLFLNTLSFVCPWCASQQ

SEQ ID NO: 42 - DNA, 22 bp, Artificial Sequence
  primer
ggctcgagcatggagatacacc

SEQ ID NO: 43 - DNA, 28 bp, Artificial Sequence
  primer
ggggactagtttatggtttctgagaaca

SEQ ID NO: 44 - DNA, 31 bp, Artificial Sequence
  primer
gggggctagccctcctttgattagtatattc

SEQ ID NO: 45 - DNA, 28 bp, Artificial Sequence
  primer
ctccctcgagatcataatttacttcatc

SEQ ID NO: 46 - DNA, 55 bp, Artificial Sequence
  primer
gactacaaggacgatgaccgacaagtgataacccgggatctaaataaatccgttt

SEQ ID NO: 47 - DNA, 27 bp, Artificial Sequence
  primer
cccgtcgaccagctcttcttggtgaag

SEQ ID NO: 48 - DNA, 25 bp, Artificial Sequence
  primer
gcggatcccatggagatacacctac

SEQ ID NO: 49 - DNA, 22 bp, Artificial Sequence
  primer
gctctagattatggtttctgag

SEQ ID NO: 50 - DNA, 31 bp, Artificial Sequence
  primer
ggggtctagacctcctttgattagtatattc

SEQ ID NO: 51 - DNA, 45 bp, Artificial Sequence
  primer
atcttcgctatctgtcgccgcggcgcgtgcttcagtttgttgcgc

SEQ ID NO: 52 - DNA, 45 bp, Artificial Sequence
  primer
gcgcaacaaactgaagcagcggccgcggcgacagatagcgaagat

SEQ ID NO: 53 - DNA, 42 bp, Artificial Sequence
  primer
tgtaggtgtatctccatgctcgagagctaggcgatcaatttc

SEQ ID NO: 54 - DNA, 42 bp, Artificial Sequence
  primer
ggaattgatcgcctagctctcgagcatggagatacacctaca

SEQ ID NO: 55 - DNA, 42 bp, Artificial Sequence
  primer
aaacggatttatttagatcccgggttatggtttctgagaaca

SEQ ID NO: 56 - DNA, 42 bp, Artificial Sequence
  primer
tgttctcagaaaccataacccgggatctaaataaatccgttt

SEQ ID NO: 57 - DNA, 28 bp, Artificial Sequence
  primer
gggggtcgaccagctcttcttggtgaag

SEQ ID NO: 58 - protein, 9 aa, Artificial Sequence
  phycoerythrin (PE)-conjugated E7 peptide
RAHYNIVTF

SEQ ID NO: 59 - DNA, 6523 bp, Artificial Sequence
  the plasmid pAdv142
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaacccctaacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcgcggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 60 - DNA, 36 bp, Artificial Sequence
  Adv271-actAF1
cggaattcggatccgcgccaaatcattggttgattg

SEQ ID NO: 61 - DNA, 37 bp, Artificial Sequence
  Adv272-actAR1
gcgagtcgacgtcggggttaatcgtaatgcaattggc

SEQ ID NO: 62 - DNA, 35 bp, Artificial Sequence
  Adv273-actAF2
gcgagtcgacccatacgacgttaattcttgcaatg

SEQ ID NO: 63 - DNA, 39 bp, Artificial Sequence
  Adv274-actAR2
gatactgcagggatccttcccttctcggtaatcagtcac

SEQ ID NO: 64 - DNA, 19 bp, Artificial Sequence
  primer 3
tgggatggccaagaaattc

SEQ ID NO: 65 - DNA, 22 bp, Artificial Sequence
  primer 4
ctaccatgtcttccgttgcttg

SEQ ID NO: 66 - DNA, 28 bp, Artificial Sequence
  Her-2-Chimera (F)
tgatctcgagacccacctggacatgctc

SEQ ID NO: 67 - DNA, 49 bp, Artificial Sequence
  HerEC1-EC2F (Junction)
ctaccaggacacgattttgtggaagaatatccaggagtttgctggctgc

SEQ ID NO: 68 - DNA, 49 bp, Artificial Sequence
  HerEC1-EC2R (Junction)
gcagccagcaaactcctggatattcttccacaaaatcgtgtcctggtag

SEQ ID NO: 69 - DNA, 50 bp, Artificial Sequence
  HerEC2-ICIF (Junction)
ctgccaccagctgtgcgcccgagggcagcagaagatccggaagtacacga

SEQ ID NO: 70 - DNA, 50 bp, Artificial Sequence
  HerEC2-ICIR (Junction)
tcgtgtacttccggatcttctgctgccctcgggcgcacagctggtggcag

SEQ ID NO: 71 - DNA, 39 bp, Artificial Sequence
  Her-2-Chimera (R)
gtggcccgggtctagattagtctaagaggcagccatagg

SEQ ID NO: 72 - DNA, 28 bp, Artificial Sequence
  Her-2-EC1(F)
ccgcctcgaggccgcgagcacccaagtg

SEQ ID NO: 73 - DNA, 31 bp, Artificial Sequence
  Her-2-EC1(R)
cgcgactagtttaatcctctgctgtcacctc

SEQ ID NO: 74 - DNA, 28 bp, Artificial Sequence
  Her-2-EC2(F)
ccgcctcgagtacctttctacggacgtg

SEQ ID NO: 75 - DNA, 30 bp, Artificial Sequence
  Her- 2- EC2(R)
cgcgactagtttactctggccggttggcag

SEQ ID NO: 76 - DNA, 31 bp, Artificial Sequence
  Her-2-Her-2-IC1(F)
ccgcctcgagcagcagaagatccggaagtac

SEQ ID NO: 77 - DNA, 30 bp, Artificial Sequence
  Her-2-IC1(R)
cgcgactagtttaagccccttcggagggtg

SEQ ID NO: 78 - DNA, 7075 bp, Artificial Sequence
  pAdv164 sequence
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaagaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagactaatctagacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcgcggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 79 - protein, 9 aa, Artificial Sequence
  HLA-A2 restricted epitopes
HLYQGCQVV

SEQ ID NO: 80 - protein, 9 aa, Artificial Sequence
  HLA-A2 restricted epitopes
KIFGSLAFL

SEQ ID NO: 81 - protein, 9 aa, Artificial Sequence
  HLA-A2 restricted epitopes
RLLQETELV

SEQ ID NO: 82 - DNA, 2040 bp, Artificial Sequence
  nucleotide encoding LLO fused to a PSA protein
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaacccc

SEQ ID NO: 83 - protein, 680 aa, Artificial Sequence [sequence omitted]

SEQ ID NO: 84 - protein, 97 aa, Artificial Sequence
  E7 protein
HGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 85 - protein, 540 aa, Artificial Sequence
  truncated LLO fused to an E7 protein
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDLEHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 86 - DNA, 1263 bp, Artificial Sequence
  nucleic acid encoding Her-2 chimeric protein
gagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaagaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagactaa

SEQ ID NO: 87 - protein, 419 aa, Artificial Sequence
  Her-2 chimeric protein
THLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKNIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLD

SEQ ID NO: 88 - DNA, 2586 bp, Artificial Sequence
  open reading frame encoding tLLO fused to cHER2
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaagaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagac

SEQ ID NO: 89 - protein, 862 aa, Artificial Sequence
  a recombinant protein comprising tLLO fused to a cHER2
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDLETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKNIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLD